(19) United States
(12) Patent Application Publication
(10) Pub. No.: US 2013/0059840 A1
(43) Pub. Date: Mar. 7, 2013

(54) SULFONAMIDO PYRROLIDINE COMPOUNDS WHICH INHIBIT BETA-SECRETASE ACTIVITY AND METHODS OF USE THEREOF

(71) Applicants: Geoffrey M. Bilcer, Edmond, OK (US); Sudha V. Ankala, Edmond, KS (US)

(72) Inventors: Geoffrey M. Bilcer, Edmond, OK (US); Sudha V. Ankala, Edmond, KS (US)

(73) Assignee: CoMentis, Inc., South San Francisco, CA (US)

(21) Appl. No.: 13/498,559

(22) PCT Filed: Oct. 4, 2010
(86) PCT No.: PCT/US10/51363

§ 371 Date: Nov. 7, 2012

Related U.S. Application Data
(60) Provisional application No. 61/248,814, filed on Oct. 5, 2009.
(60) Provisional application No. 61/249,976, filed on Oct. 8, 2009.
(60) Provisional application No. 61/250,449, filed on Oct. 9, 2009.

(51) Int. Cl. C07D 417/14; A61P 25/28; C12N 9/99; A61K 31/541; A61K 31/427

(52) U.S. Cl. 514/222.2; 544/3; 548/204; 514/365; 435/184

(57) ABSTRACT

Described herein are novel beta-secretase inhibitors and methods for their use, including methods of treating Alzheimer's disease.

### CROSS REFERENCE TO RELATED APPLICATIONS

This application claims priority benefit of U.S. Provisional Application No. 61/248,814, filed on Oct. 5, 2009; U.S. Provisional Application No. 61/249,976 filed Oct. 8, 2009; and U.S. Provisional Application No. 61/250,449, filed on Oct. 9, 2009. The content of each of these applications is hereby incorporated by reference in their entireties as if they were set forth in full below.

### BACKGROUND OF THE INVENTION

Alzheimer's disease is a progressive mental deterioration in a human resulting, inter alia, in loss of memory, confusion and disorientation. Alzheimer's disease accounts for the majority of senile dementias and is a leading cause of death in adults (Anderson, R. N., Natl. Vital Stat. Rep. 49:1-87 (2001), the teachings of which are incorporated herein in their entirety). Histologically, the brain of persons afflicted with Alzheimer's disease is characterized by a distortion of the intracellular neurofibrils and the presence of senile plaques composed of granular or filamentous argentophilic masses with an amyloid protein core, largely due to the accumulation of β-amyloid protein (Aβ) in the brain. Aβ accumulation plays a role in the pathogenesis and progression of the disease (Selkoe, D. J., Nature 399: 23-31 (1999)) and is a proteolytic fragment of amyloid precursor protein (APP). APP is cleaved initially by β-secretase followed by γ-secretase to generate Aβ (Lin, X., et al., Proc. Natl. Acad. Sci. USA 97:1456-1460 (2000); De Stropper, B., et al., Nature 391:387-390 (1998)). Inhibitors of β-secretase are described in U.S. Pat. No. 7,214,715, US 2007/0032470, WO 2006/110/668; WO 2002/02520; WO 2002/02505; WO 2002/02518; WO 2002/02512; WO 2003/040096; WO 2003/072535; WO 2003/050073; WO 2005/030709; WO 2004/050619; WO 2004/080376; WO 2004/043916; WO 2006/110668; Stachel, S. J., J. Med. Chem. 47, 6447-6450 (2004); Stachel, S. J., Bioorg. Med. Chem. Lett. 16, 641-644 (2006); and Varghese, J., Curr. Top. Med. Chem. 6: 569-578 (2006).

There is a need to develop effective compounds and methods for the treatment of Alzheimer's disease. The present invention fulfills these and other needs.

### BRIEF SUMMARY OF THE INVENTION

Described herein are novel β-secretase inhibitor compounds and methods for their use, including methods of treating Alzheimer's disease.

In another aspect, the β-secretase inhibitor compounds can be employed in methods to mediate memapsin 2 activity, e.g., decrease memapsin 2 activity, decrease hydrolysis of a β-secretase site of a memapsin 2 substrate, and/or decrease the accumulation of β-amyloid protein relative to the amount of memapsin 2 activity, hydrolysis of a β-secretase site, and accumulation of β-amyloid protein, respectively, in the absence of the β-secretase inhibitor.

In another aspect, are provided pharmaceutical formulations comprising a β-secretase inhibitor compound or a β-secretase inhibitor compound in combination with a pharmaceutically acceptable carrier.

In another aspect, the β-secretase inhibitor compounds can be employed in the treatment of diseases or conditions associated with β-secretase activity, hydrolysis of a β-secretase site of a β-amyloid precursor protein, and/or β-amyloid protein accumulation. Typically, a mammal is treated for the disease or condition. In an exemplary embodiment, the disease is Alzheimer's disease.

In one aspect, is provided a compound having the formula (I):

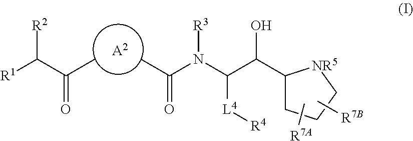

wherein
R¹ is A¹-L¹-; and
R² is hydrogen, —N(R⁸)R⁹, —S(O)₂R¹¹, —C(O)R¹², or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
or wherein R¹ and R² together with the nitrogen to which they are bonded form a 5-membered heterocycloalkyl ring substituted with A¹-L¹- and R⁶;
A¹ is an optionally substituted heteroaryl;
A² is a moiety selected from cycloalkylene, heterocycloalkylene, arylene, and heteroarylene, wherein the moiety is substituted with a cyclic sulfonamido;
R³ and R⁵ are each independently hydrogen, —N(R⁸)R⁹, —S(O)₂R¹¹, —C(O)R¹², or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
L¹ and L⁴ are each independently a bond, —N(R¹⁷)—, —S(O)_{q}—, or an optionally substituted alkylene;
R⁴, R⁶, R^{7A} and R^{7B} are each independently hydrogen, halogen, —OH, —NO₂, —N(R⁸)R⁹, —OR¹⁰, —S(O)ₙR¹¹, —C(O)R¹², or an optionally substituted moiety selected from alkyl, cycloalkyl, cycloalkyl-alkyl, -alkyl-OR₁₀, -alkyl-N(R⁸)R⁹, heterocycloalkyl, heterocycloalkyl-alkyl, aryl, aralkyl, heteroaryl, and heteroaralkyl;
or wherein R^{7A} and R^{7B} together form an optionally substituted cycloalkyl ring;
R⁸ is independently hydrogen, —C(O)R¹³, —S(O)₂R¹⁴, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R⁹ is independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R¹⁰ is independently —C(O)R¹³, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R¹¹ is independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl, wherein if n is 2, then R¹¹ can also be —NR¹⁵R¹⁶, and wherein if n is 1 or 2, then R¹¹ is not hydrogen;
R¹² and R¹³ are each independently hydrogen, —N(R¹⁸)R¹⁹, —OR¹⁹, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R¹⁴ is independently hydrogen, —N(R¹⁸)R¹⁹, or an optionally substituted moiety selected from alkyl, cycloalkyl, cycloalkyl-alkyl, heterocycloalkyl, heterocycloalkyl-alkyl, aryl, aralkyl, heteroaryl, or heteroaralkyl;
R¹⁵, R¹⁶, R¹⁷, R¹⁸, and R¹⁹ are each independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl; and
n and q are each independently 0, 1, or 2;

or a pharmaceutically acceptable salt or solvate thereof.

In one embodiment, the β-secretase inhibitor compound includes any one, any combination, or all of the compounds of Example 2 and/or Table 1; or a pharmaceutically acceptable salt or solvate thereof. In some embodiments, the compound has a memapsin 2 Kᵢ of less than about 250 nM. In some embodiments, the compound has an apparent memapsin 2 Kᵢ of less than about 250 nM as measured by inhibition of memapsin 2 catalytic activity toward the fluorogenic substrate FS-2 (MCA-SEVNLDAEFR-DNP; SEQ ID NO.: 2). In some embodiments, the compound is capable of inhibiting cellular Aβ production with an IC50 of less than about 750 nM, or less than about 250 nM. In some embodiments, the compound has a memapsin 1 Kᵢ and/or cathepsin D Kᵢ of greater than about 300 nM. In some embodiments, the compound has an apparent memapsin 1 Kᵢ and/or apparent cathepsin D Kᵢ of greater than about 300 nM, as measured by the substrate peptide NH₃-ELDLAVEFWHDR-CO₂ (SEQ ID NO.: 1). In some embodiments, the compound has a CYP 3A Kᵢ of greater than about 1 μM, or greater than 5 μM, or greater than 10 μM, as determined by the metabolism of midazolam. In some embodiments, the compound is capable of selectively reducing memapsin 2 catalytic activity relative to memapsin 1 catalytic activity. In some embodiments, the compound is capable of selectively reducing memapsin 2 catalytic activity relative to cathepsin D catalytic activity. In some embodiments, the compound is capable of selectively reducing memapsin 2 catalytic activity relative to CYP3A catalytic activity. In some of these embodiments, the relative reduction is greater than about 5-fold. In other embodiments, the reduction is greater than about 10-fold. In another embodiment, the β-secretase inhibitor compound (a) has a memapsin 2 Kᵢ of less than about 750 nM (or less than about any one of 250 nM, 100 nM, 50 nM, or 10 nM); (b) is capable of inhibiting cellular Aβ production with an IC50 of less than about 1 μM (or less than about any one of 500 nM, 250 nM, 100 nM, 40 nM, or 10 nM); (c) is capable of selectively reducing memapsin 2 catalytic activity relative to memapsin 1 or cathepsin D catalytic activity by greater than about 5-fold (or greater than about 10-fold, or about 100-fold), and/or (d) is capable of selectively reducing memapsin 2 catalytic activity relative to CYP3A catalytic activity by greater than about 5-fold (or greater than about 10-fold, or about 100-fold). In some embodiments, the compound has a hepatic intrinsic clearance in liver microsomes of less than about 700 mL/min/kg, or less than about 400 mL/min/kg, as measured by LC/MS/MS.

In another aspect, is provided any one of the β-secretase inhibitor compounds is present in substantially pure form.

In another aspect, provided are formulations comprising any one of the compounds described herein and a carrier (e.g., a pharmaceutically acceptable carrier). In some embodiments, the formulation is suitable for administration to an individual.

In another aspect, provided are formulations comprising an effective amount of any one of the compounds described herein and a carrier (e.g., a pharmaceutically acceptable carrier).

In another aspect, provided are methods of treating Alzheimer's disease in an individual in need thereof, comprising administering to the individual an effective amount of any one of the compounds described herein (e.g., any compound of formula I, II, III, Example 2 and/or Table 1), or a pharmaceutically acceptable salt or solvate thereof. In some embodiments, the individual has one or more symptoms of Alzheimer's disease. In some embodiments, the individual has been diagnosed with Alzheimer's disease.

In another aspect, provided are methods of treating of a condition mediated by memapsin 2 catalytic activity, comprising administering to the individual an effective amount of a compound of any one of the compounds described herein, or a pharmaceutically acceptable salt or solvate thereof. In some embodiments, the individual has one or more symptoms of the condition mediated by memapsin 2 catalytic activity. In some embodiments, the individual has been diagnosed with condition mediated by memapsin 2 catalytic activity.

In another aspect, provided are methods of reducing memapsin 2 catalytic activity, comprising contacting memapsin 2 with an effective amount of any one of the compounds described herein. In some variations, the memapsin 2 beta-secretase is contacted in a cell. In some embodiments, the cell is contacted in vivo. In some embodiments, the cell is contacted in vitro.

In another aspect, provided are methods of selectively reducing memapsin 2 catalytic activity relative to memapsin 1 catalytic activity, comprising contacting memapsin 2 with an effective amount of a compound of any one of the compounds described herein in the presence of memapsin 1.

In another aspect, provided are methods of selectively reducing memapsin 2 catalytic activity relative to cathepsin D catalytic activity, comprising contacting memapsin 2 with an effective amount of any one of the compounds described herein in the presence of cathepsin D.

In another aspect, provided are methods of selectively reducing memapsin 2 catalytic activity relative to memapsin 1 catalytic activity and cathepsin D catalytic activity, comprising contacting memapsin 2 with an effective amount of any one of the compounds described herein in the presence of memapsin 1 and cathepsin D.

In another aspect, provided are methods of selectively reducing memapsin 2 catalytic activity relative to CYP3A4 catalytic activity, comprising contacting memapsin 2 with an effective amount of any one of the compounds described herein in the presence of CYP3A4.

In another aspect, provided are methods of selectively reducing memapsin 2 catalytic activity relative to memapsin 1 catalytic activity, cathepsin D catalytic activity, and CYP3A4 catalytic activity, comprising contacting memapsin 2 with an effective amount of any one of the compounds described herein in the presence of memapsin 1, cathepsin D, and CYP3A4.

In another aspect, provided are methods of treating Glaucoma in an individual in need thereof, comprising administering to the individual an effective amount of any one of the compounds described herein. In some embodiments, the individual has one or more symptoms of glaucoma. In some embodiments, the individual has been diagnosed with Glaucoma.

In another aspect is provided any one of the compounds described herein or a pharmaceutically acceptable salt or solvate thereof for use as a medicament.

Another aspect is provided the use of any one of the compounds described herein or a pharmaceutically acceptable salt or solvate thereof for the manufacture of a medicament for the treatment or prevention of a condition mediated by memapsin 2 catalytic activity. In another aspect is provided the use of one or more described herein or a pharmaceutically acceptable salt or solvate thereof for the treatment or prevention of a condition mediated by memapsin 2 catalytic activity. In some variations, the condition is Alzheimer's disease.

In another aspect is provided kits for the treatment or prevention in an individual with Alzheimer's disease, comprising any one of the compounds described herein or a pharmaceutically acceptable salt or solvate thereof; and packaging. In some embodiments, the kit comprises a formulation of any one of the compounds described herein or a pharmaceutically acceptable salt or solvate thereof; and packaging.

In another aspect is provided kits for the treatment or prevention in an individual of a condition mediated by memapsin 2 catalytic activity, comprising any one of the compounds described herein or a pharmaceutically acceptable salt or solvate thereof; and packaging. In some embodiments, the kit comprises a formulation of any one of the compounds described herein or a pharmaceutically acceptable salt or solvate thereof; and packaging.

In some embodiments, the compound is not any compound specifically disclosed in U.S. provisional application No. 61/104,434 (filed Oct. 10, 2008); 61/175,624; 61/163,407 (filed Mar. 25, 2009); and/or 61/163,411 (filed Mar. 25, 2009). The disclosures of U.S. provisional application Nos. 61/104,434; 61/175,624; 61/163,407; and 61/163,411 as they relate to the compounds disclosed therein are hereby incorporated by reference in their entirety.

### DETAILED DESCRIPTION OF THE INVENTION

### Abbreviations and Definitions

The abbreviations used herein have their conventional meaning within the chemical and biological arts, unless otherwise specified.

Nomenclature of some compounds described herein may be identified using ChemDraw Ultra Version 10.0, available from CambridgeSoft®.

Where substituent groups are specified by their conventional chemical formula, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e. unbranched) or branched chain, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms. In some embodiments, an alkyl group will have from 1 to 6 carbon atoms. In some embodiments, the alkylene groups are methylene and methylmethylene.

The term “cycloalkyl” by itself or in combination with other terms, represents, unless otherwise stated, cyclic versions of “alkyl.” Additionally, cycloalkyl may contain multiple rings, but excludes aryl and heteroaryl groups. Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like. The term “cycloalkylene” by itself or as part of another substituent means a divalent radical derived from a cycloalkyl, as exemplified, but not limited, by -cyclohexyl-.

The term “heterocycloalkyl,” by itself or in combination with other terms, represents a stable saturated or unsaturated cyclic hydrocarbon radical containing of at least one carbon atom and at least one annular heteroatom selected from the group consisting of O, N, P, Si and S, and wherein the nitrogen and sulfur atoms may optionally be oxidized and the nitrogen heteroatom may optionally be quaternized. The heteroatom(s) O, N, P, S and Si may be placed at any interior position of the heterocycloalkyl group or at the position at which the heterocycloalkyl group is attached to the remainder of the molecule. Additionally, heterocycloalkyl may contain multiple rings, but excludes aryl and heteroaryl groups. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The term “heterocycloalkylene” by itself or as part of another substituent means a divalent radical derived from a heterocycloalkyl, as exemplified, but not limited, by

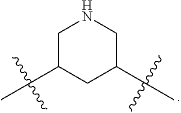 [SMILES: *C1CNCC(*)C1]

The term “cycloalkyl-alkyl” and “heterocycloalkyl-alkyl” designates an alkyl-substituted cycloalkyl group and alkyl-substituted heterocycloalkyl, respectively, where the alkyl portion is attached to the parent structure. Non-limiting examples include cyclopropyl-ethyl, cyclobutyl-propyl, cyclopentyl-hexyl, cyclohexyl-isopropyl, 1-cyclohexenyl-propyl, 3-cyclohexenyl-t-butyl, cycloheptyl-heptyl, norbornyl-methyl, 1-piperidinyl-ethyl, 4-morpholinyl-propyl, 3-morpholinyl-t-butyl, tetrahydrofuran-2-yl-hexyl, tetrahydrofuran-3-yl-isopropyl, and the like. Cycloalkyl-alkyl and heterocycloalkyl-alkyl also include substituents in which a carbon atom of the alkyl group (e.g., a methylene group) has been replaced by, for example, an oxygen atom (e.g., cyclopropoxymethyl, 2-piperidinyloxy-t-butyl, and the like).

The term “aryl” means, unless otherwise stated, a polyunsaturated, aromatic, hydrocarbon substituent. Aryl may contain additional fused rings (e.g., from 1 to 3 rings), including additionally fused aryl, heteroaryl, cycloalkyl, and/or heterocycloalkyl rings. Examples of aryl groups include, but are not limited to, phenyl, 1-naphthyl, 2-naphthyl, and 4-biphenyl. The term “heteroaryl” refers to aryl groups (or rings) that contain from one to four annular heteroatoms selected from N, O, and S, wherein the nitrogen and sulfur atoms are optionally oxidized, and the nitrogen atom(s) are optionally quaternized. A heteroaryl group can be attached to the remainder of the molecule at an annular carbon or annular heteroatom. Heteroaryl may contain additional fused rings (e.g., from 1 to 3 rings), including additionally fused aryl, heteroaryl, cycloalkyl, and/or heterocycloalkyl rings. Non-limiting examples of heteroaryl groups are 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for substituted aryl and heteroaryl ring systems are described below.

The term “arylene” and “heteroarylene” means a divalent radical derived from an aryl and heteroaryl, respectively. Each of the two valencies of arylene and heteroarylene may be located at any portion of the ring (e.g.,

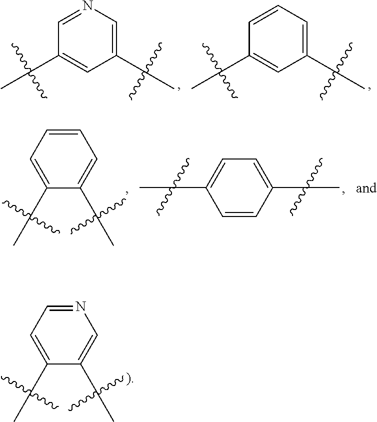 [SMILES: *c1cncc(*)c1 ; *c1cccc(*)c1 ; *c1ccccc1* ; *c1ccc(*)cc1 ; *c1ccncc1*]

Non-limiting examples of arylene include phenylene, biphenylene, naphthylene, and the like. Examples of heteroarylene groups include, but are not limited to, pyridinylene, oxazolylene, thioazolylene, pyrazolylene, pyranylene, and furanylene.

The term “aralkyl” designates an alkyl-substituted aryl group, where the alkyl portion is attached to the parent structure. Examples are benzyl, phenethyl, phenylvinyl, phenylallyl, pyridylmethyl, and the like. “Heteroaralkyl” designates a heteroaryl moiety attached to the parent structure via an alkyl residue. Examples include furanylmethyl, pyridinylmethyl, pyrimidinylethyl, and the like. Aralkyl and heteroaralkyl also include substituents in which a carbon atom of the alkyl group (e.g., a methylene group) has been replaced by, for example, an oxygen atom (e.g., phenoxymethyl, 2-pyridyloxymethyl, 3-(1-naphthyloxy)propyl, and the like).

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “substituted” refers to the replacement of one or more hydrogen atoms of a moiety with a monovalent or divalent radical. “Optionally substituted” indicates that the moiety may be substituted or unsubstituted. A moiety lacking the terms “optionally substituted” and “substituted” is intended an unsubstituted moiety (e.g., “phenyl” is intended an unsubstituted phenyl unless indicated as a substituted phenyl or an optionally substituted phenyl).

The terms, “pharmaceutically effective amount,” “therapeutically effective amount,” “effective amount,” and cognates of these terms, as used herein refer to an amount that results in a desired pharmacological and/or physiological effect for a specified condition (e.g., disease, disorder, etc.) or one or more of its symptoms and/or to completely or partially prevent the occurrence of the condition or symptom thereof and/or may be therapeutic in terms of a partial or complete cure for the condition and/or adverse effect attributable to the condition. In reference to conditions mediated by memapsin 2 beta-secretase, a pharmaceutically or therapeutically effective amount comprises an amount sufficient to, among other things, cause antagonism of memapsin 2 beta-secretase. In reference to glaucoma, a pharmaceutically or therapeutically effective amount comprises an amount sufficient to, among other things, decrease intraocular pressure; and/or halt, reverse, and/or diminish the loss of retinal ganglion cells (RGCs). In certain embodiments, the pharmaceutically effective amount is sufficient to prevent the condition, as in being administered to an individual prophylactically.

The “pharmaceutically effective amount” or “therapeutically effective amount” will vary depending on the composition being administered, the condition being treated/prevented, the severity of the condition being treated or prevented, the age and relative health of the individual, the route and form of administration, the judgment of the attending medical or veterinary practitioner, and other factors appreciated by the skilled artisan in view of the teaching provided herein.

A “pharmaceutically suitable carrier” or “pharmaceutically acceptable carrier,” as used herein refers to pharmaceutical excipients, for example, pharmaceutically, physiologically, acceptable organic, or inorganic carrier substances suitable for enteral or parenteral application which do not deleteriously react with the extract.

When used with respect to methods of treatment/prevention and the use of the compounds and compositions thereof described herein, an individual “in need thereof” may be an individual who has been diagnosed with or previously treated for the condition to be treated. With respect to prevention, the individual in need thereof may also be an individual who is at risk for a condition (e.g., a family history of the condition, life-style factors indicative of risk for the condition, etc.).

In some variations, the individual has been identified as having one or more of the conditions described herein. Identification of the conditions as described herein by a skilled physician is routine in the art and may also be suspected by the individual or others, for example, due to loss of memory in the case of Alzheimer's, exhibiting the symptoms of schizophrenia, etc., and due to a decrease and/or loss of contrast sensitivity or vision in the case of Glaucoma.

In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions), lifestyle or habits.

In some embodiments, the individual is a mammal, including, but not limited to, bovine, horse, feline, rabbit, canine, rodent, or primate. In some embodiments, the mammal is a primate. In some embodiments, the primate is a human. In some embodiments, the individual is human, including adults, children and premature infants. In some embodiments, the individual is a non-mammal. In some variations, the primate is a non-human primate such as chimpanzees and other apes and monkey species. In some embodiments, the mammal is a farm animal such as cattle, horses, sheep, goats, and swine; pets such as rabbits, dogs, and cats; laboratory animals including rodents, such as rats, mice, and guinea pigs; and the like. Examples of non-mammals include, but are not limited to, birds, and the like. The term “individual” does not denote a particular age or sex.

“Pharmaceutically acceptable salts” are those salts which retain the biological activity and which can be administered as drugs or pharmaceuticals to and individual (e.g., a human).

As used herein, “isomer” includes all stereoisomers of the compounds referred to in the formulas herein, including enantiomers, diastereomers, as well as all conformers, rotomers, and tautomers.

A “transition state isostere,” or “isostere,” as used herein, is a compound comprising the hydroxyethylamine linking group —CH(OH)—CH₂—NH—. This isostere is also referred to herein as a “hydroxyethylamine isostere.” The hydroxyethylamine linking group may be found between a pair of natural or non-natural amino acids of a peptide. A hydroxyethylamine group is an isostere of the transition state of hydrolysis of an amide bond.

“Amyloid precursor protein,” or “APP,” as used herein, refers to a β-amyloid precursor comprising a β-secretase site.

“Memapsin-2,” as used herein, refers to proteins identified by National Center for Biotechnology Information (“NCBI”) accession number NP_{—}036236 (sometimes referred to as “β-site APP-cleaving enzyme 1” or “BACE-1” or generically as “β-secretase” or “beta-secretase”), including homologs, isoforms and subdomains thereof that retain proteolytic activity. Sequence identities of active memapsin 2 proteins and protein fragments (and nucleic acid coding sequences thereof) have been previously disclosed and discussed in detail in U.S. Application No. 20040121947, and International Application No. PCT/USO2/34324 (Publication No. WO 03/039454), which are herein incorporated by reference for all purposes in their entirety.

“Memapsin-1,” as used herein, refers to proteins identified by National Center for Biotechnology Information (“NCBI”) accession number NP_{—}036237 (sometimes referred to as “β-site APP-cleaving enzyme 2” or “BACE-2”) and/or those previously disclosed and discussed in detail in see U.S. Patent Application Publication No. 20040121947, and International Application No. PCT/USO2/34324 (Publication No. WO 03/039454), incorporated by reference herein in their entirety for all purposes, including homologs, isoforms and subdomains thereof that retain proteolytic activity.

“Cathepsin D,” as used herein, refers to proteins identified by National Center for Biotechnology Information (“NCBI”), for example accession number NP_{—}599161, and/or proteins identified by Enzyme Commission number EC3.4.23.5, including that from any species, homologs, isoforms, and subdomains thereof that retain proteolytic activity.

A “β-secretase site” is an amino acid sequence that is cleaved by an active memapsin 2 or active fragment thereof. Specific β-secretase sites have also been previously set forth and discussed in detail in U.S. Application No. 20040121947, and International Application No. PCT/USO2/34324 (Publication No. WO 03/039454), which are herein incorporated by reference for all purposes in their entirety, and include the Swedish mutation sequence, and the native β-amyloid precursor protein cleavage sequence. Thus, β-secretase inhibitors may be tested for their ability to decrease the hydrolysis of the β-secretase site of a substrate, such as the β-amyloid precursor protein, compounds of β-amyloid precursor protein, or fragments of β-amyloid precursor protein.

A “beta-secretase inhibitor” (i.e. β-secretase inhibitor) refers to a compound capable of reducing the proteolytic activity of memapsin-2 relative to the activity in the absence of inhibitor.

“Cytochrome P450 3A4” or “CYP3A4,” as used herein refers to proteins identified by Genbank Sequence Accession Number: AF280107; HGNC:2637; Enzyme ID: 1.1.1.161, e.g., which can be found in the product InVitroCYP™M-class™ Human Liver Microsomes from Celsis.

Reference to “about” a value or parameter herein includes (and describes) variations that are directed to that value or parameter per se. For example, description referring to “about X” includes description of “X”.

The terms “a” or “an,” as used in herein means one or more.

### I. β-SECRETASE INHIBITORS

In one aspect, is provided compounds that mediate (e.g., inhibit) the catalytic activity of the β-secretase enzyme (memapsin 2). These compounds may be referred to herein as “β-secretase inhibitor compounds,” or “memapsin 2β-secretase inhibitors.” In this aspect, the compounds have the formula (I):

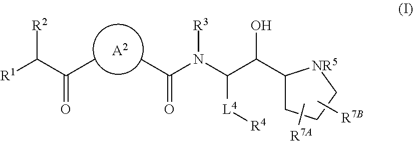

wherein
R¹ is A¹-L¹-; and
R² is hydrogen, —N(R⁸)R⁹, —S(O)₂R¹¹, —C(O)R¹², or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
or wherein R¹ and R² together with the nitrogen to which they are bonded form a 5-membered heterocycloalkyl ring substituted with A¹-L¹- and R⁶;
A¹ is an optionally substituted heteroaryl;
A² is an optionally substituted moiety selected from cycloalkylene, heterocycloalkylene, arylene, and heteroarylene, wherein the moiety is substituted with a cyclic sulfonamido;
R³ and R⁵ are each independently hydrogen, —N(R⁸)R⁹, —S(O)₂R¹¹, —C(O)R¹², or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
L¹ and L⁴ are each independently a bond, —N(R¹⁷)—, —S(O)_{q}—, or an optionally substituted alkylene;
R⁴, R⁶, R^{7A} and R^{7B} are each independently hydrogen, halogen, —OH, —NO₂, —N(R⁸)R⁹, —OR¹⁰, —S(O)ₙR¹², —C(O)R¹², or an optionally substituted moiety selected from alkyl, cycloalkyl, cycloalkyl-alkyl, -alkyl-OR¹⁰, -alkyl-N(R⁸)R⁹, heterocycloalkyl, heterocycloalkyl-alkyl, aryl, aralkyl, heteroaryl, and heteroaralkyl;
or wherein R^{7A} and R^{7B} together form an optionally substituted cycloalkyl ring;
R⁸ is independently hydrogen, —C(O)R¹³, —S(O)₂R¹⁴, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R⁹ is independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R¹⁰ is independently —C(O)R¹³, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R¹¹ is independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl, wherein if n is 2, then R¹¹ can also be —NR¹⁵R¹⁶, and wherein if n is 1 or 2, then R¹¹ is not hydrogen;
R¹² and R¹³ are each independently hydrogen, —N(R¹⁸)R¹⁹, —OR¹⁹, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R¹⁴ is independently hydrogen, —N(R¹⁸)R¹⁹, or an optionally substituted moiety selected from alkyl, cycloalkyl, cycloalkyl-alkyl, heterocycloalkyl, heterocycloalkyl-alkyl, aryl, aralkyl, heteroaryl, or heteroaralkyl;
R¹⁵, R¹⁶, R¹⁷, R¹⁸, and R¹⁹ are each independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl; and
n and q are each independently 0, 1, or 2;

or a pharmaceutically acceptable salt or solvate thereof.

In any embodiment described herein, A² may be substituted with a cyclic sulfonamido.

The substituents on an optionally substituted moiety of formula (I) (e.g., substituents on any optionally substituted alkyl, cycloalkyl, cycloalkyl-alkyl, heterocycloalkyl, heterocycloalkyl-alkyl, aryl, aralkyl, heteroaryl, and/or heteroaralkyl) may be one, two, three, or more groups selected from, but not limited to, hydroxyl, nitro, amino (e.g., —NH₂ or dialkyl amino), imino, cyano, halo (such as F, Cl, Br, I), haloalkyl (such as —CCl₃ or —CF₃), thio, sulfonyl, thioamido, amidino, imidino, oxo, oxamidino, methoxamidino, imidino, guanidino, sulfonamido, carboxyl, formyl, alkyl, alkoxy, alkoxy-alkyl, alkylcarbonyl, alkylcarbonyloxy (—OCOR), aminocarbonyl, arylcarbonyl, aralkylcarbonyl, carbonylamino, heteroarylcarbonyl, heteroaralkyl-carbonyl, alkylthio, aminoalkyl, cyanoalkyl, carbamoyl (—NHCOOR— or —OCONHR—), urea (—NHCONHR—), aryl and the like, where R is any suitable group, e.g., alkyl or alkylene. In some embodiments, the optionally substituted moiety is optionally substituted only with select radicals, as described herein. In some embodiments, the above groups (e.g., alkyl groups) are optionally substituted with, for example, alkyl (e.g., methyl or ethyl), haloalkyl (e.g., —CCl₃, —CH₂CHCl₂ or —CF₃), cycloalkyl (e.g., —C₃H₅, —C₄H₇, —C₅H₉), amino (e.g., —NH₂ or dialkyl amino), alkoxy (e.g., methoxy), heterocycloalkyl (e.g., as morpholine, piperazine, piperidine, azetidine), hydroxyl, and/or heteroaryl (e.g., oxazolyl). In some embodiments, a substituent group is itself optionally substituted. In some embodiments, a substituent group is not itself substituted. The group substituted onto the substitution group can be, for example, carboxyl, halo, nitro, amino, cyano, hydroxyl, alkyl, alkenyl, alkynyl, alkoxy, aminocarbonyl, —SR, thioamido, —SO₃H), —SO₂R or cycloalkyl, where R is any suitable group, e.g., a hydrogen or alkyl.

In some of these embodiments, A¹ is an optionally substituted 5 to 7 membered heteroaryl (e.g., wherein the heteroaryl is attached to L₁ at the 1, 2, 3, 4, or 5 position and/or wherein the heteroaryl is substituted at the 1, 2, 3, 4, and/or 5 position(s)). In other embodiments, A¹ is an optionally substituted 5-membered heteroaryl (e.g., wherein the heteroaryl is attached to L₁ at the 1, 2, 3, 4, or 5 position and/or wherein the heteroaryl is substituted at the 1, 2, 3, 4, and/or 5 position(s)).

In some of these embodiments, A¹ is an optionally substituted moiety selected form the group consisting of pyrazolyl, furanyl, imidazolyl, isoxazolyl, oxadiazolyl, oxazolyl, pyrrolyl, pyridyl, pyrimidyl, pyridazinyl, thiazolyl, triazolyl, thienyl, dihydrothieno-pyrazolyl, thianaphthenyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, isoindolyl, acridinyl, benzoisazolyl, pyrazinyl, pyrrolinyl, indolyl, and benzodiazepinyl.

In some of these embodiments, A¹ is an optionally substituted moiety selected form the group consisting of pyridyl (e.g., an optionally substituted 3-pyridyl, such as a 3-(5-substituted)pyridyl), thiazolyl (e.g., an optionally substituted 2-thiazolyl or a an optionally substituted 4-thiazolyl, such as a 2-(4-substituted)thiazolyl or a 4-(2-substituted)thiazolyl), oxazolyl (e.g., an optionally substituted 2-oxazolyl or an optionally substituted 4-oxazolyl, such as a 2-(4-substituted)oxazolyl or a 4-(2-substituted)oxazolyl), imidazolyl, pyrazolyl, isoxazolyl, pyrimidyl, oxadiazolyl, pyranyl, and furanyl. In some embodiments, A¹ is an optionally substituted moiety selected form the group consisting of thiazolyl (e.g., an optionally substituted 2-thiazolyl or a an optionally substituted 4-thiazolyl, such as a 2-(4-substituted)thiazolyl or a 4-(2-substituted)thiazolyl), oxadiazolyl, and oxazolyl (e.g., an optionally substituted 2-oxazolyl or an optionally substituted 4-oxazolyl, such as a 2-(4-substituted)oxazolyl or a 4-(2-substituted)oxazolyl). In some embodiments, A¹ is an optionally substituted pyridyl (e.g., an optionally substituted 3-pyridyl, such as a 3-(5-substituted)pyridyl). In some embodiments, A¹ is an optionally substituted thiazolyl (e.g., an optionally substituted 2-thiazolyl or a an optionally substituted 4-thiazolyl, such as a 2-(4-substituted)thiazolyl or a 4-(2-substituted)thiazolyl). In some embodiments, A¹ is an optionally substituted oxazolyl (e.g., an optionally substituted 2-oxazolyl or an optionally substituted 4-oxazolyl, such as a 2-(4-substituted)oxazolyl or a 4-(2-substituted)oxazolyl). In some embodiments, A¹ is an optionally substituted oxadiazolyl. In some embodiments, A¹ is an optionally substituted imidazolyl. In some embodiments, A¹ is an optionally substituted pyrazolyl. In some embodiments, A¹ is an optionally substituted isoxazolyl. In some embodiments, A¹ is an optionally substituted pyrimidyl. In some embodiments, A¹ is an optionally substituted furanyl. In some embodiments, A¹ is an optionally substituted 2-thiazolyl. In some embodiments, A¹ is an optionally substituted 2-oxazoyl.

The substituents on an optionally substituted A¹ of formula (I) may be one, two, three, or more groups selected from, but not limited to, hydroxyl, nitro, amino, imino, cyano, halo, haloalkyl, thiol, thioalkyl, sulfonyl, thioamido, amidino, oxo, oxamidino, methoxamidino, imidino, guanidino, sulfonamido, carboxyl, formyl, alkyl, cycloalkyl, alkoxy, alkoxy-alkyl, alkylcarbonyl, alkylcarbonyloxy, aminocarbonyl, aryl, heteroaryl, arylcarbonyl, aralkylcarbonyl, carbonylamino, heteroarylcarbonyl, heteroaralkyl-carbonyl, alkylthio, aminoalkyl, cyanoalkyl, carbamoyl, and urea.

In some embodiments, substituents on an optionally substituted A¹ may be one, two, three, or more groups selected from, but not limited to, hydroxyl, halo (such as F, Cl, Br, I), C₁-C₆ alkyl (e.g., methyl, ethyl, propyl, isopropy) or C₁-C₆ alkoxy (methoxy, ethoxy, propoxy, isopropoxy, wherein each C₁-C₆ alkyl and C₁-C₆ alkoxy is optionally substituted with 1-3 halogens (e.g., —CF₃, —CHF₂, —CH₂F, —OCH₂F, OCHF₂). In some embodiments, A¹ is pyridyl, substituted with one or more —OCH₃. In some embodiments, A¹ (e.g., thiazoyl) is substituted with alkyl, such as methyl (e.g., at the 1, 2, 3, or 4 position of A¹). In some of these embodiments, the alkyl (e.g., methyl) is optionally substituted with 1-3 halogens (e.g., —CF₃, —CHF₂, —CH₂F).

In some of these embodiments, L¹ is a bond or an optionally substituted alkylene. In other embodiments, L¹ is —N(R¹⁷)—, —S(O)_{q}—, or an optionally substituted alkylene. In other embodiments, L¹ is —N(R′⁷)— or —S(O)_{q}—. In other embodiments, L¹ is —N(R′⁷)-. In other embodiments, L¹ is —S(O)_{q}—. In other embodiments, L¹ is a bond. In other embodiments, L¹ is an optionally substituted alkylene. In other embodiments, L¹ is an optionally substituted C₁-C₆ alkylene. In other embodiments, L¹ is a C₁-C₆ alkylene (e.g., methylene or methylmethylene). In other embodiments, L¹ is a branched C₁-C₆ alkylene (e.g., methylmethylene). In other embodiments, L¹ is methylene. In other embodiments, L¹ is methylmethylene.

In some embodiments, substituents on an optionally substituted L¹ may be one, two, three, or more groups selected from, but not limited to, hydroxyl, halo (such as F, Cl, Br, I), C₁-C₆ alkyl (e.g., methyl, ethyl, propyl, isopropy) or C₁-C₆ alkoxy (methoxy, ethoxy, propoxy, isopropoxy, wherein each C₁-C₆ alkyl and C₁-C₆ alkoxy is optionally substituted with 1-3 halogens (e.g., —CF₃, —CHF₂, —CH₂F, —OCH₂F, OCHF₂).

In some of these embodiments, the compound has the formula (II):

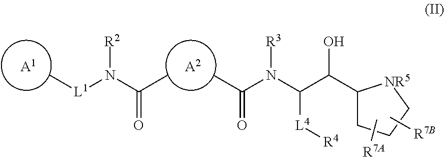

or a pharmaceutically acceptable salt or solvate thereof; wherein A¹, A², L¹, L⁴, R², R³, R⁴, R⁵, R^{7A}, and R^{7B} are as defined above in the discussion of Formula (I) and wherein the A² moiety is substituted with a cyclic sulfonamido.

In some of these embodiments, R² is hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl. In some embodiments, R² is hydrogen, or an optionally substituted moiety selected from alkyl, cycloalkyl, and cycloalkyl-alkyl. In some embodiments, R² is hydrogen or an optionally substituted alkyl. In some embodiments, R² is hydrogen or an optionally substituted C₁-C₆ alkyl. In some embodiments, R² is hydrogen. In some embodiments, R² is an optionally substituted C₁-C₆ alkyl. In some embodiments, R² is an optionally substituted C₁-C₃ alkyl. In some embodiments, R² is an optionally substituted C₃-C₆ cycloalkyl. In some embodiments, R² is methyl.

In some embodiments, substituents on an optionally substituted R² may be one, two, three, or more groups selected from, but not limited to, hydroxyl, halo (such as F, Cl, Br, I), C₁-C₆ alkyl (e.g., methyl, ethyl, propyl, isopropy) or C₁-C₆ alkoxy (methoxy, ethoxy, propoxy, isopropoxy, wherein each C₁-C₆ alkyl and C₁-C₆ alkoxy is optionally substituted with 1-3 halogens (e.g., —CF₃, —CHF₂, —CH₂F, —OCH₂F, OCHF₂). In some embodiments, substituents on an optionally substituted R² are selected from methyl and cyclopropyl.

In some embodiments, the compound has the formula (III):

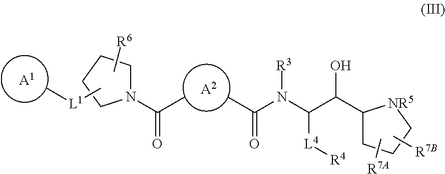

or a pharmaceutically acceptable salt or solvate thereof; wherein A¹, A², L¹, L⁴, R³, R⁴, R⁵, R⁶, R^{7A}, and R^{7B} are as defined above in the discussion of Formula (I). In some embodiments, the A² moiety is substituted with a cyclic sulfonamido.

In some embodiments, the A¹-L¹- moiety is substituted on the pyrrolidine heterocycloalkyl ring according to the formula:

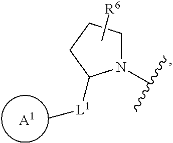

such as

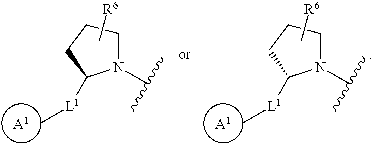

In some of these embodiments, L¹ is a bond, and A¹ is substituted on the pyrrolidine heterocycloalkyl ring according to the formula:

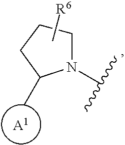

such as

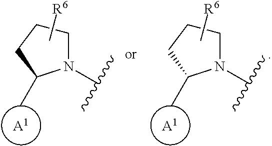

In some embodiments, the A¹-L¹- moiety is substituted on the pyrrolidine heterocycloalkyl ring according to the formula:

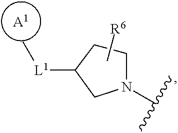

such as

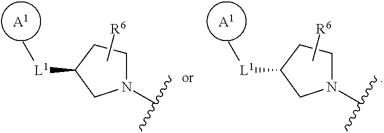

In some embodiments, L¹ is a bond, and A¹ is substituted on the pyrrolidine heterocycloalkyl ring according to the formula:

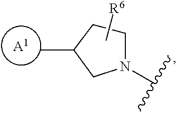

such as

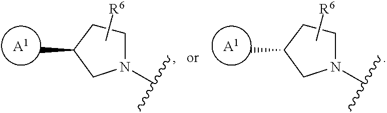

In some embodiments, R⁶ is substituted on the pyrrolidine heterocycloalkyl ring according to the formula:

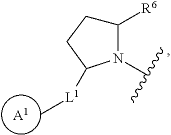

such as

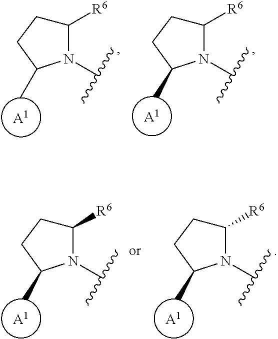

In some embodiments, R⁶ is substituted on the pyrrolidine heterocycloalkyl ring according to the formula:

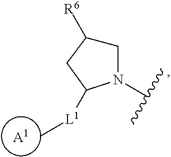

such as

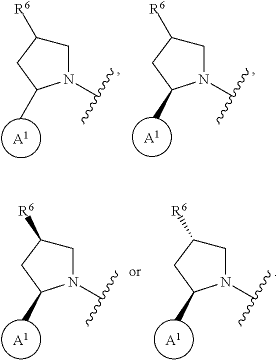

In some embodiments, R⁶ is substituted on the pyrrolidine heterocycloalkyl ring according to the formula:

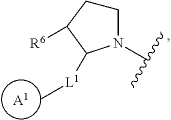

such as

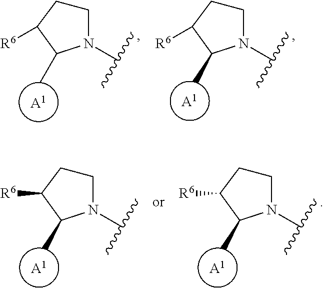

In some embodiments, R⁶ is hydrogen, halogen, —OH, —N(R⁸)R⁹, —OR¹⁰, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl. In some embodiments, R⁶ is hydrogen, or an optionally substituted moiety selected from aryl, aralkyl, heteroaryl, and heteroaralkyl. In some embodiments, R⁶ is hydrogen, halogen (e.g., F or Cl), an optionally substituted alkyl (e.g., haloalkyl), or an optionally substituted —OR¹⁰ (e.g., an optionally substituted —O-alkyl, such as methoxy, ethoxy, propoxy, isopropoxy, or halogenated variants thereof). In some embodiments, R⁶ is hydrogen, F, an optionally substituted (C₁-C₄)alkyl (e.g., methyl, ethyl, propyl, butyl, —CF₃, —CHF₂, —CH₂F), an optionally substituted —O—(C₁-C₄)alkyl (e.g., —O—(C₁-C₄)alkyl, such as methoxy, ethoxy, propoxy, or isopropoxy, substituted with 1, 2, or 3 fluoro groups, such as —OCH₂F, OCHF₂). In some embodiments, R⁶ is hydrogen or halogen. In some embodiments, R⁶ is halogen. In some embodiments, R⁶ is hydrogen.

In some embodiments of formula I, II, and III, A² is an optionally substituted arylene, an optionally substituted heteroarylene. In some embodiments, A² is an optionally substituted moiety selected from the group consisting of phenylene, pyridinylene, oxazolylene, thioazolylene, pyrazolylene, pyranylene, and furanylene.

In some of these embodiments, A² has the formula:

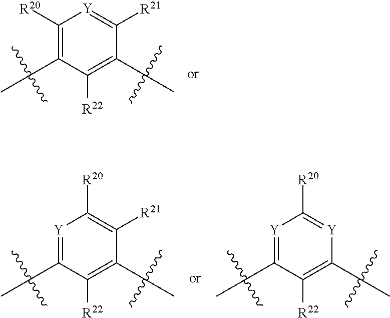

wherein
R²⁰, R²¹, and R²² are independently hydrogen, halogen, —N(R²⁴)R²⁵, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl; and
Y is —N═ or —C(R²³)═, wherein R²³ is hydrogen, halogen, —NO₂, —N(R²⁴)R²⁵, —OR²⁶, —S(O)ₜR²⁷, or —C(O)R²⁸, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
wherein
   t is selected from 0, 1, and 2;
   R²⁴ and R²⁵ are independently hydrogen, —C(O)R²⁹, or —S(O₂)R³⁰, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
   wherein
      R²⁹ is independently hydrogen, —N(R³¹)R³², or —OR³³, an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
      wherein
         R³¹, R³², and R³³ are independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl; and
      R³⁰ is hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
   R²⁶ is hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
   R²⁷ is —N(R³⁴)R³⁵, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
   wherein
   R³⁴ and R³⁵ are each independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl; and
   R²⁸ is —OR³⁶, —N(R³⁷)R³⁸, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
   wherein
      R³⁶, R³⁷, and R³⁸ are each independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
or a pharmaceutically acceptable salt or solvate thereof. In some embodiments, at least one of R²⁰, R²¹ and R²² is a cyclic sulfonamido. In some embodiments, R²³ is a cyclic sulfonamido.

In other of these embodiments, A² has the formula:

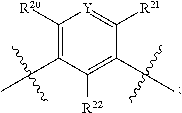

wherein R²⁰, R²¹, and R²² are defined above.

In other of these embodiments, A² has the formula:

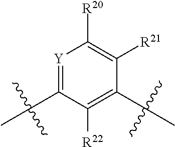

wherein R²⁰, R²¹, and R²² are as defined above.

In other of these embodiments, A² has the formula:

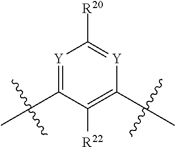

wherein R²⁰, R²¹, and R²² are as defined above.

In some of these embodiments, A² has the formula.

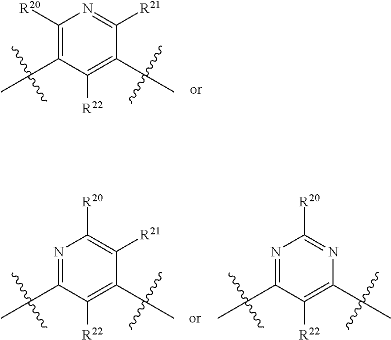

wherein R²⁰, R²¹, and R²² are as defined above.

In other of these embodiments, A² has the formula:

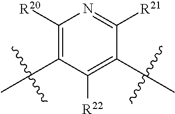

wherein R²⁰, R²¹, and R²² are as defined above.

In other of these embodiments, A² has the formula:

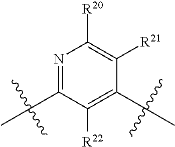

wherein R²⁰, R²¹, and R²² are as defined above.

In other of these embodiments, A² has the formula:

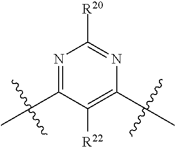

wherein R²⁰ and R²² are as defined above.

In some of these embodiments, Y is —C(R²³)═. In other embodiments, Y is —N═.

In some of these embodiments, A² has the formula:

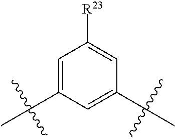

wherein R²³ is as defined above.

In some of these embodiments, R²³ is hydrogen, halogen, —N(R²⁴)R²⁵, —OR²⁶, —S(O)ₜR²⁷, —C(O)R²⁸, or an optionally substituted heterocycloalkyl. In other embodiments, R²³ is hydrogen, —N(R²⁴)R²⁵ (e.g., —N(alkyl)alkylsulfonamido, such as N-methyl-methanesulfonamido), or an optionally substituted heterocycloalkyl (e.g., an optionally substituted cyclic sulfonamido). In other embodiments, R²³ is hydrogen or —N(R²⁴)R²⁵ (e.g., —N(alkyl)alkylsulfonamido, such as N-methyl-methanesulfonamido). In other embodiments, R²³ is hydrogen. In other embodiments, R²³ is —N(R²⁴)R²⁵ (e.g., —N(alkyl)alkylsulfonamido, such as N-methyl-methanesulfonamido) or an optionally substituted heterocycloalkyl (e.g., a cyclic sulfonamido). In other embodiments, R²³ is —N(R²⁴)R²⁵ (e.g., —N(alkyl)alkylsulfonamido, such as N-methyl-methanesulfonamido). In other embodiments R²³ is an optionally substituted heterocycloalkyl (e.g., an optionally substituted cyclic sulfonamido, such as an optionally substituted

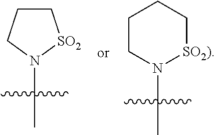

In some embodiments, R²³ is

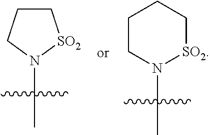 [SMILES: *N1CCCS1(=O)=O ; *N1CCCCS1(=O)=O]

In some embodiments, R²³ is

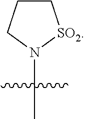 [SMILES: *N1CCCS1(=O)=O]

In some embodiments, R²³ is

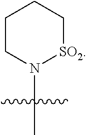 [SMILES: *N1CCCCS1(=O)=O]

In other embodiments, R²³ is —OR²⁶. In other embodiments, R²³ is —S(O)ₜR²⁷. In other embodiments, R²³ is —C(O)R²⁸. In some embodiments, R²³ is hydrogen, an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl. In some embodiments, R²³ is an optionally substituted moiety selected from alkyl, cycloalkyl, and heterocycloalkyl. In some embodiments, R²³ is an optionally substituted alkyl. In some embodiments, R²³ is an optionally substituted C₁-C₆ alkyl. In some embodiments, R²³ is methyl. In some embodiments, R²³ is an optionally substituted cycloalkyl. In some embodiments, R²³ is an optionally substituted heterocycloalkyl. In some embodiments, R²³ is an optionally substituted moiety selected from cycloalkyl, cycloalkyl-alkyl, heterocycloalkyl, heterocycloalkyl-alkyl, aryl, aralkyl, heteroaryl, and heteroaralkyl. In some embodiments, R²³ is an optionally substituted moiety selected from aryl, aralkyl, heteroaryl, and heteroaralkyl. In some embodiments, R²³ is an optionally substituted moiety selected from aryl and heteroaryl. In some embodiments, R²³ is an optionally substituted aryl. In some embodiments, R²³ is an optionally substituted heteroaryl.

In some embodiments, R²³ is an optionally substituted moiety selected from 1,1-dioxoisothiazolidinyl, 1,1-dioxo-1,2-thiazinanyl, pyridyl, phenyl, thiazolyl, oxazolyl, oxadiazolyl, imidazolyl, pyrazolyl, isoxazolyl, pyrimidyl, pyranyl, and furanyl. In some embodiments, R²³ is an optionally substituted moiety selected from thiazolyl, oxadiazolyl, and oxazolyl. In some embodiments, R²³ is an optionally substituted phenyl. In some embodiments, R²³ is an optionally substituted pyridyl. In some embodiments, R²³ is an optionally substituted thiazolyl. In some embodiments, R²³ is an optionally substituted oxazolyl. In some embodiments, R²³ is an optionally substituted oxadiazolyl. In some embodiments, R²³ is an optionally substituted imidazolyl. In some embodiments, R²³ is an optionally substituted pyrazolyl. In some embodiments, R²³ is an optionally substituted isoxazolyl. In some embodiments, R²³ is an optionally substituted pyrimidyl. In some embodiments, R²³ is an optionally substituted pyranyl. In some embodiments, R²³ is an optionally substituted furanyl. In some embodiments, R²³ is an optionally substituted 2-thiazolyl. In some embodiments, R²³ is an optionally substituted 2-oxazoyl. In some embodiments, R²³ is an optionally substituted 1,1-dioxoisothiazolidinyl, e.g. 1,1-dioxoisothiazolidin-2-yl. In some embodiments, R²³ is an optionally substituted 1,1-dioxo-1,2-thiazinanyl, e.g. 1,1-dioxo-1,2-thiazinan-2-yl.

The substituents on an optionally substituted R²³ may be one, two, three, or more groups selected from, but not limited to, hydroxyl, nitro, amino, imino, cyano, halo, haloalkyl, thiol, thioalkyl, sulfonyl, thioamido, amidino, oxo, oxamidino, methoxamidino, imidino, guanidino, sulfonamido, carboxyl, formyl, alkyl, cycloalkyl, alkoxy, alkoxy-alkyl, alkylcarbonyl, alkylcarbonyloxy, aminocarbonyl, aryl, heteroaryl, arylcarbonyl, aralkylcarbonyl, carbonylamino, heteroarylcarbonyl, heteroaralkyl-carbonyl, alkylthio, aminoalkyl, cyanoalkyl, carbamoyl, and urea.

In some embodiments, substituents on an optionally substituted R²³ may be one, two, three, or more groups selected from, but not limited to, hydroxyl, halo (such as F, Cl, Br, I), C₁-C₆ alkyl (e.g., methyl, ethyl, propyl, isopropy) or C₁-C₆ alkoxy (methoxy, ethoxy, propoxy, isopropoxy, wherein each C₁-C₆ alkyl and C₁-C₆ alkoxy is optionally substituted with 1-3 halogens (e.g., —CF₃, —CHF₂, —CH₂F, —OCH₂F, OCHF₂).

In some of these embodiments, R²⁴ and R²⁵ are independently hydrogen, or an optionally substituted moiety selected from alkyl and heteroalkyl. In some embodiments, R²⁴ and R²⁵ are independently hydrogen, or an optionally substituted alkyl. In some embodiments, at least one of R²⁴ and R²⁵ is hydrogen. In some embodiments, wherein R²⁴ and R²⁵ are hydrogen. In some embodiments, at least one of R²⁴ and R²⁵ is an optionally substituted alkyl. In some embodiments, R²⁴ and R²⁵ are independently an optionally substituted alkyl. In some embodiments, at least one of R²⁴ and R²⁵ is methyl. In some embodiments, R²⁴ and R²⁵ are independently hydrogen, an optionally substituted alkyl, —C(O)R²⁹, or —S(O₂)R³⁰. In some embodiments, one of R²⁴ and R²⁵ is —C(O)R²⁹ or —S(O₂)R³⁰. In some embodiments, one of R²⁴ and R²⁵ is —C(O)R²⁹. In some embodiments, one of R²⁴ and R²⁵ is —S(O₂)R³⁰.

In some of these embodiments, R²⁹ is independently hydrogen, an optionally substituted alkyl, —N(R³¹)R³², or —OR³³. In some embodiments, R²⁹ is independently hydrogen, or an optionally substituted alkyl. In some embodiments, R²⁹ is hydrogen. In some embodiments, R²⁹ is an optionally substituted alkyl. In some embodiments, R²⁹ is methyl. In some embodiments, R²⁹ is independently —N(R³¹)R³², or —OR³³. In some embodiments, R²⁹ is —N(R³¹)R³². In some embodiments, R²⁹ is —OR³³.

In some of these embodiments, R³¹, R³², and R³³ are independently hydrogen, or an optionally substituted alkyl.

In some of these embodiments, R³⁰ is hydrogen, an optionally substituted alkyl. In some embodiments, R³⁰ is an optionally substituted alkyl. In some embodiments, R³⁰ is methyl.

In some of these embodiments, R²⁰, R²¹, and R²² are independently hydrogen, or an optionally substituted C₁-C₁₀ alkyl. In some embodiments, R²⁰, R²¹ and R²² are independently hydrogen, or an optionally substituted C₁-C₆ alkyl. In some embodiments, at least one of R²⁰, R²¹, and R²² is hydrogen. In some embodiments, R²⁰, R²¹, R²² are hydrogen.

In some of these embodiments, R²² is hydrogen. In some embodiments, R²² is hydrogen; and R²⁰ and R²¹ are independently hydrogen, or an optionally substituted C₁-C₆ alkyl. In some embodiments, R²² is hydrogen; and R²⁰ and R²¹ are independently hydrogen or methyl. In some embodiments, R²² is hydrogen and one of R²⁰ and R²¹ is methyl. In some embodiments, at least on of R²⁰, R₂₁, R²² is —N(R)R²⁵. In some embodiments, R²⁰ is —N(R²⁴)R²⁵. In some embodiments, R²¹ is —N(R²⁴)R²⁵. In some embodiments, R²² is —N(R²⁴)R²⁵.

In some of these embodiments, R³ is hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl. In some embodiments, R³ is hydrogen, or an optionally substituted moiety selected from alkyl, cycloalkyl, and cycloalkyl-alkyl. In some embodiments, R³ is hydrogen or an optionally substituted alkyl. In some embodiments, R³ is hydrogen or an optionally substituted C₁-C₆ alkyl. In some embodiments, R³ hydrogen. In some embodiments, R³ is an optionally substituted C₁-C₆ alkyl. In some embodiments, R³ is methyl.

In some embodiments, substituents on an optionally substituted R³ may be one, two, three, or more groups selected from, but not limited to, hydroxyl, halo (such as F, Cl, Br, I), C₁-C₆ alkyl (e.g., methyl, ethyl, propyl, isopropy) or C₁-C₆ alkoxy (methoxy, ethoxy, propoxy, isopropoxy, wherein each C₁-C₆ alkyl and C₁-C₆ alkoxy is optionally substituted with 1-3 halogens (e.g., —CF₃, —CHF₂, —CH₂F, —OCH₂F, OCHF₂).

In some of these embodiments, R⁴ is hydrogen. In some embodiments, R⁴ is an optionally substituted moiety selected from alkyl and heteroalkyl. In some embodiments, R⁴ is an optionally substituted moiety selected from cycloalkyl, heterocycloalkyl, aryl, and heteroaryl. In some embodiments, R⁴ is an optionally substituted moiety selected from cycloalkyl and heterocycloalkyl. In some embodiments, R⁴ is an optionally substituted moiety selected from aryl and heteroaryl. In some embodiments, R⁴ is an optionally substituted aryl (e.g., phenyl, 3,5-difluorophenyl or 3-fluorophenyl). In some embodiments, R⁴ is an optionally substituted heteroaryl. In some embodiments, R⁴ is phenyl, optionally substituted with one or more halogens. In some embodiments, R⁴ is phenyl, 3,5-difluorophenyl, or 3-fluorophenyl. In some embodiments, R⁴ is phenyl or 3-fluorophenyl. In some embodiments, R⁴ is phenyl. In some embodiments, R⁴ is 3,5-difluorophenyl. In some embodiments, R⁴ is 3-fluorophenyl.

In some embodiments, substituents on an optionally substituted R⁴ may be one, two, three, or more groups selected from, but not limited to, hydroxyl, halo (such as F, Cl, Br, I), C₁-C₆ alkyl (e.g., methyl, ethyl, propyl, isopropy) or C₁-C₆ alkoxy (methoxy, ethoxy, propoxy, isopropoxy, wherein each C₁-C₆ alkyl and C₁-C₆ alkoxy is optionally substituted with 1-3 halogens (e.g., —CF₃, —CHF₂, —CH₂F, —OCH₂F, —OCHF₂).

In some of these embodiments, L⁴ is a bond, or an optionally substituted alkylene. In some embodiments, L⁴ is a bond. In some embodiments, L⁴ is an optionally substituted alkylene. In some embodiments, L⁴ is an optionally substituted C₁-C₆ alkylene. In some embodiments, L⁴ is a C₁-C₆ alkylene. In some embodiments, L⁴ is methylene (e.g., when L⁴-R⁴ is (e.g., —CH₂-phenyl or —CH₂-difluorophenyl).

In some embodiments, substituents on an optionally substituted L⁴ may be one, two, three, or more groups selected from, but not limited to, hydroxyl, halo (such as F, Cl, Br, I), C₁-C₆ alkyl (e.g., methyl, ethyl, propyl, isopropy) or C₁-C₆ alkoxy (methoxy, ethoxy, propoxy, isopropoxy, wherein each C₁-C₆ alkyl and C₁-C₆ alkoxy is optionally substituted with 1-3 halogens (e.g., —CF₃, —CHF₂, —CH₂F, —OCH₂F, —OCHF₂).

In some of these embodiments, R⁵ is hydrogen, —C(O)R¹², or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl. In some embodiments, R⁵ is hydrogen, —C(O)^{t}Bu, or an optionally substituted moiety selected from alkyl, cycloalkyl, and cycloalkyl-alkyl. In some embodiments, R⁵ is hydrogen, or an optionally substituted alkyl. In some embodiments, R⁵ is hydrogen, or an optionally substituted C₁-C₆ alkyl. In some embodiments, R⁵ is hydrogen. In some embodiments, R⁵ is an optionally substituted C₁-C₆ alkyl. In some embodiments, R⁵ is a C₁-C₆ alkyl. In some embodiments, R⁵ is an optionally substituted C₁-C₃ alkyl. In some embodiments, R⁵ is a C₁-C₃ alkyl. In some embodiments, R⁵ is methyl.

In some embodiments, substituents on an optionally substituted R⁵ may be one, two, three, or more groups selected from, but not limited to, hydroxyl, halo (such as F, Cl, Br, I), C₁-C₆ alkyl (e.g., methyl, ethyl, propyl, isopropy) or C₁-C₆ alkoxy (methoxy, ethoxy, propoxy, isopropoxy, wherein each C₁-C₆ alkyl and C₁-C₆ alkoxy is optionally substituted with 1-3 halogens (e.g., —CF₃, —CHF₂, —CH₂F, —OCH₂F, —OCHF₂).

In some of these embodiments, R^{7A} and R^{7B} are substituted on the pyrrolidine heterocycloalkyl ring according to the formula:

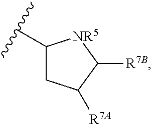

such as

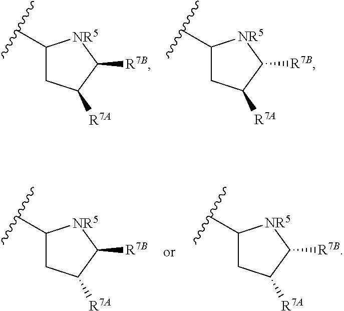

In other of these embodiments, R^{7A} and R^{7B} are substituted on the pyrrolidine heterocycloalkyl ring according to the formula:

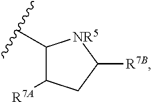

such as,

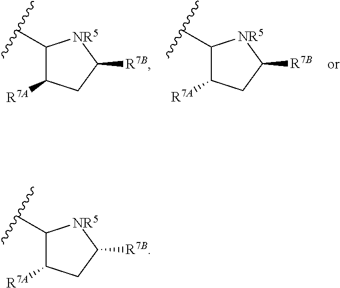

In other of these embodiments, R^{7A} and R^{7B} are substituted on the pyrrolidine heterocycloalkyl ring according to the formula:

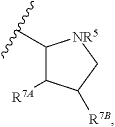

such as

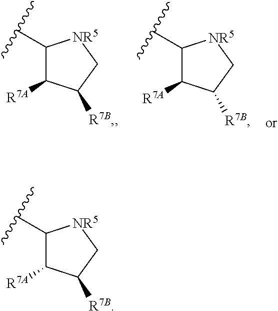

In other of these embodiments, R^{7A} and R^{7B} are substituted on the same carbon atom of the pyrrolidine heterocycloalkyl ring. In some embodiments, R^{7A} and R^{7B} are substituted on the pyrrolidine heterocycloalkyl ring according to the formula:

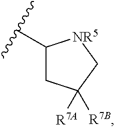

such as

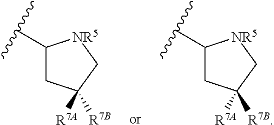

In some embodiments, R^{7A} and R^{7B} are substituted on the pyrrolidine heterocycloalkyl ring according to the formula:

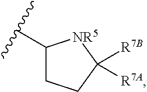

such as

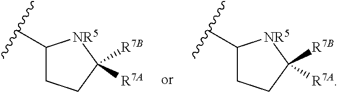

In some embodiments, R^{7A} and R^{7B} are substituted on the pyrrolidine heterocycloalkyl ring according to the formula:

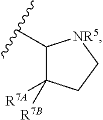

such as

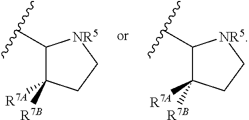

In some embodiments, at least one of R^{7A} and R^{7B} is hydrogen. In some embodiments, R^{7A} is hydrogen. In some embodiments, R^{7B} is hydrogen. In some embodiments, R^{7A} is hydrogen and R^{7B} is other than hydrogen. In some embodiments, R^{7B} is hydrogen and R^{7A} is other than hydrogen.

In some embodiments, R^{7A} and R^{7B} are independently hydrogen, halogen, —OH, —N(R⁸)R⁹, —OR¹⁰, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl.

In some embodiments, R^{7A} and R^{7B} are independently hydrogen, halogen, —OH, —N(R⁸)R⁹, —OR¹⁰, or an optionally substituted moiety selected from alkyl, -alkyl-OR¹⁰, and -alkyl-N(R⁸)R⁹. In some embodiments, R^{7A} and R^{7B} are independently hydrogen, halogen, or an optionally substituted moiety selected from alkyl, -alkyl-OR¹⁰ (e.g., —CH₂O-phenyl), and -alkyl-N(R⁸)R⁹ (e.g., —CH₂N(R⁸)-phenyl). In some embodiments, R^{7A} and R^{7B} are independently hydrogen, alkyl, or an optionally substituted moiety selected from -alkyl-OR¹⁰ (e.g., —CH₂O-phenyl), -alkyl-N(R⁸)R⁹ (e.g., —CH₂N(R⁸)-phenyl). In some embodiments, at least one of R^{7A} and R^{7B} is an optionally substituted moiety selected from -alkyl-OR¹⁰ (e.g., —CH₂O-phenyl, —CH(alkyl)O-phenyl), -alkyl-N(R⁸)R⁹ (e.g., —CH₂N(R⁸)-phenyl). In some embodiments, at least one of R^{7A} and R^{7B} is an optionally substituted -alkyl-OR¹⁰ (e.g., optionally substituted —CH₂O-phenyl or —CH(alkyl)O-phenyl). In some embodiments, at least one of R^{7A} and R^{7B} is an optionally substituted -alkyl-N(R⁸)R⁹ (e.g., optionally substituted —CH₂N(R⁸)-phenyl, such as —CH₂N(alkyl)-phenyl).

In some embodiments, R^{7A} and R^{7B} are independently hydrogen, halogen, —OH, —OR¹⁰, or an optionally substituted moiety selected from alkyl, aryl, aralkyl, heteroaryl, and heteroaralkyl. In some embodiments, R^{7A} and R^{7B} are independently hydrogen, or an optionally substituted moiety selected from alkyl, aryl, aralkyl, heteroaryl, and heteroaralkyl. In some embodiments, R^{7A} and R^{7B} are independently hydrogen, or an optionally substituted alkyl. In some embodiments, R^{7A} and R^{7B} are independently hydrogen, —OH, —NO₂, —N(R⁸)R⁹, —OR¹⁰, —S(O)ₙR¹¹, —C(O)R¹². In some embodiments, R^{7A} and R^{7B} are independently hydrogen, —OH, —NO₂, —N(R⁸)R⁹, —OR¹⁰, —SR¹¹.

In some embodiments, at least one of R^{7A} and R^{7B} is —N(R⁸)R⁹, —OR¹⁰, or —SR¹¹. In some embodiments, R^{7A} and R^{7B} are independently hydrogen, —OH, —OR¹⁰, or an optionally substituted aryl. In some embodiments, at least one of R^{7A} and R^{7B} is —OR¹⁰ (e.g., an optionally substituted moiety selected from —O-alkyl (e.g., —O—C₁-C₆ alkyl, for example, an unsaturated alkyl such as —OCH₂CHCH₂ or a saturated alkyl such as —OCH(CH₃)₂), —O-cycloalkyl, —O-alkyl-cycloalkyl, —O-heterocycloalkyl, —O-alkyl-heterocycloalkyl, —O-aryl, —O-aralkyl, —O-heteroaryl, and —O-heteroaralkyl). In some embodiments, at least one of R^{7A} and R^{7B} is an optionally substituted —O-alkyl-aryl (e.g., an optionally substituted —O—CH₂Ph, or —O—CHCH₂Ph, such as a 3-substituted —O—CH₂Ph, or —O—CHCH₂Ph) or an optionally substituted —O-alkyl-heteroaryl (e.g., —O—CH₂-heteroaryl and/or wherein the heteroaryl is selected from pyridyl, thiazolyl, oxazolyl, oxadiazolyl, imidazolyl, pyrazolyl, isoxazolyl, pyrimidyl, and furanyl). In some embodiments, at least one of R^{7A} and R^{7B} is halogen (e.g., F, Cl, Br, I). In some embodiments, R^{7A} is halogen (e.g., F, Cl, Br, I). In some embodiments, R^{7B} is halogen (e.g., F, Cl, Br, I).

In some embodiments, at least one of R^{7A} and R^{7B} is an optionally heterocycloalkyl. In some embodiments, at least one of R^{7A} and R^{7B} is an optionally substituted moiety selected from aryl (e.g., a 3-substituted phenyl) and heteroaryl. In some embodiments, at least one of R^{7A} and R^{7B} is an optionally substituted aryl (e.g., a 3-substituted phenyl). In some embodiments, at least one of R^{7A} and R^{7B} is an optionally substituted heteroaryl. In some embodiments, at least one of R^{7A} and R^{7B} is an optionally substituted moiety selected from C₁-C₆ alkyl, C₅-C₇ cycloalkyl, 5 to 7 membered heterocycloalkyl, 6-membered aryl, and 5 to 7 membered heteroaryl.

In some embodiments, at least one of R^{7A} and R^{7B} is an optionally substituted moiety selected from phenyl (e.g., a 3-substituted phenyl), pyrazolyl (e.g., an optionally substituted 3-pyrazolyl, an optionally substituted 4-pyrazolyl, or an optionally substituted 5-pyrazolyl such as a 3-(5-substituted)pyrazolyl, a 4-(1-substituted)pyrazolyl, or a 5-(3-substituted)pyrazolyl), furanyl, imidazolyl, isoxazolyl (e.g., an optionally substituted 3-isoxazolyl or an optionally substituted 5-isoxazolyl, such as a 3-(5-substituted)isoxazolyl or a 3-(5-substituted)isoxazolyl), oxadiazolyl, oxazolyl (e.g., an optionally substituted 2-oxazolyl or an optionally substituted 4-oxazolyl, such as a 2-(4-substituted)oxazolyl or a 4-(2-substituted)oxazolyl), pyrrolyl, pyridyl (e.g., a an optionally substituted 3-pyridyl, such as a 3-(5-substituted)pyridyl), pyrimidyl, pyridazinyl, thiazolyl (e.g., an optionally substituted 2-thiazolyl or an optionally substituted 4-thiazolyl, such as a 2-(4-substituted)thiazolyl or a 4-(2-substituted)thiazolyl), triazolyl, thienyl, dihydrothieno-pyrazolyl, thianaphthenyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, isoindolyl, acridinyl, benzoisazolyl, dimethylhydantoin, pyrazinyl, tetrahydrofuranyl, pyrrolinyl, pyrrolidinyl, morpholinyl, indolyl, diazepinyl, azepinyl, thiepinyl, piperidinyl, and oxepinyl.

In some embodiments, at least one of R^{7A} and R^{7B} is an optionally substituted alkyl, or R^{7A} and R^{7B} together form an optionally substituted cycloalkyl ring. In some embodiments, R^{7A} and R^{7B} are selected from hydrogen, optionally substituted alkyl, or R^{7A} and R^{7B} together form an optionally substituted C₃-C₇ cycloalkyl ring (e.g., fused or spiro C₃-C₇ cycloalkyl ring). In some embodiments, R^{7A} and R^{7B} together form an optionally substituted C₄-C₆ cycloalkyl ring (e.g., fused or spiro C₄-C₆ cycloalkyl ring). In some embodiments, R^{7A} and R^{7B} together form an optionally substituted cyclohexyl ring (e.g., fused or spiro cyclohexyl ring).

In some embodiments, at least one of R^{7A} and R^{7B} is an optionally substituted moiety selected from pyridyl (e.g., a an optionally substituted 3-pyridyl, such as a 3-(5-substituted)pyridyl), phenyl (e.g., a 3-substituted phenyl), thiazolyl (e.g., an optionally substituted 2-thiazolyl or an optionally substituted 4-thiazolyl, such as a 2-(4-substituted)thiazolyl or a 4-(2-substituted)thiazolyl), oxazolyl (e.g., an optionally substituted 2-oxazolyl or an optionally substituted 4-oxazolyl, such as a 2-(4-substituted)oxazolyl or a 4-(2-substituted)oxazolyl), oxadiazolyl, imidazolyl, pyrazolyl (e.g., an optionally substituted 3-pyrazolyl, an optionally substituted 4-pyrazolyl, or an optionally substituted 5-pyrazolyl such as a 3-(5-substituted)pyrazolyl, a 4-(1-substituted)pyrazolyl, or a 5-(3-substituted)pyrazolyl), isoxazolyl (e.g., an optionally substituted 3-isoxazolyl or an optionally substituted 5-isoxazolyl, such as a 3-(5-substituted)isoxazolyl or a 3-(5-substituted)isoxazolyl), pyrimidyl, and furanyl. In some embodiments, at least one of R^{7A} and R^{7B} is an optionally substituted moiety selected from pyridyl, and phenyl. In some embodiments, at least one of R^{7A} and R^{7B} is an optionally substituted pyridyl. In some embodiments, at least one of R^{7A} and R^{7B} is an optionally substituted phenyl. In some embodiments, at least one of R^{7A} and R^{7B} is a phenyl substituted with one or more fluoro groups.

The substituents on an optionally substituted R^{7A} and R^{7B} may be one, two, three, or more groups selected from, but not limited to, hydroxyl, nitro, amino, imino, cyano, halo, haloalkyl, thiol, thioalkyl, sulfonyl, thioamido, amidino, oxo, oxamidino, methoxamidino, imidino, guanidino, sulfonamido, carboxyl, formyl, alkyl, cycloalkyl, alkoxy, alkoxy-alkyl, alkylcarbonyl, alkylcarbonyloxy, aminocarbonyl, aryl, heteroaryl, arylcarbonyl, aralkylcarbonyl, carbonylamino, heteroarylcarbonyl, heteroaralkyl-carbonyl, alkylthio, aminoalkyl, cyanoalkyl, carbamoyl, and urea.

In some embodiments, substituents on an optionally substituted R^{7A} and R^{7B} may be one, two, three, or more groups selected from, but not limited to, hydroxyl, halo (such as F, Cl, Br, I), C₁-C₆ alkyl (e.g., methyl, ethyl, propyl, isopropy) or C₁-C₆ alkoxy (methoxy, ethoxy, propoxy, isopropoxy, wherein each C₁-C₆ alkyl and C₁-C₆ alkoxy is optionally substituted with 1-3 halogens (e.g., —CF₃, —CHF₂, —CH₂F, —OCH₂F, OCHF₂).

In some of these embodiments, n is 0 or 2. In other embodiments, n is 1 or 2. In other embodiments, n is 0. In other embodiments, n is 1. In other embodiments, n is 2.

In some of these embodiments, q is 0 or 2. In other embodiments, q is 1 or 2. In other embodiments, q is 0. In other embodiments, q is 1. In other embodiments, q is 2.

In some embodiments, the compound is a compound of formula (II), wherein A¹ is an optionally substituted heteroaryl (e.g., a 5-membered heteroaryl); A² is an optionally substituted arylene (e.g., optionally substituted phenylene), or an optionally substituted heteroarylene (e.g., pyridylene); L¹ and L⁴ are each independently an optionally substituted alkylene (e.g., methylene or methylmethylene); R² and R³ are each independently hydrogen, or an optionally substituted alkyl; R⁴ is an optionally substituted aryl (e.g., phenyl, 3,5-difluorophenyl, or 3-fluorophenyl), R⁵ is a hydrogen, an optionally substituted alkyl, or —C(O)R¹² (e.g., —C(O)O^{t}Bu); and R^{7A} and R^{7B} are each independently hydrogen, halogen, —OH, —N(R⁸)R⁹, —OR¹⁰, or an optionally substituted moiety selected from alkyl, cycloalkyl, cycloalkyl-alkyl, -alkyl-OR¹⁰, -alkyl-N(R⁸)R⁹, heterocycloalkyl, heterocycloalkyl-alkyl, aryl, aralkyl, heteroaryl, and heteroaralkyl; or a pharmaceutically acceptable salt or solvate thereof.

In some embodiments, the compound is a compound of formula (II), wherein A¹ is an optionally substituted thiazolyl (e.g., an optionally substituted 2-thiazolyl or an optionally substituted 4-thiazolyl) or an optionally substituted oxazolyl (e.g., an optionally substituted 2-oxazolyl or an optionally substituted 4-oxazolyl); A² is an optionally substituted phenylene; L¹ and L⁴ are each independently alkylene (e.g., methylene or methylmethylene); R² is hydrogen or an optionally substituted C₁-C₃ alkyl; R³, R⁵, and R^{7B} are each hydrogen; R⁴ is an optionally substituted aryl (e.g., phenyl, 3,5-difluorophenyl, or 3-fluorophenyl); and R^{7A} is hydrogen, halogen, —OH, —N(R⁸)R⁹, —OR¹⁰, or an optionally substituted moiety selected from alkyl, -alkyl-OR¹⁰, -alkyl-N(R⁸)R⁹, heterocycloalkyl, heterocycloalkyl-alkyl, aryl, aralkyl, heteroaryl, and heteroaralkyl; or a pharmaceutically acceptable salt or solvate thereof.

In some embodiments, the compound is a compound of formula (II), wherein A¹ is an optionally substituted 2-thiazolyl (e.g., 2-(4-substituted)thiazolyl); A² is

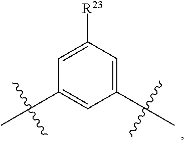

wherein R²³ is hydrogen, N-methyl-methanesulfonamido, or an optionally substituted moiety selected from alkyl (e.g., an alkyl optionally substituted with one, two, three or more halogens), heteroaryl (e.g., a heteroaryl optionally substituted with a C₁-C₄ alkyl, wherein the C₁-C₄ alkyl may be optionally substituted with two, three or more halogens), and heterocycloalkyl (e.g., an optionally substituted cyclic sulfonamido, such as an optionally substituted

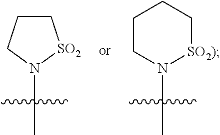

L¹ and L⁴ are each methylene; R² is methyl; R³, R⁵, R^{7A}, and R^{7B} are each hydrogen; R⁴ is phenyl, 3,5-difluorophenyl, or 3-fluorophenyl; or a pharmaceutically acceptable salt or solvate thereof. In some of these embodiments, R²³ is hydrogen, N-methyl-methanesulfonamido,

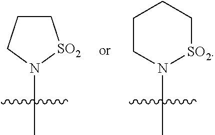 [SMILES: *N1CCCS1(=O)=O ; "or" ; *N1CCCCS1(=O)=O]

In some embodiments, the compound is a compound of formula (II), wherein A¹ is an optionally substituted 2-oxazolyl (e.g., such as a 2-(4-substituted)oxazolyl); A² is

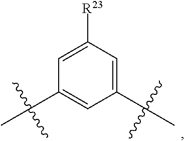

wherein R²³ is hydrogen, N-methyl-methanesulfonamido, or an optionally substituted moiety selected from alkyl (e.g., an alkyl optionally substituted with one, two, three or more halogens), heteroaryl (e.g., a heteroaryl optionally substituted with a C₁-C₄ alkyl, wherein the C₁-C₄ alkyl may be optionally substituted with two, three or more halogens), and heterocycloalkyl (e.g., an optionally substituted cyclic sulfonamido, such as an optionally substituted

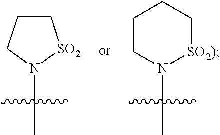

L¹ and L⁴ are each methylene; R² is methyl; R³, R⁵, R^{7A}, and R^{7B} are each hydrogen; R⁴ is phenyl, 3,5-difluorophenyl, or 3-fluorophenyl; or a pharmaceutically acceptable salt or solvate thereof. In some of these embodiments, R²³ is hydrogen, N-methyl-methanesulfonamido,

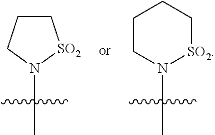 [SMILES: *N1CCCS1(=O)=O ; "or" ; *N1CCCCS1(=O)=O]

In some embodiments, the compound has the formula:

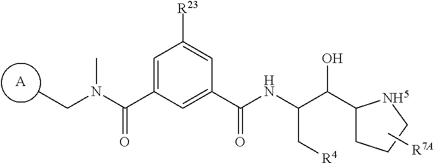

wherein, A¹ is an optionally substituted heteroaryl; R²³ is a cyclic sulfonamido; R⁵ is hydrogen or t-butyoxycarbonyl; R⁴ is an optionally substituted aryl; and R^{7A} is hydrogen, halogen, —OH, —N(R⁸)R⁹, —OR¹⁰, or an optionally substituted moiety selected from alkyl, cycloalkyl, cycloalkyl-alkyl, -alkyl-OR¹⁰, -alkyl-N(R⁸)R⁹, heterocycloalkyl, heterocycloalkyl-alkyl, aryl, aralkyl, heteroaryl, and heteroaralkyl, —OH, or —OBn; or a pharmaceutically acceptable salt or solvate thereof. In some of these embodiments, A¹ is an optionally substituted thiazolyl or an optionally substituted oxazolyl; R⁵ is hydrogen; R⁴ is an optionally substituted phenyl; and R^{7A} is hydrogen, halogen, —OH, —N(R⁸)R⁹, —OR¹⁰, -alkyl-OR¹⁰ or an optionally substituted alkyl; or a pharmaceutically acceptable salt or solvate thereof. In some of these embodiments, A¹ is an 2-(4-methyl)thiazolyl or 2-(4-methyl)oxazolyl; R²³ is

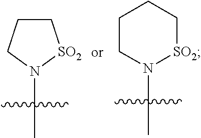 [SMILES: *N1CCCS1(=O)=O ; *N1CCCCS1(=O)=O]

R⁵ is hydrogen; R⁴ is an phenyl, 3,5-difluorophenyl, or 3-fluorophenyl; and R^{7A} is hydrogen or —OR¹⁰; or a pharmaceutically acceptable salt or solvate thereof.

In some embodiments, the compound has the formula:

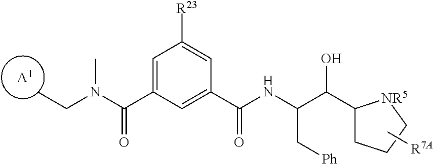

wherein, A¹ is thiazolyl; R²³ is hydrogen, —N(CH₃)SO₂Me, 1,1-dioxoisothiazolidinyl, 1,1-dioxo-1,2-thiazinanyl, oxazolyl or pyrrolyl; R⁵ is hydrogen or t-butyoxycarbonyl; and R^{7A} is hydrogen, —OH, or —OBn; or a pharmaceutically acceptable salt or solvate thereof. In some embodiments, R²³ is 1,1-dioxoisothiazolidin-2-yl or 1,1-dioxo-1,2-thiazinan-2-yl.

In some embodiments, the compound is a compound of formula (III), wherein A¹ is an optionally substituted heteroaryl (e.g., a 5-membered heteroaryl); A² is an optionally substituted arylene (e.g., phenylene), or an optionally substituted heteroarylene (e.g., pyridylene); L¹ is a bond; L⁴ is an optionally substituted alkylene (e.g., optionally substituted methylene; R³ is hydrogen, or an optionally substituted alkyl; R⁴ is an optionally substituted aryl (e.g., phenyl, 3,5-difluorophenyl, or 3-fluorophenyl); R⁵ is a hydrogen, an optionally substituted alkyl, or —C(O)R¹² (e.g., —C(O)O^{t}Bu); R⁶ is a hydrogen, halogen, —OR¹⁰, an optionally substituted alkyl; and R^{7A} and R^{7B} are each independently hydrogen, halogen, —OH, —N(R⁸)R⁹, —OR¹⁰, or an optionally substituted moiety selected from alkyl, cycloalkyl, cycloalkyl-alkyl, -alkyl-OR¹⁰, -alkyl-N(R⁸)R⁹, heterocycloalkyl, heterocycloalkyl-alkyl, aryl, aralkyl, heteroaryl, and heteroaralkyl; or a pharmaceutically acceptable salt or solvate thereof.

In some embodiments, the compound is a compound of formula (III), wherein A¹ is an optionally substituted thiazolyl (e.g., an optionally substituted 2-thiazolyl or an optionally substituted 4-thiazolyl) or an optionally substituted oxazolyl (e.g., an optionally substituted 2-oxazolyl or an optionally substituted 4-oxazolyl); A² is an optionally substituted phenylene; L¹ is a bond; L⁴ is an optionally substituted alkylene (e.g., methylene); R³, R⁵, and R^{7B} are each hydrogen; R⁴ is an optionally substituted aryl (e.g., phenyl, 3,5-difluorophenyl, or 3-fluorophenyl); R⁶ is a hydrogen, halogen (e.g., F), an optionally substituted —O(C₁-C₅)alkyl (e.g., methyl, ethyl, propyl, optionally substituted with 1, 2, or 3 fluoro groups), an optionally substituted (C₁-C₅)alkyl (e.g., methoxy, ethyloxy, propoxy, optionally substituted with 1, 2, or 3 fluoro groups); and R^{7A} is hydrogen, halogen, —OH, —N(R⁸)R⁹, —OR¹⁰, or an optionally substituted moiety selected from alkyl, -alkyl-OR¹⁰, -alkyl-N(R⁸)R⁹, heterocycloalkyl, heterocycloalkyl-alkyl, aryl, aralkyl, heteroaryl, and heteroaralkyl; or a pharmaceutically acceptable salt or solvate thereof.

In some embodiments, the compound is a compound of formula (III), wherein A¹ is an optionally substituted 2-thiazolyl (e.g., 2-(4-substituted)thiazolyl); A² is

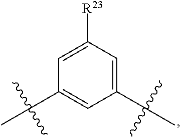

wherein R²³ is hydrogen, or an optionally substituted moiety selected from alkyl (e.g., an alkyl optionally substituted with one, two, three or more halogens), heteroaryl (e.g., a heteroaryl optionally substituted with a C₁-C₄ alkyl, wherein the C₁-C₄ alkyl may be optionally substituted with two, three or more halogens), and heterocycloalkyl (e.g., an optionally substituted cyclic sulfonamido, such as an optionally substituted

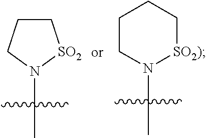

L¹ is a bond; L⁴ is methylene; R³, R⁵, R⁶, R^{7A}, and R^{7B} are each hydrogen; R⁴ is phenyl, 3,5-difluorophenyl, or 3-fluorophenyl; or a pharmaceutically acceptable salt or solvate thereof. In some of these embodiments, R²³ is selected from oxazolyl (e.g., 2-oxazolyl), pyrazyl (e.g., 2-pyrazyl), hydrogen, an optionally substituted methyl (e.g., di-fluoro methyl), N-methyl-methanesulfonamido,

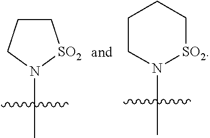 [SMILES: *N1CCCS1(=O)=O ; *N1CCCCS1(=O)=O]

In some embodiments, the compound is a compound of formula (III), wherein A¹ is an optionally substituted 2-oxazolyl (e.g., such as a 2-(4-substituted)oxazolyl); A² is

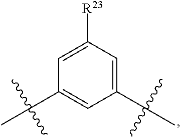

wherein R²³ is hydrogen, or an optionally substituted moiety selected from alkyl (e.g., an alkyl optionally substituted with one, two, three or more halogens), heteroaryl (e.g., a heteroaryl optionally substituted with a C₁-C₄ alkyl, wherein the C₁-C₄ alkyl may be optionally substituted with two, three or more halogens), and heterocycloalkyl (e.g., an optionally substituted cyclic sulfonamido, such as an optionally substituted

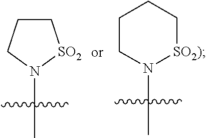

L¹ is a bond; L⁴ is methylene; R³, R⁵, R⁶, R^{7A}, and R^{7B} are each hydrogen; R⁴ is phenyl, 3,5-difluorophenyl, or 3-fluorophenyl; or a pharmaceutically acceptable salt or solvate thereof. In some of these embodiments, R²³ is selected from oxazolyl (e.g., 2-oxazolyl), pyrazyl (e.g., 2-pyrazyl), hydrogen, an optionally substituted methyl (e.g., di-fluoro methyl), N-methyl-methanesulfonamido,

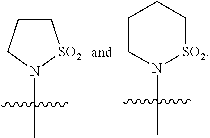 [SMILES: *N1CCCS1(=O)=O ; *N1CCCCS1(=O)=O]

In some embodiments, the compound has the formula:

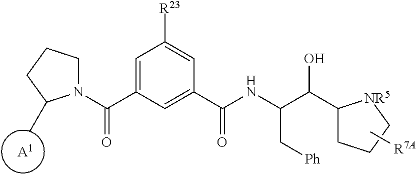

wherein, A¹ is an optionally substituted heteroaryl; R²³ is a cyclic sulfonamido; R⁵ is hydrogen or t-butyoxycarbonyl; R⁴ is an optionally substituted aryl; and R^{7A} is hydrogen, halogen, —OH, —N(R⁸)R⁹, —OR¹⁰, or an optionally substituted moiety selected from alkyl, cycloalkyl, cycloalkyl-alkyl, -alkyl-OR¹⁰, -alkyl-N(R⁸)R⁹, heterocycloalkyl, heterocycloalkyl-alkyl, aryl, aralkyl, heteroaryl, and heteroaralkyl, —OH, or —OBn; or a pharmaceutically acceptable salt or solvate thereof. In some of these embodiments, A¹ is an optionally substituted thiazolyl or an optionally substituted oxazolyl; R⁵ is hydrogen; R⁴ is an optionally substituted phenyl; and R^{7A} is hydrogen, halogen, —OH, —N(R⁸)R⁹, —OR¹⁰, -alkyl-OR¹⁰ or an optionally substituted alkyl; or a pharmaceutically acceptable salt or solvate thereof. In some of these embodiments, A¹ is an 2-(4-methyl)thiazolyl or 2-(4-methyl)oxazolyl; R²³ is

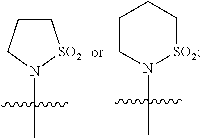 [SMILES: *N1CCCS1(=O)=O ; *N1CCCCS1(=O)=O]

R⁵ is hydrogen; R⁴ is an phenyl, 3,5-difluorophenyl, or 3-fluorophenyl; and R^{7A} is hydrogen or —OR¹⁰; or a pharmaceutically acceptable salt or solvate thereof.

In some embodiments, the compound has the formula:

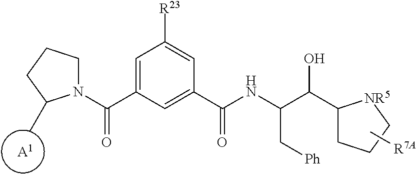

wherein, A¹ is thiazolyl or oxazolyl; R²³ is hydrogen, methyl, difluoromethyl, —N(CH₃)SO₂Me, 1,1-dioxoisothiazolidinyl, 1,1-dioxo-1,2-thiazinanyl, oxazolyl, pyrrolyl, pyridyl, or pyrazinyl; R⁵ is hydrogen or t-butyoxycarbonyl; and R^{7A} is hydrogen, —OH, or —OBn; or a pharmaceutically acceptable salt or solvate thereof. In some embodiments, R²³ is 1,1-dioxoisothiazolidin-2-yl or 1,1-dioxo-1,2-thiazinan-2-yl.

In some embodiments are provided any one, any combination, or all of the compounds of Table 1.

In some embodiments, the compounds described herein (e.g., any compound of formula I, II, III, Example 2 and/or Table 1) is in substantially pure form. Unless otherwise stated, “substantially pure” intends a preparation of the compound that contains no more than 15% impurity, wherein the impurity intends compounds other than the indicated inhibitor compound, but does not include other forms of the inhibitor compound (e.g., different salt form or a different stereoisomer, conformer, rotamer, or tautomer of the compound depicted). In one variation, a preparation of substantially pure compound is provided wherein the preparation contains no more than 25% impurity, or no more than 20% impurity, or no more than 10% impurity, or no more than 5% impurity, or no more than 3% impurity, or no more than 1% impurity, or no more than 0.5% impurity. In some embodiments, the compound is present with no more than 15% or no more than 10% or no more than 5% or no more than 3% or no more than 1% of the total amount of compound in a different stereochemical form (e.g., when the an S,S compound no more than 15% or no more than 10% or no more than 5% or no more than 3% or no more than 1% of the total R,R; S,R; and R,S form is present).

The compounds described herein (e.g., any compound of formula I, II, III, Example 2 and/or Table 1) and methods of using the same, unless otherwise stated, include all solvate and/or hydrate forms. In some embodiments, the compounds described herein can exist in unsolvated forms as well as solvated forms (i.e., solvates). The compounds may also include hydrated forms (i.e., hydrates).

The compounds described herein (e.g., any compound of formula I, II, III, Example 2 and/or Table 1), as well as methods of using such salts of the compounds, unless otherwise stated, include all salt forms of the compounds. The compounds also include all non-salt forms of any salt of a compound described herein, as well as other salts of any salt of a compound described herein. In some embodiments, the salts of the compounds are pharmaceutically acceptable salts. The desired salt of a basic functional group of a compound may be prepared by methods known to those of skill in the art by treating the compound with an acid. The desired salt of an acidic functional group of a compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, bismuth salts, and calcium salts; ammonium salts; and aluminum salts. Examples of organic salts of acid compounds include, but are not limited to, procaine, dibenzylamine, N-ethylpiperidine, N,N′-dibenzylethylenediamine, trimethylamine, and triethylamine salts. Examples of inorganic salts of base compounds include, but are not limited to, hydrochloride and hydrobromide salts. Examples of organic salts of base compounds include, but are not limited to, tartrate, citrate, maleate, fumarate, and succinate. In some embodiments, the compounds in the salt form of hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures), succinates, benzoates and salts with amino acids such as glutamic acid. In some embodiments, the compounds described herein exist as a citrate salt (e.g., mono citrate, hydrogen citrate, or dihydrogen citrate) and/or a mesylate salt (e.g., dimesylate). These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, also provided are compounds which are in a prodrug form. Prodrugs of the compounds described herein are those compounds that readily undergo chemical changes under physiological conditions to provide the desired compound (e.g., any compound of formula I, II, III, Example 2 and/or Table 1). Additionally, prodrugs can be converted to the compounds described herein by chemical or biochemical methods in an ex vivo environment. For example, prodrugs can be slowly converted to the compounds described herein when placed in a transdermal patch reservoir with a suitable enzyme or chemical reagent.

Metabolites of the compounds are also embraced. Metabolites may include primary metabolites and/or secondary metabolites. However, metabolites of substances which occur naturally in subjects are excluded from the claimed compounds.

Unless stereochemistry is explicitly indicated in a chemical structure or chemical name, the chemical structure or chemical name is intended to embrace all possible stereoisomers, conformers, rotomers, and tautomers of the compound depicted. For example, a compound containing a chiral carbon atom is intended to embrace both the (R) enantiomer and the (S) enantiomer. A compound containing multiple chiral carbon atoms (for example, both carbons within the hydroxyethylamine isostere) is intended to embrace all enantiomers and diastereomers (including (R,R), (S,S), (R,S), and (R,S) isomers). When a compound is explicitly indicated in a particular stereochemical arrangement (e.g., 2S,3R for the hydroxyethylamine isostere), the compound may, in other embodiments, be described in another specific stereochemical arrangement (e.g., 2R,3S for the hydroxyethylamine isostere) and/or a mixture of stereochemical arrangements.

A composition may contain the compound as mixtures of such stereoisomers, where the mixture may be enantiomers (e.g., S,S and R,R) or diastereomers (e.g., S,S and R,S or S,R) in equal or unequal amounts. A composition may contain the compound as a mixture of 2 or 3 or 4 such stereoisomers in any ratio of stereoisomers.

In some embodiments, are provided compounds of formula I having the formula (Ib):

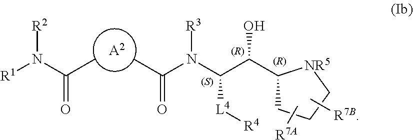

In Formula (Ib), A¹, A², L¹, L⁴, R², R³, R⁴, R⁵, R⁶, R^{7A}, and R^{7B} are as defined above in the discussion of Formula (I). In some embodiments, the A² moiety is substituted with a cyclic sulfonamido.

In some embodiments, are provided compounds of formula I having the formula (Ic):

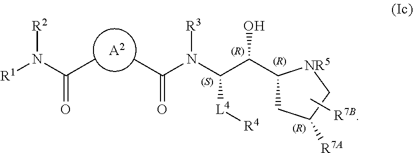

In Formula (Ic), A¹, A², L¹, L⁴, R², R³, R⁴, R⁵, R⁶, R^{7A}, and R^{7B} are as defined above in the discussion of Formula (I). In some embodiments, the A² moiety is substituted with a cyclic sulfonamido.

In some embodiments, are provided compounds of formula I having the formula (IIb):

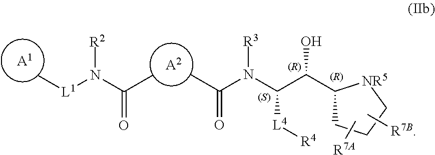

In Formula (IIb), A¹, A², L¹, L⁴, R², R³, R⁴, R⁵, R^{7A}, and R^{7B} are as defined above in the discussion of Formula (I) and (II). In some embodiments, the A² moiety is substituted with a cyclic sulfonamido.

In some embodiments, are provided compounds of formula I having the formula (IIc):

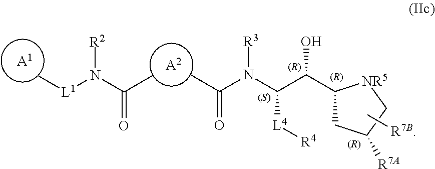

In Formula (IIc), A¹, A², L¹, L⁴, R², R³, R⁴, R⁵, R^{7A}, and R^{7B} are as defined above in the discussion of Formula (I) and (II). In some embodiments, the A² moiety is substituted with a cyclic sulfonamido.

In some embodiments, t are provided compounds of formula I having the formula (IIIb):

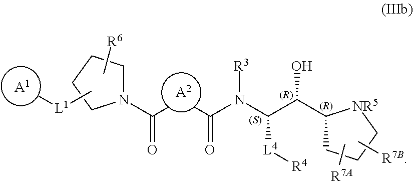

In Formula (IIb), A¹, A², L¹, L⁴, R³, R⁴, R⁵, R⁶, R^{7A}, and R^{7B} are as defined above in the discussion of Formula (I) and (III). In some embodiments, the A² moiety is substituted with a cyclic sulfonamido.

In some embodiments, are provided compounds of formula I having the formula (IIIc):

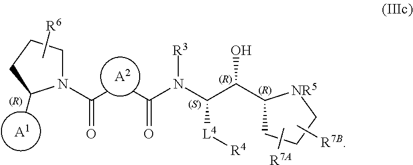

In Formula (IIIc), A¹, A², L⁴, R³, R⁴, R⁵, R⁶, R⁷, and R^{7B} are as defined above in the discussion of Formula (I) and (III). In some embodiments, the A² moiety is substituted with a cyclic sulfonamido.

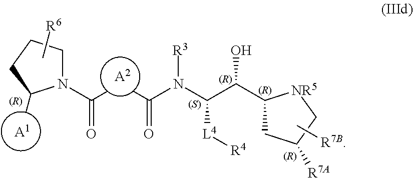

In Formula (IIId), A¹, A², L⁴, R³, R⁴, R⁵, R⁶, R^{7A}, and R^{7B} are as defined above in the discussion of Formula (I) and (III). In some embodiments, the A² moiety is substituted with a cyclic sulfonamido.

The compounds herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds herein, whether radioactive or not, are contemplated.

Included in all uses of the compounds disclosed herein (e.g., any compound of formula I, II, III, Example 2, and/or Table 1), is any or all of the stereochemical, enantiomeric, diastereomeric, conformational, rotomeric, tautomeric, isotopic, solvate, hydrate, salt, and pharmaceutically acceptable salts of the compounds as described.

### A. Carrier Moieties

In U.S. Application No. 20040121947, and International Application No. PCT/USO2/34324 (Publication No. WO 03/039454), which are herein incorporated by reference for all purposes, isostere β-secretase inhibitors with and without a carrier moiety were shown to effectively reduce Aβ production in tg2576 mice expressing the Swedish mutation of the human amyloid precursor protein (Hsiao, K., et al., Science 274, 99-102 (1996)). Thus, one of skill in the art will recognize that the compounds herein may be administered with or without a carrier moiety.

A “carrier moiety,” as used herein, refers to a chemical moiety covalently or non-covalently attached to a β-secretase inhibitor compound herein that enhances the ability of the compound to traverse the blood-brain barrier (BBB). The β-secretase inhibitors herein may be attached or conjugated to the carrier moiety by covalent interactions (e.g., peptide bonds) or by non-covalent interactions (e.g., ionic bonds, hydrogen bonds, van der Waals attractions). A covalently attached carrier moiety may be attached to any appropriate site on the compounds herein (e.g., a hydroxyl group, amino group, thiol group, carboxylate group). One or more carrier moieties may be used on a compound herein. Multiple carrier moieties on a compound may be identical (e.g. multiple peptidyl carrier moieties) or different (e.g., a lipophilic carrier moiety and a peptidyl carrier moiety). Attachment of multiple carrier moieties on a compound herein may be identical (e.g., both covalently attached) or different (e.g., one covalently attached and one non-covalently attached).

The blood-brain barrier is a permeability barrier that exists between the extracellular fluid in the brain and the blood in the capillary lumen. The barrier stems from structural differences between the capillaries in the brain and capillaries found in other tissues. Most significant among the structural differences of brain capillaries are the tight junctions between endothelial cells. These specialized tight junctions create a very high trans-endothelial electrical resistance of 1500-2000 ohms/cm² compared to 3-33 ohms/cm² in capillary endothelial cells lying outside the brain, reducing the aqueous based para-cellular diffusion observed in other organs (Brightman, M. in Bradbury MWB (ed.) Physiology and Pharmacology of the blood-brain barrier. Handbook of experimental pharmacology 103, Springer-Verlag, Berlin, (1992); Lo, E. H., et al., Brain Res. Rev., 38:140-148, (2001)). Thus, in some embodiments, the compounds herein are covalently attached to a carrier moiety (represented by the symbol Y in the formulae above).

Any appropriate carrier moiety may be used herein. Useful carrier moieties include, for example, lipophilic carrier moieties, enzymatic substrate carrier moieties, peptidyl carrier moieties, and nanoparticle carrier moieties. Carrier moieties may also include an oligosaccharide unit or other molecule linked to the compound by phosphoester or lipid-ester or other hydrolyzable bonds which are cleaved by glycosidases, phosphatases, esterases, lipases, or other hydrolases in the lysosomes and endosomes. The carrier moieties may contain guanidine, amino, or imidazole functional groups.

1. Lipophilic Carrier Moieties

Lipophilic carrier moieties increase the overall lipophilicity of a compound, thereby aiding in passage through the BBB. Lipophilicity can be quantified using any suitable approach known in the art. For example, the partition coefficient between octanol and water (log P_{o/w}) may be measured thereby indicating the degree of lipophilicity. In some embodiments, the lipophilic carrier moiety has a log P_{o/w} of 1.5-2.5. Lipophilic carrier moieties are widely known in the art and are discussed in detail, for example, in Lambert, D. M., Eur J Pharm Sci., 11:S15-27 (2000). Exemplary lipophilic carrier moieties used to increase the lipophilicity of a compound include modified and unmodified diglycerides, fatty acids, and phospholipids.

Some lipophilic carrier moieties undergo enzyme mediated oxidation after traversing the BBB, resulting in a hydrophilic membrane impermeable form of the carrier moiety that remains trapped behind the BBB (Bodor et al., Pharmacol Ther 76:1-27 (1997); Bodor et al., American Chemical Society, Washington, D.C. pp 317-337 (1995); Chen et al., J Med Chem 41:3773-3781 (1998); Wu et al., J Pharm Pharmacol 54:945-950 (2002)). Exemplary lipophilic carrier moieties that undergo enzyme mediated oxidation include 1,4-dihydrotrigonelline (Palomino et al., J Med Chem, 32:622-625 (1989)); alkyl phosphonate carrier moieties that have been successfully used to transport testosterone and zidovudine across the blood-brain barrier (Somogyi, G., et al., Int J Pharm, 166:15-26 (1998)); and the lipophilic dihydropyridine carrier moieties that are enzymatically oxidized to the ionic pyridinium salt (Bodor et al., Science, 214(18):1370-1372 (1981)).

2. Peptidyl Carrier Moieties

Peptidyl carrier moieties are moieties partially or wholly composed of a peptide (including polypeptides, proteins, antibodies, and antibody fragments) used to aid in the transport of compounds across the BBB (Wu et al., J Clin Invest 100:1804-1812 (1997); U.S. Pat. No. 4,801,575; Pardridge et al., Adv Drug Deliv Rev, 36:299-321 (1999)).

Peptidyl carrier moieties may interact with specific peptide transport systems, receptors, or ligands, that target the corresponding ligand or receptor on an endothelial cell of the BBB. Specific transport systems may include either carrier-mediated or receptor-mediated transport across the BBB (U.S. Pat. App. No. 20040110928). Exemplary peptidyl carrier moieties include insulin (Pardridge et al., Nat Rev Drug Discov, 1:131-139 (2002)); small peptides such as enkephalin, thyrotropin-releasing hormone, arginine-vasopressin (Bergley, J Pharm Pharmacol, 48:136-146 (1996)), Banks et al., Peptides, 13:1289-1294 (1992)), Han et al., AAPS Pharm. Si., 2:E6 (2000)); chimeric peptides such as those described in WO-A-89/10134; amino acid derivatives such as those disclosed in U.S. Pat. App. No. 20030216589; tat peptide (Schwarze, S. R., et al., Science 285:1569-1572 (1999); polyarginine peptide (Wender, P. A., et al., Proc. Natl. Acad. Sci. USA 97:13003-13008 (2000)); insulin-like-growth factor-1; insulin-like-growth factor-2; transferrin; leptin; low-density lipoprotein (Pardridge, Nat. Rev. Drug Discov. 1:131-139 (2002); Colma et al., Pharm. Res. 17:266-274 (2000); Pardridge, Endocrine Rev, 7:314-330 (1986); Golden, et al., Clin Invest, 99:14-18 (1997); Bickel et al., Adv. Drug Deliv. Rev. 46(1-3):247-79 (2001)); and basic fibroblast growth factor (bFGF) (U.S. Pat. App. No. 20040102369).

U.S. Application No. 20040121947, and International Application No. PCT/USO2/34324 (Publication No. WO 03/039454), disclose that confocal microscopic images of cells incubated with a fluorescent tat-conjugated isosteric β-secretase inhibitor showed uneven distribution inside cells. Some high fluorescence intensity was associated with the endosome and lysosome intracellular vesicular structures. This indicated that the tat carrier moiety may have been modified by proteases within the lysosome or endosome resulting in an inhibitor that was unable to exit the lysosomal or endosomal compartment. Lysosomes and endosomes contain many proteases, including hydrolase such as cathepsins A, B, C, D, H and L. Some of these are endopeptidase, such as cathepsins D and H. Others are exopeptidases, such as cathepsins A and C, with cathepsin B capable of both endo- and exopeptidase activity. The specificities of these proteases are sufficiently broad to hydrolyze a tat peptide away from the inhibitor compound, thus, hydrolyzing the carrier peptide away from the isosteric inhibitor. Thus, it has been shown that tat and other carrier peptides may be particularly useful for specific delivery of isosteric inhibitors to lysosomes and endosomes. When administered to a mammal by a mechanism such as injections, the conjugated compound will penetrate cells and permeate to the interior of lysosomes and endosomes. The proteases in lysosomes and endosomes will then hydrolyze tat, thereby preventing to escape from lysosomes and endosomes.

The carrier peptide may be tat or other basic peptides, such as oligo-L-arginine, that are hydrolyzable by lysosomal and endosomal proteases. Specific peptide bonds susceptible for the cleavage of lysosomal or endosomal proteases may be installed, thereby facilitating the removal of the carrier compound from the inhibitor. For example, dipeptides Phe-Phe, Phe-Leu, Phe-Tyr and others are cleaved by cathepsin D.

In one embodiment, the peptidyl carrier molecule includes cationic functional groups, such as the tat-peptide (Schwarze, S. R., et al., Science 285: 1569-1572 (1999)), or nine arginine residues (Wender, P. A., et al., Proc. Natl. Acad. Sci. USA 97:13003-13008 (2000)). Useful cationic functional groups include, for example, guanidine, amino, and imidazole functional groups. Thus, cationic functional groups also include amino acid side chains such as side chains of lysine, arginine, and histidine residues. In some embodiments, the peptidyl carrier molecule may include from 1-10 cationic functional groups. When a compound herein is conjugated or attached to a carrier moiety, the resulting conjugate may be referred to herein as a “Carrier Peptide-Inhibitor” conjugate or “CPI.” The CPI conjugate can be administered to an in vitro sample or to a mammal thereby serving as a transport vehicle for a compound or compounds herein into a cell in an in vitro sample or in a mammal. The carrier moieties and CPI conjugates result in an increase in the ability of the compounds herein to effectively penetrate cells and the blood brain barrier to inhibit memapsin 2 from cleaving APP to subsequently generate Aβ.

Adsorptive-meditated transcytosis (AME) provides an alternative mechanism whereby peptidyl carrier moieties may cross the BBB. AME differs from other forms of transcytosis in that the initial binding of the carrier moiety to the luminal plasma membrane is mediated through either electrostatic interactions with anionic sites, or specific interactions with sugar residues. Uptake through AME is determined by the C-terminal structure and basicity of the carrier moiety. Exemplary adsorptive peptidyl carrier moieties include peptides and proteins with basic isoeletric points (cationic proteins), and some lectins (glycoprotein binding proteins). See Tamai, I., et al., J. Pharmacol. Exp. Ther. 280:410-415 (1997); Kumagai, A. K., et al., J. Biol. Chem. 262: 15214-15219 (1987).

Peptidyl carrier moieties also include antibody carrier moieties. Antibody carrier moieties are carrier moieties that include an antibody or fragment thereof. Typically, the antibody or antibody fragment is, or is derived from, a monoclonal antibody. Antibody carrier moieties bind to cellular receptors, or transporters expressed on the luminal surface of brain capillary endothelial cells (U.S. Patent App No. 20040101904). Exemplary antibodies, or fragments thereof, include MAb 83-14 that binds to the human insulin receptor (Pardridge et al., Pharm Res. 12:807-816 (1995)); anti-transferrin antibody (Li, J. Y., et al., Protein Engineering 12:787-796 (1999)); and monoclonal antibodies that mimic an endogenous protein or peptide which is known to cross the BBB as discussed above.

3. Nanoparticle Carrier Moieties

Nanoparticle carrier moieties are solid colloidal carriers generally less than a micron in diameter or length. The compound may be encapsulated in, adsorbed onto, or covalently linked to the surface of the nanoparticle carrier moiety. Nanoparticle carrier moieties have been used to successfully deliver a variety of compounds to the brain, including hexapeptide dalagrin, an enkephalin compound; loperamide; tubocerarine; and doxorubicin (Ambikanandan, et al., J. Pharm Pharmaceut Sci 6(2):252-273 (2003)). In addition to aiding transport into the brain, nonionic detergents such as polysorbate-80, which can be used to coat the nanoparticle, may be used to inhibit the efflux pump. Zordan-Nudo, T., et al., Cancer Res, 53:5994-6000 (1993). Exemplary materials for the manufacture of nanoparticle carrier moieties include polyalkylcyanoacrylate (PACA) (Bertling et al., Biotechnol. Appl. Biochem. 13: 390-405 (1991)); polybutylcyanoacrylate (PBCA) (Chavany et al., Pharm. Res. 9: 441-449 (1992)); polybutylcyanoacrylate with the peptide-drug complex absorbed onto the surface and coated with polysorbate 80 (Kreuter, J., et al., Brain Res, 674:171-174 (1995), Kreuter, J., Adv Drug Deliv Rev, 47:65-81, (2001), Kreuter, J., Curr Med Chem, 2:241-249 (2002)); polyisohexylcyanoacrylate (PIHCA) (Chavany et al., Pharm. Res. 11: 1370-1378 (1994)); polyhexylcyanoacrylate (PHCA) (Zobel et al., Antisense Nucleic Acid Drug Dev. 7:483-493 (1997)); and PEGylated polycyanoacrylate (Pilar, C., et al., Pharm Res 18(8):1157-1166 (2001)).

4. Linker Moieties

Linker moieties may be used to attach the carrier moiety to the β-secretase inhibitors herein. For example, steric hinderance between the compound and the carrier can be prevented using polymer technology (e.g., PEGylation) in conjunction with the linker molecule to introduce a long spacer arm (Yoshikawa, T., et al., J Pharmacol Exp Ther, 263:897-903, 1992). Linker moieties may be cleavable or non-cleavable.

Cleavable linker molecules include a cleavable moiety. Any appropriate cleavable moiety may be useful herein, including for example, phosphoesters, esters, disulfides, and the like. Cleavable moieties also include those moieties capable of being cleaved by biological enzymes, such as peptidases, glycosidases, phosphatases, esterases, lipases, or other hydrolases. Cleavable linker molecules are especially useful where the carrier moiety interferes with the biological activity of the compound. Exemplary cleavable linker molecules include N-succinimidyl-3-2-pyridyldithioproprionate (SPDP), or N-hydrosuccinimide (NHS).

Non-cleavable linker molecules are those that involve the attachment of a carrier moiety to the compound through a linkage that is generally stable to biological conditions and enzymes. Non-cleavable linker molecules are typically used when the carrier moiety does not interfere with the biological activity of the compound. Exemplary non-cleavable linker molecules include thio-ether (e.g., m-maleimidobenzoyl N-hydroxysuccinimide ester (MBS)); amide (e.g., N-hydrosuccinimide (NHS-XX—); extended amide (e.g., N-hydrosuccinimide polyethylene glycol (NHS-PEG); and extended hydrazide linkages (e.g., hydrazide-PEG-biotin-); avidin-biotin; and PEG linkers (Ambikanandan et al., J. Pharm Pharmaceut Sci 6(2):252-273 (2003); Pardridge, Adv Drug Deliv Rev, 36:299-321 (1999); U.S. Pat. No. 6,287,792).

### II. GENERAL SYNTHETIC METHODS

The compounds herein are synthesized by an appropriate combination of generally well-known synthetic methods. Techniques useful in synthesizing the compounds herein are both readily apparent and accessible to those of skill in the relevant art in light of the teachings described herein. The discussion below is offered to illustrate certain of the diverse methods available for use in assembling the compounds herein. However, the discussion is not intended to define the scope of reactions or reaction sequences that are useful in preparing the compounds herein.

A method for synthesizing the inhibitor compounds described herein is by adapting the synthesis for N1-((1R,2S)-1-((2R,4R)-4-(benzyloxy)pyrrolidin-2-yl)-1-hydroxy-3-phenylpropan-2-yl)-N3-methyl-N3-((4-methylthiazol-2-yl)methyl)isophthalamide (9a) and N-((1R,2S)-1-((2R,4R)-4-(benzyloxy)pyrrolidin-2-yl)-1-hydroxy-3-phenylpropan-2-yl)-3-((R)-2-(4-methylthiazol-2-yl)pyrrolidine-1-carbonyl)benzamide (9b) below.

Scheme 1 shows an exemplary synthesis of an hydroxyamine pyrrolidine fragment. 1-phenyl-2-nitroethane can be coupled to (2R,4R)-tert-butyl 4-(benzyloxy)-2-formylpyrrolidine-1-carboxylate (synthesis in Experimental section) using e.g., a mild base, such as tetrabutylammonium fluoride (TBAF). The nitro group can then be transformed to an amine under reducing conditions, such as NiCl₂ and NaBH₄, to generate the desired hydroxylamine pyrrolidine fragment, such as (2R,4R)-tert-butyl 2-((1S,2S)-2-amino-1-hydroxy-3-phenylpropyl)-4-(benzyloxy)pyrrolidine-1-carboxylate (4). Alternatively, the hydroxyamine pyrrolidine fragment may be generated using Evans' chiral auxiliary oxazolidinone, as described in the Experimental section below.

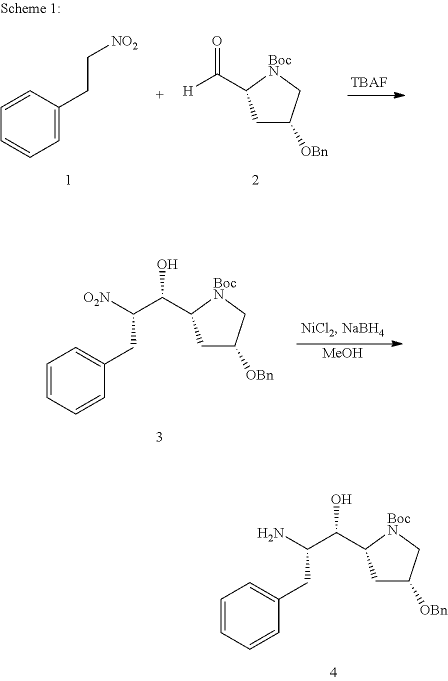

Various substituents on the pyrrolidine fragment may be synthesized, e.g., by removal of the benzyl protecting group of 4, followed by protection of the linear hydroxylamine moiety to generate (4S,5R)-benzyl 4-benzyl-5-((2R,4R)-4-hydroxypyrrolidin-2-yl)-2,2-dimethyloxazolidine-3-carboxylate. The free hydroxyl can then be transformed into a variety of functionalities using techniques known in the art (e.g., using Mitsunobu chemistry, Appel reaction, etc).

Scheme 2 shows an exemplary synthesis of desired inhibitors 9a and 9b. Partially protected isophthalate 5 can be coupled with amine 6a or 6b, (e.g., using thionyl chloride or any suitable couple agent, such as EDCI with HOBt), followed by ester hydrolysis under basic conditions (such as NaOH or LiOH) to generate 7a or 7b, respectively. A hydroxyamine pyrrolidine fragment, such as 4, can then be coupled to the free carboxylate of 7a or 7b under suitable coupling conditions (e.g., Py-BOP, or EDCI with HOBt) to generate 8a or 8b, respectively. Removal of the remaining Boc protecting group under acidic conditions (e.g., HCl) yields the corresponding inhibitor 9a or 9b.

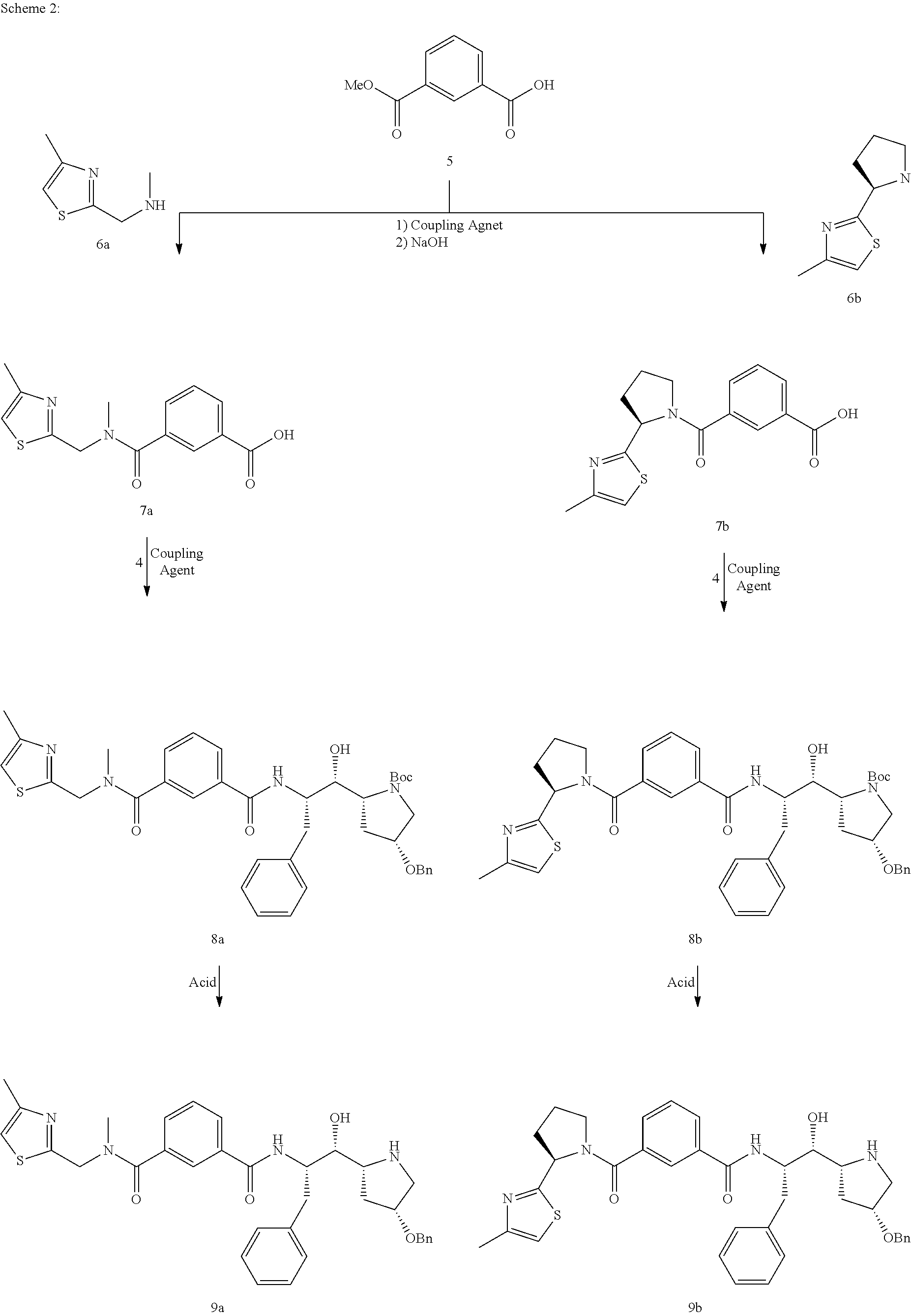

### III. BETA-SECRETASE INHIBITOR ACTIVITY

To develop useful β-secretase inhibitors, candidate inhibitors capable of selectively mediating, e.g., decreasing, memapsin 2 catalytic activity may be identified in vitro and subsequently tested for their ability to reduce the production of Aβ. The activity of the inhibitor compounds can be assayed utilizing methods known in the art and/or those methods presented herein.

Compounds that decrease memapsin 2 catalytic activity may be identified and tested using biologically active memapsin 2, either recombinant or naturally occurring. Memapsin 2 can be found in native cells, isolated in vitro, or co-expressed or expressed in a cell. Measuring the reduction in the memapsin 2 catalytic activity in the presence of an inhibitor relative to the activity in the absence of the inhibitor may be performed using a variety of methods known in the art.

For example, the compounds may be tested for their ability to cause a detectable decrease in hydrolysis of a β-secretase site of a peptide in the presence of memapsin 2. These data can be expressed, for example, as Kᵢ, Kᵢ apparent, Vᵢ/Vₒ, or percentage inhibition. Kᵢ is the inhibition equilibrium constant which indicates the ability of compounds to inhibit a given enzyme (such as memapsin 2, memapsin 1, and/or cathepsin D). Numerically lower Kᵢ values indicate a higher affinity of the compounds herein for the enzyme. The Kᵢ value is independent of the substrate, and converted from Kᵢ apparent.

Kᵢ apparent is determined in the presence of substrate according to established techniques (see, for example, Bieth, J., Bayer-Symposium V: Proteinase Inhibitors, pp. 463-469, Springer-Verlag, Berlin (1994)). The standard error for the Kᵢ apparent is the error from the nonlinear regression of the Vᵢ/Vₒ data measured at different concentrations of the compounds herein (e.g., between about 10 nM to about 1000 nM) employing well-known techniques (see, for example, Bieth, J., Bayer-Symposium V: Proteinase Inhibitors, pp. 463-469, Springer-Verlag, Berlin (1994), Ermolieff, J., et al., Biochemistry 39:12450-12456 (2000), the teachings of which are incorporated herein by reference in their entirety). Vᵢ/Vₒ depicts the ratio of initial conversion velocities of an enzyme substrate (Ermolieff, et al., Biochemistry 40:12450-12456 (2000)) by an enzyme in the absence (Vₒ) or presence (Vᵢ) of an inhibitor. A Vᵢ/Vₒ value of 1.0 indicates that a compound does not inhibit the enzyme at the concentration tested. A Vᵢ/Vₒ value less than 1.0 indicates that a compound herein inhibits enzyme activity.

In some embodiments, the compounds described herein (e.g., any compound of formula I, II, III, Example 2 and/or Table 1) are capable of reducing memapsin 2 beta-secretase activity. In some embodiments, the compounds have a memapsin 2 beta-secretase Kᵢ and/or Kᵢ apparent (e.g., using any inhibitory assay described herein) of less than about any one of 10 μM, 5 μM, 1 μM, or less than about any one of 750, 500, 400, 300, 200, 100, 50, 25, 10, 5, 2, or 1 nM; or from about 1 to 5, 1 to 10, 1 to 100, 1 to 300, 1 to 500, 1 to 1000, 100 to 500, 200 to 500, 300 to 500, 100 to 750, 200 to 750, 300 to 750, 400 to 750, 500 to 750, 100 to 1000, 250 to 1000, 500 to 1000, or 750 to 1000 nM. In some embodiments, the compounds have a memapsin 2 beta-secretase Kᵢ and/or Kᵢ apparent (e.g., using any inhibitory assay described herein) of less than about 300, 301 to 500, or greater than 501 nM.

Once compounds are identified that are capable of mediating, e.g., reducing, the hydrolysis of a β-secretase site of a peptide in the presence of memapsin 2, the compounds may be further tested for their ability to selectively inhibit memapsin 2 relative to other enzymes. Typically, the other enzyme is a peptide hydrolase, such as memapsin 1 or cathepsin D; or from another family of interest, such as Cytochrome P450 3A4 (CYP3A4). Compounds that decrease cathepsin D catalytic activity or memapsin 1 catalytic activity are tested using biologically active enzyme, either recombinant or naturally occurring. Cathepsin D or memapsin 1 catalytic activity can be found in native cells, isolated in vitro, or co-expressed or expressed in a cell. Inhibition by a compound described herein is measured using standard in vitro or in vivo assays such as those well known in the art or as otherwise described herein.

For example, selectivity of a compound may be measured by determining the extent to which memapsin 2 hydrolyzes a substrate peptide compared to the extent to which the same compound inhibits memapsin 1 and/or cathepsin D cleaving of a β-secretase site of a substrate peptide in the presence of the compound. Exemplary substrate peptides are useful in determining the activity of memapsin 2 includes APP and derivatives thereof, such as FS-2 (MCA-SEVNLDAEFR-DNP; SEQ ID NO.: 2) (Bachem Americas, Torrance, Calif.). Exemplary substrate peptides are useful in determining the activity of memapsin 1 and cathepsin D include, for example, peptides which include the sequence ELDLAVEFWHDR (SEQ ID NO.: 1). These substrate peptides can be synthesized using known peptide synthesis methods, e.g., solid-phase peptide synthesis (e.g., FMOC amino acid coupling etc.). These data can be expressed, for example, as Kᵢ, Kᵢ apparent, Vᵢ/Vₒ, or percentage inhibition and depict the inhibition of a compound for memapsin 2 catalytic activity relative to memapsin 1 or cathepsin D catalytic activity. For example, if the Kᵢ of a reaction between an inhibitor compound herein and memapsin 1 or cathepsin D is 1000 and the Kᵢ of a reaction between an inhibitor compound herein and memapsin 2 is 100, the inhibitor compound inhibits the β-secretase activity of memapsin 2 with ten-fold selectivity over memapsin 1 or cathepsin D.

The compounds described herein may be capable of selectively inhibiting memapsin 2 in the presence of Cytochrome P450 3A4 (CYP3A4). CYP3A4 plays an important role in the metabolism of xenobiotics. Inhibition of CYP3A4 can lead to unwanted drug-drug interactions by modulating the metabolism of other therapeutics. Many patients, particularly those patients in advanced age seeking treatment for conditions such as Alzheimer's disease, are prescribed multiple therapeutics for various conditions, wherein drug-drug interactions caused by inhibition of CYPAA4 would be highly undesirable. Accordingly, the ability to selectively inhibit memapsin 2 over CYP3A4 (e.g., not effect or minimally effect CYP3A4) may aid in decreasing unwanted drug-drug interactions leading to decreased toxicity and increased effectiveness of beta-secretase inhibitors. Some compounds described herein have been shown to exhibit strikingly selective inhibition of memapsin 2 in the presence of Cytochrome P450 3A4. For example, N-((1R,2S)-1-((2R,4R)-4-(benzyloxy)pyrrolidin-2-yl)-1-hydroxy-3-phenylpropan-2-yl)-3-((R)-2-(4-methyloxazol-2-yl)pyrrolidine-1-carbonyl)benzamide was determined to have an M2 Ki of approximately 50.38 nM and a CYP3A4 Ki=11.5 μM (see data below). By comparison, N-((2S,3R)-4-((5-tert-butylpyridin-3-yl)methylamino)-3-hydroxy-1-phenylbutan-2-yl)-3-methyl-5-((R)-2-(4-methyloxazol-2-yl)pyrrolidine-1-carbonyl)benzamide, which lacks a pyrrolidine ring when compared to N-((1R,2S)-1-((2R,4R)-4-(benzyloxy)pyrrolidin-2-yl)-1-hydroxy-3-phenylpropan-2-yl)-3-((R)-2-(4-methyloxazol-2-yl)pyrrolidine-1-carbonyl)benzamide, has an M2 Ki of approximately 9.29 nM and a CYP3A4 Ki=0.717 μM (see data below). In some embodiments, the compounds described herein (e.g., any compound of formula I, II, III, Example 2 and/or Table 1) are capable of selectively reducing memapsin 2 relative to CYP3A4.

In some embodiments, the compounds described herein (e.g., any compound of formula I, II, III, Example 2 and/or Table 1) are capable of selectively reducing memapsin 2 relative to memapsin 1, cathepsin D and/or CYP3A4. In some embodiments, the compounds are capable of selectively reducing memapsin 2 relative to memapsin 1, cathepsin D, and/or CYP3A4 with greater than about 2-fold selectivity, or greater than about any one of 3, 5, 7, 10, 25, 50, 75, 100, 300, 200, 500, 750, 1000, 2000, 5000, or 10000-fold selectivity. In some embodiments, the compounds have a memapsin 2 beta-secretase Kᵢ and/or Kᵢ apparent (e.g., using any inhibitory assay described herein) of less than about 10 μM, 5 μM, 1 μM, or less than about any one of 750, 500, 400, 300, 250, 200, 100, 75, 50, 25, 10, 5, 2, or 1 nM, or from about any of 1 to 5, 1 to 10, 1 to 100, 1 to 250, 1 to 500, 1 to 1000, 100 to 500, 200 to 500, 300 to 500, 100 to 750, 200 to 750, 250 to 750, 300 to 750, 400 to 750, 500 to 750, 100 to 1000, 250 to 1000, 500 to 1000, or 750 to 1000 nM; and have a memapsin 1 and/or cathepsin D Kᵢ and/or Kᵢ apparent of more than about 10 μM, 5 μM, 1 μM, or more than about any one of 750, 500, 400, 300, 200, 100, 50, 25, 10, 5, 2, or 1 nM, or from about any of 1 to 5, 1 to 10, 1 to 100, 1 to 300, 1 to 500, 1 to 1000, 100 to 500, 200 to 500, 300 to 500, 100 to 750, 200 to 750, 300 to 750, 400 to 750, 500 to 750, 100 to 1000, 250 to 1000, 500 to 1000, or 750 to 1000 nM. In some embodiments, the compounds have a memapsin 2 beta-secretase Kᵢ and/or Kᵢ apparent (e.g., using any inhibitory assay described herein) of less than about 10 μM, 5 μM, 1 μM, or less than about any one of 750, 500, 400, 300, 250, 200, 100, 50, 25, 10, 5, 2, or 1 nM, or from about any of 1 to 5, 1 to 10, 1 to 100, 1 to 250, 1 to 500, 1 to 1000, 100 to 500, 200 to 500, 300 to 500, 100 to 750, 200 to 750, 250 to 750, 400 to 750, 500 to 750, 100 to 1000, 250 to 1000, 500 to 1000, or 750 to 1000 nM; and have a CYP3A4 Kᵢ and/or Kᵢ apparent of more than about 100 μM, 50 μM, 25 μM, 10 μM, 5 μM, 1 μM, or more than about any one of 750, 500, 400, 300, 200, 100, 50, 25, 10, 5, 2, or 1 nM, or from about any of 1 to 5, 1 to 10, 1 to 100, 1 to 300, 1 to 500, 1 to 1000, 100 to 500, 200 to 500, 300 to 500, 100 to 750, 200 to 750, 300 to 750, 400 to 750, 500 to 750, 100 to 1000, 250 to 1000, 500 to 1000, or 750 to 1000 nM.

Compounds demonstrating the ability to cause a detectable decrease in hydrolysis of a β-secretase site of a peptide in the presence of memapsin 2 (or, in addition, selectivity of action toward memapsin 2), may be tested in cell models or animal models for their ability to cause a detectable decrease in the amount or production of β-amyloid protein (Aβ). For example, isosteric inhibitors of memapsin 2 have been tested for their ability to decrease Aβ production in cultured cells (see U.S. Patent Application Publication No. 20040121947, International Application No. PCT/US02/34324 (Publication No. WO 03/039454), and International Application No. PCT/US06/13342 (Publication No. WO 06/110668, the contents of which are hereby incorporated by reference)). Briefly, inhibitors may be added to a culture of cells (e.g., human embryonic kidney (HEK293) cells, HeLa cells, Chinese hamster ovary cells, or neuroblastoma line M17 cells) stably transfected with a nucleic acid constructs that encode human APP Swedish mutant (or London mutation or double mutant) and, if needed, a nucleic acid construct encoding human memapsin 2. Immunoprecipitation of Aβ followed by SDS-gel electrophoresis allows detection and quantitation of the amount of Aβ produced in the presence and absence of inhibitor.

In addition to cell cultures, animal models may be used to test inhibitors of memapsin 2 for their ability to decrease Aβ production. For example, an animal (e.g., tg2576 mice) expressing the Swedish mutation of the human amyloid precursor protein (Hsiao, K., et al., Science 274, 99-102 (1996) may be injected intraperitoneally with an inhibitor. The plasma may then be collected and Aβ levels determined by capture ELISA (BioSource International, Camarillo, Calif.).

In some embodiments, the compounds described herein (e.g., any compound of formula I, II, III, Example 2 and/or Table 1) are capable of reducing cellular Aβ production. In some embodiments, the compounds are capable of reducing cellular Aβ production with a IC50 (e.g., using an Aβ inhibitory assay described herein) of less than about 10 μM, 5 μM, 1 μM, or less than about 750, 500, 400, 300, 200, 100, 50, 25, 10, 5, 2, or 1 nM, or from about 1 to 5, 1 to 10, 1 to 100, 1 to 300, 1 to 500, 1 to 1000, 100 to 500, 200 to 500, 300 to 500, 100 to 750, 200 to 750, 300 to 750, 400 to 750, 500 to 750, 100 to 1000, 250 to 1000, 500 to 1000, or 750 to 1000 nM. In some embodiments, the compounds are capable of reducing cellular Aβ production with a IC50 (e.g., using an Aβ inhibitory assay described herein) of less than 1 μM, between 1 and 5 μM, or greater than 5 μM.

The presence of inhibitors in organs of animal models or within cellular compartments may be ascertained using a fluorescent tag conjugated to the inhibitor and visualization via confocal microscopy (see U.S. Patent Application Publication No. 20040121947, and International Application No. PCT/USO2/34324 (Publication No. WO 03/039454), the contents of which are hereby incorporated by reference in their entireties).

The sample obtained from the mammal can be a fluid sample, such as a plasma or serum sample; or can be a tissue sample, such as a brain biopsy. The amount of β-amyloid protein or a decrease in the production of β-amyloid protein can be measured using standard techniques (e.g., western blotting and ELISA assays).

Further examples of assays for identifying memapsin 2-β-secretase inhibitors are set forth in the Examples section below. Other methods for assaying the activity of memapsin 2, memapsin 1, cathepsin D, and CYP3A4 and the activity of agents that decrease the activity of these enzymes are known in the art. The selection of appropriate assay methods is well within the capabilities of those of skill in the art, particularly in view of the teaching provided herein.

### IV. HEPATIC INTRINSIC CLEARANCE IN LIVER MICROSOMES

The compounds herein (e.g., any compound of formula I, II, III, Example 2 and/or Table 1) may have one or more favorable pharmacokinetic properties. For example, the ability for a beta-secretase inhibitor compound to resist hepatic clearance in an individual will result in the compound being available as a therapeutic for a longer duration, which may aid in e.g., lower dosage and/or less frequent dosing. Accordingly, beta-secretase inhibitor compounds with decreased hepatic clearance may have the advantages of potentially decreasing toxicity and may improve patient compliance. Some compounds described herein have been shown to exhibit strikingly lower hepatic intrinsic clearance properties. For example, N-((1R,2S)-1-((2R,4R)-4-(benzyloxy)pyrrolidin-2-yl)-1-hydroxy-3-phenylpropan-2-yl)-3-((R)-2-(4-methyloxazol-2-yl)pyrrolidine-1-carbonyl)benzamide was determined to have a hepatic intrinsic clearance in liver microsomes of approximately 337 mL/min/kg (see data below). By comparison, N-((2S,3R)-4-((5-tert-butylpyridin-3-yl)methylamino)-3-hydroxy-1-phenylbutan-2-yl)-3-methyl-5-((R)-2-(4-methyloxazol-2-yl)pyrrolidine-1-carbonyl)benzamide, which lacks a pyrrolidine ring when compared to N-((1R,2S)-1-((2R,4R)-4-(benzyloxy)pyrrolidin-2-yl)-1-hydroxy-3-phenylpropan-2-yl)-3-((R)-2-(4-methyloxazol-2-yl)pyrrolidine-1-carbonyl)benzamide, has a hepatic intrinsic clearance in liver microsomes of greater than 1000 mL/min/kg (see data below).

In some embodiments, the compounds described herein (e.g., any compound of formula I, II, III, Example 2 and/or Table 1) have a hepatic intrinsic clearance in liver microsomes of less than any of about 1000 mL/min/kg, 900 mL/min/kg, 800 mL/min/kg, 700 mL/min/kg, 600 mL/min/kg, 500 mL/min/kg, 300 mL/min/kg, 200 mL/min/kg, 150 mL/min/kg, 100 mL/min/kg, 75 mL/min/kg, 50 mL/min/kg, or 25 mL/min/kg, as measured by LC/MS/MS (see Examples section for assay details).

### V. FORMULATIONS

In another aspect, are provided formulations (e.g., pharmaceutical formulations) comprising a memapsin 2β-secretase inhibitor compound (e.g., any compound of formula I, II, III, Example 2 and/or Table 1) with a carrier, such as a pharmaceutically acceptable carrier. The formulations may include optical isomers, diastereomers, or pharmaceutically acceptable salts of the inhibitors disclosed herein. The memapsin 2β-secretase inhibitor included in the formulation may be covalently attached to a carrier moiety, as described above. Alternatively, the memapsin 2β-secretase inhibitor included in the formulation is not covalently linked to a carrier moiety.

Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which preferably do not deleteriously react with the intended compound of use.

The compounds described herein can be administered alone or can be coadministered to the individual. Coadministration is meant to include simultaneous or sequential administration of the compounds individually or in combination (more than one compound). Thus, the preparations can also be combined, when desired, with other active substances related to the treatment of a specified condition (e.g., to reduce metabolic degradation).

The β-secretase inhibitors described herein (e.g., any compound of formula I, II, III, Example 2 and/or Table 1) can be prepared and administered in a wide variety of oral, parenteral and topical dosage forms. Thus, the compounds herein can be administered by injection (e.g., intravenously, intramuscularly, intracutaneously, subcutaneously, intraduodenally, or intraperitoneally). Also, the compounds described herein can be administered by inhalation, for example, intranasally. Additionally, the compounds herein can be administered transdermally. Compounds herein may also be administered locally (e.g., ocular administration such as topical eye drops or ointment). It is also envisioned that multiple routes of administration (e.g., intramuscular, oral, transdermal) can be used to administer the inhibitor compounds described herein. Accordingly, also provided are pharmaceutical formulations comprising a pharmaceutically acceptable carrier or excipient and one or more inhibitor compounds described herein (e.g., any compound of formula I, II, III, Example 2 and/or Table 1).

For preparing pharmaceutical formulations from the compounds described herein, pharmaceutically acceptable carriers can be either solid or liquid. Solid form preparations include powders, tablets, pills, capsules, cachets, suppositories, and dispersible granules. A solid carrier can be one or more substance, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds herein are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compounds herein can also be incorporated into liposomes or administered via transdermal pumps or patches. Pharmaceutical admixtures suitable for use herein are well-known to those of skill in the art and are described, for example, in Pharmaceutical Sciences (17th Ed., Mack Pub. Co., Easton, Pa.) and WO 96/05309, the teachings of both of which are hereby incorporated by reference.

Ocular administration preparations (e.g., in use of glaucoma treatment) include, but are not limited to, formulations in saline, optionally with additional carriers, stabilizers, etc. know to those of skill in the art.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Also provided are unit dosage forms comprising the formulations described herein. These unit dosage forms can be stored in a suitable packaging in single or multiple unit dosages and may also be further sterilized and sealed. For example, the pharmaceutical formulation (e.g., a dosage or unit dosage form of a pharmaceutical formulation) may include (i) an in inhibitor (e.g., any compound of formula I, II, III, Example 2 and/or Table 1) and (ii) a pharmaceutically acceptable carrier. In some embodiments, the formulation also includes one or more other compounds (or pharmaceutically acceptable salts thereof). In various variations, the amount of inhibitor compound in the formulation is included in any of the following ranges: about 5 to about 50 mg, about 20 to about 50 mg, about 50 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. In some embodiments, the amount of compound in the formulation (e.g., a dosage or unit dosage form containing any compound of formula I, II, III, Example 2 and/or Table 1) is in the range of about 5 mg to about 500 mg, such as about 30 mg to about 300 mg or about 50 mg to about 200 mg, of the compound.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The formulations described may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

A. Effective Dosages

Pharmaceutical formulations described include formulations wherein the active ingredient (e.g., any compound of formula I, II, III, Example 2 and/or Table 1) is contained in an effective amount, i.e., in an amount effective to achieve its intended purpose. The actual amount effective for a particular application will depend, inter alia, on the condition being treated. For example, when administered in methods to treat Alzheimer's disease, such compositions will contain an amount of active ingredient effective to achieve the desired result (e.g., decreasing β-secretase activity or β-amyloid production). Determination of an effective amount of a compound herein is well within the capabilities of those skilled in the art, especially in light of the detailed disclosure herein.

The dosage and frequency (single or multiple doses) administered to a mammal can vary depending upon a variety of factors, including a disease that results in increased activity of memapsin 2 or increased accumulation of β-amyloid protein, whether the mammal suffers from another disease, and its route of administration; size, age, sex, health, body weight, body mass index, and diet of the recipient; nature and extent of symptoms of the disease being treated (e.g., Alzheimer's disease), kind of concurrent treatment, complications from the disease being treated or other health-related problems. Other therapeutic regimens or agents can be used in conjunction with the methods and compounds described herein. Adjustment and manipulation of established dosages (e.g., frequency and duration) are well within the ability of those skilled in the art.

For any compound described herein, the effective amount can be initially determined from cell culture assays. Target concentrations will be those concentrations of active compound(s) that are capable of reducing the activity of memapsin 2 activity, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring memapsin 2 inhibition and adjusting the dosage upwards or downwards, as described above. Adjusting the dose to achieve maximal efficacy in humans based on the methods described above and other methods as are well-known in the art is well within the capabilities of the ordinarily skilled artisan, particularly in view of the teaching provided herein.

Dosages may be varied depending upon the requirements of the individual and the compound being employed. The dose administered to an individual, should be sufficient to affect a beneficial therapeutic response in the individual over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In one embodiment, the dosage range is 0.001% to 10% w/v. In another embodiment, the dosage range is 0.1% to 5% w/v.

Additional examples of dosages which can be used are an effective amount within the dosage range of about 0.1 μg/kg to about 300 mg/kg, or within about 1.0 μg/kg to about 40 mg/kg body weight, or within about 1.0 μg/kg to about 20 mg/kg body weight, or within about 1.0 μg/kg to about 10 mg/kg body weight, or within about 10.0 μg/kg to about 10 mg/kg body weight, or within about 100 μg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight. Compounds herein may be administered in a single daily dose, or the total daily dosage may be administered in divided dosage of two, three or four times daily.

Utilizing the teachings provided herein, an effective prophylactic or therapeutic treatment regimen can be planned which does not cause substantial toxicity and yet is entirely effective to treat the clinical symptoms demonstrated by the particular individual. This planning should involve the careful choice of active compound by considering factors such as compound potency, relative bioavailability, individual body weight, presence and severity of adverse side effects, preferred mode of administration and the toxicity profile of the selected agent.

B. Kits

Also provided are kits for administration of the compounds described herein (e.g., any compound of formula I, II, III, Example 2 and/or Table 1, formulations, and dosage forms thereof).

In certain embodiments the kits may include a dosage amount of at least one formulation as disclosed herein. Kits may further comprise suitable packaging and/or instructions for use of the formulation. Kits may also comprise a means for the delivery of the formulation thereof.

The kits may include other pharmaceutical agents for use in conjunction with the one or more compounds described herein (e.g., any compound of formula I, II, III, Example 2 and/or Table 1). In some variations, the pharmaceutical agent(s) may be one or more anti-psychotic drugs. These agents may be provided in a separate form, or mixed with the compounds described herein, provided such mixing does not reduce the effectiveness of either the pharmaceutical agent or compound described herein and is compatible with the route of administration. Similarly the kits may include additional agents for adjunctive therapy or other agents known to the skilled artisan as effective in the treatment or prevention of the conditions described herein.

The kits may optionally include appropriate instructions for preparation and administration of the composition, side effects of the composition, and any other relevant information. The instructions may be in any suitable format, including, but not limited to, printed matter, videotape, computer readable disk, optical disc or directions to internet-based instructions.

In another aspect, are provided kits for treating an individual who suffers from or is susceptible to the conditions described herein are provided, comprising a first container comprising a dosage amount of a formulation as disclosed herein, and instructions for use. The container may be any of those known in the art and appropriate for storage and delivery of intravenous formulation. In certain embodiments the kit further comprises a second container comprising a pharmaceutically acceptable carrier, diluent, adjuvant, etc. for preparation of the composition to be administered to the individual.

Kits may also be provided that contain sufficient dosages of the inhibitor (including formulation thereof) as disclosed herein to provide effective treatment for an individual for an extended period, such as 1-3 days, 1-5 days, a week, 2 weeks, 3, weeks, 4 weeks, 6 weeks, 8 weeks, 3 months, 4 months, 5 months, 6 months, 7 months, 8 months, 9 months or more.

Kits may also include multiple doses of the compound and instructions for use and packaged in quantities sufficient for storage and use in pharmacies, for example, hospital pharmacies and compounding pharmacies.

The kits may include the compounds as described herein (e.g., any compound of formula I, II, III, Example 2 and/or Table 1) packaged in either a unit dosage form or in a multi-use form. The kits may also include multiple units of the unit dose form. In certain embodiments, are provided the compound described herein in a unit dose form. In other embodiments the compositions may be provided in a multi-dose form (e.g., a blister pack, etc.).

C. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g., Fingl et al., In: THE PHARMACOLOGICAL BASIS OF THERAPEUTICS, Ch. 1, p. 1, 1975. The exact formulation, route of administration and dosage can be chosen by the individual physician in view of the individual's condition and the particular method in which the compound is used.

### VI. METHODS OF REDUCING THE ACTIVITY OF MEMAPSIN 2 BETA-SECRETASE

In another aspect, the β-secretase inhibitor compounds herein can be employed in methods to decrease memapsin 2 activity, decrease hydrolysis of a β-secretase site of a memapsin 2 substrate, and/or decrease the accumulation of β-amyloid protein relative to the amount of memapsin 2 activity, hydrolysis of a β-secretase site, and accumulation of β-amyloid protein, respectively, in the absence of the β-secretase inhibitor.

In an exemplary embodiment, a method of reducing memapsin 2 activity is provided. The method includes contacting a memapsin 2 with a β-secretase inhibitor compound herein. The memapsin 2 may be contacted in any appropriate environment (e.g., in vitro, in vivo). The memapsin 2 activity is decreased relative the amount of activity in the absence of β-secretase inhibitor.

In another exemplary embodiment, a method is provided of selectively mediating (e.g., reducing) memapsin 2 activity using an inhibitor described herein (e.g., any compound of formula I, II, III, Example 2 and/or Table 1). Selective reduction of the activity of memapsin 2 means that memapsin 2 is not only reduced relative to its activity in the absence of inhibitor, but is reduced to a greater extent as compared to the reduction in activity due to inhibitor action against another enzyme, such as a peptide hydrolase (e.g., cathepsin D, memapsin 1) and/or Cytochrome P450 3A4. For example, as described above, the reduction in activity of an enzyme may be expressed in terms of the inhibitory constant (Kᵢ). Where an inhibitor selectively reduces the activity of memapsin 2, the Kᵢ of the reaction between an inhibitor compound described herein and memapsin 2 is less than the Kᵢ of the reaction between an inhibitor compound herein and another peptide hydrolase and/or Cytochrome P450 3A4.

In some embodiments, the Kᵢ of the reaction between an inhibitor compound (e.g., any compound of formula I, II, III, Example 2 and/or Table 1) and memapsin 2 is less than the Kᵢ of the reaction between an inhibitor compound and another peptide hydrolase (e.g., cathepsin D, memapsin 1). In some related embodiments, the inhibitor selectively reduces the activity of memapsin 2 as compared to memapsin 1. In other related embodiments, the inhibitor selectively reduces the activity of memapsin 2 as compared to cathepsin D. In some embodiments, the Kᵢ of the reaction between an inhibitor compound (e.g., any compound of formula I, II, III, Example 2 and/or Table 1) and memapsin 2 is less than the Kᵢ of the reaction between an inhibitor compound and Cytochrome P450 3A4. In an exemplary embodiment, the Kᵢ of the reaction between an inhibitor compound herein and memapsin 2 is at least 2 times less than the Kᵢ of the reaction between an inhibitor compound herein and another peptide hydrolase and/or Cytochrome P450 3A4. In another exemplary embodiment, the Kᵢ of the reaction between an inhibitor compound herein and memapsin 2 is at least 3, 5, 7, 10, 25, 50, 75, 100, 300, 200, 500, 750, 1000, 2000, 5000, or 10000 times less than the Kᵢ of the reaction between an inhibitor compound herein and another peptide hydrolase and/or Cytochrome P450 3A4.

Thus, provided are methods of selectively reducing the activity of memapsin 2. The methods include contacting a memapsin 2 with a β-secretase inhibitor compound (e.g., any compound of formula I, II, III, Example 2 and/or Table 1). In a related embodiment, the method includes contacting the memapsin 2 with a β-secretase inhibitor in the presence of memapsin 1. In an alternative related embodiment, the method includes contacting the memapsin 2 with a β-secretase inhibitor in the presence of cathepsin D. In yet another related embodiment, the method includes contacting the memapsin 2 with a β-secretase inhibitor in the presence of cathepsin D and memapsin 1. In yet another embodiment, the method includes contacting the memapsin 2 with a β-secretase inhibitor in the presence of Cytochrome P450 3A4. In still another related embodiment, the method includes contacting the memapsin 2 with a β-secretase inhibitor in the presence of cathepsin D, memapsin 1, and Cytochrome P450 3A4.

In some embodiments, the activity of memapsin-2β-secretase may be determined by measuring the hydrolysis of a β-secretase site of a β-secretase substrate. Thus, described are methods of decreasing the hydrolysis of a β-secretase site of a β-secretase substrate by contacting a memapsin 2 with a β-secretase inhibitor compound (e.g., any compound of formula I, II, III, Example 2 and/or Table 1). In some embodiments, the hydrolysis of a β-secretase site is decreased relative the amount of hydrolysis in the absence of the inhibitor. In other embodiments, the hydrolysis is selectively reduced as compared to hydrolysis by memapsin 1 and/or cathepsin D. Thus, a method of selectively decreasing hydrolysis of a β-secretase site of a β-amyloid precursor protein relative to memapsin 1 and/or cathepsin D in a sample is provided. The method includes contacting a memapsin 2 with a β-secretase inhibitor compound.

In another embodiment, are provided methods of decreasing the amount of β-amyloid protein in a sample by contacting the memapsin 2 with an inhibitor compound (e.g., any compound of formula I, II, III, Example 2 and/or Table 1). The amount of β-amyloid protein in a sample is decreased relative the amount of β-amyloid protein in the sample in the absence of the inhibitor. Thus, the accumulation of β-amyloid protein is thereby decreased.

Memapsin 2 may be contacted in any suitable environment or any suitable sample. For example, memapsin 2 may be contacted in vitro, within a cell, or within a mammal. Typically, in vitro solutions are selected such that the components do not substantially interfere with the enzymatic activity of memapsin 2 (e.g., aqueous solutions). In some embodiments, the in vitro solution includes a biological sample, such as a mammalian sample. Exemplary mammalian samples include plasma or serum samples and tissue samples, such as a brain biopsy. Any appropriate cell or cellular sample may be selected in which to contact the memapsin 2 with the inhibitor. The cell may contain endogenous memapsin 2 or recombinant memapsin 2 as previously described (see U.S. Patent Application Publication No. 20040121947 (the contents of which are hereby incorporated by reference), and International Application No. PCT/USO2/34324 (Publication No. WO 03/039454)). Exemplary cells include human embryonic kidney (HEK293) cells, HeLa cells, Chinese hamster ovary cells, or neuroblastoma line M17 cells Hela cells, 293 cells. In an exemplary embodiment, the compounds herein are administered to a mammal to inhibit the hydrolysis of a β-secretase site of a β-amyloid precursor protein (e.g., a mouse, rabbit or human).

### VII. METHODS OF TREATING ALZHEIMER'S DISEASE

In another aspect, the β-secretase inhibitor compounds herein can be employed in the treatment of diseases or conditions associated with and/or mediated by β-secretase activity, hydrolysis of a β-secretase site of a β-amyloid precursor protein, and/or β-amyloid protein accumulation. Typically, a mammal is treated for the disease or condition. In an exemplary embodiment, the disease is Alzheimer's disease.

Thus, in some embodiments, are provided a method of treating Alzheimer's disease in a mammal comprising the step of administering to the mammal in need thereof an effective amount of a β-secretase inhibitor (e.g., any compound of formula I, II, III, Example 2 and/or Table 1). In some embodiments, the individual has one or more symptoms of Alzheimer's disease. In some embodiments, the individual has been diagnosed with Alzheimer's disease. The mammals treated with the inhibitors may be human primates, nonhuman primates or non-human mammals (e.g., rodents, canines). In one embodiment, the mammal is administered a compound herein that reduces β-secretase activity (inhibits memapsin 1 and memapsin 2 activity). In another embodiment, the mammal is administered a compound that selectively reduces memapsin 2 activity. In a related embodiment, the compound has minimal or no effect on reducing memapsin 1 activity. Therefore, also provided is a method of treating Alzheimer's disease in a subject in need thereof, the method comprising administering to the subject an effective amount of a β-secretase inhibitor compound. In an exemplary embodiment, the β-secretase inhibitor compound is part of a pharmaceutical formulation, as described above.

The inhibitor compounds herein can be employed in the treatment of diseases or conditions in an individual associated with β-secretase activity (e.g., memapsin 2 activity), which can halt, reverse or diminish the progression of the disease or condition, in particular Alzheimer's disease. In some embodiments, the individual has one or more symptoms of the disease or condition associated with β-secretase activity. In some embodiments, the individual has been diagnosed with disease or condition associated with β-secretase activity. In addition to compounds that decrease memapsin 2 activity, compounds that selectively reduce memapsin 2 activity are useful to treat diseases or conditions or biological processes associated with memapsin 2 activity rather than diseases or conditions or biological processes associated with both memapsin 2 activity and another peptide hydrolase (such as cathepsin D or memapsin 1).

For example, both memapsin 1 and memapsin 2 cleave amyloid precursor protein (APP) at a β-secretase site to form β-amyloid protein (also referred to herein as Aβ or (β-amyloid protein). Thus, both memapsin 1 and memapsin 2 have β-secretase activity (Hussain, I., et al., J. Biol. Chem. 276:23322-23328 (2001)). However, the β-secretase activity of memapsin 1 is significantly less than the β-secretase activity of memapsin 2 (Hussain, I., et al., J. Biol. Chem. 276:23322-23328 (2001)). Memapsin 2 is localized in the brain, and pancreas, and other tissues (Lin, X., et al., Proc. Natl. Acad. Sci. USA 97.1456-1460 (2000)) and memapsin 1 is localized preferentially in placentae (Lin, X., et al., Proc. Natl. Acad. Sci. USA 97:1456-1460 (2000)). Alzheimer's disease is associated with the accumulation of Aβ in the brain as a result of cleaving of APP by β-secretase (also referred to herein as memapsin 2, ASP2 and BACE). Thus, methods employing the compounds which selectively inhibit memapsin 2 activity relative to memapsin 1 activity may be important in the treatment of memapsin 2-related diseases, such as Alzheimer's disease. Selective inhibition of memapsin 2 activity makes the compounds herein suitable drug candidates for use in the treatment of Alzheimer's disease.

### VIII. METHODS OF TREATING GLAUCOMA

In another aspect, the β-secretase inhibitor compounds herein can be employed in the treatment of diseases associated with vision loss (e.g., glaucoma). In some embodiments, are provided a method of treating glaucoma (e.g. closed-angle glaucoma and open-angle glaucoma) in an individual comprising the step of administering to the individual in need thereof an effective amount of the β-secretase inhibitors herein (e.g., any compound of formula I, II, III, Example 2 and/or Table 1). In an exemplary embodiment, the β-secretase inhibitor compound is part of a pharmaceutical formulation, as described above.

In some aspects, the inhibitor compounds herein (e.g., any compound of formula I, II, III, Example 2 and/or Table 1) can be employed in the treatment of diseases or conditions associated with β-secretase activity, which can halt, reverse or diminish the progression of glaucoma (e.g. closed-angle glaucoma and open-angle glaucoma). In some embodiments, the inhibitor compounds herein can be used to halt, reverse or diminish the loss of retinal ganglion cells (RGCs). In other embodiments, compounds herein (e.g., any compound of formula I, II, III, Example 2 and/or Table 1) are employed to improve or decrease intraocular pressure (IOP).

Compounds described herein (e.g., any compound of formula I, II, III, Example 2 and/or Table 1) may be used to treat glaucoma by one of several known routes of administration, including, but not limited to, orally (e.g., in tablet or capsule form), parenterally (e.g., injected into the anterior chamber, intravenous, intramuscular, or subcutaneous), or locally (e.g., topical eye drops or ointment). Compounds herein may also be formulated for sustained release during glaucoma treatment.

Additional embodiments for treating glaucoma with compounds herein (e.g., any compound of formula I, II, III, Example 2 and/or Table 1) are described by adapting one or more of the methods in Guo, et. al. Proc. Natl. Acad. Sci., 14, 13444-13449 (2007); Yamamoto, et. al., Neuroscience Letters, 370, 61-64 (2004); and/or Urcola et. al., Exp. Eye Research, 83, 429-437 (2006). The content of these applications are hereby incorporated by reference in its entireties.

A. Methods of Administering Beta-Secretase Inhibitors to the CNS

The inhibitor compounds of herein (e.g., any compound of formula I, II, III, Example 2 and/or Table 1) may be administered to the CNS through either invasive or non-invasive methods. Non-invasive methods of administration include those methods that do not require the use of a mechanical or physical means to breach the integrity of the blood-brain barrier. Typically, non-invasive methods include the use of immunoliposomes, blood-brain barrier disruption (BBBD), or the olfactory pathway.

Immunoliposomes are liposomes with antibodies or antibody fragments that bind to receptors or transporters expressed on brain capillary endothelial cells attached to the surface of the liposome. An exemplary immunoliposome combines polymer (e.g., PEGylation) technology with that of chimeric peptide technology. For example, the β-secretase inhibitor may be packaged into a unilamellar lipid vesicle containing a PEG²⁰⁰⁰ derivative that contains a reactive groups at one end, for attachment to a complementary reactive group of an antibody or fragment thereof. Complementary reactive groups are well known in the art and, include, for example, amine and activated carboxylic acids, thiols and maleimides, and the like (Ambikanandan et al., J. Pharm Pharmaceut Sci 6(2):252-273 (2003); Huwyler et al., Proc. Natl. Acad. Sci. USA, 93:14164-14169 (1996); and Huwyler et al., J Pharmcol Exp Ther. 282:1541-1546 (1997); and U.S. Pat. No. 6,372,250, all of which are herein incorporated by reference for all purposes in their entirety).

Blood-brain barrier disruption is a temporal loss of the integrity of the tight junctions between endothelial cells that comprise the blood brain barrier. Typically, the compound is administered via systemic or intercarotid injection in conjuction with transient blood-brain barrier disruption (BBBD). Exemplary agents useful for inducing BBBD include solvents such as dimethyl sulfoxide (DMSO); ethanol (EtOH); metals (e.g., aluminum); X-irradiation; induction of pathological conditions (e.g., hypertension, hypercapnia, hypoxia, or ischemia); anti-neoplastic agents (e.g., VP-16, cisplatin, hydroxyurea, fluorouracil and etoposide); or concurrent systemic administration of the convulsant drug metrazol and the anti-convulsant drug pentobarbital (Ambikanandan et al., J. Pharm Pharmaceut Sci 6(2):252-273 (2003)); vasoactive leukotrienes (Black et al., J Neurosurg, 81(5):745-751 (1994)); intracarotid infusion of bradykinin, histamine, or the synthetic bradykinin compound RMP-7 (Miller et al., Science 297:1116-1118 (2002), Matsukado, et al., Neurosurgery 39:125-133 (1996), Abbott, et al., Mol Med Today 2:106-113 (1996), Emerich et al., Clin Pharmacokinet 40:105-123 (2001)); hyaluronidase (U.S. Patent Application Publication No. 20030215432, Kreil, et al. Protein Sci., 4(9):1666-1669 (1995)); and intercarotid injection of inert hypertonic solutions such as mannitol, or arabinose (Neuwelt, E. A., et al., in Neuwelt E A (ed), Implications of the Blood Brain Barrier and its Manipulation: Clinical Aspects. Vol. 2, Plenum Press, New York, (1989), Neuwelt, et al., J Nucl Med, 35:1831-1841 (1994), Neuwelt et al., Pediatr Neurosurg 21:16-22 (1994), Kroll et al., Neurosurg, 42:1083-1099 (1998), Rapoport, Cell Mol Neurobiol 20:217-230 (2000), and Doran et al., Neurosurg 36:965-970, (1995)).

Olfactory pathway administration is the intranasal delivery of the compound to the olfactory nerves in the upper third of the nasal passages. After intranasal delivery, the compound is transported back along the sensory olfactory neurons to yield significant concentrations in the cerebral spinal fluid (CSF) and olfactory bulb (Thorne et al., Brain Res, 692 (1-2):278-282 (1995); Thorne et al., Clin Pharmacokinet 40:907-946 (2001); Illum, Drug Discov Today 7:1184-1189 (2002); U.S. Pat. No. 6,180,603; U.S. Pat. No. 6,313,093; and U.S. Patent Application Publication No. 20030215398).

Invasive methods of administration are those methods that involve a physical breach of the blood-brain barrier typically through a mechanical or physical means to introduce the compound into the CSF, or directly into the parenchyma of the brain. Typically, invasive methods of administration may include injection or surgical implantation of the compound.

In injection methods, a needle is used to physically breach the BBB and deliver the compound directly into the CSF. Exemplary injection methods include intraventricular, intrathecal, or intralumbar routes of administration and may also involve infusion of the compound through a reservoir external to the body (Krewson et al., Brain Res 680:196-206 (1995); Harbaugh et al., Neurosurg. 23(6):693-698 (1988); Huang et al., J Neurooncol 45:9-17 (1999); Bobo et al., Proc Natl Acad Sci USA 91:2076-2082 (1994); Neuwalt et al., Neurosurg. 38(4):1129-1145 (1996)).

In surgical implantation methods, the compound is placed directly into the parenchyma of the brain. Exemplary surgical implantation methods may include incorporation of the compound into a polyanhydride wafer placed directly into the interstitium of the brain (Bremet al., Sci Med 3(4):1-11 (1996); Brem et al., J Control Release 74:63-67 (2001)).

### IX. CRYSTALLIZED COMPLEXES

In another aspect, is provided a crystallized complex containing a memapsin 2 protein and a β-secretase inhibitor herein. Memapsin 2 proteins useful in forming co-crystals with isostere compounds (e.g., memapsin 2 protein fragments, transmembrane proteins, etc.) have been previously discussed in detail (see U.S. Patent Application Publication No. 20040121947, and International Application No. PCT/US02/34324 (Publication No. WO 03/039454)). These memapsin 2 proteins are equally useful in forming crystallized complexes with β-secretase inhibitors described herein (e.g., any compound of formula I, II, III, Example 2 and/or Table 1).

The crystallized complex may be formed employing techniques described in U.S. Patent Application Publication No. 20040121947, and International Application No. PCT/USO2/34324 (Publication No. WO 03/039454). Briefly, a nucleic acid construct encoding the protein is generated, is expressed in a host cell, such as a mammalian host cell (e.g., Hela cell, 293 cell) or a bacterial host cell (e.g., E. coli), is purified and is crystallized with a compound or compounds herein. The diffraction resolution limit of the crystallized protein can be determined, for example, by x-ray diffraction or neutron diffraction techniques.

In an exemplary embodiment, the crystallized protein may have an x-ray diffraction resolution limit not greater than about 4.0Δ. The crystallized protein may also have an x-ray diffraction resolution limit not greater than about 4.0Δ, about 3.5Δ, about 3.0Δ, about 2.5Δ, about 2.0Δ, about 1.5Δ, about 1.0Δ, or about 0.5Δ. In some embodiments, the crystallized protein may also have an x-ray diffraction resolution limit not greater than about 2Δ. The diffraction resolution limit of the crystallized protein can be determined employing standard x-ray diffraction techniques.

In another exemplary embodiment, the β-secretase inhibitor of the crystallized complex is in association with said protein at an S₃′ binding pocket, an S₄′ binding pocket and/or an S₄ binding pocket. S₃′, S₄′, and S₄ binding pockets are discussed in detail in U.S. Patent Application Publication No. 20040121947, and International Application No. PCT/USO2/34324 (Publication No. WO 03/039454).

The terms and expressions which have been employed herein are used as terms of description and not of limitation, and there is no intention in the use of such terms and expressions of excluding equivalents of the features shown and described, or portions thereof, it being recognized that various modifications are possible. Moreover, any one or more features of any embodiment described herein may be combined with any one or more other features of any other embodiment described herein, without departing from the envisioned scope. For example, the features of the β-secretase inhibitors described herein are equally applicable to the methods of treating disease states and/or the pharmaceutical formulations described herein. All publications, patents, and patent applications cited herein are hereby incorporated by reference in their entirety for all purposes.

### IX. EXAMPLES

### Example I

### Preparation of Selected Beta-Secretase Inhibitors and Precursor Compounds

The described synthesis of Beta-Secretase inhibitors and precursor compounds is related to WO 2006/110668, filed on Apr. 10, 2006 and entitled “Compounds Which Inhibit Beta-Secretase Activity and Methods of Use Thereof,” the content of which is incorporated herein by reference in its entirety, and particularly with respect to the synthetic methods described therein, e.g., paragraphs 150-153 and paragraphs 215-285; and U.S. Provisional Patent Application No. 60/952,198, filed on Jul. 26, 2007 and entitled “Compounds Which Inhibit Beta-Secretase Activity and Methods of Use Thereof,” the content of which is incorporated herein by reference in its entirety, and particularly with respect to the synthetic methods described therein, e.g., paragraphs 83-86 and paragraphs 161-354.

The precursor compounds synthesized below are useful in the methods of making compounds provided herein. Using the guidance provided, (for example, in the Exemplary Syntheses of Scheme 1) one skilled in the art will immediately recognize that the exemplified synthesis of the below precursor compounds may be modified using well known techniques and the teaching provided herein to arrive at a wide variety of inhibitor compounds (e.g., compounds of Example 2). Certain starting materials described, and some precursor compounds not described, may be commercially available and purchased from, for example, Sigma-Aldrich, Alfa Aesar, or Ryan Scientific.

NMR spectra were collected on a Varian Mercury model VX-300 NMR spectrometer. NMR solvents were purchased from Cambrige Isotope Laboratories.

Solvents used in the synthesis of inhibitor compounds were purchased from Aldrich, VWR, and EMD. Solvents were ACS Reagent Grade or higher, and used without further purification.

### Example 1.1

### Synthesis of Amine Building Blocks

### Example 1.1.1

### 4-methylthiazol-2-yl)methanamine

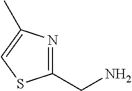

Methylthiazole (1.0 g, 10.1 mmol) in tetrahydrofuran (THF) at −78° C. was treated with n-BuLi (1.6 M, 7.56 mL) for 30 min, N,N-dimethylformamide (DMF) (1.4 mL, 18.2 mmol) was added dropwise. The resulting reaction mixture was warmed to r.t. After the starting material disappeared (by TLC), the reaction mixture was recooled to 0° C. and LAH (0.69 g, 18.5 mmol) was added. The mixture was warmed to r.t. and stirred for 1 h, the reaction was quenched with aqueous NH₄Cl, diluted with EtOAc. The organic solution was separated, extracted twice with EtOAc, dried with Na₂SO₄, and concentrated. The residue was purified with flash chromatography to give the corresponding alcohol as a light yellow oil. ¹H-NMR: (300 MHz, CDCl₃), d: 6.89 (s, 1H); 4.95 (s, 2H); 2.48 (s, 3H).

Methylthiazole methanol (0.57 g, 4.4 mmol) was treated with mesyl chloride (0.42 mL, 5.4 mmol) and triethylamine at 0° C. in dichloromethane. The resulting mixture was stirred for 20 minutes followed by quenching with aqueous NH₄Cl. Evaporation of the solvent from the organic layer and flash chromatography of the residue afforded the corresponding mesylate as an oil. The mesylate (0.25 g, 1.2 mmol) was then dissolved in DMF and sodium azide (0.62 g, 9.6 mmol) was added. The mixture was heated to reflux for 2 hours followed by cooling and washing with aqueous NH₄Cl. Evaporation of the solvent from the organic layer resulted in the corresponding azide. The azide (0.14 g, 0.91 mmol) was dissolved in ethyl acetate, Pd(OH)₂ (0.07 g) was added, and the suspension was stirred under a hydrogen atmosphere for 5 hours. The suspension was filtered through Celite. Evaporation of the solvent and flash chromatography of the residue afforded the desired methylthiazole methylamine as a yellow oil. ¹H-NMR: (300 MHz, CDCl₃), d: 6.74 (m, 1H); 4.09 (m, 2H); 2.37 (s, 3H).

Using an alternative synthetic route, NaBH₄ (0.75 g, 19.9 mmol, 1.3 eq) was added to a stirred solution of 4-methylthiazole-2-carbaldehyde (Aldrich, 1.7 ml, 2.0 g, 15.3 mmol, 1 eq) in 30 ml anhydrous MeOH at 0° C. After 45 min the solvent was removed in vacuo. The residue was diluted with saturated aqueous NH₄Cl and extracted with EtOAc (×3). The combined organics were washed with brine (×1) and dried over Na₂SO₄. The inorganics were filtered off, and the solvent was removed in vacuo. Purification via flash chromatography yielded (4-methylthiazol-2-yl)methanol in quantitative yield.

Diphenylphosphoryl azide (DPPA) (1.2 eq) and 1,8-Diazabicyclo(5.4.0)undec-7-ene (DBU) (1.2 eq) were added to a stirred solution of (4-methylthiazol-2-yl)methanol (1 eq) in 7 ml anh. toluene under Ar. After stirring overnight, the solvent was removed in vacuo. Purification via flash chromatography yielded 2-(azidomethyl)-4-methylthiazole.

2-(azidomethyl)-4-methylthiazole was dissolved in 5 ml MeOH. Pd(OH)₂ (20% by wt. on carbon) was added and the mixture was stirred vigorously under H₂ overnight. The mixture was filtered through Celite, and the filter cake rinsed with MeOH. The solvent was removed in vacuo yielding (4-methylthiazol-2-yl)methanamine.

### Example 1.1.2

### N-methyl-1-(4-methylthiazol-2-yl)methanamine

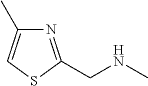

Ti(OⁱPR)₄ (1.3 eq) was added with stirring to MeNH₂ (2.0 M in MeOH, 3 eq) at 0° C. under Ar. After 15 min. 4-methylthiazole-2-carbaldehyde (1 eq) was added, and the solution was stirred for 2-3 h. NaBH₄ (1.4 eq, in batches if large scale) was added and stirred at 0° C. to RT overnight, followed by solvent removal in vacuo. The residue was diluted with water/CH₂Cl₂, and a white ppt formed. The mixture was then filtered through Celite to remove the white ppt and the layers were separated. The aqueous layer was extracted with CH₂Cl₂ (×3) and the combined organics were dried over Na₂SO₄. The inorganics were filtered off, and the solvent was removed in vacuo to give the crude product. Purification via column chromatography yielded the pure product in 80-90% yield.

### Example 1.2

### Synthesis of Cyclic Amine Building Blocks

### Example 1.2.1

### (R)-4-methyl-2-(pyrrolidin-2-yl)thiazole

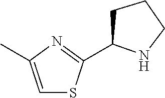

To a solution of the commercially available (R)-1-(benzyloxycarbonyl)pyrrolidine-2-carboxylic acid (Synthetech, 9.97 g, 40.0 mmoles) in 1,4-dioxane (60 mL) was added Pyridine (2 mL), (Boc)₂O (11.35 mL, 52 mmoles) and NH₄HCO₃ (3.98 g, 50.4 mmoles) and stirred for 12 h. All solvent was evaporated, diluted with EtOAc and washed with water, 5% H₂SO₄ and brine. The organic layer was dried over anhydrous Na₂SO₄ and concentrated. (R)-benzyl 2-carbamoylpyrrolidine-1-carboxylate was generated in quantitative yield and used in the following step without further purification.

To a solution of (R)-benzyl 2-carbamoylpyrrolidine-1-carboxylate (9.97 g, 40.0 mmoles) in 1,2-dimethoxyethane (2000 mL) was added Lawesson's reagent (8.9 g, 0.55 mmoles) and stirred for 4 h. All solvent was evaporated, diluted with 100 mL of saturated NaHCO₃ and extracted with ether (2×200 mL). The combined organic layers was dried over anhydrous Na₂SO₄ and concentrated. Crude (R)-benzyl 2-carbamothioylpyrrolidine-1-carboxylate was carried on to the next step without further purification.

To a solution of (R)-benzyl 2-carbamothioylpyrrolidine-1-carboxylate (˜40 mmoles) in EtOH (120 mL) was added chloroacetone (4.7 mL, 60 mmoles) and heated at 75° C. for 6 h. The reaction was cooled to room temperature and poured into 100 mL of saturated aq. NaHCO₃ solution. Ethanol was evaporated under reduced pressure and the aqueous layer was extracted with ethyl acetate (2×200 mL). The combined organic layers was dried over Na₂SO₄ and concentrated. The residue was chromatographed on silica gel (35% ethyl acetate/80% hexane) to generate (R)-benzyl 2-(4-methylthiazol-2-yl)pyrrolidine-1-carboxylate in 86% yield after three steps.

HBr in AcOH (60 mL) was added to (R)-benzyl 2-(4-methylthiazol-2-yl)pyrrolidine-1-carboxylate (neat) at room temperature. After 1 h, ether (150 mL) was added slowly with vigorous stirring. Stirring was continued for 10 min and allowed to settle for 5-10 min. The supernatant was decanted. This process was repeated 3-4 times until the supernatant was colourless. The semi-solid was dissolved in water (50 mL) and brought to P^{H}˜8 with 1N LiOH and extracted with 5% MeoH/95% CHCl₃ (3×100 mL) to yield 4.0 g of (R)-4-methyl-2-(pyrrolidin-2-yl)thiazole.

### Example 1.2.2

### (S)-4-methyl-2-(pyrrolidin-2-yl)thiazole

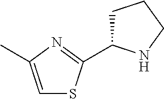

(S)-4-methyl-2-(pyrrolidin-2-yl)thiazole was prepared following the same procedure as in the preparation of (R)-4-methyl-2-(pyrrolidin-2-yl)thiazole starting from the commercially available Cbz-L-proline (Aldrich).

### Example 1.2.3

### (R)-4-methyl-2-(pyrrolidin-2-yl)oxazole

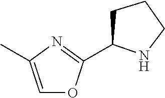

To a solution of L-Serine methyl ester hydrochloride (Aldrich, 5.0 g, 32.0 mmoles), in CH₂Cl₂ (150 mL) at 0° C., were added Et₃N (4.88 mL, 35.2 mmoles), Cbz-D-Proline (8.01 g, 32.0 mmoles) and DCC (7.26 g, 35.2 mmoles) sequentially. The reaction was allowed to warm to room temperature and stirred overnight. All the solvent was evaporated and the residue was triturated with ethyl acetate and the precipitate was filtered off. The filtrate was concentrated under low pressure and chromatographed on silica gel (70% ethyl acetate/30% chloroform) to yield 8.5 g of (R)-benzyl 2-((S)-3-hydroxy-1-methoxy-1-oxopropan-2-ylcarbamoyl)pyrrolidine-1-carboxylate.

Deoxo-flour (4.5 mL, 24.16 mmoles) was added drop-wise to a solution of (R)-benzyl 2-((S)-3-hydroxy-1-methoxy-1-oxopropan-2-ylcarbamoyl)pyrrolidine-1-carboxylate (8.5 g, 22.0 mmoles) in CH₂Cl₂ (150 mL) at −20° C. The solution was stirred for 30 min and BrCCl₃ (7.8 mL, 79.0 mmoles) was added drop-wise followed by DBU (11.8 mL, 79 mmoles). The reaction was stirred at 2-3° C., for 10 h., quenched with Satd. Aq. NaHCO₃ solution and extracted with ethyl actetate. The organic layer was concentrated and chromatographed on silica gel (10% ethyl acetate/90% chloroform) to yield 6.95 of (R)-methyl 2-(1-(benzyloxycarbonyl)pyrrolidin-2-yl)oxazole-4-carboxylate.

To a solution of (R)-methyl 2-(1-(benzyloxycarbonyl)pyrrolidin-2-yl)oxazole-4-carboxylate (6.95 g, 21.1 mmoles) in THF (50 mL) at 0° C., was added LiBH₄ (32 mL, 2.0M in THF, 63.2 mmoles). The reaction was allowed to warm to room temperature and stirred for 3 h. Ethyl acetate (25 mL) was added drop-wise and stirred for 30 min. The reaction was cooled to 0° C. and 50 mL of 1N HCl was added drop-wise and diluted with 100 mL of water. It was then extracted with ethyl acetate, dried on Na₂SO₄, concentrated, and chromatographed on silica gel (3% MeOH/97% chloroform) to yield 4.1 g of (R)-benzyl 2-(4-(hydroxymethyl)oxazol-2-yl)pyrrolidine-1-carboxylate.

To a solution of (R)-benzyl 2-(4-(hydroxymethyl)oxazol-2-yl)pyrrolidine-1-carboxylate (1.1 g, 3.64 mmoles) in hexamethylphosphoraminde (HMPA) (18 mL), was added methyltriphenoxyphosphonium iodide (3.29 g, 7.28 mmoles) and stirred for 30 min. Then Na(CN)BH₃ was added and the reaction was heated at 50° C. for 3 h and poured into 100 mL of ice-cold water and extracted with ether (2×100 mL). The organic layer was dried on Na₂SO₄, concentrated, and chromatographed on silica gel (50% ethyl acetate/50% hexanes) to yield 180 mg of (R)-benzyl 2-(4-methyloxazol-2-yl)pyrrolidine-1-carboxylate.

HBr in AcOH (60 mL) was added to (R)-benzyl 2-(4-methyloxazol-2-yl)pyrrolidine-1-carboxylate (neat) at room temperature. After 1 h, ether (20 mL) was added slowly with vigorous stirring. Stirring was continued for 10 min and allowed to settle for 5-10 min. The supernatant was decanted. This process was repeated 3-4 times until the supernatant was colourless. The semi-solid was dissolved in water (50 mL) and brought to pH ˜8 with 1N LiOH and extracted with 5% MeoH/95% CHCl₃

### Example 1.3

### Synthesis of Isophthalate Building Blocks

### Example 1.3.1

### 3-(methoxycarbonyl)-5-(N-methylmethylsulfonamido)benzoic acid

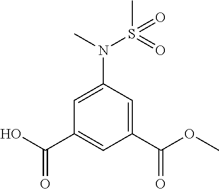 [SMILES: COC(=O)c1cc(C(=O)O)cc(N(C)S(C)(=O)=O)c1]

To a stirred solution of dimethyl 5-aminoisophthalate (2.09 g, 10 mmol) in dichloromethane (30 mL), pyridine (2.43 mL, 30 mmol) was added at room temperature. At 0° C., methanesulfonyl chloride (0.86 mL, 11 mmol) was added and the resulting mixture was stirred overnight at room temperature. The reaction mixture was then concentrated under reduced pressure and ethyl acetate (50 mL) was added. The resulting white precipitate was filtered and washed with hexanes to give dimethyl 5-(methylsulfonamido)isophthalate in 95% (2.715 g) yield as a white solid.

To a stirred suspension of NaH (0.24 g, 10 mmol, 60% in oil dispersion) in 10 mL of DMF was added dimethyl 5-(methylsulfonamido)isophthalate (1.435 g, 5 mmol) followed by iodomethane (0.62 mL, 10 mmol) at room temperature. After 5 h, the reaction was quenched by H₂O (25 mL). Then the reaction mixture was extracted with EtOAc, further washed with H₂O to remove excess of DMF, dried over anhydrous Na₂SO₄ and concentrated. The crude product thus obtained was washed with hexanes to give dimethyl 5-(N-methylmethylsulfonamido)isophthalate as a white solid in 91% (1.37 g) yield.

Dimethyl 5-(N-methylmethylsulfonamido)isophthalate (0.842 g, 2.8 mmol) was dissolved in THF:MeOH (1:1) (8 mL) and H₂O (3 mL). Solid NaOH (0.112 g, 2.8 mmol) was added and stirred at room temperature for 18 h. The reaction mixture was concentrated under reduced pressure. Saturated NaHCO₃ (10 mL) was added to the reaction mixture and extracted with toluene (to remove <10% unreacted starting material). The aqueous solution was acidified with dilute HCl (10%), extracted with EtOAc, and dried over anhydrous Na₂SO₄. The solvent was evaporated and dried under reduced pressure to give 3-(methoxycarbonyl)-5-(N-methylmethylsulfonamido)benzoic acid as a white solid (75%, 0.598 g), which was used without further purification.

### Example 1.3.2

### 5-fluoroisophthalic acid

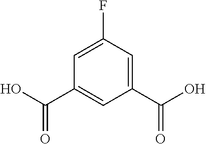 [SMILES: O=C(O)c1cc(F)cc(C(=O)O)c1]

To a gently refluxing solution of 1.9 g (15.3 mmol) of 5-fluoro-m-xylene in about 13.5 mL of pyridine and about 9.5 mL of water was added 13.8 g (87.3 mmol) of KMnO₄ in several portions. The mixture was refluxed for about 7 h, followed by the addition of sodium sulfite to quench the excess KMnO₄. The warm mixture was filtered, and 1N HCl was added to a pH=3. The filtrate was washed with EtOAc, saturated with NaCl, and extracted with the extract of a mixture of (80 mL CHCl₃: 10 mL MeOH: 10 mL H₂O) 3-4 times. The combined extracts were dried over sodium sulfate, filtered, and concentrated to give about 400 mg (14% yield) of 5-fluoroisophthalic acid as a pale yellow solid.

### Example 1.3.3

### 3-(methoxycarbonyl)-5-methylbenzoic acid

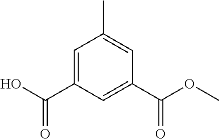

To 5-methylisophthalic acid (Aldrich, 5 g, 27.7) in MeOH (37.5 ml)/THF (112.5 ml), conc. H₂SO₄ (1.25 ml) was added and stirred at 65° C. for 8 h. Reaction mixture was cooled to room temperature and solvent removed. Then reaction mixture was diluted with water and extracted with ethylacetate. Crude residue was column chromatographed to yield 2.5 g of 3-(methoxycarbonyl)-5-methylbenzoic acid as a white solid.

### Example 1.3.4

### dimethyl 5-(oxazol-2-yl)isophthalate

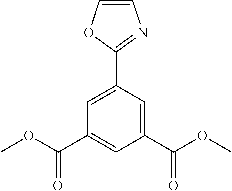

To a stirred solution of oxazole ((0.28 mL, 4.2 mmol) in THF (10 mL) at −78° C. was added nBuLi (2.8 mL 1.6 N solution in hexane, 4.4 mmol). ZnCl₂ (20 mL 0.5M soln, 10 mmol) was added after 30 min and the reaction mixture was warmed up to 0° C. for 1 h. To the resulting mixture was added dimethyl 5-iodoisophthalate (1.28 g, 4.0 mmol) and Pd(PPh₃)₄ and was heated at reflux for 5 h. The reaction mixture was cooled to room temperature and diluted with EtOAc and H₂O. The layers were separated and the organic layer was washed with, brine, dried with Na₂SO₄ and concentrated under reduced pressure. The residue was purified by column chromatography (20% EtOAc in hexanes) to provide dimethyl 5-(oxazol-2-yl)isophthalate (568 mg, 54%).

### Example 1.3.5

### 3-(methoxycarbonyl)-5-(oxazol-5-yl)benzoic acid

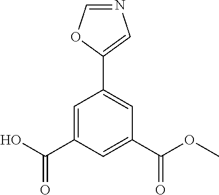 [SMILES: COC(=O)c1cc(C(=O)O)cc(-c2cnco2)c1]

To a stirred solution of diethyl 5-hydroxyisophthalate (4.0 g, 15.9 mmol) in HOAc (40 mL) was added a solution of CAN (19 g, 34.9 mmol) in H₂O (40 mL) dropwise. The reaction mixture was heated at 70° C. for 6 h during which time the color of the solution turned from red to colorless. The reaction mixture was cooled to room temperature and dilute with H₂O and was extracted with EtOAc. The combined organic layer was washed with saturated aqueous NaHCO₃, brine, dried with Na₂SO₄ and concentrated under reduced pressure to provide diethyl 5-formylisophthalate (3.93 g, 99%) as a white solid. ¹H NMR (CDCl₃): d 10.17 (s, 1H), 8.95-8.96 (m, 1H), 8.74-8.75 (m, 2H), 4.50 (q, J=7.2 Hz, 4H), 1.47 (t, J=7.2 Hz, 6H).

To a stirred solution of diethyl 5-formylisophthalate (529 mg, 2.1 mmol) and p-toluenesulfonylmethyl isocyanide (483 mg, 2.5 mmol) in DME (15 mL) and MeOH (15 mL) was added K₂CO₃. The resulting mixture was heated to reflux for 4 h and cooled to room temperature. The solvent was removed and the residue was dissolved in EtOAc and H₂O. The layers were separated and the aqueous layer was extracted with EtOAc (2×20 mL). The combined organic layer was washed with brine, dried with Na₂SO₄ and concentrated under reduced pressure to provide 9 (103 mg, 19%). ¹H NMR (CDCl₃): d 8.63 (s, 1H), 8.49 (s, 2H), 8.00 (s, 1H), 7.54 (s, 1H), 4.00 (s, 6H).

### Example 1.3.6

### dimethyl 5-(1H-pyrrol-1-yl)isophthalate

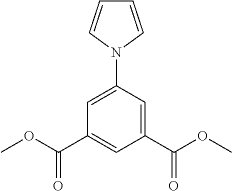

2,5-dimethoxytetrahydrofuran (0.74 ml, 0.76 g, 5.74 mmol, 1.2 eq) was added to a stirred suspension of dimethyl 5-aminoisophthalate (1.0 g, 4.78 mmol, 1 eq) in 7 ml acetic acid under Ar. The mixture was heated to reflux at 135° C. After 45 min the reaction was cooled to RT, and the solvent was removed in vacuo. The residue was stirred in saturated aqueous NaHCO₃/EtOAc overnight. The layers were separated. The organic layer was washed with saturated aqueous NaHCO₃ (×1), water (×2), brine (×1), and dried over Na₂SO₄. The inorganics were filtered off, and the solvent was removed in vacuo. Purification via flash chromatography yielded 0.288 g (1.11 mmol, 23% yield) of the product. A significant amount of crude product was also collected.

### Example 1.3.7

### dimethyl 5-(pyridin-2-yl)isophthalate

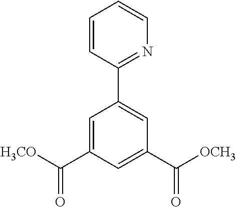 [SMILES: COC(=O)c1cc(C(=O)OC)cc(-c2ccccn2)c1]

To dimethyl 5-iodoisophthalate (Matrix Scientific, 800 mg, 2.5 mmol) in THF (20 ml), 2-pyridine boronic acid N-phenyldiethanol amine ester (Aldrich, 1.8 g, 6.6 mmol), K₂CO₃ (912 mg, 6.6 mmol), triphenyl phosphine (173 mg, 0.66 mmol) were added followed by Pd(OAc)₂ and cuprous iodide (251 mg, 1.32 mmol). After refluxing for 24 h, reaction mixture was filtered through a pad of celite. Residual solvent was evaporated on a rotavap under reduced pressure and the crude was dissolved in ethyl acetate. Insoluble material was filtered off and the remaining residue was evaporated to dryness and column purified (60% ethylacetate/40% hexanes) to yield 400 mg of dimethyl 5-(pyridin-2-yl)isophthalate as yellow solid.

### Example 1.3.8

### dimethyl 5-(pyrazin-2-yl)isophthalate

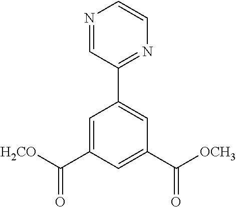

To dimethyl 5-bromoisophthalate (617 mg, 2.26 mmol) in toluene (10 ml), 2-tributylstannyl pyrazine (1 g, 2.71 mmol) was added followed by Pd(PPh₃)₄ (102 mg, 0.09 mmol). Then reaction mixture was refluxed for 22 h. Then the reaction mixture was filtered through celite and volatiles were removed under vacuum. Crude residue was column chromatographed (50% ethylacetate/50% Hexanes) to obtain 455 mg of dimethyl 5-(pyrazin-2-yl)isophthalate as a pale yellow solid.

### Example 1.3.9

### dimethyl 5-morpholinoisophthalate

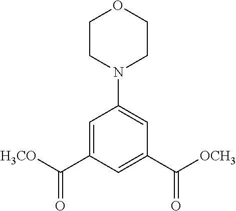 [SMILES: COC(=O)c1cc(C(=O)OC)cc(N2CCOCC2)c1]

To dimethyl 5-bromoisophthalate (Matrix Scientific; 1.0 g, 3.66 mmol) in toluene (10 ml), morpholine (sigma-aldrich) (351 mgg, 4.03 mmol) was added followed by BINAP (sigma-aldrich) (100 mg, 0.16 mmol), cesium carbonate (sigma-aldrich) (1.7 g, 5.12 mmol) and Pd(OAc)₂ (sigma-aldrich) (25 mg, 0.11 mmol). Then reaction mixture was heated at 80° C. for 48 h. Then the reaction mixture was filtered through celite and volatiles were removed under vacuum. Crude residue was partitioned between ethyl acetate and water. Organic layer was washed with water, brine, dried and concentrated. Resultant residue was column chromatographed (30% ethylacetate/70% Hexanes) to obtain 550 mg of dimethyl 5-morpholinoisophthalate as a pale yellow syrup.

### Example 1.3.10

### 2′,4′-difluorobiphenyl-3,5-dicarboxylic acid

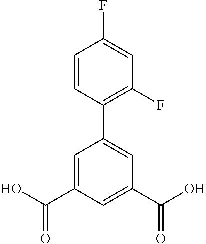 [SMILES: O=C(O)c1cc(C(=O)O)cc(-c2ccc(F)cc2F)c1]

A solution containing dimethyl 5-bromoisophthalate (1.7 g, 6.3 mmol), 2,4-difluorophenylboronic acid (1.0 g, 6.3 mmol) and Na₂CO₃ (25 mL, 1M aqueous solution) in DMF (30 mL) was degassed under Ar for 10 min. Pd(PPh₃)₄ (727 mg, 0.63 mmol) was added and the mixture was degassed for 2 min. The resulting mixture was heated to 85° C. for 4 h and cooled to room temperature. The mixture was diluted with NH₄Cl and extracted with EtOAc (3×30 mL). The aqueous layer was acidified to pH 3 with 1N HCl and extracted with EtOAc. The combined organic layer was washed with brine, dried with Na₂SO₄ and concentrated under reduced pressure. The residue was purified by column chromatography (10% methanol in chloroform) to provide 2′,4′-difluoro-5-(methoxycarbonyl)biphenyl-3-carboxylic acid (107 mg) as a colorless oil and 2′,4′-difluorobiphenyl-3,5-dicarboxylic acid (533 mg) as an off white solid.

### Example 1.3.11

### 3-methoxy-5-(methoxycarbonyl)benzoic acid

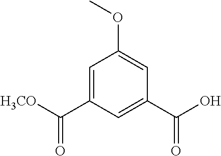

1 N NaOH (0.9 eq) was added to a stirred solution of dimethyl 5-methoxyisophthalate (Aldrich, 1 eq) in 1:3 MeOH/THF (volume of MeOH^{˜} volume of NaOH). After stirring overnight, the solvent was removed via rotary evaporation and the residue was diluted with saturated aqueous NaHCO₃. The mixture was extracted with EtOAc (×2). The aqueous layer was adjusted to pH^{˜} 3 with concentrated HCl, and extract with EtOAc (×3). The appropriate organics were combined, washed with water (×1), brine (×1), and dried over Na₂SO₄. The inorganics were filtered off, and the solvent was removed via rotary evaporation yielding the product.

### Example 1.3.12

### dimethyl 5-(4-chlorobutanamido)isophthalate

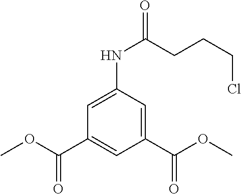

1 drop of Et₃N (catalytic) was added to a stirred solution of 4-chlorobutanoic acid (0.029 ml, 0.35 g, 2.87 mmol 1.2 eq) in SOCl₂ (2 ml, 3.27 g, 27.5 mmol, 11.5 eq) and the mixture was heated to 80° C. After 1.5 h the reaction was cooled to room temperature, and the solvent was removed in vacuo. The flask was evacuated and back-filled with Ar (×3). The residue was dissolved in 2 ml anhydrous CH₂Cl₂. The resulting solution was added dropwise to a stirred suspension of dimethyl 5-aminoisophthalate in 8 ml anhydrous CH₂Cl₂. After 1 h Et₃N (1 ml, 0.73 g, 7.17 mmol, 3 eq) was added. After 2 h the solvent was removed in vacuo, and the resulting residue was dissolved EtOAc. The organic layer was washed with saturated aqueous NaHCO₃ (×2), water (×3), brine (×1), and dried over Na₂SO₄. The inorganics were filtered off, and the solvent was removed in vacuo. Purification via flash chromatography yielded 0.6353 g (2.0 mmol, 85% yield) of the product.

### Example 1.3.13

### dimethyl 5-(2-oxopyrrolidin-1-yl)isophthalate

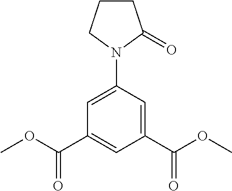

A solution of dimethyl 5-(4-chlorobutanamido)isophthalate (0.635 g, 2.02 mmol, 1 eq) dissolved in 5 ml anhydrous DMF was added dropwise to a stirred suspension of NaH (60% dispersion in oil, 0.101 g, 2.53 mmol, 1.25 eq) in 2 ml anhydrous DMF at 0° C. under Ar. The reaction was stirred at 0° C. to room temperature overnight. After stirring overnight the reaction was heated to 100° C. for 19 h. After cooling to room temperature the reaction was poured into ice-water to quench. The mixture was extracted with EtOAc (×1). The organic layer was washed with water (×4), brine (×1), and dried over Na₂SO₄. The inorganics were filtered off, and the solvent was removed in vacuo. Purification via flash chromatography yielded 0.3487 g (1.26 mmol, 62% yield) of the product.

### Example 1.3.14

### dimethyl 5-(dimethylamino)isophthalate

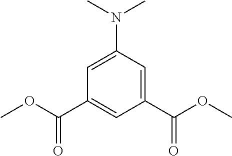

CH₂O (aq, 37%) (3.2 ml, 3.49 g, 43.0 mmol, 6 eq) was added to a stirred solution of the diester (1.5 g, 7.17 mmol, 1 eq) in CH₃CN (50 ml) at 0° C. After 15 min NaBH₃CN (1.09 g, 16.49 mmol, 2.3 eq) was added. The reaction was adjusted to pH^{˜}7 with HOAc. Stir at 0° C. to RT overnight. The solvent was removed in vacuo, and the residue was partitioned between EtOAc and saturated aqueous NaHCO₃. The layers were separated. The organic layer was washed with water (×3), brine (×1), and dried over Na₂SO₄. The inorganics were filtered off, and the solvent was removed in vacuo. Purification via flash chromatography yielded 1.62 g (6.83 mmol, 95% yield) of dimethyl 5-(dimethylamino)isophthalate.

### Example 1.3.15

### dimethyl 5-(trifluoromethoxy)isophthalate

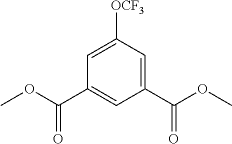

1,3-dibromo-5-(trifluoromethoxy)benzene (Aldrich, 0.6 g, 1.9 mmol, 1 eq) was dissolved in anhydrous DMF (4 ml) under Ar. After degassing with Ar for 5 min Pd(PPh₃)₄ (0.6502 g, 0.56 mmol, 30 mol %) and Zn(CN)₂ (0.2422 g, 2.06 mmol, 1.1 eq) were added sequentially to the reaction. The mixture was heated to 85° C. with stirring overnight. After cooling to 0° C. the reaction was diluted with Et₂O and quenched with excess NH₄OH. After stirring for 1 h at 0° C. the layers were separated. The organic layer was washed with water (×4), brine (×1), and dried over Na₂SO₄. The inorganics were filtered off, and the solvent was removed via rotary evaporation. Purification via flash chromatography yielded 0.3126 g (1.5 mmol, 78% yield) of the 5-(trifluoromethoxy)isophthalonitrile.

5-(trifluoromethoxy)isophthalonitrile (0.3126 g, 1.5 mmol, 1 eq) in EtOH (6 ml) was treated with 1N KOH (6 ml, 6 mmol, 4 eq) and refluxed at 80° C. overnight. After cooling to room temperature the volatiles were removed via rotary evaporation. The mixture was adjusted to pH=1-2 with concentrated HCl. The solution was extracted with 10% MeOH in CHCl₃ (×4). The combined organics were dried over Na₂SO₄. The inorganics were filtered off, and the solvent was removed via rotary evaporation yielding crude 5-(trifluoromethoxy)isophthalic acid which was used without purification.

MeOH (0.24 ml, 0.19 g, 6 mmol, 4 eq) was added to a stirred suspension of the crude 5-(trifluoromethoxy)isophthalic acid (˜1.5 mmol) in anhydrous CH₂Cl₂ (10 ml) under Ar. After cooling to 0° C. the mixture was treated sequentially with 1,3-Dicyclohexylcarbodiimide (DCC, 0.6499 g, 3.15 mmol, 2.1 eq) and 4-Dimethylaminopyridine (DMAP, 0.0183 g, 0.15 mmol, 10 mol %). After stirring overnight the reaction was diluted with saturated aqueous NaHCO₃ and the resulting mixture was filtered through cotton. The layers were separated, and the organic layer was dried over Na₂SO₄. The inorganics were filtered off, and the solvent was removed via rotary evaporation. Purification via flash chromatography yielded 0.265 g (0.95 mmol, 64% yield from 5-(trifluoromethoxy)isophthalonitrile) of dimethyl 5-(trifluoromethoxy)isophthalate as a colorless oil.

### Example 1.3.16

### dimethyl 3′-(trifluoromethyl)biphenyl-3,5-dicarboxylate

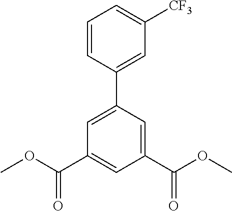

Dimethyl 5-bromoisophthalate (Commercial source: Matrix Scientific) (1.106 g, 4.05 mmol) in toluene (15 ml), m-trifluoromethyl phenyl boronic acid (Commercial source: sigma-aldrich) (1.0 g, 5.26 mmol) was added followed by S-Phos (commercial source: Alfa-Aesar) (66 mg, 0.16 mmol), K₃PO₄ (commercial source: Alfa-Aesar) (1.72 g, 8.10 mmol) and Pd(OAc)₂ (commercial source: sigma-aldrich) (18 mg, 0.08 mmol). Then reaction mixture was heated at 90° C. for 1.5 h, then filtered through celite and volatiles were removed under vacuum. The crude residue was partitioned between diethyl ether and water. The organic layer was washed with 1N sodium hydroxide solution, water, and brine, then dried with anhydrous sodium sulfate concentrated. The resultant residue was purified by column chromatography (10% ethylacetate/90% Hexanes) to obtain 1.0 g of dimethyl 3′-(trifluoromethyl)biphenyl-3,5-dicarboxylate as a white solid.

### Example 1.3.17

### dimethyl 5-(4,4-difluoropiperidin-1-yl)isophthalate

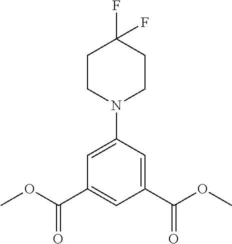

To dimethyl 5-bromoisophthalate (Commercial source: Matrix Scientific) (1.57 g, 5.77 mmol) in toluene (15 ml) was added 4,4-difluoropiperidine HCl salt (Commercial source: sigma-aldrich) (1.0 g, 6.34 mmol) followed by BINAP (commercial source: sigma-aldrich) (162 mg, 0.26 mmol), cesium carbonate (commercial source: sigma-aldrich) (4.5 g, 13.85 mmol) and Pd(OAc)₂ (commercial source: sigma-aldrich) (39 mg, 0.173 mmol). Then reaction mixture was heated at 80° C. for 48 h, then filtered through celite and volatiles were removed under vacuum. The crude residue was partitioned between diethyl ether and water. The organic layer was washed with 6N HCl, water, brine and dried with anhydrous sodium sulfate. The resultant residue was concentrated and purified by column chromatography (20% ethylacetate/80% Hexanes) to obtain 800 mg of dimethyl 5-(4,4-difluoropiperidin-1-yl)isophthalate as a pale yellow syrup.

### Example 1.3.18

### dimethyl 5-cyclopropylisophthalate

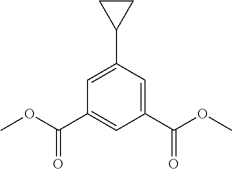

A round bottom flask was charged with Pd(OAc)₂ (commercial source: sigma-aldrich) (41 mg, 0.18 mmol), XPhos (commercial source: sigma-aldrich) (175 mg, 0.366 mmol), potassiumcyclopropyltrifluoroborate (commercial source: sigma-aldrich) (1.57 g, 10.98 mmol), and K₃PO₄ (commercial source: Alfa-Aesar) (5.83 g, 27.45 mmol). Then, under Argon, dimethyl 5-bromoisophthalate (2.5 g, 9.15 mmol) and toluene/H₂O (3:1) (40 mL) were added by syringe, and the reaction was stirred at 100° C. for 24 h, cooled to room temperature, and diluted with H₂O. The reaction mixture was extracted with ethyl acetate. The organic layer was dried (Na₂SO₄). The solvent was removed in vacuo, and the crude product was purified by silica gel column chromatography (elution with hexane/EtOAc 90:10) to yield 1.1 g of dimethyl 5-cyclopropylisophthalate as a pale yellow solid.

### Example 1.3.19

### dimethyl 5-(trifluoromethyl)isophthalate

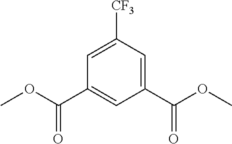

A mixture of Methyl-2,2-difluoro-2-(fluorosulfonyl)acetate (Commercial source: sigma-aldrich) (3.5 ml, 27.49 mmol), copper iodide (Commercial source: sigma-aldrich) (2.74 g, 14.37 mmol) and dimethyl 5-iodoisophthalate (Commercial source: Matrix Scientific) (4.0 g, 12.5 mmol) in DMF (25 ml) was stirred under argon atmosphere for 6 h at 70° C. The reaction was then cooled to room temperature and diluted with dichloromethane (DCM), washed with water, dried with Na₂SO₄, and concentrated to provide a syrup. Purification was done by column chromatography (10% ethylacetate/90% Hexanes) to give 1.5 g of pure dimethyl 5-(trifluoromethyl)isophthalate as a white solid.

### Example 1.3.20

### dimethyl 5-(trifluoromethyl)isophthalate

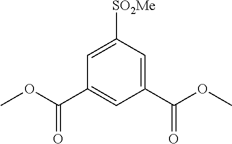

To dimethyl 5-iodoisophthalate (Commercial source: Matrix Scientific) (3.2 g, 9.99 mmol) was added sodium methane sulfinate (Commercial source: sigma-aldrich) (1.22 g, 11.99 mmol) in DMSO (20 ml), N,N′-dimethylethylenediamine (Commercial source: sigma-aldrich) (88 mg, 0.99 mmol) and (CuOTf)₂. PhH (Commercial source: sigma-aldrich) (251 mg, 0.499 mmol). The reaction mixture was heated at 110° C. for 24 h, then was cooled to room temperature and diluted with ethyl acetate. The precipitated solids were filtered and the filtrate was washed with water, brine and dried. The crude residue was purified by column chromatography to provide dimethyl 5-(trifluoromethyl)isophthalate as a pale yellow solid.

### Example 1.3.21

### dimethyl 5-(1H-imidazol-1-yl)isophthalate

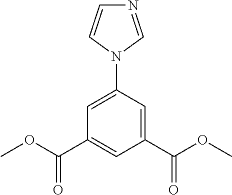

A mixture of imidazole (Commercial source: sigma-aldrich) (580 mg, 8.53 mmol), potassium carbonate (5.18 g, 37.5 mmol), copper iodide (Commercial source: sigma-aldrich) (357 mg, 1.87 mmol), L-Proline (431 mg, 3.75 mmol) and dimethyl 5-iodoisophthalate (Commercial source: Matrix Scientific) (3.0 g, 9.37 mmol) in DMSO (25 ml) was stirred under argon atmosphere for 24 h at 110° C. The reaction was cooled to room temperature then diluted with ethylacetate and filtered through celite. The filtrate was washed with water and organic layer dried with Na₂SO₄ and concentrated to afford a syrup. The concentrate was purified by column chromatography (90% ethylacetate/10% Hexanes) to provide 500 mg of dimethyl 5-(1H-imidazol-1-yl)isophthalate as a white solid.

### Example 1.3.22

### dimethyl 5-(thiazinanyl-S,S-dioxide)isophthalate

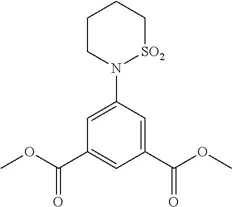

A round bottom flask was charged with 1,4-butane-sultam (1.07 g, 7.91 mmol) (commercial source: Combi-blocks), palladium acetate (15 mg, 0.066 mmol) (Commercial source: sigma-aldrich), Xantphos (57 mg, 0.099 mmol) (Commercial source: sigma-aldrich) and cesium carbonate (3.0 g, 9.23 mmol) (Commercial source: sigma-aldrich). Dioxane (15 ml) was added, followed by dimethyl 5-bromoisophthalate (1.8 g, 6.59 mmol)(Commercial source: Matrix Scientific). The flask was then heated to 100° C. for 30 h and then cooled to room temp and diluted with dichloromethane. The slurry was filtered through celite pad. The volatiles were removed and the crude material was chromatographed (90% ethyl acetate/10% Hexanes) to obtain 700 mg of dimethyl 5-(thiazinanyl-S,S-dioxide)isophthalate as a pale yellow solid.

### Example 1.3.23

### dimethyl 5-(1-methyl-1H-pyrazol-4-yl)isophthalate

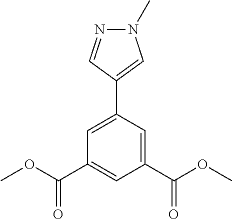

To a mixture of dimethyl 5-bromoisophthalate (2.1 g, 8.0 mmol) (Commercial source: Matrix Scientific), 1-Methyl-4-pyrazoelboronic acid pinacol ester (2.0 g, 9.61 mmol) (Commercial source: sigma-aldrich) and K₂CO₃ (3.32 g, 24.0 mmol) (Commercial source: sigma-aldrich) in 40 ml of dioxane and 16 ml of water dichloro[1,1′-bis(diphenylphosphino)ferrocene]palladium (II) dichloromethane adduct (653 mg, 0.80 mmol) (Commercial source: sigma-aldrich) was added. The reaction mixture was heated at 80° C. for 6 h, then concentrated in vacuo. The residue was purified by flash column chromatography with 70% ethyl acetate/30% Hexanes to obtain 1.3 g of dimethyl 5-(1-methyl-1H-pyrazol-4-yl)isophthalate as a brown solid.

### Example 1.3.23

### dimethyl 5-(1,1-dioxidoisothiazolidin-2-yl)isophthalate

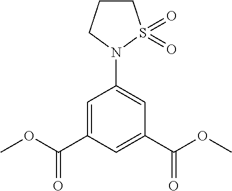

Aqueous ammonia (excess) was added into an ice-cooled solution of 3-chloropropanesulfonyl chloride (commercial source: Sigma-Aldrich) (5 g, 28.24 mmol) in DCM (20 ml). The reaction solution was stirred overnight, and then, 50 ml of water was added to the reaction mixture. The DCM layer was separated and the aqueous layer was extracted with 100 ml of DCM. The combined organic layers were dried over sodium sulfate and concentrated to dryness under reduced pressure. The residue was recrystallized from n-hexane to obtain 3.3 g of 3-chloropropanesulfonamide.

To 3-chloropropanesulfonamide (3.3 g, 20.88 mmol) in EtOH (30 ml), sodium ethoxide (1.42 g, 20.88 mmol) was added. The reaction mixture was the refluxed for 72 h. Then EtOH was removed and the crude residue was triturated with DCM. The organic layer was concentrated and purified by column chromatography to yield 1.6 g of isothiazolidine 1,1-dioxide.

A round bottom flask was charged with isothiazolidine 1,1-dioxide (1.6 g, 13.2 mmol), palladium acetate (247 mg, 1.1 mmol) (Commercial source: sigma-aldrich), Xantphos (955 mg, 1.65 mmol) (Commercial source: sigma-aldrich) and cesium carbonate (5.02 g, 15.40 mmol) (Commercial source: sigma-aldrich). Dioxane (40 ml) was added, followed by dimethyl 5-bromoisophthalate (3.0 g, 11.0 mmol)(Commercial source: Matrix Scientific). The flask was then heated to 100° for 6 h and then cooled to room temperature and diluted with dichloromethane. The slurry was filtered through celite pad. The volatiles were removed and the crude material was chromatographed (90% ethyl acetate/10% Hexanes) to obtain 2.51 g of dimethyl 5-(1,1-dioxidoisothiazolidin-2-yl)isophthalate.

### Example 1.4

### Isophthalate/Amine Coupling

### Example 1.4.1

### (R)-3-(2-(4-methylthiazol-2-yl)pyrrolidine-1-carbonyl)benzoic acid

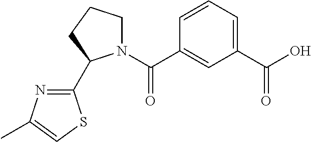 [SMILES: Cc1csc([C@H]2CCCN2C(=O)c2cccc(C(=O)O)c2)n1]

To mono-methyl isopthalate (Aldrich, 124 mg, 0.7 mmol) in CH₂Cl₂ (4 mL) at room temperature, thionyl chloride (5 ml) was added and reaction mixture was refluxed for 2 h. Then the volatiles were removed on a rotavap under reduced pressure. To that mixture, (R)-4-methyl-2-(pyrrolidin-2-yl)thiazole was added followed by triethylamine (1 drop). The reaction mixture was stirred at rt for 3 h, then diluted with ethyl acetate, washed with water, brine, and dried. Crude residue was purified by column chromatography to yield 155 mg of (R)-methyl 3-(2-(4-methylthiazol-2-yl)pyrrolidine-1-carbonyl)benzoate.

To the solution of (R)-methyl 3-(2-(4-methylthiazol-2-yl)pyrrolidine-1-carbonyl)benzoate (155 mg, 0.49 mmol) in THF (5 mL) was added 1N LiOH (2 mL) and the reaction mixture was stirred at rt for 1 h. Then the volatiles were removed on a rotavap under reduced pressure. Then reaction mixture was diluted with water, acidified with 1N HCl to pH ˜3 and extracted with ethyl acetate. Organic layer was dried and evaporated to yield 132 mg of the acid (R)-3-(2-(4-methylthiazol-2-yl)pyrrolidine-1-carbonyl)benzoic acid.

### Example 1.4.2

### (R)-3-fluoro-5-(2-(4-methylthiazol-2-yl)pyrrolidine-1-carbonyl)benzoic acid

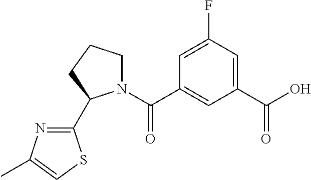 [SMILES: Cc1csc([C@H]2CCCN2C(=O)c2cc(F)cc(C(=O)O)c2)n1]

Following general coupling reaction conditions described, (R)-4-methyl-2-(pyrrolidin-2-yl)thiazole (182 mg, 1.1 mmol) and 5-fluoroisophthalic acid (210 mg, 1.1 mmol) were coupled to provide (R)-3-fluoro-5-(2-(4-methylthiazol-2-yl)pyrrolidine-1-carbonyl)benzoic acid (232.4 mg, 63%) as an off white solid.

### Example 1.4.3

### (R)-methyl 3-(hydroxymethyl)-5-(2-(4-methylthiazol-2-yl)pyrrolidine-1-carbonyl)benzoate

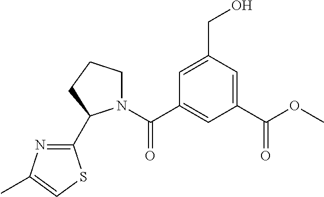

A solution of (R)-4-methyl-2-(pyrrolidin-2-yl)thiazole (511 mg, 3.037 mmol) and 3-(hydroxymethyl)-5-(methoxycarbonyl)benzoic acid (702.5 mg, 3.34 mmol) in DCM (50 mL) were added diisopropylethylamine (3 mL, excess), HOBt (410 mg, 3.34 mmol) and EDCI (754.1 mg, 3.948 mmol). The resulting solution was stirred at room temperature for overnight. The reaction mixture was diluted with chloroform, washed with sodium bicarbonate saturated aqueous solution and separated. The aqueous layer was extracted one more time with chloroform. The combined organic layers were concentrated to give a residue, which was purified with flash chromatography to produce the desired compound (840 mg). ¹H NMR (300 MHz, CDCl₃), d: 8.011 (m, 1.5H), 7.876 (br, 0.5H), 7.683 (m, 1H), 6.749 (m, 1H), 5.579 (m, 0.7H), 5.061 (br, 0.3H), 4.641 (br, 1.2H), 4.525 (br, 0.8H), 3.875 (m, 3H), 3.692 (m, 1H), 3.457 (m, 1H), 2.345 (m, 5H), 2.034 (m, 2H).

### Example 1.4.4

### 3-(methyl((4-methylthiazol-2-yl)methyl)carbamoyl)benzoic acid

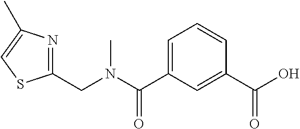 [SMILES: Cc1csc(CN(C)C(=O)c2cccc(C(=O)O)c2)n1]

Mono-Methyl isophthalate (0.054 g, 0.30 mmol) was treated with EDCI (0.064 g, 0.33 mmol), HOBt (0.046 g, 0.34 mmol), DIPEA (0.07 mL, 0.4 mmol), and methylthiazole methylamine (0.046 g, 0.36 mmol). The resulting mixture was stirred at room temperature for 15 h under argon followed by quenching with water. The layers were separated and the aqueous layer was extracted with CHCl₃ (2×20 mL). The combined organic layers were dried with Na₂SO₄ and concentrated under reduced pressure. The resulting oil was dissolved in THF (5 mL) to which was added 3 mL of 1.0N LiOH_{(aq)}. The resulting mixture was stirred rapidly for 1.5 h. The volatiles were removed via rotary evaporation and the resulting aqueous solution was extracted with CHCl₃ (×3). The aqueous solution was then acidified to pH 1 with 1N HCl_{(aq)} and extracted with CHCl₃ (×3). The combined organic layers were dried with Na₂SO₄ and concentrated under reduced pressure to provide the corresponding isophthalic acid. This product (0.042 g, 0.11 mmol) was dissolved in DMF and treated with NaH (0.015 g, 0.62 mmol) and MeI (0.04 mL, 0.64 mmol) and stirred overnight. The volatiles were removed via rotary evaporation and the resulting solution was diluted with 1N LiOH and extracted with CHCl₃ (×3). The aqueous solution was then acidified to pH 1 with 1N HCl_{(aq)} and extracted with CHCl₃ (×3). The combined organic layers were dried with Na₂SO₄ and concentrated under reduced pressure to provide N-Methyl-N-(4-methyl-thiazol-2-ylmethyl)-isophthalamic acid. ¹H-NMR: (300 MHz, CDCl₃), d: 8.16 (m, 2H), 7.70 (m, 1H), 7.51 (m, 1H), 6.91 (s, 1H), 5.05 (s, 1.5H), 4.75 (s, 0.5H), 3.2-3.0 (m, 3H), 2.46 (s, 3H).

### Example 1.4.5

### (R)-3-(N-methylmethylsulfonamido)-5-(1-phenylethylcarbamoyl)benzoic acid

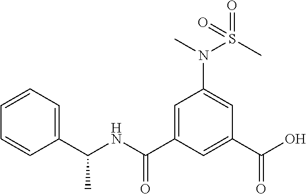 [SMILES: C[C@@H](NC(=O)c1cc(C(=O)O)cc(N(C)S(C)(=O)=O)c1)c1ccccc1]

To a stirred solution of 3-(methoxycarbonyl)-5-(N-methylmethan-5-ylsulfonamido)benzoic acid (0.215 g, 0.75 mmol), EDC (0.172 g, 0.9 mmol), HOBt (0.122 g, 0.9 mmol) in DMF/CH₂Cl₂ (1:5 mL) at room temperature was added α-methylbezylamine (0.1 mL, 0.75 mmol) followed by diisopropylethylamine (0.5 mL). The reaction mixture was stirred at room temperature for 16 h. Then water was added and the reaction mixture was extracted with EtOAc. The organic layers were dried over Na₂SO₄ and concentrated. The crude product thus obtained was purified by silica gel flash column chromatography (3% MeOH in CHCl₃) to provide the corresponding amide 10 (0.343 g) which was dissolved in THF:MeOH (1:1) (6 mL) and H₂O (2 mL). Solid NaOH (80 mg, 2.0 mmol) was added and stirred at room temperature for 6 h. The reaction mixture was concentrated under reduced pressure. Saturated NaHCO₃ (10 mL) solution was added to the reaction mixture and extracted with toluene (to remove organic impurities). The aqueous reaction mixture was acidified with diluted HCl (10%), extracted with EtOAc, dried over anhydrous Na₂SO₄. The solvent was evaporated and dried under reduced pressure to give 3-(N-methylmethan-5-ylsulfonamido)-5-((1-phenylethyl)carbomoyl)benzoic acid (0.198 g, 60%,) as a white solid.

### Example 1.4.6

### 3-(methyl((4-methylthiazol-2-yl)methyl)carbamoyl)-5-(N-methyl-methylsulfonamido)benzoic acid

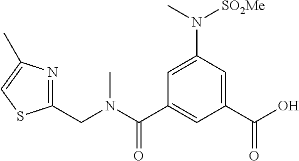 [SMILES: Cc1csc(CN(C)C(=O)c2cc(C(=O)O)cc(N(C)S(C)(=O)=O)c2)n1]

To a stirred solution of 3-(methoxycarbonyl)-5-(N-methylmethylsulfonamido)benzoic acid (0.393 g, 1.37 mmol), N-methyl-1-(4-methylthiazol-2-yl)methanamine (185 mg, 1.3 mmol) in DCM were added triethylamine (1 mL, excess), Py-BOP (784 mg, 1.507 mmol) at room temperature. The reaction mixture was stirred at room temperature for 16 h. Then water was added and the reaction mixture was extracted with EtOAc. The organic layers were dried over Na₂SO₄ and concentrated. The crude product thus obtained was purified by silica gel flash column chromatography (2% MeOH in ethyl acetate) to provide the corresponding amide (0.510 g) which was dissolved in THF:MeOH (1:1) (15:15 mL) and H₂O (2 mL) Solid NaOH (146 mg, 3.645 mmol) was added and stirred at 50° C. for 1 hour. The reaction mixture was concentrated under reduced pressure. Saturated NaHCO₃ (10 mL) solution was added to the reaction mixture and extracted with toluene (to remove organic impurities). The aqueous reaction mixture was acidified with diluted HCl (10%), extracted with EtOAc, dried over anhydrous Na₂SO₄. The solvent was evaporated and dried under reduced pressure to give the crude 3-(methyl((4-methylthiazol-2-yl)methyl)carbamoyl)-5-(N-methyl-methylsulfonamido)benzoic acid which was used directly in the next step.

### Example 1.4.7

### (R)-3-(2-(4-methylthiazol-2-yl)pyrrolidine-1-carbonyl)-5-(oxazol-2-yl)benzoic acid

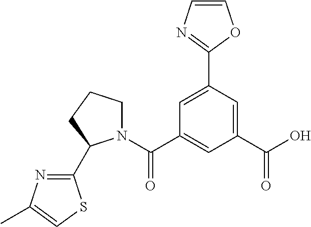 [SMILES: Cc1csc([C@H]2CCCN2C(=O)c2cc(C(=O)O)cc(-c3ncco3)c2)n1]

EDCI.HCl (0.372 g, 1.9 mmol, 1.3 eq) and HOBT.H₂O (0.202 g, 1.5 mmol, 1.0 eq) were added to a stirred solution of 3-(methoxycarbonyl)-5-(oxazol-2-yl)benzoic acid (0.37 g, 1.5 mmol, 1 eq) in 8 ml anhydrous CH₂Cl₂ at 0° C. under Ar. The resulting solution was treated with a solution of DIPEA (0.78 mL, 4.5 mmol, 3.0 eq) and (R)-4-methyl-2-(pyrrolidin-2-yl)thiazole (0.252, 1.5 mmol, 1.0 eq) in 2 ml anhydrous CH₂Cl₂. The reaction was stirred at 0° C. to room temperature overnight. The solvent was removed via rotary evaporation. The residue was quenched with water, and the resulting mixture was extracted with EtOAc (×1). The organic layer was washed with water (×2), brine (×1), and dried over Na₂SO₄. The inorganics were filtered off, and the solvent was removed via rotary evaporation. Purification via flash chromatography on silica gel yielded 0.8308 g (2.5 mmol, 65% yield) of (R)-methyl 3-(2-(4-methylthiazol-2-yl)pyrrolidine-1-carbonyl)-5-(oxazol-2-yl)benzoate.

1N LiOH (3.0 mL) was added to a stirred solution of (R)-methyl 3-(2-(4-methylthiazol-2-yl)pyrrolidine-1-carbonyl)-5-(oxazol-2-yl)benzoate (0.38 g, 2.5 mmol, 1 eq) in THF (3 ml) and. After stirring for 2 h, the medium was adjusted to pH^{˜} 3 with 1N HCl and extracted with 10% MeOH/90% EtOAc/(×2). The organics were combined and dried over Na₂SO₄. The inorganics were filtered off, and the solvent removed via rotary evaporation yielding 0.28 g (76% yield) of the product (R)-3-(2-(4-methylthiazol-2-yl)pyrrolidine-1-carbonyl)-5-(oxazol-2-yl)benzoic acid.

### Example 1.4.8

### (R)-2-(2-(4-methylthiazol-2-yl)pyrrolidine-1-carbonyl)isonicotinic acid

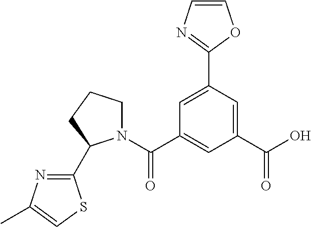 [SMILES: Cc1csc([C@H]2CCCN2C(=O)c2cc(C(=O)O)cc(-c3ncco3)c2)n1]

To a stirring mixture of 95.6 mg (0.528 mmol) of 4-(methoxycarbonyl)picolinic acid (commercially available from 3R-Chem), 111 mg (0.577 mmol) of EDCI and 80.7 mg (0.597 mmol) of HOBt in 5 mL of CH₂Cl₂ was added 92.7 mg (0.551 mmol) (R)-4-methyl-2-(pyrrolidin-2-yl)thiazole (J-Star Research, Inc.) and 400 μL of diisopropylethylamine in 10 mL of CH₂Cl₂. After the solution was stirred at r.t. for about 52 h, CHCl₃ and H₂O were added. The aqueous layer was extracted with CHCl₃, and the combined extracts were washed with H₂O (2×) and brine, dried over Na₂SO₄, filtered, and concentrated. Purification by flash silica gel chromatography (CombiFlash, 100% EtOAc) provided (R)-methyl 2-(2-(4-methylthiazol-2-yl)pyrrolidine-1-carbonyl)isonicotinate as an orange oil with some impurity.

A solution of 45.9 mg (0.139 mmol) of (R)-methyl 2-(2-(4-methylthiazol-2-yl)pyrrolidine-1-carbonyl)isonicotinate and 110 μL of 2N NaOH (aqueous) in 2 mL of THF and 1 mL of MeOH was stirred at r.t. for 5 h. Additional 2N NaOH (20 μL) was added, and after 45 min., the solution was concentrated. The pH was adjusted to 2 with 1N HCl, and water was also added. The aqueous layer was extracted with the extract of (40 mL of CHCl₃: 5 mL of H₂O: 5 mL of MeOH) 3×. The combined extracts were dried over Na₂SO₄, filtered, and concentrated to provide (R)-2-(2-(4-methylthiazol-2-yl)pyrrolidine-1-carbonyl)isonicotinic acid, which was used in the next reaction without further purification.

### Example 1.5

### Coupled Amide Modifications

### Example 1.5.1

### (R)-methyl 3-formyl-5-(2-(4-methylthiazol-2-yl)pyrrolidine-1-carbonyl)benzoate

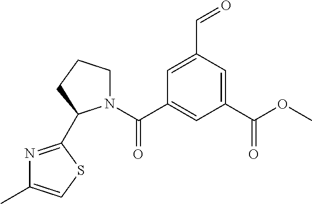

To a solution of (R)-methyl 3-(hydroxymethyl)-5-(2-(4-methylthiazol-2-yl)pyrrolidine-1-carbonyl)benzoate (560 mg, 1.554 mmol) in DCM (60 mL), Dess-Martin periodinane (790.8 mg, 1.864 mmol) was added at rt. After stirring for 2 hrs, the mixture was poured into a mixture of aqueous 1 M Na₂S₂O₃ (30 mL) and aqueous saturated NaHCO₃ (30 mL), and it was extracted with DCM three times. The combined organic layers were concentrated in vacuum and the residue was purified by flash silica chromatography to give the product (530 mg). ¹H NMR (CDCl₃): d: 10.094, 9.933 (s, s, 1H), 8.592-7.908 (m, 3H), 6.796 (s, 1H), 5.661 (m, 0.65H), 5.083 (m, 0.35H), 3.969-3.743 (m, 4H), 3.515 (m, 1H), 2.429-2.308 (m, 5H), 2.145-1.939 (m, 2H).

### Example 1.5.2

### (R)-methyl 3-(fluoromethyl)-5-(2-(4-methylthiazol-2-yl)pyrrolidine-1-carbonyl)benzoate

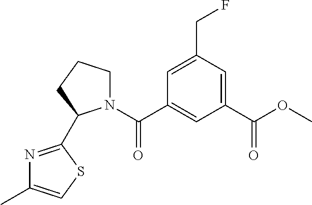

(R)-methyl 3-(hydroxymethyl)-5-(2-(4-methylthiazol-2-yl)pyrrolidine-1-carbonyl)benzoate (280 mg, 0.777 mmol) in dry DCM (40 mL) at −78° C. was added [Bis(2-methoxyethyl)amino]sulfur trifluoride (0.17 mL, 0.932 mmol) slowly and stirred at the same temperature for 2 hrs, then warmed to room temperature for overnight. The reaction was carefully quenched with aqueous saturated NaHCO₃, extracted with chloroform three times. The combined organic solvent was dried with anhydrous Na₂SO₄, removed in vacuum and the residue was purified by silica gel chromatography to afford monofluoride (177 mg). ¹H NMR (CDCl₃): d: 8.211-7.784 (m, 2.7H), 7.420 (s, 0.3H), 6.778 (s, 1H), 5.645-5.076 (m, 3H), 3.929-3.741 (m, 4H), 3.519 (m, 1H), 2.428-2.325 (m, 5H), 2.088-1.930 (m, 2H).

### Example 1.5.3

### (R)-3-(difluoromethyl)-5-(2-(4-methylthiazol-2-yl)pyrrolidine-1-carbonyl)benzoic acid

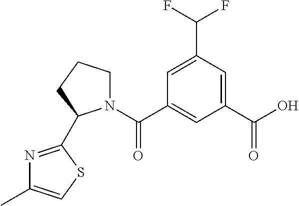 [SMILES: Cc1csc([C@H]2CCCN2C(=O)c2cc(C(=O)O)cc(C(F)F)c2)n1]

To a solution of (R)-methyl 3-formyl-5-(2-(4-methylthiazol-2-yl)pyrrolidine-1-carbonyl)benzoate (530 mg, 1.47 mmol) in CH₂Cl₂ (50 mL) at −78° C. was added [Bis(2-methoxyethyl)amino]sulfur trifluoride (0.46 mL, 2.49 mmol) slowly, then a couple drops of ethanol was added, and the mixture was stirred at same temperature for 2 hr. The resulting mixture was warmed to room temperature and stirred overnight. The solution was slowly poured into saturated NaHCO₃, extracted with methylene chloride three times, dried (Na₂SO₄), filtered, and evaporated in vacuo. Flash chromatography on silica gel afforded the pure (R)-methyl 3-(difluoromethyl)-5-(2-(4-methylthiazol-2-yl)pyrrolidine-1-carbonyl)benzoate (442 mg). ¹H NMR (CDCl₃): d: 8.330-7.919 (m, 2.7H), 7.528 (s, 0.3H), 6.902-6.368 (m, 3H), 5.638 (m, 0.7H), 5.048 (m, 0.3H), 3.946-3.746 (m, 4H), 3.488 (m, 1H), 2.412-2.312 (m, 5H), 2.112-1.950 (m, 2H).

Solid NaOH (63.14 mg, 1.578 mmol) was added to the solution of the above ester (460 mg, 1.212 mmol) in THF/MeOH/H₂O (15 mL/15 mL/2 mL) and stirred at 50° C. for 1 hour. The reaction mixture was concentrated under reduced pressure. Diluted with water and acidified with diluted HCl (10%), extracted with EtOAc, dried over anhydrous Na₂SO₄. The solvent was evaporated and dried under reduced pressure to give the pure (R)-3-(difluoromethyl)-5-(2-(4-methylthiazol-2-yl)pyrrolidine-1-carbonyl)benzoic acid as a white solid which was used directly for next step reaction without further identification.

### Example 1.6

### Hydroxy Amine Pyrrolidine Synthesis

### Example 1.6.1

### (R)-tert-butyl 2-((1S,2S)-2-amino-1-hydroxy-3-phenylpropyl)pyrrolidine-1-carboxylate

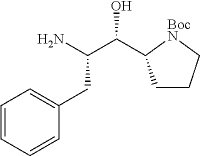 [SMILES: CC(C)(C)OC(=O)N1CCC[C@@H]1[C@@H](O)[C@@H](N)Cc1ccccc1]

To a stirred solution of (S)-2-(dibenzylamino)-3-phenylpropan-1-ol (5 g, 15 mmol) in DMSO (20 mL) at 0° C. was added Et₃N (8.4 mL, 60 mmol) and SO₃.Py. The resulting mixture was stirred for 1 h and diluted with H₂O (20 mL) and EtOAc (30 mL) The layers were separated and the aqueous layer was extracted with EtOAc (2×30 mL). The combined organic layer was washed with H₂O, 5% citric acid, brine, dried with Na₂SO₄ and concentrated under reduced pressure. The residue was purified by column chromatography (10% EtOAc in hexanes) to provide (S)-2-(dibenzylamino)-3-phenylpropanal (4.42 g, 90%).

To a stirred solution of (−)-sparteine (1.8 g, 7.7 mmol) in ether (30 mL) at −78° C. was added sec-BuLi (7.2 mL, 10 mmol) dropwise followed by N-Boc-pyrrolidine (1.3 g, 7.7 mmol) in ether. The resulting mixture was stirred at −78° C. for 2 h and (S)-2-(dibenzylamino)-3-phenylpropanal (3.8 g, 11.5 mmol) in ether was added slowly. The reaction mixture was stirred for 20 min and HoAc (1 mL) was added and warmed up to r.t. H₂O was added and the layers were separated. The aqueous layer was extracted with EtOAc (2×30 mL). The combined organic layer was washed with 5% citric acid, brine, dried with Na₂SO₄ and concentrated under reduced pressure. The residue was purified by column chromatography (30% EtOAc in hexanes) to provide (R)-tert-butyl 2-((1S,2S)-2-(dibenzylamino)-1-hydroxy-3-phenylpropyl)pyrrolidine-1-carboxylate (1.6 g, 43%) as a pale yellow foamy solid.

A hydrogen balloon was put on a stirred solution of (R)-tert-butyl 2-((1S,2S)-2-(dibenzylamino)-1-hydroxy-3-phenylpropyl)pyrrolidine-1-carboxylate (1.5 g, 3.0 mmol), Pd(OH)₂ (500 mg) in MeOH (30 mL). The stirring was continued for 17 h and the resulting mixture was filtered through a pad of Celite. The filtrate was concentrated under reduced pressure to provide the product (R)-tert-butyl 2-((1S,2S)-2-amino-1-hydroxy-3-phenylpropyl)pyrrolidine-1-carboxylate (950 mg, 99%) as an off-white solid.

### Example 1.6.2

### (2R,4R)-tert-butyl 2-((1S,2S)-2-amino-1-hydroxy-3-phenylpropyl)-4-(benzyloxy)pyrrolidine-1-carboxylate

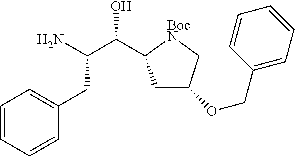 [SMILES: CC(C)(C)OC(=O)N1C[C@H](OCc2ccccc2)C[C@@H]1[C@@H](O)[C@@H](N)Cc1ccccc1]

Acetyl chloride (2.45 mL, 34.53 mmoles) was slowly added to MeOH (25 mL) in a reaction flask under inert atmosphere. To this was added a solution of Cis-4-Hydroxy-D-proline (3.235 g, 24.67 mmol) and refluxed for 8 h. The reaction mixture was cooled to room temperature, and poured into ether (200 mL). The precipitated solid was suction filtered and dried to yield (2R,4R)-methyl 4-hydroxypyrrolidine-2-carboxylate hydrochloride in quantitative yield. This was taken forward without purification.

To a solution of (2R,4R)-methyl 4-hydroxypyrrolidine-2-carboxylate hydrochloride (4.4 g, 24.67 mmol) in Acetone and water (3:2, 30 mL) were added Et₃N (6.8 mL, 49.28 mmol), DMAP (150 mg, 1.2 mmol). Then (Boc)₂O (8.0 mL, 34.54 mmol) was added slowly and the reaction was stirred overnight. All the acetone was removed and diluted with EtOAc and washed with 0.5 N HCl, water, brine, dried and concentrated to yield 6.0 g of (2R,4R)-1-tert-butyl 2-methyl 4-hydroxypyrrolidine-1,2-dicarboxylate (quantitative).

To a solution of (2R,4R)-1-tert-butyl 2-methyl 4-hydroxypyrrolidine-1,2-dicarboxylate (3.1 g, 12.64 mmol) in DMF (20 mL) at 0° C., were added BnBr (3.3 mL, 27.81 mmol) followed by Ag₂O (3.22 g, 13.90 mmol) and stirred for 36 h. Then 50 mL of ether was added to the reaction mixture and filtered. The filtrate was further diluted with ether and washed with water, brine, dried and concentrated. Purification with 30% EtOAc/70% Hexanes yielded 3.59 g (85%) of (2S,4R)-1-tert-butyl 2-methyl 4-(benzyloxy)pyrrolidine-1,2-dicarboxylate.

To a solution of (2S,4R)-1-tert-butyl 2-methyl 4-(benzyloxy)pyrrolidine-1,2-dicarboxylate (3.59 g, 10.7 mmol) in THF (25 mL) was added LiBH₄ (6.4 mL, 2.0 M in THF, 12.84 mmol) at 0° C. and reaction was allowed to warm to room temperature and stirred overnight. The reaction was then cooled to 0° C. and 30 mL of water was added slowly followed by a drop-wise addition of 1NHCl until PH˜4. Then it was extracted with EtOAc and washed with satd. NaHCO₃, brine, dried and concentrated. Purification with 40% EtOAc/70% Hexanes yielded 2.9 g (88%) of (2S,4R)-tert-butyl 4-(benzyloxy)-2-(hydroxymethyl)pyrrolidine-1-carboxylate.

To a solution of (2S,4R)-tert-butyl 4-(benzyloxy)-2-(hydroxymethyl)pyrrolidine-1-carboxylate (910 mg, 2.97 mmol) in DMSO at 0° C. was added Et₃N (1.65 mL, 11.89 mmol) and SO₃.Py (947 mg, 5.94 mmol). The reaction was warmed to RT and stirred for 30 min, diluted with ether and washed with 5% aq. citric acid, brine and dried to give quantitative yield of the (2S,4R)-tert-butyl 4-(benzyloxy)-2-formylpyrrolidine-1-carboxylate. This compound was then taken forward with either method described below without purification.

Method A: ⁿBuLi (1.6 M in hexanes, 7.6 ml, 12.2 mmol, 1.5 eq) was added slowly to a stirred solution of (S)-4-isopropyloxazolidin-2-one (Aldrich, 1.5 g, 11.6 mmol, 1 eq) in 20 ml anhydrous THF at −78° C. After 10 min 3-phenylpropanoyl chloride (Aldrich, 1.9 ml, 2.15 g, 12.8 mmol, 1.1 eq) was added dropwise. The reaction was warmed to 0° C. After 1 h the reaction was quenched with saturated aqueous NH₄Cl. The reaction was stirred at 0° C. to room temperature overnight. The reaction was partitioned between water/EtOAc, and the layers were separated. The organic layer was washed with water (×2), brine (×1), and dried over Na₂SO₄. The inorganics were filtered off, and the solvent was removed via rotary evaporation. Purification via flash chromatography on silica gel yielded 2.73 g (10.44 mmol, 90% yield) of (S)-4-isopropyl-3-(3-phenylpropanoyl)oxazolidin-2-one.

Bu₂BOTf (1.0 M in CH₂Cl₂, 9.7 ml, 9.7 mmol, 1.1 eq) was added to a stirred solution of (S)-4-isopropyl-3-(3-phenylpropanoyl)oxazolidin-2-one (2.2943 g, 8.78 mmol, 1 eq) in 40 ml anhydrous CH₂Cl₂ at 0° C. under Ar. After 5 min DIPEA (1.76 ml, 1.3 g, 10.97 mmol, 1.15 eq) was added very slowly. After 1 h the reaction was cooled to −78° C. A solution of (2S,4R)-tert-butyl 4-(benzyloxy)-2-formylpyrrolidine-1-carboxylate (2.6810 g, 8.78 mmol, 1 eq; synthesis described above) in 5 ml anhydrous CH₂Cl₂ was added dropwise. The reaction was stirred at −78° C. to room temperature overnight. The reaction was cooled to 0° C. and treated with pH=7 phosphate buffer (30 ml) followed by 30% aqueous H₂O₂ (2.6 ml). After 1 h the layers were separated. The aqueous layer was extracted with CH₂Cl₂ (×1). The combined organics were dried over Na₂SO₄. The inorganics were filtered off, and the solvent was removed via rotary evaporation. Purification via flash chromatography on silica gel yielded 3.243 g (5.72 mmol, 65% yield) of (4R)-tert-butyl 2-((1S,2S)-2-benzyl-1-hydroxy-3-((S)-4-isopropyl-2-oxooxazolidin-3-yl)-3-oxopropyl)-4-(benzyloxy)pyrrolidine-1-carboxylate.

(4R)-tert-butyl 2-((1S,2S)-2-benzyl-1-hydroxy-3-((S)-4-isopropyl-2-oxooxazolidin-3-yl)-3-oxopropyl)-4-(benzyloxy)pyrrolidine-1-carboxylate (2.4511 g, 4.33 mmol, 1 eq) was dissolved in THF/water (20 ml: 5 ml). The solution was capped with a rubber septum and cooled to 0° C. H₂O₂ (30% aqueous, 4.4 ml, 4.9 g, 43.3 mmol, 10 eq) was added dropwise with stirring. The solution was treated with a solution of LiOH.H₂O (0.3629 g, 8.65 mmol, 2 eq) dissolved in water (4 ml), and the cooling bath was removed. After 7-8 h the reaction was cooled to 0° C. and quenched with excess Na₂SO₃ (2M, 25 ml). After 30 min the solution was carefully adjusted to pH^{˜} 2 with 1N HCl and extracted with CH₂Cl₂ (×2). The combined organics were washed with brine (×1) and dried over Na₂SO₄. The inorganics were filtered off, and the solvent was removed via rotary evaporation. Purification via flash chromatography on silica gel followed by recrystallization from CH₂Cl₂/hexanes yielded 1.3299 g (2.9 mmol, 67% yield) of (2S,3S)-2-benzyl-3-((4R)-4-(benzyloxy)-1-(tert-butoxycarbonyl)pyrrolidin-2-yl)-3-hydroxypropanoic acid.

DPPA (0.66 ml, 0.84 g, 3.07 mmol, 1.05 eq) was added to a stirred solution of (2S,3S)-2-benzyl-3-((4R)-4-(benzyloxy)-1-(tert-butoxycarbonyl)pyrrolidin-2-yl)-3-hydroxypropanoic acid (1.3299 g, 2.9 mmol, 1 eq) in 20 ml anhydrous toluene under Ar. After heating to 80° C. Et₃N (0.45 ml, 0.32 g, 3.21 mmol, 1.1 eq) was added. After 2 h the solvent was removed via rotary evaporation. The residue was diluted with water and extracted with CH₂Cl₂ (×2). The combined organics were dried over Na₂SO₄. The inorganics were filtered off, and the solvent was removed via rotary evaporation. Purification via flash chromatography on silica gel yielded 1.2471 g (2.8 mmol, 94% yield) of (4R)-tert-butyl 2-((4S,5S)-4-benzyl-2-oxooxazolidin-5-yl)-4-(benzyloxy)pyrrolidine-1-carboxylate.

Ba(OH)₂.8H₂O (0.7567 g, 2.4 mmol, 5 eq) was added to a stirred solution of (4R)-tert-butyl 2-((4S,5S)-4-benzyl-2-oxooxazolidin-5-yl)-4-(benzyloxy)pyrrolidine-1-carboxylate (0.2.26 g, 0.48 mmol, 1 eq) in 1,4-dioxane/water (4 ml:2 ml). The reaction was heated to reflux at 105° C. After 3 h reaction was cooled to room temperature. The mixture was diluted with CH₂Cl₂/brine and filtered. The layers were separated. The aqueous layer was extracted with CH₂Cl₂ (×1). The combined organics were dried over Na₂SO₄. The inorganics were filtered off, and the solvent was removed via rotary evaporation. Purification via flash chromatography on silica gel yielded 0.0827 g (0.21 mmol, 43% yield) of (2R,4R)-tert-butyl 2-((1S,2S)-2-amino-1-hydroxy-3-phenylpropyl)-4-(benzyloxy)pyrrolidine-1-carboxylate.

Method B: 1-phenyl-2-nitroethane was prepared by mixing nitromethane (21.03 g, 0.344 mol) and benzaldehyde (33.24 g, 0.313 mol) in methanol (100 mL) at 0° C. An aqueous solution of sodium hydroxide (15.66 g/40 mL of water) was added to the stirring solution over a period of 30 minutes. The stirring was continued for another hour in the temperature range of 0° C. The mixture was diluted with water (100 mL) and poured over crushed ice containing 32 mL of conc. HCl. The yellow solid precipitated out and was extracted with ether three times. The combined organic layers were washed with water, saturated aqueous sodium bicarbonate, brine and concentrated to provide a brown to yellow solid which was recrystallized from a small amount of EtOH to yield 25 g of a yellow solid. The yellow solid (24.7 g) was dissolved in dimethylsulfoxide (100 mL) and acetic acid (20 mL) at room temperature using a water bath to keep the temperature was added portionwise sodium borohydride (3.76 g) over 1.5 h. The resulting solution was stirred for another half hour, dilute with ethyl acetate (300 mL) and wash with water (200 mL), saturated aqueous sodium bicarbonate, saturated aqueous sodium chloride, dried, concentrated and purified (silica gel chromatography) to provide 1-phenyl-2-nitroethane as a pale yellow liquid (21 g, 85%). ¹H NMR (300 MHz, CDCl₃), d: 7.301 (m, 5H), 4.656 (m, 2H), 3.364 (t, J=7.5 Hz, 2H).

To an ice-cold solution of 1-phenyl-2-nitroethane (2.1 g, 13.73 mmol) in THF (15 mL) was added tetrabutylammonium fluoride (6.9 mL of 1.0 M solution in THF). After the resulting solution was stirred for 5 minutes, (2S,4R)-tert-butyl 4-(benzyloxy)-2-formylpyrrolidine-1-carboxylate (2.1 g, 6.867 mmol) in THF (10 mL) was added slowly and stirred 90 min, diluted with ethyl acetate, washed with water (3×50 mL), saturated aqueous sodium chloride, dry (magnesium sulfate) and purified (silica gel chromatography, eluting with hexanes and ethyl acetate) to give (2R,4R)-tert-butyl 4-(benzyloxy)-2-((1R,2S)-1-hydroxy-2-nitro-3-phenylpropyl)pyrrolidine-1-carboxylate as a syrup (1.1 g, 35%).

To a solution of (2R,4R)-tert-butyl 4-(benzyloxy)-2-((1R,2S)-1-hydroxy-2-nitro-3-phenylpropyl)pyrrolidine-1-carboxylate (0.4 g, 1.189 mmol) in methanol at 0° C. were added nickel chloride hexahydrate (0.0154 g, 0.12 mmol) and sodium borohydride (0.225 g, 5.945 mmol) portionwise over 1 min. The resulting mixture was stirred for 30 min, then concentrated, diluted with ethyl acetate, washed with water and filtered through celite. The organic layers were separated and washed with saturated aqueous sodium chloride, dried (magnesium sulfate), and concentrated to give a syrup which was purified with flash chromatography to the desired (2R,4R)-tert-butyl 2-((1S,2S)-2-amino-1-hydroxy-3-phenylpropyl)-4-(benzyloxy)pyrrolidine-1-carboxylate as a white solid (170 mg).

### Example 1.6.3

### (2R,5S)-tert-butyl 2-((1S,2S)-2-amino-1-hydroxy-3-phenylpropyl)-5-phenylpyrrolidine-1-carboxylate

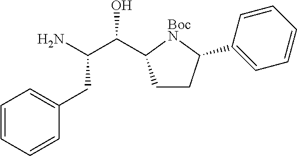 [SMILES: CC(C)(C)OC(=O)N1[C@@H]([C@@H](O)[C@@H](N)Cc2ccccc2)CC[C@H]1c1ccccc1]

4-Methylmorpholine (0.17 ml, 0.16 g, 1.57 mmol, 1.1 eq) was added to a stirred solution of (2R,5S)-1-(tert-butoxycarbonyl)-5-phenylpyrrolidine-2-carboxylic acid (NeoMPS, 0.4167 g, 1.43 mmol, 1 eq) in anhydrous 1,2-Dimethoxyethane (2 ml) at 0° C. under Ar. ⁱButylchloroformate (0.21 ml, 0.21 g, 1.57 mmol, 1.1 eq) was added dropwise to the resulting solution. After 30 min the mixture was filtered under Ar into an ice-cooled flask. NaBH₄ (0.0812 g, 2.15 mmol, 1.5 eq) dissolved in water (2 ml) was added and the reaction was swirled until gas evolution ceased. The reaction was diluted with water and extracted with EtOAc (×1). The organic layer was washed with water (×2), brine (×1), and dried over Na₂SO₄. The inorganics were filtered off, and the solvent was removed via rotary evaporation. Purification via flash chromatography on silica gel yielded 0.3658 g (1.32 mmol, 92% yield) of (2R,5S)-tert-butyl 2-(hydroxymethyl)-5-phenylpyrrolidine-1-carboxylate.

(2R,5S)-tert-butyl 2-(hydroxymethyl)-5-phenylpyrrolidine-1-carboxylate (0.3658 g, 1.32 mmol, 1 eq) was dissolved with stirring in anhydrous DMSO (2 ml) under Ar. The solution was cooled to 0° C. and the resulting solid was treated with Et₃N (0.74 ml, 0.5 g, 5.27 mmol, 4 eq) followed by SO₃.pyridine (0.4198 g, 2.64 mmol, 2 eq). After 30 min the cooling bath was removed. After stirring at room temperature for 30 min the reaction was diluted with Et₂O and the layers were separated. The organic layer was washed with 5% aqueous citric acid (×4), brine (×1), and dried over Na₂SO₄. The inorganics were filtered off, and the solvent was removed via rotary evaporation yielding 0.3213 g (1.16 mmol, 88% yield) of (2R,5S)-tert-butyl 2-formyl-5-phenylpyrrolidine-1-carboxylate.

The desired amine, (2R,5S)-tert-butyl 2-((1S,2S)-2-amino-1-hydroxy-3-phenylpropyl)-5-phenylpyrrolidine-1-carboxylate, was then generated using the method described above for (2R,4R)-tert-butyl 2-((1S,2S)-2-amino-1-hydroxy-3-phenylpropyl)-4-(benzyloxy)pyrrolidine-1-carboxylate.

### Example 1.6.4

### (2R,4S)-tert-butyl 2-((1S,2S)-2-amino-1-hydroxy-3-phenylpropyl)-4-fluoropyrrolidine-1-carboxylate

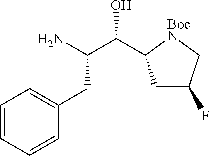 [SMILES: CC(C)(C)OC(=O)N1C[C@@H](F)C[C@@H]1[C@@H](O)[C@@H](N)Cc1ccccc1]

Thionyl chloride (9.7 mL, 133.46 mmol) was added to an ice-cold suspension of (2R,4R)-4-hydroxypyrrolidine-2-carboxylic acid (7 g, 53.382 mmol) in anhydrous methanol (110 mL) Stirred 10 min, then warm to room temperature for overnight. Concentrated, added methanol, and concentrated again. The crystal-like solid was rinsed with diethyl ether twice and dried under vacuum to give crude (2R,4R)-methyl 4-hydroxypyrrolidine-2-carboxylate hydrochloride which was used directly to the next step.

To a solution of (2R,4R)-methyl 4-hydroxypyrrolidine-2-carboxylate hydrochloride in acetone/water (120 mL/80 mL) at 0° C. were added triethylamine (24 mL), DMAP (0.706 g, 5.078 mmol) and di-tert-butyl dicarbonate (22.5 g, 102.888 mmol) slowly. The resulting mixture was stirred and warmed to room temperature for overnight. The solvent was removed under vacuum and diluted with ethyl acetate, washed with aqueous 0.5 M HCl solution, water, saturated aqueous sodium bicarbonate, brine, dried (sodium sulfate), filtered and concentrated to give a syrup which was purified with flash chromatography to produce (2R,4R)-1-tert-butyl 2-methyl 4-hydroxypyrrolidine-1,2-dicarboxylate as a white solid (12.83 g, 98% in two steps). ¹H NMR (300 MHz, CDCl₃), d: 4.370 (m, 2H), 3.838 (d, J=5.1 Hz, 3H), 3.739 (m, 1H), 3.556 (m, 1H), 2.256 (m, 1H), 2.134 (m, 1H), 1.500 (d, J=12.3 Hz, 9H).

To a solution of (2R,4R)-1-tert-butyl 2-methyl 4-hydroxypyrrolidine-1,2-dicarboxylate (750 mg, 3.058 mmol) in dichloromethane (50 mL) at −78° C. was added Deoxo-Fluor (0.73 mL, 3.975 mmol). The resulting mixture was stirred and warmed to room temperature for overnight, then was cooled in ice bath diluted with chloroform and quenched with saturated sodium bicarbonate solution. Warmed to room temperature, separated and dried (magnesium sulfate), concentrated and purified with silica gel chromatography to provide (2S,4S)-1-tert-butyl 2-methyl 4-fluoropyrrolidine-1,2-dicarboxylate as an oil (540 mg, 72%). ¹H NMR (300 MHz, CDCl₃), d: 5.193 (m, 0.5H), 5.019 (m, 0.5H), 4.417 (m, 1H), 3.933 (m, 1H), 3.838 (d, J=5.1 Hz, 3H), 3.589 (m, 1H), 2.516 (m, 1H), 2.111 (m, 1H), 1.500 (d, J=12.3 Hz, 9H).

To an ice cold solution of (2S,4S)-1-tert-butyl 2-methyl 4-fluoropyrrolidine-1,2-dicarboxylate (530 mg, 2.143 mmol) in THF (20 mL) was added lithium borohydride (2M THF solution, 1.6 mL). The resulting solution was stirred and warmed to room temperature overnight. Cooled in ice bath, slowly added diluted acetic acid (0.3 mL in 60 mL of water), extracted with ethyl acetate. Washed extract with saturated aqueous sodium bicarbonate solution, saturated aqueous sodium chloride, dried (sodium sulfate), concentrated and purified with silica gel chromatography to provide (2S,4S)-tert-butyl 4-fluoro-2-(hydroxymethyl)pyrrolidine-1-carboxylate as a thick oil (460 mg). ¹H NMR (300 MHz, CDCl₃), d: 5.193 (m, 0.5H), 5.019 (m, 0.5H), 4.127 (m, 1H), 3.933-3.764 (m, 2H), 3.598-3.346 (m, 2H), 2.335 (m, 2H), 1.477 (s, 9H).

To an ice-cold solution of (2S,4S)-tert-butyl 4-fluoro-2-(hydroxymethyl)pyrrolidine-1-carboxylate (0.460 g, 2.098 mmol) in DMSO (4 mL) was added triethylamine (1.2 mL, 8.4 mmol) and sulfurtrioxide-pyridine complex (0.670 g, 4.196 mmol). The resulting mixture was stirred 30 min, warmed to room temperature and stir 30 min, diluted with diethyl ether and washed with 5% aqueous citric acid, saturated aqueous sodium chloride, dried (magnesium sulfate) and concentrated to provide (2S,4S)-tert-butyl 4-fluoro-2-formylpyrrolidine-1-carboxylate as an oil (350 mg) that was used in the next step without further purification.

To an ice-cold solution of 1-phenyl-2-nitroethane (487 mg, 3.222 mmol) in THF (10 mL) was added tetrabutylammonium fluoride (2.9 mL of 1.0 M solution in THF). After the resulting solution was stirred for 5 minutes, (2R,4S)-tert-butyl 4-fluoro-2-formylpyrrolidine-1-carboxylate (350 mg, 1.611 romol) in THF (6 mL) was added slowly and stirred 90 min, diluted with ethyl acetate, washed with water (3×50 mL), saturated aqueous sodium chloride, dry (magnesium sulfate) and purify (silica gel chromatography, eluting with hexanes and ethyl acetate) to give the desired compound (2R,4S)-tert-butyl 4-fluoro-2-((1R,2S)-1-hydroxy-2-nitro-3-phenylpropyl)pyrrolidine-1-carboxylate as white solid (0.17 g, 22%). ¹H NMR (300 MHz, CDCl₃), d: 7.534-7.311 (m, 5H), 5.423 (s, 0.5H), 5.232 (s, 0.5H), 4.803 (s, 2H), 4.411 (m, 1H), 4.187 (m, 1H), 3.611-3.258 (m, 4H), 2.385 (m, 2H), 1.623 (s, 9H).

The desired amine, (2R,4S)-tert-butyl 2-((1S,2S)-2-amino-1-hydroxy-3-phenylpropyl)-4-fluoropyrrolidine-1-carboxylate, is then generated using the method described above for (2R,4R)-tert-butyl 2-((1S,2S)-2-amino-1-hydroxy-3-phenylpropyl)-4-(benzyloxy)pyrrolidine-1-carboxylate using NiCl₂ and NaBH₄ in methanol.

### Example 1.6.5

### (R)-tert-butyl 5-((1S,2S)-2-amino-1-hydroxy-3-phenylpropyl)-2,2-dimethylpyrrolidine-1-carboxylate

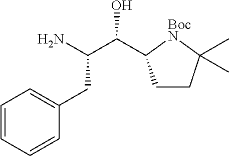 [SMILES: CC(C)(C)OC(=O)N1[C@@H]([C@@H](O)[C@@H](N)Cc2ccccc2)CCC1(C)C]

NaBH₄ (0.65 g, 17.1 mmol, 1.5 eq) was added portionwise to a stirred solution of NiCl₂.XH₂O (0.74 g, 5.7 mmol, 0.5 eq) in anhydrous MeOH (60 ml) under Ar. The resulting mixture was sonicated for 30 min. Methyl 4-methyl-4-nitropentanoate (Aldrich, 1.8 ml, 2.0 g, 11.4 mmol, 1 eq) was added dropwise with stirring. Additional NaBH₄ (1.3 g, 34.3 mmol, 3 eq) was added portionwise and the reaction was stirred over the weekend. The mixture was filtered through Celite and the volatiles were removed via rotary evaporation. The residue was partitioned between CH₂Cl₂ and saturated aqueous NaHCO₃. The layers were separated. The aqueous layer was extracted with CH₂Cl₂ (×1). The combined organics were dried over Na₂SO₄. The inorganics were filtered off, and the solvent was removed via rotary evaporation. Purification via flash chromatography yielded 0.7115 g (6.3 mmol, 55% yield) of 5,5-dimethylpyrrolidin-2-one.

A mixture of LiAlH₄ (0.2863 g, 7.5 mmol, 1.2 eq) in anhydrous THF (7.5 ml) under Ar was heated to 60° C. with stirring. A solution of 5,5-dimethylpyrrolidin-2-one (0.7115 g, 6.3 mmol, 1 eq) in anhydrous THF (3 ml) was added dropwise through the reflux condenser. The reaction was stirred at 60° C. overnight. The reaction was cooled to 0° C. and quenched by the sequential dropwise addition of water (2 ml) and 1N NaOH (1 ml). The mixture was filtered through Celite and diluted with Et₂O. The layers were separated, and the organic layer was dried over Na₂SO₄. The inorganics were filtered off, and the solvent was carefully removed via rotary evaporation yielding 0.4385 g (4.4 mmol, 70% yield) of the volatile-2,2-dimethylpyrrolidine product.

A stirred solution of 2,2-dimethylpyrrolidine (0.4385 g, 4.4 mmol, 1 eq) in anhydrous CH₂Cl₂ (6 ml) under Ar was treated sequentially with Et₃N (1.2 ml, 0.89 g, 8.8 mmol, 2 eq) and DMAP (0.0270 g, 0.22 mmol, 5 mol %). (Boc)₂O (1.2 ml, 1.16 g, 5.3 mmol, 1.2 eq) was added dropwise. The reaction was stirred overnight. The reaction was washed with 0.1 N HCl (×1), brine (×1), and dried over Na₂SO₄. The inorganics were filtered off, and the solvent was carefully removed via rotary evaporation. Purification via flash chromatography followed by careful removal of the solvent via rotary evaporation yielded 0.3585 g (1.8 mmol, 41% yield) of the volatile tert-butyl 2,2-dimethylpyrrolidine-1-carboxylate product.

The (R)-tert-butyl 5-((1S,2S)-2-amino-1-hydroxy-3-phenylpropyl)-2,2-dimethylpyrrolidine-1-carboxylate was then prepared via the same sequence as the unsubstituted pyrrolidines described herein.

### Example 1.6.6

### (2R,5R)-tert-butyl 2-((1S,2S)-2-amino-1-hydroxy-3-phenylpropyl)-5-methylpyrrolidine-1-carboxylate

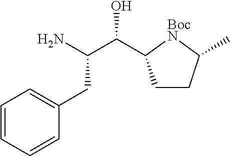 [SMILES: C[C@@H]1CC[C@H]([C@@H](O)[C@@H](N)Cc2ccccc2)N1C(=O)OC(C)(C)C]

SOCl₂ (0.14 ml, 0.23 g, 1.9 mmol, 10 mol %) was added dropwise to a stirred suspension of (R)-5-oxopyrrolidine-2-carboxylic acid (Aldrich, 2.5 g, 19.4 mmol, 1 eq) in anhydrous MeOH (20 ml) under Ar. A homogeneous solution gradually formed. After stirring overnight the volatiles were removed via rotary evaporation. The residue was adjusted to pH>7 with saturated aqueous NaHCO₃ and extracted with CH₂Cl₂ (×3). The combined organics were dried over Na₂SO₄. The inorganics were filtered off, and the solvent was removed via rotary evaporation. Purification via flash chromatography yielded (R)-methyl 5-oxopyrrolidine-2-carboxylate with a small amount of impurity.

A stirred solution of (R)-methyl 5-oxopyrrolidine-2-carboxylate (1.1876 g, 8.3 mmol, 1 eq) in anhydrous CH₂Cl₂ (20 ml) under Ar was treated with DMAP (0.1014 g, 0.83 mmol, 10 mol %) and Et₃N (1.3 ml, 0.92 g, 9.13 mmol, 1.1 eq). (Boc)₂O (2.86 ml, 2.7 g, 12.4 mmol, 1.5 eq) was added dropwise. The reaction was stirred overnight. The reaction was diluted with brine, and the layers were separated. The organic layer was dried over Na₂SO₄. The inorganics were filtered off, and the solvent was removed via rotary evaporation. Purification via flash chromatography yielded 1.3133 g (5.4 mmol, 65% yield) of (R)-1-tert-butyl 2-methyl 5-oxopyrrolidine-1,2-dicarboxylate.

A stirred solution of (R)-1-tert-butyl 2-methyl 5-oxopyrrolidine-1,2-dicarboxylate (1.3133 g, 5.4 mmol, 1 eq) in anhydrous THF (6 ml) under Ar was cooled to approximately −50° C. MeMgBr (3.0 M in Et2O, 2.16 ml, 6.5 mmol, 1.2 eq) was added dropwise. After 2 h the reaction was transferred to the freezer (approximately −20° C.) to sit overnight. The reaction was quenched with NH₄Cl, adjusted to pH=2-3 with 1N HCl, and extracted with EtOAc (×2). The combined organics were washed with brine (×1) and dried over Na₂SO₄. The inorganics were filtered off, and the solvent was removed via rotary evaporation. Purification via the flash system yielded 1.1717 g (4.5 mmol, 84% yield) of (R)-methyl 2-(tert-butoxycarbonylamino)-5-oxohexanoate.

TFA (2 ml, large excess) was added to a stirred solution of (R)-methyl 2-(tert-butoxycarbonylamino)-5-oxohexanoate (1.1717 g, 4.5 mmol, 1 eq) in anhydrous CH₂Cl₂ (2 ml) under Ar. After 5 h the volatiles were removed via rotary evaporation to yield (R)-methyl 5-methyl-3,4-dihydro-2H-pyrrole-2-carboxylate which was used without purification.

The above (R)-methyl 5-methyl-3,4-dihydro-2H-pyrrole-2-carboxylate in EtOH (12 ml) was shaken with 10% Pd/C (0.15 g) under 60 psi of H₂ overnight. The mixture was filtered through Celite, and the EtOH was removed via rotary evaporation to yield crude (2R,5R)-methyl 5-methylpyrrolidine-2-carboxylate which was used without purification.

A stirred solution of crude (2R,5R)-methyl 5-methylpyrrolidine-2-carboxylate (−4.5 mmol) in anhydrous CH₂Cl₂ (10 ml) under Ar was treated sequentially with Et₃N (1.3 ml, 0.9 g, 9.0 mmol, 2 eq) and DMAP (0.0275 g, 0.225 mmol, 5 mol %). (Boc)₂O (1.14 ml, 1.08 g, 4.95 mmol, 1.1 eq) was added dropwise, and the reaction was stirred over the weekend. The reaction was washed with 0.1N HCl (×1), brine (×1), and dried over Na₂SO₄. The inorganics were filtered off, and the solvent was removed via rotary evaporation. Purification via flash chromatography yielded 0.7829 g (3.2 mmol, 72% yield from the keto ester) of (2R,5S)-1-tert-butyl 2-methyl 5-methylpyrrolidine-1,2-dicarboxylate.

(2R,5R)-tert-butyl 2-((1S,2S)-2-amino-1-hydroxy-3-phenylpropyl)-5-methylpyrrolidine-1-carboxylate was then synthesized from (2R,5S)-1-tert-butyl 2-methyl 5-methylpyrrolidine-1,2-dicarboxylate in a similar manner to the synthesis of (2R,4R)-tert-butyl 2-((1S,2S)-2-amino-1-hydroxy-3-phenylpropyl)-4-(benzyloxy)pyrrolidine-1-carboxylate described herein.

### Example 1.6.7

### (2R,4R)-tert-butyl 4-(allyloxy)-2-(4S,5S)-4-benzyl-2,2-dimethyloxazolidin-5-yl)pyrrolidine-1-carboxylate

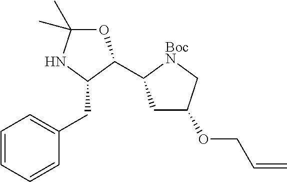 [SMILES: C=CCO[C@@H]1C[C@H]([C@H]2OC(C)(C)N[C@H]2Cc2ccccc2)N(C(=O)OC(C)(C)C)C1]

To a solution of 4-(R)-Benzyloxy-2-(R)-(1-(R)-hydroxy-2-(S)-amino-3-phenylpropyl)-pyrrolidine-1-carboxylic acid tert-butyl ester (2.12 g, 4.97 mmol) in anhydride DMF at −78° C. were added Teoc-O-succinimdyl (1.35 g, 5.22 mmol) and triethylamine (1 mL, 7.455 mmol). The resulting mixture was stirred for 1 h then warmed to room temperature and stirred for overnight. The reaction mixture was poured into water extracted with ethyl acetate, washed with water. The organic layers were separated and dried (sodium sulfate), and concentrated to give syrup which was purified to give the pure desired Teoc-protected compound (2.45 g, 86% in two steps). %). ¹H NMR (300 MHz, CDCl₃), d: 7.400-7.233 (m, 10H), 4.594 (s, 2H), 4.153-4.012 (m, 4H), 3.883-3.411 (m, 5H), 3.102 (m, 1H), 2.933 (m, 1H), 2.360 (m, 1H), 2.115 (m, 1H), 2.145 (m, 1H), 1.466 (s, 4H), 1.239 (s, 5H), 0.890 (m, 2H), 0.006 (s, 9H).

To a solution of the above Teoc-protected compound (2.81 g, 4.93 mmol) in anhydrous benzene were added dimethoxypropane (3 mL, 24.61 mmol) and PPTS (133 mg, 2.46 mmol). The resulting mixture was heated to 80° C. for 2 h, concentrated to get yellow syrup, which was directly purified with column to give the pure desired Teoc-protected 2,2-dimethyloxazolidine compound (2.75 g, 92%). ¹H NMR (300 MHz, CDCl₃), d: 7.349-7.114 (m, 10H), 4.643-4.451 (m, 2H), 4.211-4.107 (m, 4H), 3.710 (m, 2H), 3.407 (m, 1H), 2.994 (m, 1H), 2.587 (m, 1H), 1.992 (m, 1H), 1.747 (s, 3H), 1.610 (m, 1H), 1.469 (m, 12H), 0.388 (m, 2H), 0.084 (s, 9H).

The above compound (2.70 g, 4.42 mmol) in ethyl acetate was hydrogenated with Pd(OH)₂ (600 mg, 20% on charcoal) under balloon pressure for overnight. The resulting mixture was filtered through celite, evaporated the solvent to give a white solid (2.7 g, quantitative yield) which was directly used for the next step without further purification. ¹H NMR (300 MHz, CDCl₃), d: 7.209 (m, 5H), 4.535 (m, 1H), 4.359-4.213 (m, H), 4.101 (m, 1H), 3.865 (m, 1H), 3.4210 (m, 2H), 2.786 (m, 1H), 2.305 (m, 1H), 2.108 (m, 1H), 1.776 (s, 2H), 1.576 (s, 6H), 1.447 (s, 9H), 0.846 (m, 0.6H), 0.596 (m, 1.4H), 0.015 (m, 9H).

To a solution of the alcohol (761 mg, 1.46 mmol) and allyl iodide (0.2 mL, 2.19 mmol) in anhydride DMF at 0° C. was added sodium hydride (76 mg, 60% in mineral oil) slowly. The resulting mixture was warmed to room temperature and stirred for one hour, diluted with ethyl acetate and poured into a separation funnel charged with water. The organic layer was washed with water two times, brine one time and concentrated to get light yellow syrup, which was purified with column to give the pure desired allyl ether (760 m g, 93%). ¹H NMR (300 MHz, CDCl₃), d: 7.242 (m, 5H), 5.892 (m, 1H), 5.308-5.137 (m, 2H), 4.207-4.077 (m, 5H), 3.914 (m, 1H), 3.717 (m, 2H), 3.354 (m, 1H), 3.002 (m, 1H), 2.559 (m, 2H), 1.969 (m, 1H), 1.748 (s, 3H), 1.660 (m, 1H), 1.498 (s, 12H), 0.392 (m, 2H), 0.083 (s, 9H).

To a solution of the allyl ether (86.4 mg, 0.154 mmol) in acetonitril were added TBAF (0.15 mL, 1 M in THF) and potassium fluoride (18 mg, 0.31 mmol). The resulting mixture was heated up to 50° C. and stirred for overnight, diluted with ethyl acetate and poured into a separation funnel charged with saturated sodium bicarbonate solution. The organic layer was washed with water and brine, concentrated to provide (2R,4R)-tert-butyl 4-(allyloxy)-2-((4S,5S)-4-benzyl-2,2-dimethyloxazolidin-5-yl)pyrrolidine-1-carboxylate, which was directly used for the next step reaction without further purification (60 m g, 93%). ¹H NMR (300 MHz, CDCl₃), d: 7.195 (m, 5H), 5.914 (m, 1H), 5.339-5.164 (m, 2H), 4.116-3.887 (m, 4H), 3.610-3.420 (m, 2H), 3.334-2.889 (m, 3H), 2.643-2.073 (m, 3H), 1.463 (m, 15H).

### Example 1.6.8

### (2R,4R)-tert-butyl 2-((1S,2S)-2-amino-1-hydroxy-3-phenylpropyl)-4-hydroxypyrrolidine-1-carboxylate

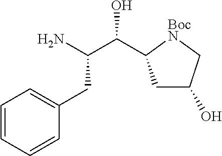 [SMILES: CC(C)(C)OC(=O)N1C[C@H](O)C[C@@H]1[C@@H](O)[C@@H](N)Cc1ccccc1]

To a solution of 4-(R)-Benzyloxy-2-(R)-(1-(R)-hydroxy-2-(S)-nitro-3-phenylpropyl)-pyrrolidine-1-carboxylic acid tert-butyl ester (2.2 g, 4.82 mmol) in ethyl acetate/methanol (2:1) was hydrogenated with Pd(OH)₂ (1.2 mg, 20% on charcoal) under balloon pressure for overnight. The resulting mixture was filtered through celite, evaporated the solvent to give syrup (1.67 g) which was directly used for the next step without further purification and characterization. To the syrup in methanol at 0° C. were added nickel chloride hexahydrate (1.18 g, 9.12 mmol) and sodium borohydride (1.04 g, 27.34 mmol) portionwise over 5 min. The resulting mixture was stirred for 30 min at room temperature, then quenched with 10 mL of water, concentrated, diluted with ethyl acetate, washed with water and filtered through celite. The organic layers was separated and washed with saturated aqueous sodium chloride, dried (sodium sulfate), and concentrated to give a syrup which was purified to give a white solid of (2R,4R)-tert-butyl 2-((1S,2S)-2-amino-1-hydroxy-3-phenylpropyl)-4-hydroxypyrrolidine-1-carboxylate (1.27 g, 73% overall for two step reactions). ¹H NMR (300 MHz, CDCl₃+CD₃OD), d: 7.357-7.150 (m, 5H), 4.233 (m, 2H), 3.742 (m, 1H), 3.544-3.403 (m, 2H), 3.243-3.058 (m, 2H), 2.496-2.339 (m, 2H), 2.055 (m, 1H), 1.464 (s, 9H).

### Example 1.6.9

### (2R,4R)-tert-butyl 2-((4S,5S)-4-benzyl-2,2-dimethyloxazolidin-5-yl)-4-propoxypyrrolidine-1-carboxylate

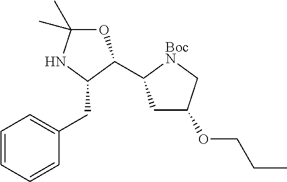 [SMILES: CCCO[C@@H]1C[C@H]([C@H]2OC(C)(C)N[C@H]2Cc2ccccc2)N(C(=O)OC(C)(C)C)C1]

To a solution of (2R,4R)-tert-butyl 2-((1S,2S)-2-amino-1-hydroxy-3-phenylpropyl)-4-hydroxypyrrolidine-1-carboxylate (619 mg, 1.84 mmol) in methanol at 0° C. were added dibenzyl dicarbonate (632 mg, 2.21 mmol) and triethylamine (0.38 mL, 2.76 mmol). The resulting mixture was stirred for 15 minutes, then warmed to room temperature for 1 h. The reaction solvent was evaporated, the mixture was diluted with chloroform, washed with 0.2 M HCl solution. The organic layers were separated and dried (sodium sulfate), and concentrated to give a syrup which was purified to provide pure (2R,4R)-tert-butyl 2-((1S,2S)-2-(benzyloxycarbonylamino)-1-hydroxy-3-phenylpropyl)-4-hydroxypyrrolidine-1-carboxylate (740 mg, 86%). ¹H NMR (300 MHz, CDCl₃), d: 7.264 (m, 10H), 5.003 (m, 2H), 4.482-3.754 (m, 4H), 3.448 (m, 2H), 3.205-2.711 (m, 2H), 2.164 (s, 2H), 1.452 (m, 5H), 1.279 (m, 4H).

To a solution of (2R,4R)-tert-butyl 2-((1S,2S)-2-(benzyloxycarbonylamino)-1-hydroxy-3-phenylpropyl)-4-hydroxypyrrolidine-1-carboxylate in anhydrous benzene were added dimethoxypropane (1 mL, 7.86 mmol) and PPTS (42.3 mg, 1.57 mmol). The resulting mixture was heated to 80° C. for 2 h, concentrated to get yellow syrup, which was directly purified with column to give pure (4S,5R)-benzyl 4-benzyl-5-((2R,4R)-1-(tert-butoxycarbonyl)-4-hydroxypyrrolidin-2-yl)-2,2-dimethyloxazolidine-3-carboxylate (570 mg, 59%).

(4S,5R)-benzyl 4-benzyl-5-((2R,4R)-1-(tert-butoxycarbonyl)-4-hydroxypyrrolidin-2-yl)-2,2-dimethyloxazolidine-3-carboxylate (240 mg, 0.47 mmol) in anhydride DMF at 0° C., was added sodium hydride (28.2 my, 60% in mineral oil) and stirred for 30 minutes at same temperature, then allyl iodide (158 mg, 0.94 mmol) was added to the reaction mixture, warmed to room temperature for overnight. The reaction was diluted with ethyl acetate and poured into a separation funnel charged with water. The organic layer was washed with water two times, brine one time and concentrated to get light yellow syrup, which was purified with column to provide pure (4S,5R)-benzyl 5-((2R,4R)-4-(allyloxy)-1-(tert-butoxycarbonyl)pyrrolidin-2-yl)-4-benzyl-2,2-dimethyloxazolidine-3-carboxylate (100 mg).

¹H NMR (300 MHz, CDCl₃), d: 7.201 (m, 10H), 5.942-5.813 (m, 1H), 5.308-5.141 (m, 2H), 4.880 (m, 1H), 4.235 (m, 3H), 4.097 (m, 3H), 3.915 (m, 1H), 3.720 (m, 3H), 3.376 (m, 1H), 2.557 (m, 2H), 1.957 (m, 1H), 1.775 (s, 3H), 1.530 (s, 3H), 1.489 (s, 9H).

To a solution of (4S,5R)-benzyl 5-((2R,4R)-4-(allyloxy)-1-(tert-butoxycarbonyl)pyrrolidin-2-yl)-4-benzyl-2,2-dimethyloxazolidine-3-carboxylate (95 mg, 0.173 mmol) in ethyl acetate was added Pd (OH)₂ (20% on charcoal, 30 mg). The resulting mixture was hydrogenated under hydrogen balloon overnight. The mixture was filtered through celite, evaporated the solvent to give a white solid of the desired (2R,4R)-tert-butyl 2-((4S,5S)-4-benzyl-2,2-dimethyloxazolidin-5-yl)-4-propoxypyrrolidine-1-carboxylate (95 mg, quantitative yield), which was directly used for the next step without further purification.

¹H NMR (300 MHz, CDCl₃), d: 7.259 (m, 5H), 4.317 (m, 1H), 3.919 (m, 2H), 3.657-3.224 (m, 4H), 2.910 (m, 2H), 2.685 (m, 1H), 2.438-1.975 (m, 2H), 1.586 (m, 2H), 1.470 (s, 9H), 1.246 (s, 3H), 0.940 (m, 6H).

### Example 1.6.10

### (R)-tert-butyl 2-((1S,2S)-2-amino-3-(3,5-difluorophenyl)-1-hydroxypropyl)pyrrolidine-1-carboxylate

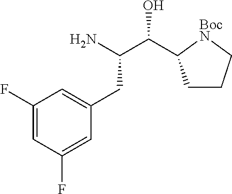 [SMILES: CC(C)(C)OC(=O)N1CCC[C@@H]1[C@@H](O)[C@@H](N)Cc1cc(F)cc(F)c1]

Following normal coupling procedure, Boc difluorophenylalanine (1.5 g, 5.0 mmol) and N,O-dimethylhydroxylamine hydrochloride (536 mg, 5.5 mmol) were coupled to provide (S)-tert-butyl 3-(3,5-difluorophenyl)-1-(methoxy(methyl)amino)-1-oxopropan-2-ylcarbamate (1.44 g, 84%) as a colorless oil.

(S)-tert-butyl 3-(3,5-difluorophenyl)-1-(methoxy(methyl)amino)-1-oxopropan-2-ylcarbamate (1.44 g, 4.2 mmol) was treated with HCl in dioxane (4.0 N, 2.1 mL, 8.4 mmol) at 0° C. The resulting solution was stirred for 4 h while warmed up to r.t. The solvent was removed under reduced pressure and the residue was diluted with CHCl₃ and saturated aqueous NaHCO₃. The layers were separated and the aqueous layer was extracted with CHCl₃. The combined organic layer was washed with brine, dried with Na₂SO₄ and concentrated under reduced pressure to provide (S)-2-amino-3-(3,5-difluorophenyl)-N-methoxy-N-methylpropanamide (1.02 g, 99%) as a yellow oil.

To a stirred solution of (S)-2-amino-3-(3,5-difluorophenyl)-N-methoxy-N-methylpropanamide (1.02 g, 4.2 mmol) in ethanol (15 mL) and H₂O (3 mL) was added K₂CO₃ (1.74 g, 12.6 mmol) and benzylbromide (1.1 mL, 9.2 mmol). The reaction mixture was stirred at r.t. for 18 h and diluted with chloroform and filtered. The filtrate was concentrated and the residue was diluted with chloroform and water. The layers were separated and the aqueous layer was extracted with CHCl₃. The combined organic layer was washed with brine, dried with Na₂SO₄ and concentrated under reduced pressure. The residue was purified by column chromatography (10% EtOAc in hexanes) to provide (S)-2-(dibenzylamino)-3-(3,5-difluorophenyl)-N-methoxy-N-methylpropanamide (455.3 mg, 26%) as a yellow oil.

To a stirred solution of (S)-2-(dibenzylamino)-3-(3,5-difluorophenyl)-N-methoxy-N-methylpropanamide (1.08 g, 2.5 mmol) in ether (40 mL) at 0° C. was added LiAlH₄ (106 mg, 2.8 mmol). The resulting solution was stirred for 1 h and quenched with sodium hydrogensulfate solution (1.0 M, 10 mL) slowly. The layers were separated and the aqueous layer was extracted with EtOAc (2×30 mL). The combined organic layer was washed with brine, dried with Na₂SO₄ and concentrated under reduced pressure to provide (S)-2-(dibenzylamino)-3-(3,5-difluorophenyl)propanal (934 mg, 99%) as a yellow oil.

To a stirred solution of (−)-sparteine (398 mg, 1.7 mmol) in ether (20 mL) at −78° C. was added sec-BuLi (1.6 mL, 2.2 mmol) dropwise followed by N-Boc-pyrrolidine (292 mg, 1.7 mmol) in ether. The resulting mixture was stirred at −78° C. for 2 h and (S)-2-(dibenzylamino)-3-(3,5-difluorophenyl)propanal (934 mg, 2.5 mmol) in ether was added slowly. The reaction mixture was stirred for 40 min and HoAc (1 mL) was added and warmed up to r.t. H₂O was added and the layers were separated. The aqueous layer was extracted with EtOAc (2×30 mL). The combined organic layer was washed with 5% citric acid, brine, dried with Na₂SO₄ and concentrated under reduced pressure. The residue was purified by column chromatography (30% EtOAc in hexanes) to provide (R)-tert-butyl 2-((1S,2S)-2-(dibenzylamino)-3-(3,5-difluorophenyl)-1-hydroxypropyl)pyrrolidine-1-carboxylate (213 mg, 23%) as a pale yellow oil.

A hydrogen balloon was put on a stirred solution of (R)-tert-butyl 2-((1S,2S)-2-(dibenzylamino)-3-(3,5-difluorophenyl)-1-hydroxypropyl)pyrrolidine-1-carboxylate (213 mg, 0.4 mmol), Pd(OH)₂ (100 mg) in MeOH (10 mL) The stirring was continued for 24 h and the resulting mixture was filtered through a pad of Celite. The filtrate was concentrated under reduced pressure to provide (R)-tert-butyl 2-((1S,2S)-2-amino-3-(3,5-difluorophenyl)-1-hydroxypropyl)pyrrolidine-1-carboxylate (157 mg, 99%) as a white solid.

### Example 1.6.11

### (R)-tert-butyl 2-((1S,2S)-2-amino-3-(3-fluorophenyl)-1-hydroxypropyl)pyrrolidine-1-carboxylate

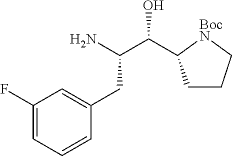 [SMILES: CC(C)(C)OC(=O)N1CCC[C@@H]1[C@@H](O)[C@@H](N)Cc1cccc(F)c1]

To a stirred solution of 3-fluorophenylalanine (2 g, 10.9 mmol) in dioxane (40 mL) and H₂O (20 mL) was added K₂CO₃ (6.0 g, 44 mmol) and benzylbromide (4.1 mL, 35 mmol). The reaction mixture was stirred at r.t. for 18 h and concentrated. The residue was diluted with EtOAc and saturated aqueous NH₄Cl. The layers were separated and the aqueous layer was extracted with EtOAc. The combined organic layer was washed with brine, dried with Na₂SO₄ and concentrated under reduced pressure. The residue was purified by column chromatography (10% EtOAc in hexanes) to provide (S)-benzyl 2-(dibenzylamino)-3-(3-fluorophenyl)propanoate (3.82 g, 77%) as a colorless oil.

To a stirred solution of (S)-benzyl 2-(dibenzylamino)-3-(3-fluorophenyl)propanoate (3.8 g, 8.4 mmol) in THF (80 mL) at 0° C. was added LiAlH₄ (637 mg, 16.8 mmol). The resulting solution was stirred for 1.5 h and quenched with water (0.6 mL), 20% NaOH (0.6 mL) and brine (2 mL) slowly. The mixture was filtered and concentrated under reduced pressure to provide (S)-2-(dibenzylamino)-3-(3-fluorophenyl)propan-1-ol (2.88 g, 99%) as a colorless oil

To a stirred solution of (S)-2-(dibenzylamino)-3-(3-fluorophenyl)propan-1-ol (2.8 g, 8.4 mmol) in DMSO (10 mL) at 0° C. was added Et₃N (4.7 mL, 34 mmol) and SO₃.Py (2.7 g, 16.8 mmol). The resulting mixture was stirred for 1 h and diluted with H₂O (20 mL) and EtOAc (30 mL). The layers were separated and the aqueous layer was extracted with EtOAc (2×30 mL). The combined organic layer was washed with H₂O, 5% citric acid, brine, dried with Na₂SO₄ and concentrated under reduced pressure to provide (S)-2-(dibenzylamino)-3-(3-fluorophenyl)propanal (2.7 g, 90%).

(R)-tert-butyl 2-((1S,2S)-2-amino-3-(3-fluorophenyl)-1-hydroxypropyl)pyrrolidine-1-carboxylate was generated from (S)-2-(dibenzylamino)-3-(3-fluorophenyl)propanal using standard procedures described herein.

### Example 1.6.12

### (S)-tert-butyl 2-((1S,2S)-2-amino-1-hydroxy-3-phenylpropyl)-3,3-difluoropyrrolidine-1-carboxylate

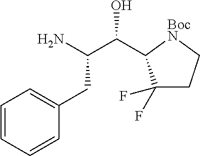 [SMILES: CC(C)(C)OC(=O)N1CCC(F)(F)[C@@H]1[C@@H](O)[C@@H](N)Cc1ccccc1]

To a stirred solution of N-Boc-3-pyrrolidinone (1.0 g, 5.4 mmol) in CH₂Cl₂ at 0° C. was added Deoxo-fluoro (3 mL, 16.2 mmol). The resulting solution was stirred for 15 h and quenched with saturated aqueous NaHCO₃. The layers were separated and the aqueous layer was extracted with CHCl₃. The combined organic layer was washed with brine, dried with Na₂SO₄ and concentrated under reduced pressure. The residue was purified by column chromatography (15% EtOAc in hexanes) to provide tert-butyl 3,3-difluoropyrrolidine-1-carboxylate (0.8 g, 71%) as an off-white solid.

To a stirred solution of dibenzyl phenylalaninol (5 g, 15 mmol) in DMSO (20 mL) at 0° C. was added Et₃N (8.4 mL, 60 mmol) and SO₃.Py. The resulting mixture was stirred for 1 h and diluted with H₂O (20 mL) and EtOAc (30 mL). The layers were separated and the aqueous layer was extracted with EtOAc (2×30 mL). The combined organic layer was washed with H₂O, 5% citric acid, brine, dried with Na₂SO₄ and concentrated under reduced pressure. The residue was purified by column chromatography (10% EtOAc in hexanes) to provide (S)-2-(dibenzylamino)-3-phenylpropanal (4.42 g, 90%).

tert-butyl 3,3-difluoropyrrolidine-1-carboxylate and (S)-2-(dibenzylamino)-3-phenylpropanal were then coupled and further treated as described herein to provide (S)-tert-butyl 2-((1S,2S)-2-amino-1-hydroxy-3-phenylpropyl)-3,3-difluoropyrrolidine-1-carboxylate.

### Example 1.6.13

### (R)-tert-butyl 2-((1S,2S)-2-amino-1-hydroxy-3-phenylpropyl)-4,4-difluoropyrrolidine-1-carboxylate

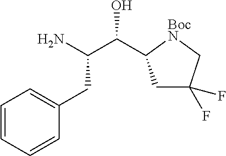 [SMILES: CC(C)(C)OC(=O)N1CC(F)(F)C[C@@H]1[C@@H](O)[C@@H](N)Cc1ccccc1]

To an ice-cold solution of (4S,5R)-2-(trimethylsilyl)ethyl 4-benzyl-5-((2R,4R)-1-(tert-butoxycarbonyl)-4-hydroxypyrrolidin-2-yl)-2,2-dimethyloxazolidine-3-carboxylate (0.65 g, 1.25 mmol) in DMSO (10 mL) was added triethylamine (0.7 mL, 5.0 mmol) and sulfurtrioxide-pyridine complex (0.397 g, 2.5 mmol). The resulting mixture was stirred 30 min, warmed to room temperature and stirred 30 min, diluted with diethyl ether and washed with water three times, dried (sodium sulfate) and concentrated to provide (4S,5R)-2-(trimethylsilyl)ethyl 4-benzyl-5-((R)-1-(tert-butoxycarbonyl)-4-oxopyrrolidin-2-yl)-2,2-dimethyloxazolidine-3-carboxylate as an oil which was purified with flash chromatography (580 mg, 91%). ¹H NMR (300 MHz, CDCl₃), d: 7.263 (m, 5H), 4.419-4.262 (m, 3H), 4.201-3.820 (m, 3H), 3.678 (m, 1H), 2.839 (m, 3H), 2.491 (m, 1H), 1.684-1.438 (m, 15H), 0.938-0.696 (m, 2H), −010 (s, 9H).

To a solution of (4S,5R)-2-(trimethylsilyl)ethyl 4-benzyl-5-((R)-1-(tert-butoxycarbonyl)-4-oxopyrrolidin-2-yl)-2,2-dimethyloxazolidine-3-carboxylate (0.34 g, 0.655 mmol) in DCM was added Deoxo-Fluoro (0.36 mL, 1.97 mmol) at room temperature. The resulting mixture was stirred overnight, diluted with chloroform and quenched with aqueous sodium bicarbonate solution. The organic was separated, dried (sodium sulfate) and concentrated to give the title compound as oil which was purified with flash chromatography to provide (R)-tert-butyl 2-((1S,2S)-2-amino-1-hydroxy-3-phenylpropyl)-4,4-difluoropyrrolidine-1-carboxylate (210 mg). ¹H NMR (300 MHz, CDCl₃), d: 7.197 (m, 5H), 4.235 (m, 3H), 3.901 (m, 2H), 3.556 (m, 1H), 3.135 (m, 1H), 2.849-2.511 (m, 3H), 2.382 (m, 1H), 1.735-1.329 (m, 15H), 0.520 (m, 2H), −0.055 (s, 9H).

### Example 1.6.14

### (R)-tert-butyl 2-((1R,2S)-2-amino-1-hydroxy-3-phenylpropyl)pyrrolidine-1-carboxylate

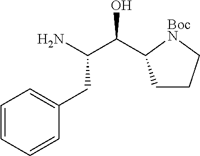 [SMILES: CC(C)(C)OC(=O)N1CCC[C@@H]1[C@H](O)[C@@H](N)Cc1ccccc1]

To a stirred solution of (R)-tert-butyl 2-((1S,2S)-2-(dibenzylamino)-1-hydroxy-3-phenylpropyl)pyrrolidine-1-carboxylate (853 mg, 1.7 mmol) in DMSO (5 mL) at 0° C. was added Et₃N (0.95 mL, 6.8 mmol) and SO₃.Py (542 mg, 3.4 mmol). The resulting mixture was warmed up to room temperature and stirred for 53 h and diluted with H₂O (20 mL) and EtOAc (30 mL). The layers were separated and the aqueous layer was extracted with EtOAc (2×15 mL). The combined organic layer was washed with H₂O, 5% citric acid, brine, dried with Na₂SO₄ and concentrated under reduced pressure. The residue was purified by column chromatography (15% EtOAc in hexanes) to provide (R)-tert-butyl 2-((S)-2-(dibenzylamino)-3-phenylpropanoyl)pyrrolidine-1-carboxylate (452 mg, 54%) as a colorless oil.

To a stirred solution of (R)-tert-butyl 2-((S)-2-(dibenzylamino)-3-phenylpropanoyl)pyrrolidine-1-carboxylate (452 mg, 0.91 mmol) in methanol (10 mL) was added NaBH₄ (41 mg, 1.1 mmol). The reaction mixture was heated to reflux for 20 h and cooled to room temperature. The solvent was removed under reduced pressure. The residue was purified by column chromatography (30% EtOAc in hexanes) to provide (1R,7aR)-1-((S)-1-(dibenzylamino)-2-phenylethyl)tetrahydropyrrolo[1,2-c]oxazol-3(1H)-one (86.4 mg, 22%) as a white solid.

To a stirred solution of (1R,7aR)-1-((S)-1-(dibenzylamino)-2-phenylethyl)tetrahydropyrrolo[1,2-c]oxazol-3(1H)-one (80 mg, 0.19 mmol) in dioxane (4 mL) and H2O (2 mL) was added Ba(OH)₂.8H₂O. The resulting mixture was heated at 105° C. for 20 h, cooled to room temperature and diluted with H₂O and CHCl₃. The mixture was filtered through a pad of Celite and the layers were separated. The aqueous layer was extracted with CHCl₃ (2×15 mL). The combined organic layer was washed with brine, dried with Na₂SO₄ and concentrated under reduced pressure to provide (1S,2S)-2-(dibenzylamino)-3-phenyl-1-((R)-pyrrolidin-2-yl)propan-1-ol (66.4 mg, 84%) as an off white solid.

To a stirred solution of (1S,2S)-2-(dibenzylamino)-3-phenyl-1-((R)-pyrrolidin-2-yl)propan-1-ol (66 mg, 0.16 mmol) in CH₂Cl₂ (5 mL) was added triethylamine (0.046 mL, 0.32 mmol) and Boc₂O (0.045 mL, 0.2 mmol) and DMAP (19 mg, 0.16 mmol). The resulting mixture was stirred at r.t. for 18 h and quenched with saturated aqueous NH₄Cl. The layers were separated and the aqueous layer was extracted with CH₂Cl₂ (2×15 mL). The combined organic layer was washed with brine, dried with Na₂SO₄ and concentrated under reduced pressure. The residue was purified by column chromatography (30% EtOAc in hexanes) to provide (R)-tert-butyl 2-((1R,2S)-2-(dibenzylamino)-1-hydroxy-3-phenylpropyl)pyrrolidine-1-carboxylate (67.9 mg, 85%) as a colorless oil.

(R)-tert-butyl 2-((1R,2S)-2-(dibenzylamino)-1-hydroxy-3-phenylpropyl)pyrrolidine-1-carboxylate was reduced to (R)-tert-butyl 2-((1R,2S)-2-amino-1-hydroxy-3-phenylpropyl)pyrrolidine-1-carboxylate using procedures described herein.

### Example 1.6.15

### (2R,4R)-tert-butyl 2-((1S,2S)-2-amino-1-hydroxy-3-phenylpropyl)-4-methoxypyrrolidine-1-carboxylate

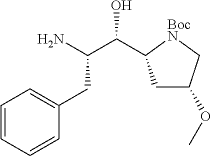 [SMILES: CO[C@@H]1C[C@H]([C@@H](O)[C@@H](N)Cc2ccccc2)N(C(=O)OC(C)(C)C)C1]

Et₃N (0.045 ml, 0.033 g, 0.33 mmol, 1.5 eq) and Teoc-O-succinimidyl (0.0591 g, 0.23 mmol, 1.05 eq) were added sequentially to a stirred solution of (2R,4R)-tert-butyl 2-((1S,2S)-2-amino-1-hydroxy-3-phenylpropyl)-4-(benzyloxy)pyrrolidine-1-carboxylate (0.0926 g, 0.22 mmol, 1 eq) in anhydrous 1,4-dioxane (2 ml) under Ar. After stirring overnight the reaction was diluted with EtAOc, washed with water (×2), brine (×1), and dried over Na₂SO₄. The inorganics were filtered off, and the solvent was removed via rotary evaporation. Purification via flash chromatography yielded 0.1178 g (0.21 mmol, 94% yield) of (2R,4R)-tert-butyl 4-(benzyloxy)-2-((1S,2S)-1-hydroxy-3-phenyl-2-(((2-(trimethylsilyl)ethoxy)carbonyl)amino)propyl)pyrrolidine-1-carboxylate.

A stirred solution of (2R,4R)-tert-butyl 4-(benzyloxy)-2-((1S,2S)-1-hydroxy-3-phenyl-2-(((2-(trimethylsilyl)ethoxy)carbonyl)amino)propyl)pyrrolidine-1-carboxylate (0.239 g, 0.42 mmol, 1 eq) in anhydrous benzene (3 ml) under Ar was treated sequentially with dimethoxypropane (0.26 ml, 0.22 g, 2.09 mmol, 5 eq) and pyridinium p-toluenesulfonate (0.0526 g, 0.209 mmol, 0.5 eq). The mixture was heated to 80° C. After 3 h the heat was turned off and the reaction was stirred at 80° C. to room temperature overnight. The mixture was filtered through cotton, and the solvent was removed via rotary evaporation. Purification via flash chromatography yielded 0.2177 g (0.36 mmol, 85% yield) of (4S,5R)-2-(trimethylsilyl)ethyl 4-benzyl-5-((2R,4R)-4-(benzyloxy)-1-(tert-butoxycarbonyl)pyrrolidin-2-yl)-2,2-dimethyloxazolidine-3-carboxylate.

20% Pd(OH)₂/C (0.022 g, 10% by wt) was added to solution of (4S,5R)-2-(trimethylsilyl)ethyl 4-benzyl-5-((2R,4R)-4-(benzyloxy)-1-(tert-butoxycarbonyl)pyrrolidin-2-yl)-2,2-dimethyloxazolidine-3-carboxylate (0.2177 g, 0.36 mmol, 1 eq) in EtOH (3 ml). The mixture was stirred vigorously under H₂ (balloon pressure). After 3 h the mixture was filtered through Celite, and the solvent was removed via rotary evaporation. Purification via flash chromatography yielded 0.1558 g (0.30 mmol, 83% yield) of (4S,5R)-2-(trimethylsilyl)ethyl 4-benzyl-5-((2R,4R)-1-(tert-butoxycarbonyl)-4-hydroxypyrrolidin-2-yl)-2,2-dimethyloxazolidine-3-carboxylate.

A solution of (4S,5R)-2-(trimethylsilyl)ethyl 4-benzyl-5-((2R,4R)-1-(tert-butoxycarbonyl)-4-hydroxypyrrolidin-2-yl)-2,2-dimethyloxazolidine-3-carboxylate (0.0906 g, 0.17 mmol, 1 eq) in anhydrous DMF (2 ml) under Ar was cooled to 0° C. with stirring. After protecting from light, the solution was treated sequentially with MeI (0.022 ml, 0.049 g, 2 eq) and NaH (60% dispersion in oil, 0.0104 g, 1.5 eq). After 1 h the cooling bath was removed. After 3 h the reaction was quenched with water and diluted with EtOAc. The layers were separated. The organic layer was washed with water (×3), brine (×1), and dried over Na₂SO₄. The inorganics were filtered off, and the solvent was removed via rotary evaporation. Purification via flash chromatography yielded 0.0797 g, 0.15 mmol, 88% yield) of (4S,5R)-2-(trimethylsilyl)ethyl 4-benzyl-5-((2R,4R)-1-(tert-butoxycarbonyl)-4-methoxypyrrolidin-2-yl)-2,2-dimethyloxazolidine-3-carboxylate.

A stirred solution of (4S,5R)-2-(trimethylsilyl)ethyl 4-benzyl-5-((2R,4R)-1-(tert-butoxycarbonyl)-4-methoxypyrrolidin-2-yl)-2,2-dimethyloxazolidine-3-carboxylate (0.0797 g, 0.15 mmol, 1 eq) in anhydrous CH₃CN (2 ml) under Ar was treated sequentially with KF (0.0260 g, 0.45 mmol, 3 eq) and TBAF (1.0 M in THF, 0.22 ml, 0.22 mmol, 1.5 eq). The resulting mixture was heated to 50° C. After 48 h the mixture was cooled to room temperature, diluted with EtOAc, and poured into saturated aqueous NaHCO₃. The layers were separated. The organic layer was washed with water (×2), brine (×1), and dried over Na₂SO₄. The inorganics were filtered off, and the solvent was removed via rotary evaporation to yield crude (2R,4R)-tert-butyl 2-((1S,2S)-2-amino-1-hydroxy-3-phenylpropyl)-4-methoxypyrrolidine-1-carboxylate which was used without purification.

### Example 1.6.16

### (2R,4R)-tert-butyl 2-((1S,2S)-2-amino-1-hydroxy-3-phenylpropyl)-4-phenoxypyrrolidine-1-carboxylate

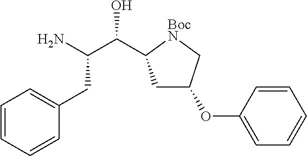 [SMILES: CC(C)(C)OC(=O)N1C[C@H](Oc2ccccc2)C[C@@H]1[C@@H](O)[C@@H](N)Cc1ccccc1]

To a solution of (2R,4R)-tert-butyl 2-((4S,5S)-4-benzyl-2-oxooxazolidin-5-yl)-4-(benzyloxy)pyrrolidine-1-carboxylate (850 mg, 1.88 mmoles) in MeOH (20 mL) was added ˜400 mg of 10% Pd/C and hydrogenated at 60 psi for 12 h. The catalyst was filtered off to yield (2R,4R)-tert-butyl 2-((4S,5S)-4-benzyl-2-oxooxazolidin-5-yl)-4-hydroxypyrrolidine-1-carboxylate quantitatively and was used for the next step without further purification.

To a solution of TPP (360 mg, 1.37 mmoles) in THF (10 mL) at 0° C. was added DIAD (0.27 mL mg, 1.37 mmoles). After 5 min, (2R,4R)-tert-butyl 2-((4S,5S)-4-benzyl-2-oxooxazolidin-5-yl)-4-hydroxypyrrolidine-1-carboxylate (360 mg, 1.15 mmoles) in THF (10 mL) was added. After another 5 min acetic acid (0.065 mL, 1.15 mmoles) was added and the reaction was stirred for 1.5 h. It was then quenched with PH˜7 buffer and extracted with EtOAc, dried on Na₂SO₄, concentrated and purified to obtain 300 mg of (2R,4S)-tert-butyl 4-acetoxy-2-((4S,5S)-4-benzyl-2-oxooxazolidin-5-yl)pyrrolidine-1-carboxylate.

K₂CO₃ (350 mg, 2.53 mmoles) was added, to a solution of (2R,4S)-tert-butyl 4-acetoxy-2-((4S,5S)-4-benzyl-2-oxooxazolidin-5-yl)pyrrolidine-1-carboxylate (300 mg, 1.15 mmoles) in MeOH (15 mL) and stirred for 2.5 h. All solvent was evaporated, diluted with EtOAc, washed with water and brine. The organics were dried on Na₂SO₄, concentrated and purified to obtain 260 mg (87% yields) of (2R,4S)-tert-butyl 2-((4S,5S)-4-benzyl-2-oxooxazolidin-5-yl)-4-hydroxypyrrolidine-1-carboxylate.

To a solution of TPP (314 mg, 1.20 mmoles) in THF (6 mL) at 0° C. was added DIAD (0.023 mL mg, 1.20 mmoles). After 5 min, (2R,4S)-tert-butyl 2-((4S,5S)-4-benzyl-2-oxooxazolidin-5-yl)-4-hydroxypyrrolidine-1-carboxylate (260 mg, 1.0 mmoles) in THF (6 mL) was added. After another 5 min phenol (0.065 mL, 1.15 mmoles) was added and the reaction was stirred for 12 days. It was then quenched with PH˜7 buffer and extracted with EtOAc, dried on Na₂SO₄, concentrated and purified to obtain 100 mg (23% yield) of (2R,4R)-tert-butyl 2-((4S,5S)-4-benzyl-2-oxooxazolidin-5-yl)-4-phenoxypyrrolidine-1-carboxylate.

Ba(OH)₂.8H₂O (315 mg, 1.0 mmol,) was added to a stirred solution of (2R,4R)-tert-butyl 2-((4S,5S)-4-benzyl-2-oxooxazolidin-5-yl)-4-phenoxypyrrolidine-1-carboxylate (100, 0.22 mmol,) in 1,4-dioxane/water (4 ml:2 ml). The reaction was heated to reflux at 105° C. After 3 h reaction was cooled to room temperature. The mixture was diluted with CH₂Cl₂/brine and filtered. The layers were separated. The aqueous layer was extracted with CH₂Cl₂ (×1). The combined organics were dried over Na₂SO₄. The inorganics were filtered off, and the solvent was removed via rotary evaporation. Purification via flash chromatography on silica gel yielded 45 mg (49% yields) of (2R,4R)-tert-butyl 2-((1S,2S)-2-amino-1-hydroxy-3-phenylpropyl)-4-phenoxypyrrolidine-1-carboxylate.

### Example 1.7

### Hydroxylamine/Isophthalate Coupling

### Example 1.7.1

### (2R,4R)-tert-butyl 4-(benzyloxy)-2-((1S,2S)-1-hydroxy-2-(3-((R)-2-(4-methylthiazol-2-yl)pyrrolidine-1-carbonyl)benzamido)-3-phenylpropyl)pyrrolidine-1-carboxylate

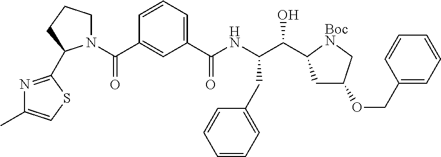 [SMILES: Cc1csc([C@H]2CCCN2C(=O)c2cccc(C(=O)N[C@@H](Cc3ccccc3)[C@H](O)[C@H]3C[C@@H](OCc4ccccc4)CN3C(=O)OC(C)(C)C)c2)n1]

HOBT.H₂O (0.0218 g, 0.16 mmol, 1.1 eq) was added to a stirred solution of (R)-3-(2-(4-methylthiazol-2-yl)pyrrolidine-1-carbonyl)benzoic acid (0.0464 g, 0.15 mmol, 1 eq) in 3 ml anhydrous CH₂Cl₂ at 0° C. under Ar. After 30 min EDCI.HCl (0.0308 g, 0.16 mmol, 1.1 eq) was added. After 2 h the resulting solution was treated with a solution of (4R)-tert-butyl 2-((1S,2S)-2-amino-1-hydroxy-3-phenylpropyl)-4-(benzyloxy)pyrrolidine-1-carboxylate (0.0625 g, 0.15 mmol, 1 eq) and DIPEA (0.064 ml, 0.047 g, 0.37 mmol, 2.5 eq) in 2 ml anhydrous CH₂Cl₂. The reaction was stirred at 0° C. to room temperature overnight. The solvent was removed via rotary evaporation. The residue was quenched with water and extracted with EtOAc (×1). The organic layer was washed with water (×2), brine (×1), and dried over Na₂SO₄. The inorganics were filtered off, and the solvent was removed via rotary evaporation. Purification via flash chromatography on silica gel yielded 0.0876 g (0.12 mmol, 81% yield) of (2R,4R)-tert-butyl 4-(benzyloxy)-2-((1S,2S)-1-hydroxy-2-(3-((R)-2-(4-methylthiazol-2-yl)pyrrolidine-1-carbonyl)benzamido)-3-phenylpropyl)pyrrolidine-1-carboxylate.

### Example 1.7.2

### (2R,4R)-tert-butyl 4-(benzyloxy)-2-((1S,2S)-2-(3-(fluoromethyl)-5-((R)-2-(4-methylthiazol-2-yl)pyrrolidine-1-carbonyl)benzamido)-1-hydroxy-3-phenylpropyl)pyrrolidine-1-carboxylate

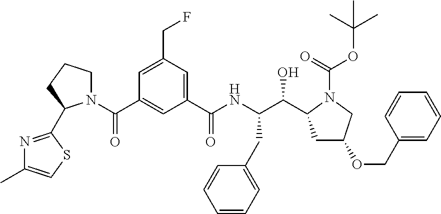 [SMILES: Cc1csc([C@H]2CCCN2C(=O)c2cc(CF)cc(C(=O)N[C@@H](Cc3ccccc3)[C@H](O)[C@H]3C[C@@H](OCc4ccccc4)CN3C(=O)OC(C)(C)C)c2)n1]

To a stirred solution of (R)-3-(fluoromethyl)-5-(2-(4-methylthiazol-2-yl)pyrrolidine-1-carbonyl)benzoic acid (131.3 mg, 0.3768 mmol) and (2R,4R)-tert-butyl 2-((1S,2S)-2-amino-1-hydroxy-3-phenylpropyl)-4-(benzyloxy)pyrrolidine-1-carboxylate (153 mg, 0.3589 mmol) in DCM was added triethylamine (1 mL, excess) and Py-BOP (205.4 mg, 0.3948 mmol) at room temperature. The reaction mixture was stirred at room temperature for 16 h. Then water was added and the reaction mixture was extracted with EtOAc. The organic layers were dried over Na₂SO₄ and concentrated. The crude product thus obtained was purified by silica gel flash column chromatography to provide (2R,4R)-tert-butyl 4-(benzyloxy)-2-((1S,2S)-2-(3-(fluoromethyl)-5-((R)-2-(4-methylthiazol-2-yl)pyrrolidine-1-carbonyl)benzamido)-1-hydroxy-3-phenylpropyl)pyrrolidine-1-carboxylate as white solid (220 mg).

### Example 1.7.3

### (R)-tert-butyl 2-((1S,2S)-1-hydroxy-2-(3-(methyl((4-methylthiazol-2-yl)methyl)carbamoyl)benzamido)-3-phenylpropyl)pyrrolidine-1-carboxylate

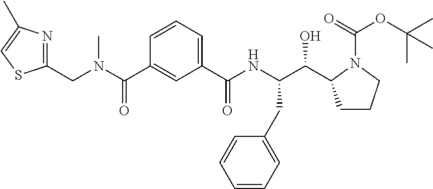 [SMILES: Cc1csc(CN(C)C(=O)c2cccc(C(=O)N[C@@H](Cc3ccccc3)[C@H](O)[C@H]3CCCN3C(=O)OC(C)(C)C)c2)n1]

Following normal coupling procedure described about, acid 3-(methyl((4-methylthiazol-2-yl)methyl)carbamoyl)benzoic acid (92.8 mg, 0.32 mmol) and amino-alcohol tert-butyl 2-((1S,2S)-2-amino-1-hydroxy-3-phenylpropyl)pyrrolidine-1-carboxylate (102 mg, 0.32 mmol) were coupled to provide (R)-tert-butyl 2-((1S,2S)-1-hydroxy-2-(3-(methyl((4-methylthiazol-2-yl)methyl)carbamoyl)benzamido)-3-phenylpropyl)pyrrolidine-1-carboxylate (54.6 mg, 29%) as a pale yellow oil.

### Example 1.7.4

### N-((1R,2S)-1-((2R,4R)-4-(allyloxy)pyrrolidin-2-yl)-1-hydroxy-3-phenylpropan-2-yl)-3-((R)-2-(4-methylthiazol-2-yl)pyrrolidine-1-carbonyl)-5-(oxazol-2-yl)benzamide

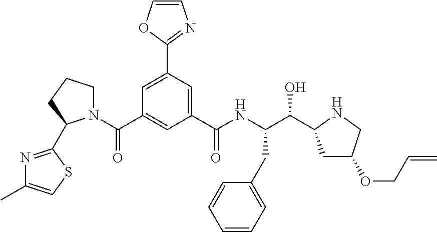 [SMILES: C=CCO[C@H]1CN[C@@H]([C@@H](O)[C@H](Cc2ccccc2)NC(=O)c2cc(C(=O)N3CCC[C@@H]3c3nc(C)cs3)cc(-c3ncco3)c2)C1]

To a stirred solution of (2R,4R)-tert-butyl 4-(allyloxy)-2-((4S,5S)-4-benzyl-2,2-dimethyloxazolidin-5-yl)pyrrolidine-1-carboxylate and (R)-3-(2-(4-methylthiazol-2-yl)pyrrolidine-1-carbonyl)-5-(oxazol-2-yl)benzoic acid (63.5 mg, 0.15 mmol) in anhydrous methylene chloride were added PyBOP reagent (82.4 mg, 0.16 mmol) and triethylamine (0.2 mL, excess) at room temperature. The reaction mixture was stirred at room temperature for 16 h. Then water was added and the reaction mixture was extracted with EtOAc. The organic layers were dried over Na₂SO₄ and concentrated. Purification of the crude product by silica gel flash column chromatography provided (2R,4R)-tert-butyl 4-(allyloxy)-2-((1S,2S)-1-hydroxy-2-(3-((R)-2-(4-methylthiazol-2-yl)pyrrolidine-1-carbonyl)-5-(oxazol-2-yl)benzamido)-3-phenylpropyl)pyrrolidine-1-carboxylate (50 mg, 44%), which was dissolved in 4 M HCl in dioxane (6 mL) and methanol (0.5 mL) at room temperature. The reaction mixture was stirred at room temperature for 16 h. Then saturated sodium bicarbonate solution was added and the mixture was extracted with chloroform three times. The organic layers were dried over Na₂SO₄ and concentrated. The crude product thus obtained after purified by basic alumina column chromatography to provide N-((1R,2S)-1-((2R,4R)-4-(allyloxy)pyrrolidin-2-yl)-1-hydroxy-3-phenylpropan-2-yl)-3-((R)-2-(4-methylthiazol-2-yl)pyrrolidine-1-carbonyl)-5-(oxazol-2-yl)benzamide.

### Example 1.7.5

### (2R,4R)-tert-butyl 4-hydroxy-2-((1S,2S)-1-hydroxy-2-(3-((R)-2-(4-methylthiazol-2-yl)pyrrolidine-1-carbonyl)-5-(oxazol-2-yl)benzamido)-3-phenylpropyl)pyrrolidine-1-carboxylate

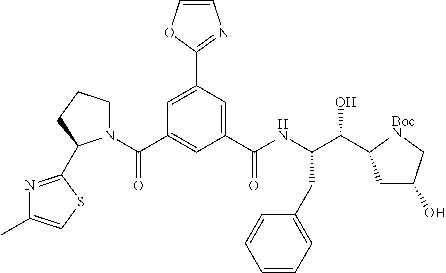 [SMILES: Cc1csc([C@H]2CCCN2C(=O)c2cc(C(=O)N[C@@H](Cc3ccccc3)[C@H](O)[C@H]3C[C@@H](O)CN3C(=O)OC(C)(C)C)cc(-c3ncco3)c2)n1]

To a stirred solution of (2R,4R)-tert-butyl 2-((1S,2S)-2-amino-1-hydroxy-3-phenylpropyl)-4-hydroxypyrrolidine-1-carboxylate (552 mg, 1.64 mmol), (R)-3-(2-(4-methylthiazol-2-yl)pyrrolidine-1-carbonyl)-5-(oxazol-2-yl)benzoic acid (723.6 mg, 1.723 mmol) in DCM were added triethylamine (1 mL, excess), EDCI (376.1 mg, 1.97 mmol) and HOBt (243.7 mg, 1.8 mmol) at room temperature. The reaction mixture was stirred at room temperature for 16 h. Then water was added and the reaction mixture was extracted with chloroform. The organic layers were separated, dried over Na₂SO₄ and concentrated. The crude product thus obtained was purified by silica gel flash column chromatography to provide (2R,4R)-tert-butyl 4-hydroxy-2-((1S,2S)-1-hydroxy-2-(3-((R)-2-(4-methylthiazol-2-yl)pyrrolidine-1-carbonyl)-5-(oxazol-2-yl)benzamido)-3-phenylpropyl)pyrrolidine-1-carboxylate as white solid (760 mg, 66%). ¹H NMR (300 MHz, CDCl₃), d: 8.483-8.255 (m, 2H), 7.951-7.674 (m, 2H), 7.262 (m, 6H), 6.801 (s, 1H), 5.632 (m, 0.7H), 5.106 (m, 0.3H), 4.375 (m, 2H), 4.228-4.040 (m, 3H), 3.684 (m, 1H), 3.461 (m, 2H), 3.262-2.893 (m, 2H), 2.454 (s, 3H), 2.387 (m, 1H), 2.238-1.905 (m, 5H), 1.455 (s, 6H), 1.325 (s, 3H),

### Example 1.8

### Post-Coupling Modifications

### Example 1.8.1

### N-((1R,2S)-1-((2R,4R)-4-(benzyloxy)pyrrolidin-2-yl)-1-hydroxy-3-phenylpropan-2-yl)-3-((R)-2-(4-methylthiazol-2-yl)pyrrolidine-1-carbonyl)benzamide

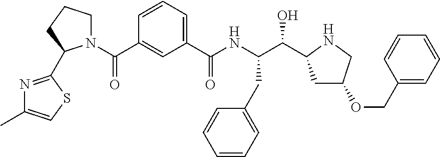 [SMILES: Cc1csc([C@H]2CCCN2C(=O)c2cccc(C(=O)N[C@@H](Cc3ccccc3)[C@H](O)[C@H]3C[C@@H](OCc4ccccc4)CN3)c2)n1]

A stirred solution of (2R,4R)-tert-butyl 4-(benzyloxy)-2-((1S,2S)-1-hydroxy-2-(3-((R)-2-(4-methylthiazol-2-yl)pyrrolidine-1-carbonyl)benzamido)-3-phenylpropyl)pyrrolidine-1-carboxylate (0.0876 g, 0.12 mmol, 1 eq) in 0.5 ml anhydrous MeOH under Ar was treated with HCl in 1,4-dioxane (4.0 M, 0.5 ml, 2.0 mmol, large excess). After 1 h the solvent was removed via rotary evaporation. The resulting residue was stirred in saturated aqueous NaHCO₃/CH₂Cl₂. After 30 min the layers were separated. The organic layer was dried over Na₂SO₄. The inorganics were filtered off, and the solvent was removed via rotary evaporation. Purification via flash chromatography on basic alumina yielded 0.0402 g (0.064 mmol, 54% yield) of N-((1R,2S)-1-((2R,4R)-4-(benzyloxy)pyrrolidin-2-yl)-1-hydroxy-3-phenylpropan-2-yl)-3-((R)-2-(4-methylthiazol-2-yl)pyrrolidine-1-carbonyl)benzamide.

### Example 1.8.2

### N1-((1R,2S)-1-hydroxy-1-((2R,4R)-4-hydroxypyrrolidin-2-yl)-3-phenylpropan-2-yl)-N3-methyl-N3-((4-methylthiazol-2-yl)methyl)isophthalamide

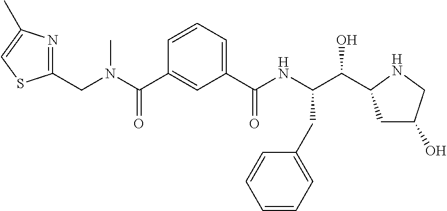 [SMILES: Cc1csc(CN(C)C(=O)c2cccc(C(=O)N[C@@H](Cc3ccccc3)[C@H](O)[C@H]3C[C@@H](O)CN3)c2)n1]

BBr₃ (1.0 M in CH₂Cl₂, 0.11 ml, 0.11 mmol, 3 eq) was added to a stirred solution of (2R,4R)-tert-butyl 4-(benzyloxy)-2-((1S,2S)-1-hydroxy-2-(3-(methyl((4-methylthiazol-2-yl)methyl)carbamoyl)benzamido)-3-phenylpropyl)pyrrolidine-1-carboxylate (0.0264 g, 0.037 mmol, 1 eq) in 2 ml anhydrous CH₂Cl₂ at 0° C. under Ar. After 40 min the reaction was quenched with MeOH (1 ml), and the solvent was removed via rotary evaporation. The residue was diluted with water, adjusted to pH^{˜}2 with 1N HCl, and extracted with CH₂Cl₂ (×2). The aqueous layer was adjusted to pH>7 with saturated aqueous NaHCO₃ and extracted with 10% MeOH in CH₂Cl₂ (×3). The combined 10% MeOH in CH₂Cl₂ fractions were dried over Na₂SO₄. The inorganics were filtered off, and the solvent was removed via rotary evaporation yielding 0.0022 g (0.004 mmol, 11% yield) of N1-((1R,2S)-1-hydroxy-1-((2R,4R)-4-hydroxypyrrolidin-2-yl)-3-phenylpropan-2-yl)-N3-methyl-N3-((4-methylthiazol-2-yl)methyl)isophthalamide.

### Example 1.8.3

### N1-((1R,2S)-1-hydroxy-3-phenyl-1-((R)-pyrrolidin-2-yl)propan-2-yl)-N3-methyl-N3-((4-methylthiazol-2-yl)methyl)isophthalamide

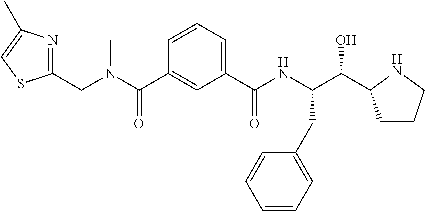 [SMILES: Cc1csc(CN(C)C(=O)c2cccc(C(=O)N[C@@H](Cc3ccccc3)[C@H](O)[C@H]3CCCN3)c2)n1]

To a stirred solution of (R)-tert-butyl 2-((1S,2S)-1-hydroxy-2-(3-(methyl((4-methylthiazol-2-yl)methyl)carbamoyl)benzamido)-3-phenylpropyl)pyrrolidine-1-carboxylate (50.8 mg, 0.09 mmol) in CH₂Cl₂ (5 mL) at 0° C. was added TFA (1 mL). The resulting solution was stirred for 4 h while warmed up to r.t. The solvent was removed under reduced pressure and the residue was diluted with CHCl₃ and saturated aqueous NaHCO₃. The layers were separated and the aqueous layer was extracted with CHCl₃. The combined organic layer was washed with brine, dried with Na₂SO₄ and concentrated under reduced pressure. The residue was purified by column chromatography (20% methanol in chloroform) to provide N1-((1R,2S)-1-hydroxy-3-phenyl-1-((R)-pyrrolidin-2-yl)propan-2-yl)-N3-methyl-N3-((4-methylthiazol-2-yl)methyl)isophthalamide.

### Example 1.8.4

### N-((1R,2 S)-1-((2R,4R)-4-(benzyloxy)pyrrolidin-2-yl)-1-hydroxy-3-phenylpropan-2-yl)-3-(fluoromethyl)-5-((R)-2-(4-methylthiazol-2-yl)pyrrolidine-1-carbonyl)benzamide

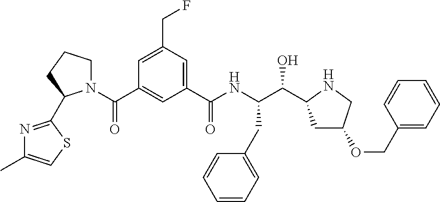 [SMILES: Cc1csc([C@H]2CCCN2C(=O)c2cc(CF)cc(C(=O)N[C@@H](Cc3ccccc3)[C@H](O)[C@H]3C[C@@H](OCc4ccccc4)CN3)c2)n1]

(2R,4R)-tert-butyl 4-(benzyloxy)-2-((1S,2S)-2-(3-(fluoromethyl)-5-((R)-2-(4-methylthiazol-2-yl)pyrrolidine-1-carbonyl)benzamido)-1-hydroxy-3-phenylpropyl)pyrrolidine-1-carboxylate was dissolved in 4 M HCl in dioxane (6 mL) and methanol (0.5 mL) at room temperature. The reaction mixture was stirred at room temperature for 16 h. Then saturated sodium bicarbonate solution was added and the mixture was extracted with chloroform three times. The organic layers were dried over Na₂SO₄ and concentrated. The crude product thus obtained after purified by basic alumina column chromatography to provide the final compound (145 mg).

### Example 1.8.5

### N1-((1R,2S)-3-(3,5-difluorophenyl)-1-hydroxy-1-((R)-pyrrolidin-2-yl)propan-2-yl)-N3-methyl-N3-((4-methylthiazol-2-yl)methyl)isophthalamide

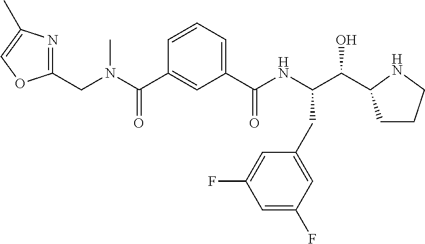 [SMILES: Cc1coc(CN(C)C(=O)c2cccc(C(=O)N[C@@H](Cc3cc(F)cc(F)c3)[C@H](O)[C@H]3CCCN3)c2)n1]

Following the normal coupling procedures described herein, 3-(methyl((4-methylthiazol-2-yl)methyl)carbamoyl)benzoic acid (38 mg, 0.13 mmol) and (R)-tert-butyl 2-((1S,2S)-2-amino-3-(3,5-difluorophenyl)-1-hydroxypropyl)pyrrolidine-1-carboxylate (48.5 mg, 0.13 mmol) were coupled to provide (R)-tert-butyl 2-((1S,2S)-3-(3,5-difluorophenyl)-1-hydroxy-2-(3-(methyl((4-methylthiazol-2-yl)methyl)carbamoyl)benzamido)propyl)pyrrolidine-1-carboxylate (76.6 mg, 94%) as a pale yellow oil.

(R)-tert-butyl 2-((1S,2S)-3-(3,5-difluorophenyl)-1-hydroxy-2-(3-(methyl((4-methylthiazol-2-yl)methyl)carbamoyl)benzamido)propyl)pyrrolidine-1-carboxylate (76 mg, 0.12 mmol) in methanol (1 mL) was treated with HCl in dioxane (4.0 N, 3 mL) at 0° C. The resulting solution was stirred for 4 h while warmed up to r.t. The solvent was removed under reduced pressure and the residue was diluted with CHCl₃ and saturated aqueous NaHCO₃. The layers were separated and the aqueous layer was extracted with CHCl₃. The combined organic layer was washed with brine, dried with Na₂SO₄ and concentrated under reduced pressure to provide N1-((1R,2S)-3-(3,5-difluorophenyl)-1-hydroxy-1-((R)-pyrrolidin-2-yl)propan-2-yl)-N3-methyl-N3-((4-methylthiazol-2-yl)methyl)isophthalamide (41 mg, 65%) as an off-white solid.

### Example 2

### Inhibitor Compounds

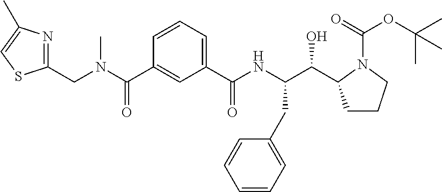 [SMILES: Cc1csc(CN(C)C(=O)c2cccc(C(=O)N[C@@H](Cc3ccccc3)[C@H](O)[C@H]3CCCN3C(=O)OC(C)(C)C)c2)n1]

(R)-tert-butyl 2-((1S,2S)-1-hydroxy-2-(3-(methyl((4-methylthiazol-2-yl)methyl)carbamoyl)benzamido)-3-phenylpropyl)pyrrolidine-1-carboxylate: 1H NMR (CDCl₃): d 7.82-7.88 (m, 2H), 7.57-7.59 (m, 1H), 7.41-7.46 (m, 1H), 7.15-7.25 (m, 5H), 6.87 (s, 1H), 4.95 (m, 2H), 4.63 (m, 2H), 3.99 (s, 2H), 3.68 (m, 1H), 3.44-3.47 (m, 1H), 3.32-3.34 (m, 1H), 2.88-3.09 (m, 2H), 3.00 (s, 3H), 2.43 (s, 3H), 2.14 (m, 1H), 1.84-1.90 (m, 3H), 1.49 (s, 9H)

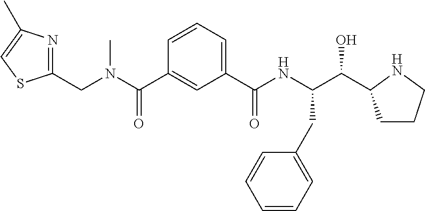 [SMILES: Cc1csc(CN(C)C(=O)c2cccc(C(=O)N[C@@H](Cc3ccccc3)[C@H](O)[C@H]3CCCN3)c2)n1]

N1-((1R,2S)-1-hydroxy-3-phenyl-1-((R)-pyrrolidin-2-yl)propan-2-yl)-N3-methyl-N3-((4-methylthiazol-2-yl)methyl)isophthalamide: (7.3 mg, 18%) as a white solid. ¹H NMR (CDCl₃): d 7.59 (m, 2H), 7.41-7.43 (m, 1H), 7.33-7.37 (m, 1H), 6.98-7.23 (m, 5H), 6.85 (s, 1H), 4.86 (m, 2H), 4.18 (m, 1H), 3.53 (m, 1H), 3.22-3.28 (m, 1H), 3.12-3.16 (m, 2H), 3.00 (m, 1H), 2.89 (s, 3H), 2.64-2.72 (m, 1H), 2.35 (s, 3H), 1.81-2.07 (m, 4H).

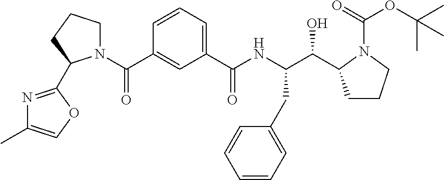 [SMILES: Cc1coc([C@H]2CCCN2C(=O)c2cccc(C(=O)N[C@@H](Cc3ccccc3)[C@H](O)[C@H]3CCCN3C(=O)OC(C)(C)C)c2)n1]

(R)-tert-butyl 2-((1S,2S)-1-hydroxy-2-(3-((R)-2-(4-methyloxazol-2-yl)pyrrolidine-1-carbonyl)benzamido)-3-phenylpropyl)pyrrolidine-1-carboxylate: 1H NMR (CDCl₃): d 7.98 (s, 1H), 7.88-7.91 (m, 1H), 7.80-7.82 (m, 1H), 7.63-7.66 (m, 1H), 7.36-7.42 (m, 1H), 7.09-7.26 (m, 5H), 5.31-5.36 (m, 1H), 4.57 (m, 1H), 4.12-4.14 (m, 1H), 4.00 (m, 1H), 3.67-3.70 (m, 2H), 3.44-3.50 (m, 2H), 3.31 (m, 1H), 2.97-3.04 (m, 1H), 2.82-2.90 (m, 1H), 2.27-2.32 (m, 1H), 2.01-2.18 (m, 3H), 2.12 (s, 3H), 1.82-1.94 (m, 4H), 1.48 (s, 9H).

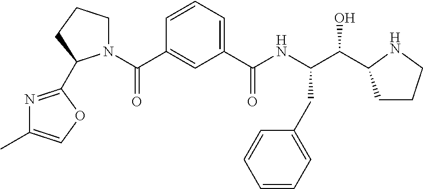 [SMILES: Cc1coc([C@H]2CCCN2C(=O)c2cccc(C(=O)N[C@@H](Cc3ccccc3)[C@H](O)[C@H]3CCCN3)c2)n1]

N-((1R,2S)-1-hydroxy-3-phenyl-1-((R)-pyrrolidin-2-yl)propan-2-yl)-3-((R)-2-(4-methyloxazol-2-yl)pyrrolidine-1-carbonyl)benzamide: 1H NMR (CDCl₃): d 7.78-7.84 (m, 2H), 7.67 (m, 1H), 7.45-7.47 (m, 1H), 7.01-7.23 (m, 6H), 5.21 (m, 2H), 4.24 (m, 2H), 3.73 (m, 2H), 3.55-3.61 (m, 1H), 3.26-3.37 (m, 2H), 3.06 (s, 2H), 2.87 (m, 1H), 2.22-2.26 (m, 1H), 1.76-2.06 (m, 7H).

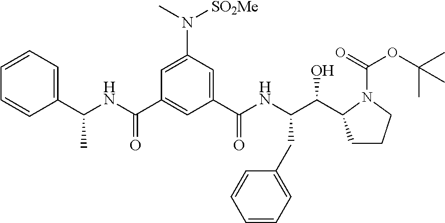 [SMILES: C[C@@H](NC(=O)c1cc(C(=O)N[C@@H](Cc2ccccc2)[C@H](O)[C@H]2CCCN2C(=O)OC(C)(C)C)cc(N(C)S(C)(=O)=O)c1)c1ccccc1]

(R)-tert-butyl 2-((1S,2S)-1-hydroxy-2-(3-(N-methylmethylsulfonamido)-5-((R)-1-phenylethylcarbamoyl)benzamido)-3-phenylpropyl)pyrrolidine-1-carboxylate: ¹H NMR (300 MHz, CDCl₃+CD₃OD), d: 8.129-7.974 (m, 3H), 7.380-7.183 (m, 10H), 5.323 (m, 1H), 4.662 (m, 1H), 4.026 (m, 1H), 3.494 (m, 1H), 3.349 (m, 5H), 2.967 (m, 2H), 2.869 (m, 3H), 2.176 (m, 2H), 1.900 (m, 2H), 1.615 (m, 3H), 1.542 (s, 9H).

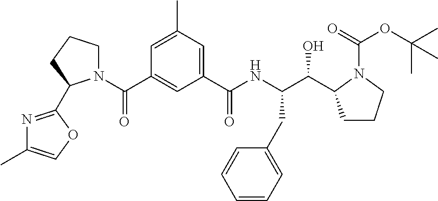 [SMILES: Cc1cc(C(=O)N[C@@H](Cc2ccccc2)[C@H](O)[C@H]2CCCN2C(=O)OC(C)(C)C)cc(C(=O)N2CCC[C@@H]2c2nc(C)co2)c1]

(R)-tert-butyl 2-((1S,2S)-1-hydroxy-2-(3-methyl-5-((R)-2-(4-methyloxazol-2-yl)pyrrolidine-1-carbonyl)benzamido)-3-phenylpropyl)pyrrolidine-1-carboxylate: ¹H NMR (300 MHz, CDCl₃+CD₃OD), d: 7.847 (m, 1H), 7.724 (m, 1H), 7.528 (m, 1H), 7.374-7.181 (m, 6H), 5.406 (m, 0.75H), 4.862 (m, 0.25H), 4.607 (m, 1H), 4.057 (m, 1H), 3.871-3.712 (m, 2H), 3.530 (m, 2H), 3.368 (m, 1H), 3.090-2.906 (m, 2H), 2.410 (s, 3H), 2.188 (s, 3H), 2.365-1.812 (m, 8H), 1.541 (s, 9H).

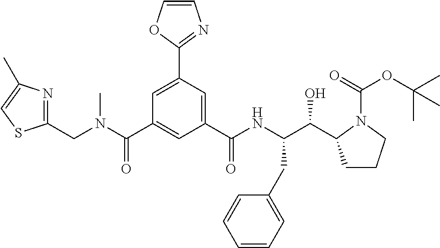 [SMILES: Cc1csc(CN(C)C(=O)c2cc(C(=O)N[C@@H](Cc3ccccc3)[C@H](O)[C@H]3CCCN3C(=O)OC(C)(C)C)cc(-c3ncco3)c2)n1]

(R)-tert-butyl 2-((1S,2S)-1-hydroxy-2-(3-(methyl((4-methylthiazol-2-yl)methyl)carbamoyl)-5-(oxazol-2-yl)benzamido)-3-phenylpropyl)pyrrolidine-1-carboxylate:

¹H NMR (300 MHz, CDCl₃+CD₃OD), d: 8.631 (s, 1H), 8.352 (m, 2H), 8.032 (s, 1H), 7.757 (s, 1H), 7.268 (m, 5H), 6.934 (s, 1H), 5.008 (s, 1.4H), 4.695 (br, 1.6H), 4.062 (m, 1H), 3.730 (m, 1H), 3.501 (m, 1H), 3.396 (m, 1H), 3.165-2.898 (m, 5H), 2.485 (s, 3H), 2.188 (m, 1H), 1.897 (m, 3H), 1.572 (s, 9H).

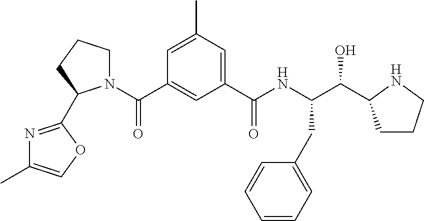 [SMILES: Cc1cc(C(=O)N[C@@H](Cc2ccccc2)[C@H](O)[C@H]2CCCN2)cc(C(=O)N2CCC[C@@H]2c2nc(C)co2)c1]

N-((1R,2S)-1-hydroxy-3-phenyl-1-((R)-pyrrolidin-2-yl)propan-2-yl)-3-methyl-5-((R)-2-(4-methyloxazol-2-yl)pyrrolidine-1-carbonyl)benzamide: ¹H NMR (300 MHz, CDCl₃+CD₃OD), d: 7.670 (s, 1H), 7.594 (s, 1H), 7.476 (s, 1H), 7.344-6.979 (m, 6H), 5.354 (m, 0.79H), 4.812 (m, 0.19H), 4.374 (m, 1H), 3.742 (m, 2H), 3.463 (m, 1H), 3.201 (m, 2H), 3.016 (m, 2H), 2.823 (m, 1H), 2.371 (s, 3H), 2.172 (s, 3H), 2.246-1.702 (m, 8H).

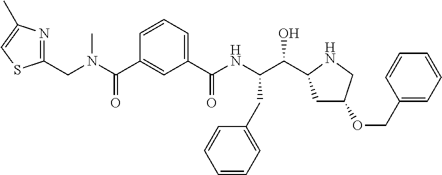 [SMILES: Cc1csc(CN(C)C(=O)c2cccc(C(=O)N[C@@H](Cc3ccccc3)[C@H](O)[C@H]3C[C@@H](OCc4ccccc4)CN3)c2)n1]

N1-((1R,2S)-1-((2R,4R)-4-(benzyloxy)pyrrolidin-2-yl)-1-hydroxy-3-phenylpropan-2-yl)-N3-methyl-N3-((4-methylthiazol-2-yl)methyl)isophthalamide: ¹H NMR (300 MHz, CDCl₃) d 7.85-7.50 (m, 3H), 7.42-7.04 (m, 13H), 6.95-6.72 (m, 2H), 4.97 (m, 1H), 4.65-4.39 (m, 4H), 4.11 (m, 1H), 3.80-3.68 (m, 1H), 3.50 (m, 2H), 3.26-2.90 (m, 7H), 2.48 (m, 3H), 2.18 (m, 2H).

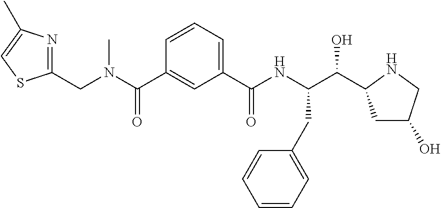 [SMILES: Cc1csc(CN(C)C(=O)c2cccc(C(=O)N[C@@H](Cc3ccccc3)[C@H](O)[C@H]3C[C@@H](O)CN3)c2)n1]

N1-((1R,2S)-1-hydroxy-1-((2R,4R)-4-hydroxypyrrolidin-2-yl)-3-phenylpropan-2-yl)-N3-methyl-N3-((4-methylthiazol-2-yl)methyl)isophthalamide: ¹H NMR (300 MHz, CDCl₃) δ 7.86-7.64 (m, 3H), 7.53-7.35 (m, 6H), 7.34-7.14 (m, 14H), 6.93 (m, 2H), 4.96 (m, 2H), 4.66 (m, 1H), 4.37 (m, 3H), 3.98 (m, 1H), 3.68 (m, 2H), 3.39-3.28 (m, 2H), 3.16-2.86 (m, 5H), 2.46 (s, 3H).

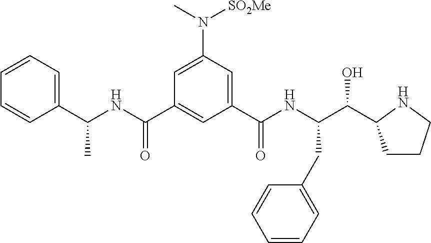 [SMILES: C[C@@H](NC(=O)c1cc(C(=O)N[C@@H](Cc2ccccc2)[C@H](O)[C@H]2CCCN2)cc(N(C)S(C)(=O)=O)c1)c1ccccc1]

N1-((1R,2S)-1-hydroxy-3-phenyl-1-((R)-pyrrolidin-2-yl)propan-2-yl)-5-(N-methylmethylsulfonamido)-N3-((R)-1-phenylethyl)isophthalamide: ¹H NMR (300 MHz, CDCl₃+CD₃OD), d: 8.065 (s, 1H), 7.910 (s, 1H), 7.734 (s, 1H), 7.380-7.122 (m, 10H), 5.259 (m, 1H), 4.348 (m, 1H), 3.721 (m, 1H), 3.234 (s, 3H), 3.166 (m, 2H), 2.946 (m, 2H), 2.791 (s, 4H), 1.855-1.675 (m, 4H), 1.538 (d, J=6.6 Hz, 3H).

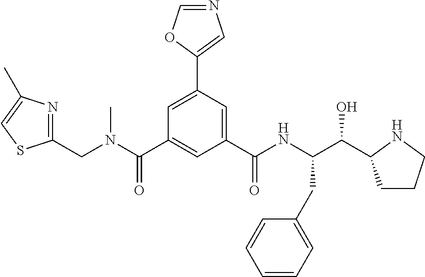 [SMILES: Cc1csc(CN(C)C(=O)c2cc(C(=O)N[C@@H](Cc3ccccc3)[C@H](O)[C@H]3CCCN3)cc(-c3cnco3)c2)n1]

N1-((1R,2S)-1-hydroxy-3-phenyl-1-((R)-pyrrolidin-2-yl)propan-2-yl)-N3-methyl-N3-((4-methylthiazol-2-yl)methyl)-5-(oxazol-5-yl)isophthalamide: ¹H NMR (CDCl₃): d 7.89 (m, 2H), 7.60-7.68 (m, 2H), 7.38 (m, 1H), 7.07-7.25 (m, 5H), 6.90 (s, 1H), 4.93 (m, 2H), 4.38 (m, 1H), 3.85 (m, 1H), 3.34-3.36 (m, 2H), 3.21-3.24 (m, 1H), 2.95-3.11 (m, 2H), 2.98 (s, 3H), 2.44 (s, 3H), 1.74-1.93 (m, 4H).

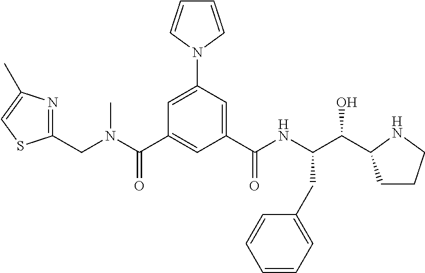 [SMILES: Cc1csc(CN(C)C(=O)c2cc(C(=O)N[C@@H](Cc3ccccc3)[C@H](O)[C@H]3CCCN3)cc(-n3cccc3)c2)n1]

N1-((1R,2S)-1-hydroxy-3-phenyl-1-((R)-pyrrolidin-2-yl)propan-2-yl)-N3-methyl-N3-((4-methylthiazol-2-yl)methyl)-5-(1H-pyrrol-1-yl)isophthalamide: ¹H NMR (CDCl₃): d 7.54-7.72 (m, 2H), 7.35-7.41 (m, 1H), 7.15-7.25 (m, 5H), 7.00 (s, 2H), 6.90 (m, 1H), 6.31 (s, 2H), 4.93 (m, 2H), 4.36 (m, 1H), 3.75 (m, 1H), 3.17-3.27 (m, 2H), 2.89-3.09 (m, 3H), 2.97 (s, 3H), 2.44 (s, 3H), 1.77-1.85 (m, 4H).

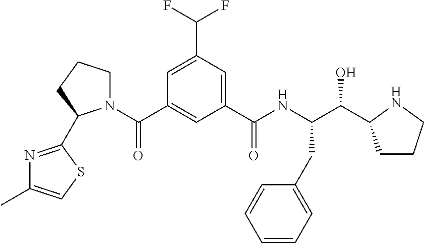 [SMILES: Cc1csc([C@H]2CCCN2C(=O)c2cc(C(=O)N[C@@H](Cc3ccccc3)[C@H](O)[C@H]3CCCN3)cc(C(F)F)c2)n1]

3-(difluoromethyl)-N-((1R,2S)-1-hydroxy-3-phenyl-1-((R)-pyrrolidin-2-yl)propan-2-yl)-5-((R)-2-(4-methylthiazol-2-yl)pyrrolidine-1-carbonyl)benzamide: ¹H NMR (300 MHz, CDCl₃+CD₃OD), d: 7.936 (s, 1H), 7.818 (s, 1H), 7.755 (s, 1H), 7.247 (m, 5H), 6.872 (s, 1H), 6.614 (m, 1H), 5.599 (m, 0.70H), 5.042 (m, 0.30H), 4.370 (m, 1H), 3.775 (m, 2H), 3.430 (m, 1H), 3.204 (m, 2H), 2.982 (m, 3H), 2.452 (s, 3H), 2.323 (m, 1H), 2.093 (m, 1H), 2.005-1.759 (m, 4H).

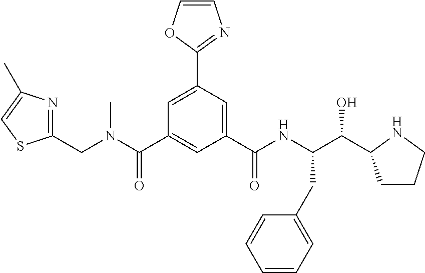 [SMILES: Cc1csc(CN(C)C(=O)c2cc(C(=O)N[C@@H](Cc3ccccc3)[C@H](O)[C@H]3CCCN3)cc(-c3ncco3)c2)n1]

N1-((1R,2S)-1-hydroxy-3-phenyl-1-((R)-pyrrolidin-2-yl)propan-2-yl)-N3-methyl-N3-((4-methylthiazol-2-yl)methyl)-5-(oxazol-2-yl)isophthalamide: ¹H NMR (300 MHz, CDCl₃+CD₃OD), d: 8.367-7.763 (m, 3H), 7.265 (m, 7H), 6.943 (s, 1H), 4.987 (s, 1.3H), 4.685 (br, 0.7H), 4.395 (s, 1H), 3.810 (m, 1H), 3.271-3.147 (m, 3H), 3.040 (s, 3H), 3.005-2.848 (m, 2H), 2.478 (s, 3H), 1.943-1.712 (m, 4H).

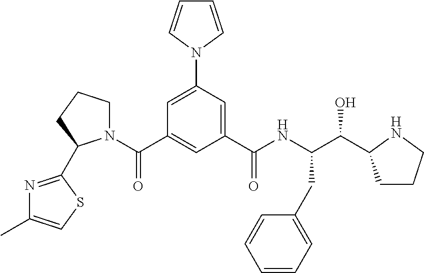 [SMILES: Cc1csc([C@H]2CCCN2C(=O)c2cc(C(=O)N[C@@H](Cc3ccccc3)[C@H](O)[C@H]3CCCN3)cc(-n3cccc3)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-(1H-pyrrol-1-yl)benzamide: 1H NMR (CDCl₃): d 7.47 (m, 2H), 7.41 (m, 1H), 7.13-7.25 (m, 6H), 6.94 (s, 1H), 6.81 (s, 1H), 6.33 (m, 2H), 5.63 (m, 1H), 4.38 (m, 1H), 3.86 (m, 1H), 3.59-3.73 (m, 2H), 3.39-3.46 (m, 1H), 3.30-3.32 (m, 1H), 3.18-3.24 (m, 1H), 2.95-3.09 (m, 2H), 2.45 (s, 3H), 2.30-2.40 (m, 2H), 2.05-2.14 (m, 1H), 1.76-1.97 (m, 5H).

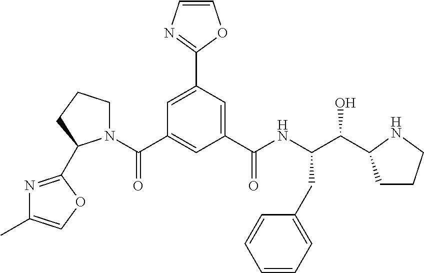 [SMILES: Cc1coc([C@H]2CCCN2C(=O)c2cc(C(=O)N[C@@H](Cc3ccccc3)[C@H](O)[C@H]3CCCN3)cc(-c3ncco3)c2)n1]

N-((1R,2S)-1-hydroxy-3-phenyl-1-((R)-pyrrolidin-2-yl)propan-2-yl)-3-((R)-2-(4-methyloxazol-2-yl)pyrrolidine-1-carbonyl)-5-(oxazol-2-yl)benzamide: ¹H NMR (CDCl₃): d 8.20-8.36 (m, 2H), 7.86-7.96 (m, 1H), 7.72-7.80 (m, 1H), 7.01-7.40 (m, 7H), 5.34-5.44 (m, 1H), 4.32-4.54 (m, 1H), 3.64-4.00 (m, 3H), 3.50-3.61 (m, 1H), 2.82-3.40 (m, 5H), 2.03 (s, 3H), 2.00-2.44 (m, 2H), 1.64-2.02 (m, 6H).

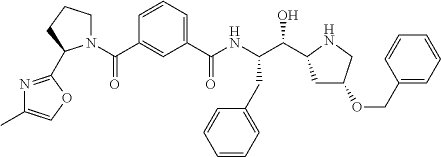 [SMILES: Cc1coc([C@H]2CCCN2C(=O)c2cccc(C(=O)N[C@@H](Cc3ccccc3)[C@H](O)[C@H]3C[C@@H](OCc4ccccc4)CN3)c2)n1]

N-((1R,2S)-1-((2R,4R)-4-(benzyloxy)pyrrolidin-2-yl)-1-hydroxy-3-phenylpropan-2-yl)-3-((R)-2-(4-methyloxazol-2-yl)pyrrolidine-1-carbonyl)benzamide: ¹H NMR (300 MHz, CDCl₃) [symbol] 7.82 (s, 1H), 7.69-7.50 (m, 2H), 7.42-7.19 (m, 11H), 6.60-6.58 (m, 1H), 5.39-5.35 (m, 0.6H), 4.80 (m, 0.2H), 4.58-4.38 (m, 3H), 4.09-4.08 (m, 1H), 3.85-3.66 (m, 2H), 3.51-3.36 (m, 2H), 3.21-3.03 (m, 3H), 2.84-2.79 (m, 1H), 2.40-1.89 (m, 8H).

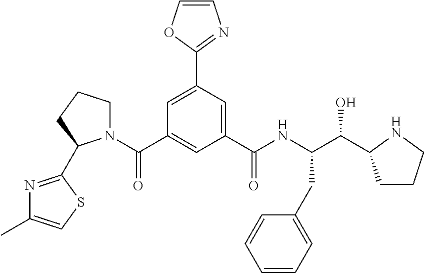 [SMILES: Cc1csc([C@H]2CCCN2C(=O)c2cc(C(=O)N[C@@H](Cc3ccccc3)[C@H](O)[C@H]3CCCN3)cc(-c3ncco3)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-(oxazol-2-yl)benzamide: ¹H NMR (CDCl₃): d 8.18-8.32 (m, 2H), 7.84-7.92 (m, 1H), 7.72-7.80 (m, 1H), 7.01-7.40 (m, 5H), 6.80-6.88 (m, 1H), 5.34-5.44 (m, 1H), 4.32-4.54 (m, 1H), 3.64-4.00 (m, 3H), 3.50-3.61 (m, 1H), 2.82-3.40 (m, 5H), 2.01 (s, 3H), 2.00-2.44 (m, 2H), 1.64-2.02 (m, 6H).

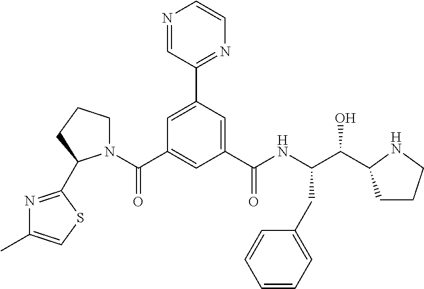 [SMILES: Cc1csc([C@H]2CCCN2C(=O)c2cc(C(=O)N[C@@H](Cc3ccccc3)[C@H](O)[C@H]3CCCN3)cc(-c3cnccn3)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-(pyrazin-2-yl)benzamide: ¹H NMR (300 MHz, CDCl₃): d 1.65-2.32 (m, 11H), 2.37-3.78 (m, 8H), 4.31-4.36 (m, 1H), 5.53-5.57 (m, 1H), 7.08-7.19 (m, 7H), 7.79-8.91 (m, 5H).

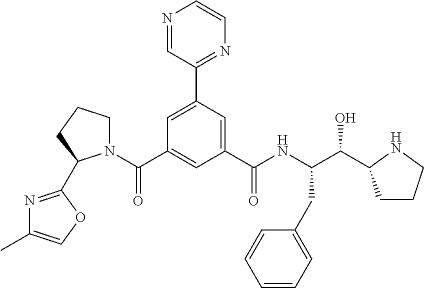 [SMILES: Cc1coc([C@H]2CCCN2C(=O)c2cc(C(=O)N[C@@H](Cc3ccccc3)[C@H](O)[C@H]3CCCN3)cc(-c3cnccn3)c2)n1]

N-((1R,2S)-1-hydroxy-3-phenyl-1-((R)-pyrrolidin-2-yl)propan-2-yl)-3-((R)-2-(4-methyloxazol-2-yl)pyrrolidine-1-carbonyl)-5-(pyrazin-2-yl)benzamide:1H NMR (300 MHz, CDCl₃): d 1.74-2.20 (m, 11H), 2.39-3.89 (m, 8H), 4.30-4.34 (m, 1H), 5.37-5.40 (m, 1H), 7.14-7.30 (m, 6H), 7.63-9.01 (m, 6H).

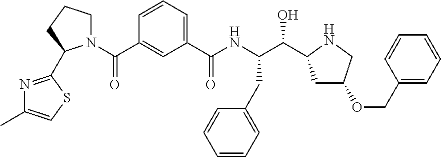 [SMILES: Cc1csc([C@H]2CCCN2C(=O)c2cccc(C(=O)N[C@@H](Cc3ccccc3)[C@H](O)[C@H]3C[C@@H](OCc4ccccc4)CN3)c2)n1]

N-((1R,2S)-1-((2R,4R)-4-(benzyloxy)pyrrolidin-2-yl)-1-hydroxy-3-phenylpropan-2-yl)-3-((R)-2-(4-methylthiazol-2-yl)pyrrolidine-1-carbonyl)benzamide: ¹H NMR (300 MHz, CDCl₃) [symbol] 7.80 (s, 1H), 7.67-7.54 (m, 2H), 7.45-7.19 (m, 12H), 6.81-6.68 (m, 1H), 6.48-6.19 (m, 1H), 5.69-5.65 (m, 0.7H), 5.09-5.07 (m, 0.2H), 4.59-4.50 (m, 2H), 4.40 (m, 1H), 3.80-3.61 (m, 2H), 3.50-3.35 (m, 2H), 3.20-3.06 (m, 3H), 2.84-2.80 (m, 1H), 2.47-2.32 (m, 5H), 2.20-2.06 (m, 4H).

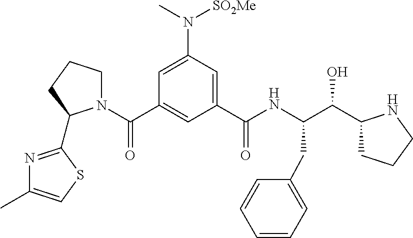 [SMILES: Cc1csc([C@H]2CCCN2C(=O)c2cc(C(=O)N[C@@H](Cc3ccccc3)[C@H](O)[C@H]3CCCN3)cc(N(C)S(C)(=O)=O)c2)n1]

N-((1R,2S)-1-hydroxy-3-phenyl-1-((R)-pyrrolidin-2-yl)propan-2-yl)-3-(N-methylmethylsulfonamido)-5-((R)-2-(4-methylthiazol-2-yl)pyrrolidine-1-carbonyl)benzamide: ¹H NMR (300 MHz, CDCl₃+CD₃OD): δ 7.732-7.354 (m, 3H), 7.181 (m, 5H), 6.772, 6.648 (m, 1H), 5.547 (m, 0.7H), 5.100 (m, 0.3H), 4.293 (m, 1H), 3.713 (m, 2H), 3.450 (m, 1H), 3.236 (s, 3H), 3.145 (m, 2H), 2.916 (m, 3H), 2.788 (s, 3H), 2.403 (s, 3H), 2.235 (m, 1H), 2.064 (m, 2H), 1.937-1.676 (m, 5H).

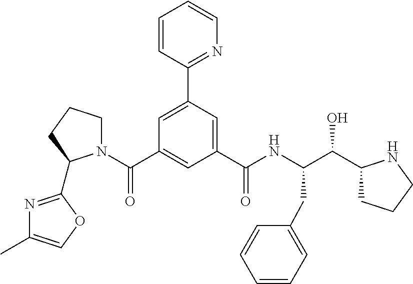 [SMILES: Cc1coc([C@H]2CCCN2C(=O)c2cc(C(=O)N[C@@H](Cc3ccccc3)[C@H](O)[C@H]3CCCN3)cc(-c3ccccn3)c2)n1]

N-((1R,2S)-1-hydroxy-3-phenyl-1-((R)-pyrrolidin-2-yl)propan-2-yl)-3-((R)-2-(4-methyloxazol-2-yl)pyrrolidine-1-carbonyl)-5-(pyridin-2-yl)benzamide: ¹H NMR (300 MHz, CDCl₃): d 1.89-2.40 (m, 11H), 2.40-4.09 (m, 8H), 4.65-4.69 (m, 1H), 5.43-5.46 (m, 1H), 7.11-7.34 (m, 7H), 7.78-8.70 (m, 6H).

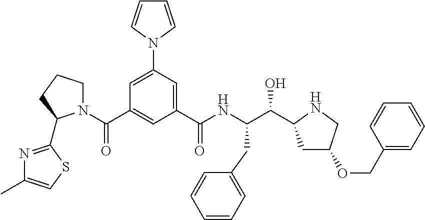 [SMILES: Cc1csc([C@H]2CCCN2C(=O)c2cc(C(=O)N[C@@H](Cc3ccccc3)[C@H](O)[C@H]3C[C@@H](OCc4ccccc4)CN3)cc(-n3cccc3)c2)n1]

N-((1R,2S)-1-((2R,4R)-4-(benzyloxy)pyrrolidin-2-yl)-1-hydroxy-3-phenylpropan-2-yl)-3-((R)-2-(4-methylthiazol-2-yl)pyrrolidine-1-carbonyl)-5-(1H-pyrrol-1-yl)benzamide: 1H NMR (300 MHz, CDCl₃): δ 7.66-7.52 (m, 2H), 7.42-7.18 (m, 14H), 7.02-6.74 (m, 4H), 6.38-6.32 (m, 2H), 5.68-5.64 (m, 0.7H), 5.11-5.08 (m, 0.3H), 4.60-4.40 (m, 3H), 4.19-4.12 (m, 2H), 3.91 (m, 1H), 3.68 (m, 2H), 3.50-3.41 (m, 4H), 3.23-3.07 (m, 3H), 2.88-2.85 (m, 1H), 2.49-2.30 (m, 5H), 2.18-1.87 (m, 7H).

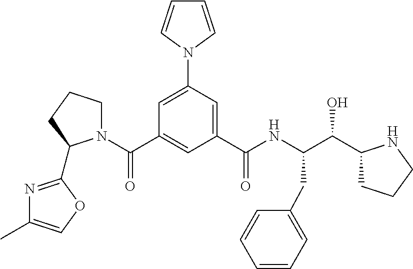 [SMILES: Cc1coc([C@H]2CCCN2C(=O)c2cc(C(=O)N[C@@H](Cc3ccccc3)[C@H](O)[C@H]3CCCN3)cc(-n3cccc3)c2)n1]

N-((1R,2S)-1-hydroxy-3-phenyl-1-((R)-pyrrolidin-2-yl)propan-2-yl)-3-((R)-2-(4-methyloxazol-2-yl)pyrrolidine-1-carbonyl)-5-(1H-pyrrol-1-yl)benzamide: ¹H NMR (CDCl₃): d 7.46 (m, 2H), 7.36 (m, 1H), 7.32 (m, 1H), 7.12-7.27 (m, 5H), 6.92 (m, 2H), 6.30 (m, 2H), 5.33 (m, 1H), 4.36 (m, 1H), 3.61-3.80 (m, 3H), 3.41-3.43 (m, 1H), 3.15-3.24 (m, 2H), 2.89-3.06 (m, 2H), 2.31-2.38 (m, 1H), 2.17 (s, 3H), 2.04-2.13 (m, 1H), 1.75-1.94 (m, 6H).

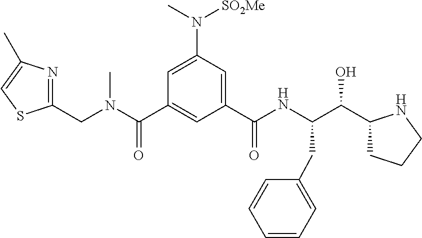 [SMILES: Cc1csc(CN(C)C(=O)c2cc(C(=O)N[C@@H](Cc3ccccc3)[C@H](O)[C@H]3CCCN3)cc(N(C)S(C)(=O)=O)c2)n1]

N1-((1R,2S)-1-hydroxy-3-phenyl-1-((R)-pyrrolidin-2-yl)propan-2-yl)-N3-methyl-5-(N-methylmethylsulfonamido)-N3-((4-methylthiazol-2-yl)methyl)isophthalamide: ¹H NMR (300 MHz, CDCl₃+CD₃OD): δ 8.255-7.621 (m, 3H), 7.217 (m, 5H), 6.925 (s, 1H), 4.956 (s, 1.2H), 4.674 (br, 0.8H), 4.360 (m, 1H), 3.864 (m, 1H), 3.335 (s, 3H), 3.443-3.200 (m, 2H), 3.054 (s, 3H), 3.118-2.951 (m, 2H), 2.891 (s, 3H), 2.842 (m, 1H), 2.472 (s, 3H), 1.919 (m, 4H).

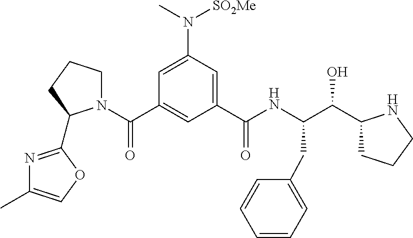 [SMILES: Cc1coc([C@H]2CCCN2C(=O)c2cc(C(=O)N[C@@H](Cc3ccccc3)[C@H](O)[C@H]3CCCN3)cc(N(C)S(C)(=O)=O)c2)n1]

N-((1R,2S)-1-hydroxy-3-phenyl-1-((R)-pyrrolidin-2-yl)propan-2-yl)-3-(N-methylmethylsulfonamido)-5-((R)-2-(4-methyloxazol-2-yl)pyrrolidine-1-carbonyl)benzamide: ¹H NMR (300 MHz, CDCl₃+CD₃OD): δ 7.789-7.433 (m, 3H), 7.200 (m, 6H), 5.313 (m, 0.7H), 4.901 (m, 0.3H), 4.321 (m, 1H), 3.783 (m, 2H), 3.496 (m, 1H), 3.283 (s, 3H), 3.246 (m, 2H), 2.984 (m, 2H), 2.838 (s, 3H), 2.802 (m, 1H), 2.373 (m, 1H), 2.161 (s, 3H), 2.083 (m, 2H), 1.908 (m, 2H), 1.787 (m, 3H).

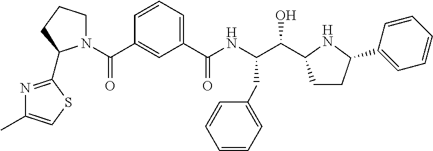 [SMILES: Cc1csc([C@H]2CCCN2C(=O)c2cccc(C(=O)N[C@@H](Cc3ccccc3)[C@H](O)[C@H]3CC[C@@H](c4ccccc4)N3)c2)n1]

N-((1R,2S)-1-hydroxy-3-phenyl-1-((2R,5S)-5-phenylpyrrolidin-2-yl)propan-2-yl)-3-((R)-2-(4-methylthiazol-2-yl)pyrrolidine-1-carbonyl)benzamide: ¹H NMR (300 MHz, CDCl₃) δ 7.90-7.84 (m, 1H), 7.70-7.60 (m, 2H), 7.42-7.21 (m, 12H), 6.82-6.36 (m, 2H), 5.67-5.63 (m, 0.7H), 5.11-5.09 (m, 0.2H), 4.51-4.47 (m, 1H), 4.23-4.18 (m, 1H), 3.96-3.66 (m, 3H), 3.51-3.46 (m, 2H), 3.28-3.08 (m, 2H), 2.47-2.33 (m, 5H), 2.28-1.90 (m, 5H), 1.80-1.67 (m, 1H).

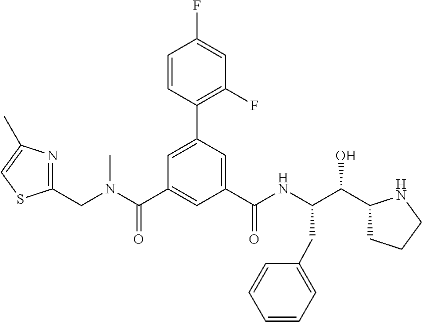 [SMILES: Cc1csc(CN(C)C(=O)c2cc(C(=O)N[C@@H](Cc3ccccc3)[C@H](O)[C@H]3CCCN3)cc(-c3ccc(F)cc3F)c2)n1]

2′,4′-difluoro-N3-((1R,2S)-1-hydroxy-3-phenyl-1-((R)-pyrrolidin-2-yl)propan-2-yl)-N5-methyl-N5-((4-methylthiazol-2-yl)methyl)biphenyl-3,5-dicarboxamide: ¹H NMR (CDCl₃): d 7.76 (m, 2H), 7.66 (m, 1H), 7.60 (m, 1H), 7.09-7.32 (m, 5H), 6.86-6.94 (m, 4H), 4.89 (m, 2H), 4.37 (m, 1H), 3.64-3.68 (m, 1H), 3.13-3.21 (m, 2H), 2.99 (s, 3H), 2.89-3.04 (m, 2H), 2.77-2.83 (m, 1H), 2.42 (s, 3H), 1.64-1.85 (m, 4H).

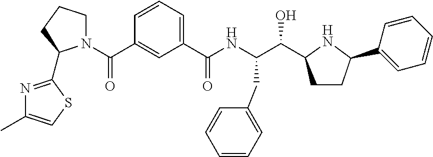 [SMILES: Cc1csc([C@H]2CCCN2C(=O)c2cccc(C(=O)N[C@@H](Cc3ccccc3)[C@H](O)[C@@H]3CC[C@H](c4ccccc4)N3)c2)n1]

N-((1R,2S)-1-hydroxy-3-phenyl-1-((2S,5R)-5-phenylpyrrolidin-2-yl)propan-2-yl)-3-((R)-2-(4-methylthiazol-2-yl)pyrrolidine-1-carbonyl)benzamide: ¹H NMR (300 MHz, CDCl₃) d 7.78 (m, 1H), 7.67-7.60 (m, 2H), 7.48-7.19 (m, 13H), 6.79-6.61 (m, 1H), 6.38-6.08 (m, 1H), 5.67-5.62 (m, 0.7H), 5.05-5.02 (m, 0.2H), 4.32-4.22 (m, 2H), 3.88 (m, 1H), 3.69-3.62 (m, 1H), 3.47-3.16 (m, 5H), 3.05-2.98 (m, 1H), 2.48-2.20 (m, 5H), 2.14-1.85 (m, 5H), 1.76-1.63 (m, 1H).

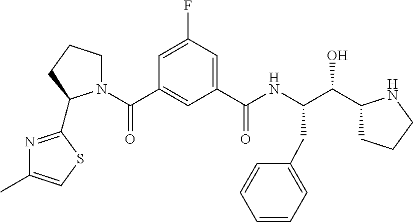 [SMILES: Cc1csc([C@H]2CCCN2C(=O)c2cc(F)cc(C(=O)N[C@@H](Cc3ccccc3)[C@H](O)[C@H]3CCCN3)c2)n1]

3-fluoro-N-((1R,2S)-1-hydroxy-3-phenyl-1-((R)-pyrrolidin-2-yl)propan-2-yl)-5-((R)-2-(4-methylthiazol-2-yl)pyrrolidine-1-carbonyl)benzamide: ¹H NMR (CDCl₃): d 7.51 (s, 1H), 7.13-7.26 (m, 7H), 6.77 (s, 1H), 5.54-5.59 (m, 1H), 4.34 (m, 1H), 3.60-3.66 (m, 2H), 3.38 (m, 1H), 3.11-3.16 (m, 2H), 2.92-2.99 (m, 2H), 2.79-2.85 (m, 1H), 2.41 (s, 3H), 2.28-2.39 (m, 2H), 2.02-2.09 (m, 1H), 1.82-1.91 (m, 1H), 1.70-1.74 (m, 4H).

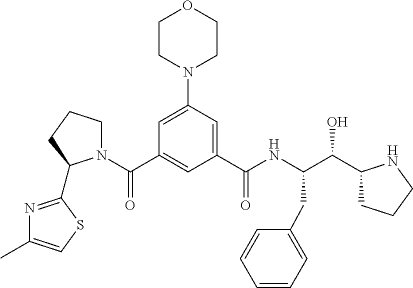 [SMILES: Cc1csc([C@H]2CCCN2C(=O)c2cc(C(=O)N[C@@H](Cc3ccccc3)[C@H](O)[C@H]3CCCN3)cc(N3CCOCC3)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-morpholinobenzamide: ¹H NMR (300 MHz, CDCl₃): d 1.82-2.41 (m, 11H), 2.80-3.90 (m, 16H), 4.26-4.30 (m, 1H), 5.46-5.49 (m, 1H), 6.76-7.37 (m, 9H).

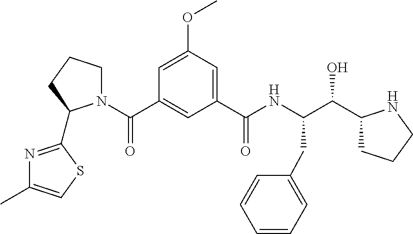 [SMILES: COc1cc(C(=O)N[C@@H](Cc2ccccc2)[C@H](O)[C@H]2CCCN2)cc(C(=O)N2CCC[C@@H]2c2nc(C)cs2)c1]

N-((1R,2S)-1-hydroxy-3-phenyl-1-((R)-pyrrolidin-2-yl)propan-2-yl)-3-methoxy-5-((R)-2-(4-methylthiazol-2-yl)pyrrolidine-1-carbonyl)benzamide: ¹H NMR (CDCl₃): d 7.06-7.25 (m, 8H), 6.75 (s, 1H), 5.55-5.59 (m, 1H), 4.34 (m, 1H), 3.72 (s, 3H), 3.57-3.65 (m, 2H), 3.37-3.41 (m, 1H), 3.10-3.22 (m, 2H), 2.89-3.03 (m, 2H), 2.76-2.78 (m, 1H), 2.41 (s, 3H), 2.24-2.34 (m, 2H), 1.99-2.05 (m, 1H), 1.80-1.91 (m, 1H), 1.63-1.78 (m, 4H).

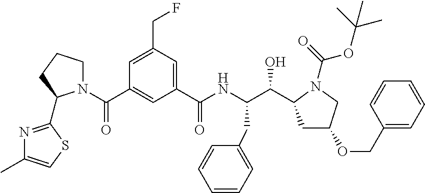 [SMILES: Cc1csc([C@H]2CCCN2C(=O)c2cc(CF)cc(C(=O)N[C@@H](Cc3ccccc3)[C@H](O)[C@H]3C[C@@H](OCc4ccccc4)CN3C(=O)OC(C)(C)C)c2)n1]

(2R,4R)-tert-butyl 4-(benzyloxy)-2-((1S,2S)-2-(3-(fluoromethyl)-5-((R)-2-(4-methylthiazol-2-yl)pyrrolidine-1-carbonyl)benzamido)-1-hydroxy-3-phenylpropyl)pyrrolidine-1-carboxylate: ¹H NMR (300 MHz, CDCl₃), d: 8.147-7.852 (m, 3H), 7.406 (m, 10H), 6.961, 6.879 (br, 1H), 5.804-5.267 (m, 3H), 4.813-4.596 (m, 3H), 4.439-4.060 (m, 3H), 3.907-3.596 (m, 3H), 3.372 (m, 1H), 3.102 (m, 1H), 2.611 (s, 3H), 2.648-2.469 (m, 3H), 2.219 (m, 2H), 2.092 (m, 1H), 1.627 (s, 9H).

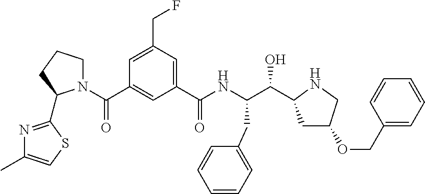 [SMILES: Cc1csc([C@H]2CCCN2C(=O)c2cc(CF)cc(C(=O)N[C@@H](Cc3ccccc3)[C@H](O)[C@H]3C[C@@H](OCc4ccccc4)CN3)c2)n1]

N-((1R,2S)-1-((2R,4R)-4-(benzyloxy)pyrrolidin-2-yl)-1-hydroxy-3-phenylpropan-2-yl)-3-(fluoromethyl)-5-((R)-2-(4-methylthiazol-2-yl)pyrrolidine-1-carbonyl)benzamide: ¹H NMR (300 MHz, CDCl₃+CD₃OD), d: 7.728-7.590 (m, 3H), 7.405-7.084 (m, 10H), 6.728 (m, 1H), 5.623 (m, 0.5H), 5.416 (s, 0.6H), 5.287 (m, 0.8H), 5.127 (s, 0.2H), 4.520 (m, 2H), 4.370 (m, 1H), 4.059 (m, 1H), 3.656 (m, 2H), 3.364 (m, 2H), 3.210-3.024 (m, 3H), 2.799 (m, 1H), 2.442 (s, 3H), 2.442-2.296 (m, 2H), 2.184-1.821 (m, 4H).

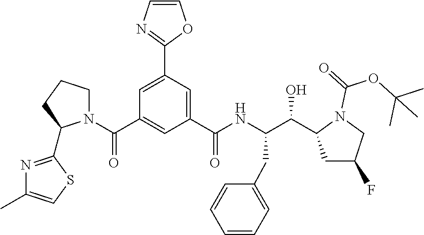 [SMILES: Cc1csc([C@H]2CCCN2C(=O)c2cc(C(=O)N[C@@H](Cc3ccccc3)[C@H](O)[C@H]3C[C@H](F)CN3C(=O)OC(C)(C)C)cc(-c3ncco3)c2)n1]

### (2R,4S)-tert-butyl 4-fluoro-2-((1S,2S)-1-hydroxy-2-(3-((R)-2-(4-methylthiazol-2-yl)pyrrolidine-1-carbonyl)-5-(oxazol-2-yl)benzamido)-3-phenylpropyl)pyrrolidine-1-carboxylate

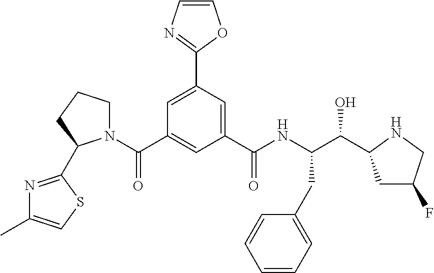 [SMILES: Cc1csc([C@H]2CCCN2C(=O)c2cc(C(=O)N[C@@H](Cc3ccccc3)[C@H](O)[C@H]3C[C@H](F)CN3)cc(-c3ncco3)c2)n1]

N-((1R,2S)-1-((2R,4S)-4-fluoropyrrolidin-2-yl)-1-hydroxy-3-phenylpropan-2-yl)-3-((R)-2-(4-methylthiazol-2-yl)pyrrolidine-1-carbonyl)-5-(oxazol-2-yl)benzamide: ¹H NMR (CDCl₃): d 8.12 (s, 2H), 7.80 (s, 1H), 7.72 (s, 1H), 7.20-7.11 (m, 6H), 6.88-6.84 (m, 1H), 6.80 (s, 1H), 5.66-5.60 (m, 1H), 5.32-5.24 (m, 0.5H), 5.12-5.06 (m, 0.5H), 4.64-4.34 (m, 1H), 3.74-3.50 (m, 3H), 3.50-3.32 (m, 1H), 3.32-2.90 (m, 4H), 2.44-1.82 (m, 11H).

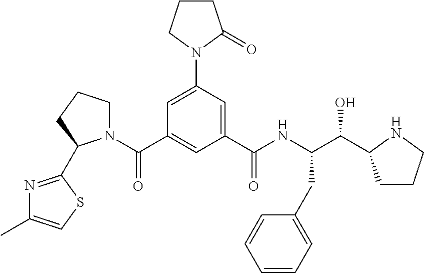 [SMILES: Cc1csc([C@H]2CCCN2C(=O)c2cc(C(=O)N[C@@H](Cc3ccccc3)[C@H](O)[C@H]3CCCN3)cc(N3CCCC3=O)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-(2-oxopyrrolidin-1-yl)benzamide: ¹H NMR (300 MHz, CDCl₃) δ 8.13 (s, 1H), 7.78-7.44 (m, 2H), 7.29-7.04 (m, 8H), 6.78-6.63 (m, 1H), 5.62-5.58 (m, 0.7H), 5.08-5.06 (m, 0.2H), 4.37 (m, 1H), 3.89-3.41 (m, 6H), 3.19-2.83 (m, 6H), 2.64-2.54 (m, 2H), 2.43-2.27 (m, 5H), 2.13-2.03 (m, 4H), 1.93-1.70 (m, 5H).

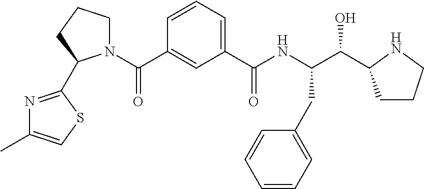 [SMILES: Cc1csc([C@H]2CCCN2C(=O)c2cccc(C(=O)N[C@@H](Cc3ccccc3)[C@H](O)[C@H]3CCCN3)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)benzamide: ¹H NMR (CDCl₃): d 7.76 (s, 1H), 7.57-7.63 (m, 1H), 7.35 (m, 1H), 7.16-7.23 (m, 6H), 6.75 (m, 1H), 5.60 (m, 1H), 4.36 (m, 1H), 3.83 (m, 1H), 3.60-3.66 (m, 2H), 3.39-3.43 (m, 1H), 3.00-3.22 (m, 4H), 2.91 (m, 1H), 2.83 (m, 1H), 2.42 (s, 3H), 2.27-2.36 (m, 2H), 2.02 (m, 1H), 1.67-1.93 (m, 3H).

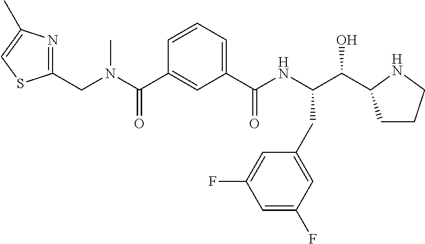 [SMILES: Cc1csc(CN(C)C(=O)c2cccc(C(=O)N[C@@H](Cc3cc(F)cc(F)c3)[C@H](O)[C@H]3CCCN3)c2)n1]

N1-((1R,2S)-3-(3,5-difluorophenyl)-1-hydroxy-1-((R)-pyrrolidin-2-yl)propan-2-yl)-N3-methyl-N3-((4-methylthiazol-2-yl)methyl)isophthalamide: ¹H NMR (CDCl₃): d 7.72 (m, 2H), 7.48 (m, 1H), 7.34-7.40 (m, 1H), 6.79-6.96 (m, 4H), 6.54-6.60 (m, 1H), 4.61-4.91 (m, 2H), 4.36 (m, 1H), 3.58 (m, 1H), 2.81-3.19 (m, 8H), 2.42 (s, 3H), 1.66-1.85 (m, 4H).

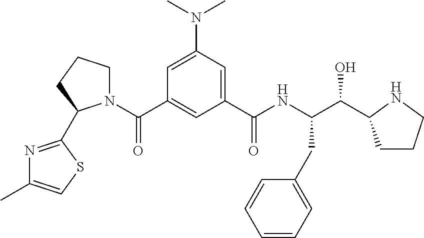 [SMILES: Cc1csc([C@H]2CCCN2C(=O)c2cc(C(=O)N[C@@H](Cc3ccccc3)[C@H](O)[C@H]3CCCN3)cc(N(C)C)c2)n1]

3-(dimethylamino)-N-((1R,2S)-1-hydroxy-3-phenyl-1-((R)-pyrrolidin-2-yl)propan-2-yl)-5-((R)-2-(4-methylthiazol-2-yl)pyrrolidine-1-carbonyl)benzamide: ¹H NMR (300 MHz, CDCl₃) δ 7.30-7.14 (m, 6H), 6.98 (s, 1H), 6.90-6.68 (m, 3H), 6.63-6.54 (m, 1H), 5.61-5.57 (m, 0.7H), 5.05-5.03 (m, 0.3H), 4.34 (m, 1H), 3.81 (m, 1H), 3.61-3.56 (m, 2H), 3.45-3.37 (m, 1H), 3.20-3.00 (m, 4H), 2.97-2.73 (m, 9H), 2.42-2.32 (m, 5H), 2.10-1.99 (m, 2H), 1.92-1.71 (m, 5H).

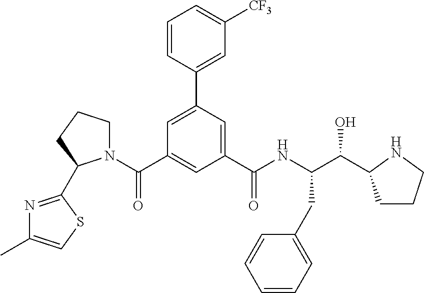 [SMILES: Cc1csc([C@H]2CCCN2C(=O)c2cc(C(=O)N[C@@H](Cc3ccccc3)[C@H](O)[C@H]3CCCN3)cc(-c3cccc(C(F)(F)F)c3)c2)n1]

N-((1R,2S)-1-hydroxy-3-phenyl-1-((R)-pyrrolidin-2-yl)propan-2-yl)-5-((R)-2-(4-methylthiazol-2-yl)pyrrolidine-1-carbonyl)-3′-(trifluoromethyl)biphenyl-3-carboxamide: ¹H NMR (300 MHz, CDCl₃): 1.68-2.38 (m, 11H), 2.42-3.18 (m, 5H), 3.22-3.45 (m, 1H), 3.47-3.68 (m, 2H), 4.26-4.41 (m, 1H), 5.60-5.64 (m, 1H), 6.77 (s, 1H), 7.18-7.79 (m, 12H).

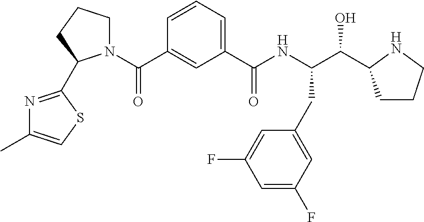 [SMILES: Cc1csc([C@H]2CCCN2C(=O)c2cccc(C(=O)N[C@@H](Cc3cc(F)cc(F)c3)[C@H](O)[C@H]3CCCN3)c2)n1]

N-((1R,2S)-3-(3,5-difluorophenyl)-1-hydroxy-1-((R)-pyrrolidin-2-yl)propan-2-yl)-3-((R)-2-(4-methylthiazol-2-yl)pyrrolidine-1-carbonyl)benzamide: ¹H NMR (CDCl₃): d 7.80 (s, 1H), 7.56-7.64 (m, 2H), 7.35 (m, 1H), 6.78-6.83 (m, 3H), 6.61 (m, 1H), 5.60-5.65 (m, 1H), 4.34 (m, 1H), 3.83 (m, 1H), 3.65-3.71 (m, 3H), 3.45-3.48 (m, 1H), 3.14-3.23 (m, 2H), 2.85-3.03 (m, 3H), 2.44 (s, 3H), 2.32-2.41 (m, 2H), 2.04-2.11 (m, 1H), 1.73-1.95 (m, 3H).

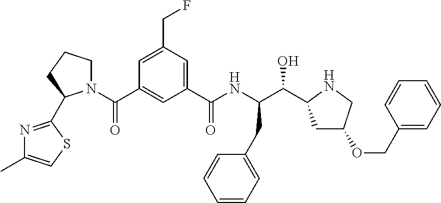 [SMILES: Cc1csc([C@H]2CCCN2C(=O)c2cc(CF)cc(C(=O)N[C@H](Cc3ccccc3)[C@H](O)[C@H]3C[C@@H](OCc4ccccc4)CN3)c2)n1]

N-((1R,2R)-1-((2R,4R)-4-(benzyloxy)pyrrolidin-2-yl)-1-hydroxy-3-phenylpropan-2-yl)-3-(fluoromethyl)-5-((R)-2-(4-methylthiazol-2-yl)pyrrolidine-1-carbonyl)benzamide: ¹H NMR (300 MHz, CDCl₃) δ 7.86-7.80 (m, 1H), 7.70-7.57 (m, 2H), 7.33-7.16 (m, 11H), 6.96-6.91 (m, 1H), 6.79-6.62 (m, 1H), 5.65-5.60 (m, 0.6H), 5.45-5.15 (m, 2H), 5.05-5.03 (m, 0.2H), 4.63-4.40 (m, 3H), 4.03-4.01 (m, 1H), 3.74-3.64 (m, 2H), 3.49-3.41 (m, 1H), 3.16-2.84 (m, 5H), 2.44-2.31 (m, 5H), 2.05-1.82 (m, 6H).

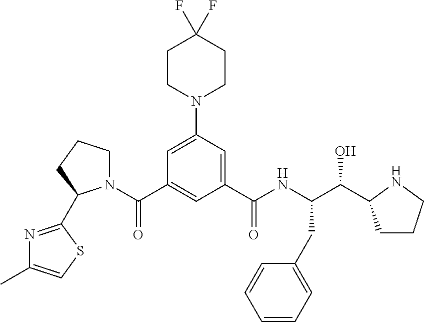 [SMILES: Cc1csc([C@H]2CCCN2C(=O)c2cc(C(=O)N[C@@H](Cc3ccccc3)[C@H](O)[C@H]3CCCN3)cc(N3CCC(F)(F)CC3)c2)n1]

3-(4,4-difluoropiperidin-1-yl)-N-((1R,2S)-1-hydroxy-3-phenyl-1-((R)-pyrrolidin-2-yl)propan-2-yl)-5-((R)-2-(4-methylthiazol-2-yl)pyrrolidine-1-carbonyl)benzamide: ¹H NMR (300 MHz, CDCl₃): 1.72-2.42 (m, 15H), 2.85-3.90 (m, 12H), 4.36-4.41 (m, 1H), 5.60-5.64 (m, 1H), 6.81 (s, 1H), 7.09-7.31 (m, 8H).

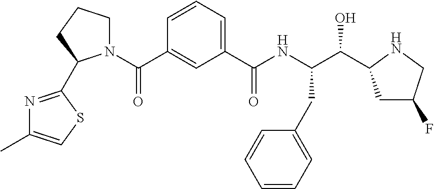 [SMILES: Cc1csc([C@H]2CCCN2C(=O)c2cccc(C(=O)N[C@@H](Cc3ccccc3)[C@H](O)[C@H]3C[C@H](F)CN3)c2)n1]

N-((1R,2S)-1-((2R,4S)-4-fluoropyrrolidin-2-yl)-1-hydroxy-3-phenylpropan-2-yl)-3-((R)-2-(4-methylthiazol-2-yl)pyrrolidine-1-carbonyl)benzamide: ¹H NMR (CDCl₃): d 7.80-7.72 (m, 1H), 7.70-7.50 (m, 2H), 7.44-7.32 (m, 1H), 7.32-7.10 (m, 5H), 6.75 (s, 1H), 6.32 (d, J=7.0 Hz 1H) 5.63-5.58 (m, 1H), 5.26-5.22 (m, 0.5H), 5.08-5.02 (m, 0.5H), 4.38-4.20 (m, 1H), 3.86-3.38 (m, 4H), 3.24-2.82 (m, 4H), 2.42-2.12 (m, 5H), 2.10-1.72 (m, 4H).

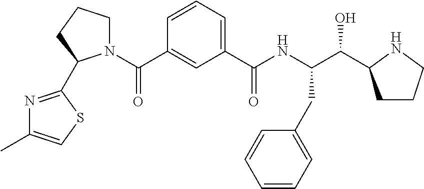 [SMILES: Cc1csc([C@H]2CCCN2C(=O)c2cccc(C(=O)N[C@@H](Cc3ccccc3)[C@H](O)[C@@H]3CCCN3)c2)n1]

N-((1R,2S)-1-hydroxy-3-phenyl-1-((S)-pyrrolidin-2-yl)propan-2-yl)-3-((R)-2-(4-methylthiazol-2-yl)pyrrolidine-1-carbonyl)benzamide: ¹H NMR (300 MHz, CDCl₃) δ 7.81 (s, 1H), 7.70-7.65 (m, 2H), 7.46-7.41 (m, 1H), 7.32-7.18 (m, 8H), 6.78-6.64 (m, 1H), 6.54-6.23 (m, 1H), 5.66-5.62 (m, 0.7H), 5.06-5.05 (m, 0.2H), 4.34 (m, 1H), 3.72-3.63 (m, 1H), 3.48-3.40 (m, 1H), 3.32-3.28 (m, 2H), 3.15-2.90 (m, 5H), 2.44-2.29 (m, 6H), 2.14-2.03 (m, 2H), 1.96-1.67 (m, 6H).

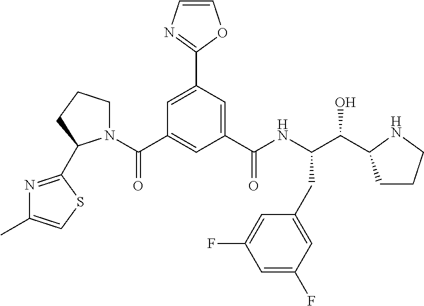 [SMILES: Cc1csc([C@H]2CCCN2C(=O)c2cc(C(=O)N[C@@H](Cc3cc(F)cc(F)c3)[C@H](O)[C@H]3CCCN3)cc(-c3ncco3)c2)n1]

N-((1R,2S)-3-(3,5-difluorophenyl)-1-hydroxy-1-((R)-pyrrolidin-2-yl)propan-2-yl)-3-((R)-2-(4-methylthiazol-2-yl)pyrrolidine-1-carbonyl)-5-(oxazol-2-yl)benzamide: ¹H NMR (CDCl₃): d 8.14 (s, 1H), 8.07 (s, 1H), 7.74 (s, 1H), 7.67 (s, 1H), 7.17 (s, 1H), 6.86-6.90 (m, 2H), 6.78 (m, 1H), 6.54-6.57 (m, 1H), 5.60-5.64 (m, 1H), 4.35 (m, 1H), 3.63-3.70 (m, 2H), 3.16-3.39 (m, 3H), 2.86-3.04 (m, 3H), 2.42 (s, 3H), 2.32-2.38 (m, 1H), 2.04-2.10 (m, 1H), 1.88-1.94 (m, 2H), 1.73-1.78 (m, 4H).

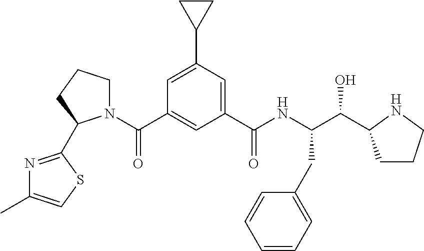 [SMILES: Cc1csc([C@H]2CCCN2C(=O)c2cc(C(=O)N[C@@H](Cc3ccccc3)[C@H](O)[C@H]3CCCN3)cc(C3CC3)c2)n1]

3-cyclopropyl-N-((1R,2S)-1-hydroxy-3-phenyl-1-((R)-pyrrolidin-2-yl)propan-2-yl)-5-((R)-2-(4-methylthiazol-2-yl)pyrrolidine-1-carbonyl)benzamide: ¹H NMR (300 MHz, CDCl₃): 0.65-0.98 (m, 4H), 1.72-2.43 (m, 12H), 2.80-3.20 (m, 5H), 3.38-3.42 (m, 1H), 3.52-3.64 (m, 2H), 4.32-4.41 (m, 1H), 5.60-5.64 (m, 1H), 6.79 (s, 1H), 7.19-7.38 (m, 7H), 7.49 (s, 1H).

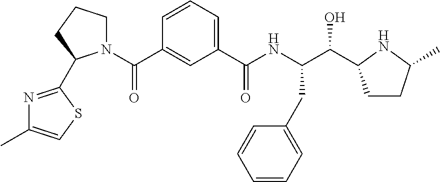

N-((1R,2S)-1-hydroxy-1-((2R,5R)-5-methylpyrrolidin-2-yl)-3-phenylpropan-2-yl)-3-((R)-2-(4-methylthiazol-2-yl)pyrrolidine-1-carbonyl)benzamide: ¹H NMR (300 MHz, CDCl₃) δ 7.77 (s, 1H), 7.65-7.59 (m, 2H), 7.40-7.16 (m, 8H), 6.77-6.64 (m, 1H), 6.46-6.15 (m, 1H), 5.63-5.59 (m, 0.8H), 5.05-5.03 (m, 0.2H), 4.42-4.32 (m, 1H), 3.70-3.62 (m, 1H), 3.55-3.39 (m, 2H), 3.23-2.99 (m, 5H), 2.42-2.29 (m, 6H), 2.13-2.01 (m, 2H), 1.96-1.80 (m, 5H), 1.32-1.16 (m, 2H), 1.10 (d, 3H).

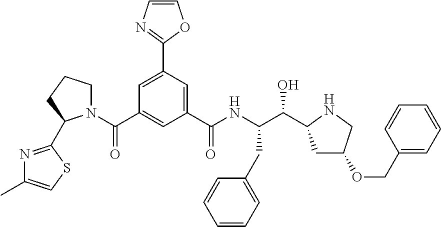 [SMILES: Cc1csc([C@H]2CCCN2C(=O)c2cc(C(=O)N[C@@H](Cc3ccccc3)[C@H](O)[C@H]3C[C@@H](OCc4ccccc4)CN3)cc(-c3ncco3)c2)n1]

N-((1R,2S)-1-((2R,4R)-4-(benzyloxy)pyrrolidin-2-yl)-1-hydroxy-3-phenylpropan-2-yl)-3-((R)-2-(4-methylthiazol-2-yl)pyrrolidine-1-carbonyl)-5-(oxazol-2-yl)benzamide: ¹H NMR (CDCl₃): d 8.22-8.23 (m, 2H), 7.81 (s, 1H), 7.68 (s, 1H), 7.19-7.34 (m, 9H), 6.77 (s, 1H), 5.62 (m, 1H), 4.44-4.56 (m, 2H), 4.39 (m, 1H), 4.03 (m, 1H), 3.65-3.68 (m, 2H), 3.38-3.42 (m, 2H), 3.02-3.17 (m, 3H), 2.70-2.76 (m, 1H), 2.43 (s, 3H), 2.35-2.38 (m, 1H), 2.03-2.11 (m, 4H), 1.93 (m, 1H).

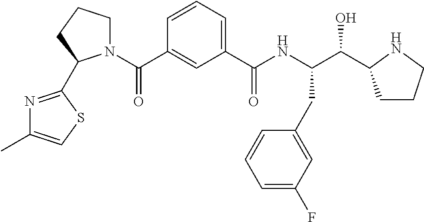 [SMILES: Cc1csc([C@H]2CCCN2C(=O)c2cccc(C(=O)N[C@@H](Cc3cccc(F)c3)[C@H](O)[C@H]3CCCN3)c2)n1]

N-((1R,2S)-3-(3-fluorophenyl)-1-hydroxy-1-((R)-pyrrolidin-2-yl)propan-2-yl)-3-((R)-2-(4-methylthiazol-2-yl)pyrrolidine-1-carbonyl)benzamide: ¹H NMR (CDCl₃): d 7.77 (s, 1H), 7.52-7.60 (m, 2H), 7.27-7.32 (m, 1H), 7.07-7.18 (m, 2H), 6.92-6.99 (m, 2H), 6.78-6.83 (m, 1H), 5.56-5.58 (m, 1H), 4.33 (m, 1H), 3.58-3.80 (m, 2H), 3.39-3.43 (m, 1H), 3.08-3.13 (m, 2H), 2.86-2.98 (m, 2H), 2.77-2.82 (m, 1H), 2.39 (s, 3H), 2.27-2.35 (m, 2H), 1.99-2.03 (m, 1H), 1.64-1.71 (m, 3H).

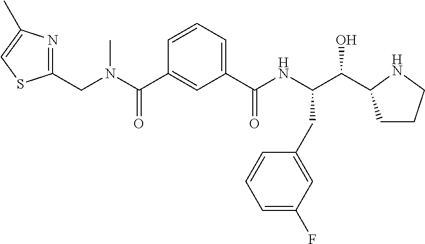 [SMILES: Cc1csc(CN(C)C(=O)c2cccc(C(=O)N[C@@H](Cc3cccc(F)c3)[C@H](O)[C@H]3CCCN3)c2)n1]

N1-((1R,2S)-3-(3-fluorophenyl)-1-hydroxy-1-((R)-pyrrolidin-2-yl)propan-2-yl)-N3-methyl-N3-((4-methylthiazol-2-yl)methyl)isophthalamide: ¹H NMR (CDCl₃): d 7.64-7.70 (m, 2H), 7.48 (m, 1H), 7.31-7.36 (m, 1H), 7.10-7.17 (m, 1H), 6.94-7.00 (m, 3H), 6.80-6.85 (m, 2H), 4.61-4.88 (m, 2H), 4.35 (m, 1H), 3.60-3.64 (m, 1H), 3.09-3.16 (m, 2H), 2.85-3.00 (m, 5H), 2.76-2.81 (m, 1H), 2.41 (s, 3H), 1.83 (m, 1H), 1.64-1.70 (m, 3H).

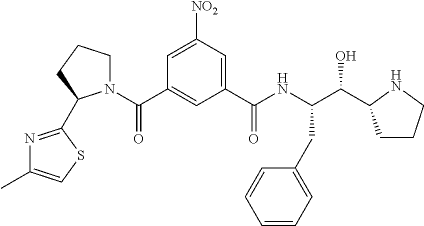 [SMILES: Cc1csc([C@H]2CCCN2C(=O)c2cc(C(=O)N[C@@H](Cc3ccccc3)[C@H](O)[C@H]3CCCN3)cc([N+](=O)[O-])c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-nitrobenzamide: ¹H NMR (300 MHz, CDCl₃): 1.73-2.42 (m, 11H), 2.83-3.27 (m, 5H), 3.26-3.28 (m, 1H), 3.67-3.72 (m, 2H), 4.33-4.48 (m, 1H), 5.57-5.61 (m, 1H), 6.79 (s, 1H), 7.16-7.33 (m, 5H), 8.11 (s, 1H), 8.35-8.41 (m, 2H).

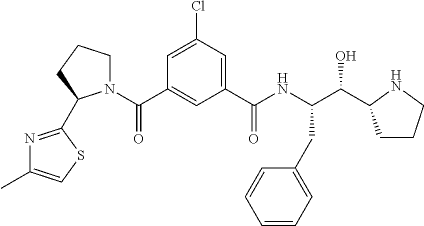 [SMILES: Cc1csc([C@H]2CCCN2C(=O)c2cc(Cl)cc(C(=O)N[C@@H](Cc3ccccc3)[C@H](O)[C@H]3CCCN3)c2)n1]

3-chloro-N-((1R,2S)-1-hydroxy-3-phenyl-1-((R)-pyrrolidin-2-yl)propan-2-yl)-5-((R)-2-(4-methylthiazol-2-yl)pyrrolidine-1-carbonyl)benzamide: ¹H NMR (300 MHz, CDCl₃): 1.68-2.43 (m, 11H), 2.85-3.22 (m, 5H), 3.38-3.46 (m, 1H), 3.59-3.83 (m, 2H), 4.28-4.44 (m, 1H), 5.57-5.62 (m, 1H), 6.80 (s, 1H), 7.17-7.32 (m, 6H), 7.30-7.61 (m, 2H).

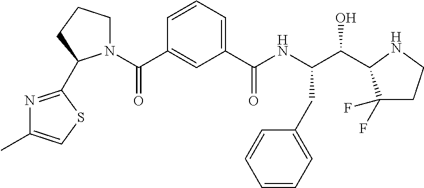 [SMILES: Cc1csc([C@H]2CCCN2C(=O)c2cccc(C(=O)N[C@@H](Cc3ccccc3)[C@H](O)[C@@H]3NCCC3(F)F)c2)n1]

N-((1S,2S)-1-(S)-3,3-difluoropyrrolidin-2-yl)-1-hydroxy-3-phenylpropan-2-yl)-3-((R)-2-(4-methylthiazol-2-yl)pyrrolidine-1-carbonyl)benzamide: ¹H NMR (CDCl₃): d 7.71 (s, 1H), 7.56-7.59 (m, 2H), 7.32-7.37 (m, 1H), 7.22-7.25 (m, 5H), 6.78 (s, 1H), 5.59-5.62 (m, 1H), 4.28 (m, 1H), 3.85 (m, 1H), 3.61-3.65 (m, 2H), 3.41-3.46 (m, 2H), 3.01-3.26 (m, 4H), 2.28-2.43 (m, 6H), 2.07 (m, 1H), 1.93 (m, 1H).

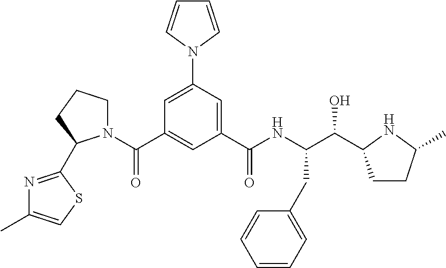

N-((1R,2S)-1-hydroxy-1-((2R,5R)-5-methylpyrrolidin-2-yl)-3-phenylpropan-2-yl)-3-((R)-2-(4-methylthiazol-2-yl)pyrrolidine-1-carbonyl)-5-(1H-pyrrol-1-yl)benzamide: ¹H NMR (300 MHz, CDCl₃) δ 7.61-7.48 (m, 2H), 7.41 (m, 1H), 7.28-7.12 (m, 7H), 6.94 (s, 2H), 6.80-6.70 (m, 2H), 6.39-6.28 (m, 2H), 5.64-5.59 (m, 0.7H), 5.06-5.04 (m, 0.3H), 4.38-4.37 (m, 1H), 3.68-3.55 (m, 2H), 3.45-3.37 (m, 1H), 3.29-3.01 (m, 5H), 2.45-2.30 (m, 6H), 2.13-2.05 (m, 2H), 1.98-1.85 (m, 4H), 1.35-1.25 (m, 2H), 1.13 (d, 3H).

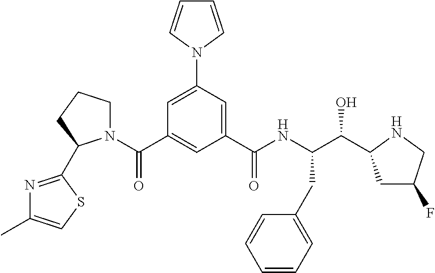 [SMILES: Cc1csc([C@H]2CCCN2C(=O)c2cc(C(=O)N[C@@H](Cc3ccccc3)[C@H](O)[C@H]3C[C@H](F)CN3)cc(-n3cccc3)c2)n1]

N-((1R,2S)-1-(2R,4S)-4-fluoropyrrolidin-2-yl)-1-hydroxy-3-phenylpropan-2-yl)-3-((R)-2-(4-methylthiazol-2-yl)pyrrolidine-1-carbonyl)-5-(1H-pyrrol-1-yl)benzamide: ¹H NMR (300 MHz, CDCl₃) δ 7.46-7.30 (m, 2H), 7.29-7.11 (m, 7H), 6.88-6.77 (m, 3H), 6.70-6.62 (m, 1H), 6.33-6.28 (m, 2H), 5.63-5.59 (m, 0.7H), 5.30 (m, 0.5H), 5.12-5.03 (m, 0.7H), 4.36-4.35 (m, 1H), 3.74-3.54 (m, 3H), 3.43-3.35 (m, 1H), 3.27-2.96 (m, 4H), 2.45-2.29 (m, 8H), 2.18-2.00 (m, 2H), 1.95-1.84 (m, 1H).

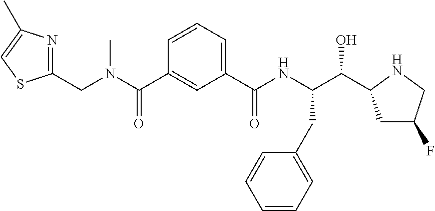 [SMILES: Cc1csc(CN(C)C(=O)c2cccc(C(=O)N[C@@H](Cc3ccccc3)[C@H](O)[C@H]3C[C@H](F)CN3)c2)n1]

N1-((1R,2S)-1-((2R,4S)-4-fluoropyrrolidin-2-yl)-1-hydroxy-3-phenylpropan-2-yl)-N3-methyl-N3-((4-methylthiazol-2-yl)methyl)isophthalamide: ¹H NMR (CDCl₃): d 7.80-7.40 (m, 4H), 7.30-7.16 (m, 5H), 6.92-6.80 (m, 1H), 6.36 (d, J=7.0 Hz, 1H), 5.26-5.22 (m, 0.5H), 5.08-5.02 (m, 0.5H), 5.00-4.92 (m, 1H), 4.38-4.30 (m, 1H), 3.70-3.52 (m, 2H), 3.24-2.90 (m, 6H), 2.40 (s, 3H), 2.42-2.12 (m, 5H).

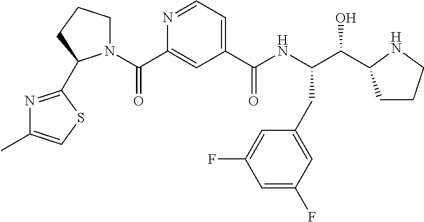 [SMILES: Cc1csc([C@H]2CCCN2C(=O)c2cc(C(=O)N[C@@H](Cc3cc(F)cc(F)c3)[C@H](O)[C@H]3CCCN3)ccn2)n1]

N-((1R,2S)-3-(3,5-difluorophenyl)-1-hydroxy-1-((R)-pyrrolidin-2-yl)propan-2-yl)-2-((R)-2-(4-methylthiazol-2-yl)pyrrolidine-1-carbonyl)isonicotinamide: ¹H NMR (300 MHz, CDCl₃) δ 8.59-8.61 (m, 1H), 8.24-8.25 (m, 1H), 7.92-7.99 (m, 1H), 7.57-7.59 (m, 1H), 6.73-6.86 (m, 4H), 6.59-6.66 (m, 1H), 5.61-5.65 (m, 1H), 4.27-4.41 (m, 1H), 3.76-3.97 (m, 1H), 3.55-3.76 (m, 2H), 3.40-3.49 (m, 1H), 3.21-3.33 (m, 1H), 2.84-3.21 (m, 4H), 2.43 (s, 3H), 2.31-2.48 (m, 1H), 1.90-2.27 (m, 1H), 1.63-1.90 (m, 6H).

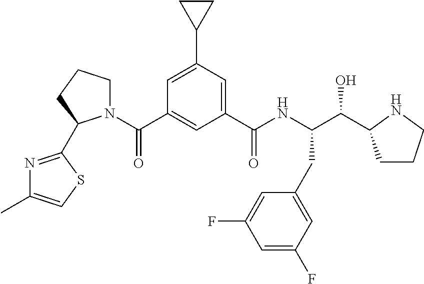 [SMILES: Cc1csc([C@H]2CCCN2C(=O)c2cc(C(=O)N[C@@H](Cc3cc(F)cc(F)c3)[C@H](O)[C@H]3CCCN3)cc(C3CC3)c2)n1]

3-cyclopropyl-N-((1R,2S)-3-(3,5-difluorophenyl)-1-hydroxy-1-((R)-pyrrolidin-2-yl)propan-2-yl)-5-((R)-2-(4-methylthiazol-2-yl)pyrrolidine-1-carbonyl)benzamide: ¹H NMR (300 MHz, CDCl₃): 0.62-0.99 (m, 4H), 1.52-2.20 (m, 6H), 2.29-2.41 (m, 5H), 2.72-3.81 (m, 9H), 4.22-4.38 (m, 1H), 5.51-5.61 (m, 1H), 6.52-6.67 (m, 5H), 7.30-7.54 (m, 2H).

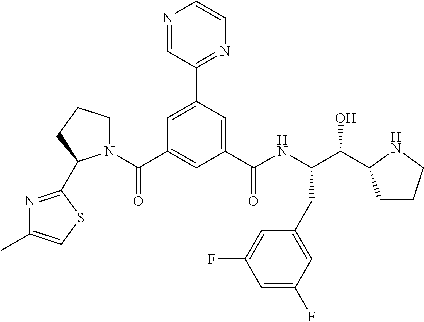 [SMILES: Cc1csc([C@H]2CCCN2C(=O)c2cc(C(=O)N[C@@H](Cc3cc(F)cc(F)c3)[C@H](O)[C@H]3CCCN3)cc(-c3cnccn3)c2)n1]

N-((1R,2S)-3-(3,5-difluorophenyl)-1-hydroxy-1-((R)-pyrrolidin-2-yl)propan-2-yl)-3-((R)-2-(4-methylthiazol-2-yl)pyrrolidine-1-carbonyl)-5-(pyrazin-2-yl)benzamide: ¹H NMR (300 MHz, CDCl₃): 1.72-2.42 (m, 11H), 2.88-3.24 (m, 3H), 3.46-3.94 (m, 5H), 4.32-4.42 (m, 1H), 5.58-5.63 (m, 1H), 6.55-6.85 (m, 3H), 7.16-7.26 (m, 1H), 7.92 (s, 1H), 8.17-8.23 (m, 2H), 8.50-8.55 (m, 2H), 8.98 (s, 1H).

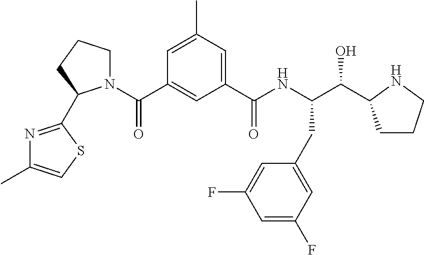 [SMILES: Cc1cc(C(=O)N[C@@H](Cc2cc(F)cc(F)c2)[C@H](O)[C@H]2CCCN2)cc(C(=O)N2CCC[C@@H]2c2nc(C)cs2)c1]

N-((1R,2S)-3-(3,5-difluorophenyl)-1-hydroxy-1-((R)-pyrrolidin-2-yl)propan-2-yl)-3-methyl-5-((R)-2-(4-methylthiazol-2-yl)pyrrolidine-1-carbonyl)benzamide: ¹H NMR (CDCl₃): d 8.50 (s, 1H), 8.38 (s, 1H), 8.22 (s, 1H), 8.18-8.02 (m, 1H), 7.80-7.22 (m, 4H), 6.58-6.40 (m, 1H), 5.32-5.20 (m, 1H), 4.80-4.40 (m, 2H), 4.40-4.32 (m, 1H), 4.20-3.98 (m, 2H), 3.96-3.60 (m, 3H), 3.33 (s, 3H), 3.20 (s, 3H), 3.28-2.86 (m, 4H), 2.86-2.50 (m, 6H).

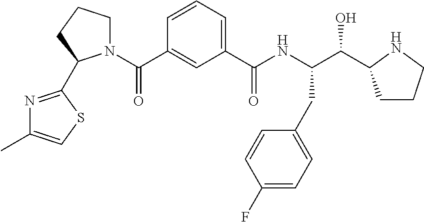 [SMILES: Cc1csc([C@H]2CCCN2C(=O)c2cccc(C(=O)N[C@@H](Cc3ccc(F)cc3)[C@H](O)[C@H]3CCCN3)c2)n1]

N-((1R,2S)-3-(4-fluorophenyl)-1-hydroxy-1-((R)-pyrrolidin-2-yl)propan-2-yl)-3-((R)-2-(4-methylthiazol-2-yl)pyrrolidine-1-carbonyl)benzamide: ¹H NMR (CDCl₃): d 7.75 (s, 1H), 7.57-7.62 (m, 2H), 7.35 (m, 1H), 7.16-7.21 (m, 2H), 6.87-6.93 (m, 2H), 6.77 (s, 1H), 5.58-5.61 (m, 1H), 4.32 (m, 1H), 3.81 (m, 1H), 3.59-3.67 (m, 2H), 3.41-3.44 (m, 1H), 3.08-3.21 (m, 2H), 2.81-3.00 (m, 4H), 2.42 (s, 3H), 2.29-2.38 (m, 2H), 2.02-2.07 (m, 1H), 1.80-1.92 (m, 1H), 1.66-1.78 (m, 2H).

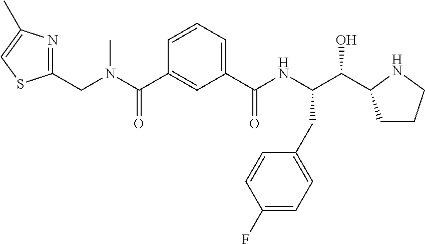 [SMILES: Cc1csc(CN(C)C(=O)c2cccc(C(=O)N[C@@H](Cc3ccc(F)cc3)[C@H](O)[C@H]3CCCN3)c2)n1]

N1-((1R,2S)-3-(4-fluorophenyl)-1-hydroxy-1-((R)-pyrrolidin-2-yl)propan-2-yl)-N3-methyl-N3-((4-methylthiazol-2-yl)methyl)isophthalamide: ¹H NMR (CDCl₃): d 7.66-7.72 (m, 2H), 7.46-7.50 (m, 1H), 7.35 (m, 1H), 7.16-7.21 (m, 2H), 6.85-6.91 (m, 4H), 4.60-4.90 (m, 2H), 4.35 (m, 1H), 3.62-3.66 (m, 1H), 3.10-3.20 (m, 2H), 2.90-2.97 (m, 5H), 2.80-2.88 (m, 1H), 2.43 (m, 3H), 1.84-1.87 (m, 1H), 1.66-1.72 (m, 3H).

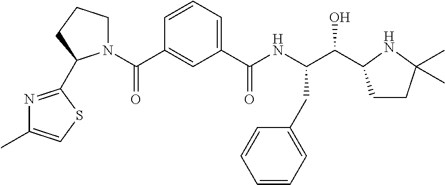

N-((1R,2S)-1-((R)-5,5-dimethylpyrrolidin-2-yl)-1-hydroxy-3-phenylpropan-2-yl)-3-((R)-2-(4-methylthiazol-2-yl)pyrrolidine-1-carbonyl)benzamide: ¹H NMR (300 MHz, CDCl₃) δ 7.84 (s, 1H), 7.70-7.61 (m, 2H), 7.45-7.40 (m, 1H), 7.37-7.16 (m, 7H), 6.78-6.65 (m, 1H), 6.28-6.04 (m, 1H), 5.65-5.61 (m, 0.7H), 5.08 (m, 0.2H), 4.34-4.37 (m, 1H), 3.73-3.65 (m, 1H), 3.51-3.43 (m, 3H), 3.15-3.09 (m, 2H), 2.44-2.30 (m, 6H), 2.15-2.04 (m, 3H), 1.93-1.88 (m, 3H), 1.66-1.50 (m, 2H), 1.28-1.13 (m, 6H).

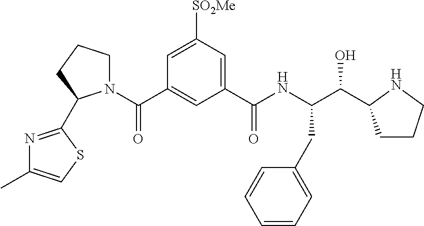 [SMILES: Cc1csc([C@H]2CCCN2C(=O)c2cc(C(=O)N[C@@H](Cc3ccccc3)[C@H](O)[C@H]3CCCN3)cc(S(C)(=O)=O)c2)n1]

N-((1R,2S)-1-hydroxy-3-phenyl-1-((R)-pyrrolidin-2-yl)propan-2-yl)-3-(methylsulfonyl)-5-((R)-2-(4-methylthiazol-2-yl)pyrrolidine-1-carbonyl)benzamide: ¹H NMR (300 MHz, CDCl₃): 1.62-2.16 (m, 6H), 2.21-2.42 (m, 5H), 2.78-3.12 (m, 8H), 3.26-3.41 (m, 1H), 3.62-3.78 (m, 2H), 4.31-4.36 (m, 1H), 5.53-5.57 (m, 1H), 6.81 (s, 1H), 7.12-7.38 (m, 5H), 8.12-8.21 (m, 3H).

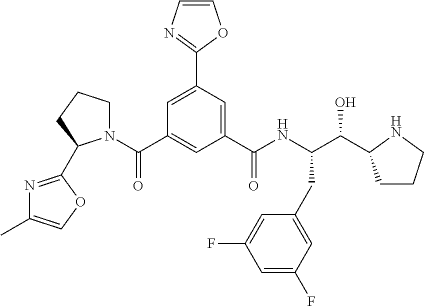 [SMILES: Cc1coc([C@H]2CCCN2C(=O)c2cc(C(=O)N[C@@H](Cc3cc(F)cc(F)c3)[C@H](O)[C@H]3CCCN3)cc(-c3ncco3)c2)n1]

N-((1R,2S)-3-(3,5-difluorophenyl)-1-hydroxy-1-((R)-pyrrolidin-2-yl)propan-2-yl)-3-((R)-2-(4-methyloxazol-2-yl)pyrrolidine-1-carbonyl)-5-(oxazol-2-yl)benzamide: ¹H NMR (300 MHz, CDCl₃), d: 8.200 (m, 2H), 7.92 (m, 1H), 7.680 (m, 1H), 7.293 (s, 1H), 7.166 (m, 1H), 6.828 (m, 2H), 6.529 (m, 1H), 5.307 (m, 0.7H), 4.757 (m, 0.3H), 4.331 (m, 1H), 3.704 (m, 2H), 3.475 (m, 2H), 3.166 (m, 2H), 2.923 (m, 1H), 2.785 (m, 1H), 2.329 (m, 1H), 2.107 (m, 4H), 1.852 (m, 3H), 1.713 (m, 3H).

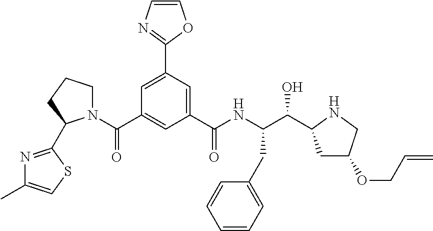 [SMILES: C=CCO[C@H]1CN[C@@H]([C@@H](O)[C@H](Cc2ccccc2)NC(=O)c2cc(C(=O)N3CCC[C@@H]3c3nc(C)cs3)cc(-c3ncco3)c2)C1]

N-((1R,2S)-1-((2R,4R)-4-(allyloxy)pyrrolidin-2-yl)-1-hydroxy-3-phenylpropan-2-yl)-3-((R)-2-(4-methylthiazol-2-yl)pyrrolidine-1-carbonyl)-5-(oxazol-2-yl)benzamide: ¹H NMR (300 MHz, CDCl₃), d: 8.344 (s, 1H), 8.248 (s, 1H), 7.873 (s, 1H), 7.723 (s, 1H), 7.259-7.089 (m, 6H), 6.782 (s, 0.8H), 6.646 (s, 0.2H), 5.878 (m, 1H), 5.577 (m, 0.7H), 5.277-5.075 (m, 2.3H), 4.329 (m, 1H), 4.019-3.828 (m, 3H), 3.703 (m, 2H), 3.485-3.341 (m, 1H), 3.221 (m, 2H), 3.073 (m, 1H), 2.917 (m, 1H), 2.674 (m, 2H), 2.410 (s, 3H), 2.294 (m, 1H), 2.136-1.863 (m, 4H).

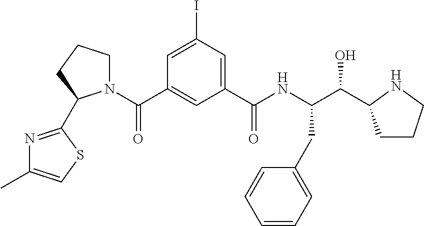 [SMILES: Cc1csc([C@H]2CCCN2C(=O)c2cc(I)cc(C(=O)N[C@@H](Cc3ccccc3)[C@H](O)[C@H]3CCCN3)c2)n1]

N-((1R,2S)-1-hydroxy-3-phenyl-1-((R)-pyrrolidin-2-yl)propan-2-yl)-3-iodo-5-((R)-2-(4-methylthiazol-2-yl)pyrrolidine-1-carbonyl)benzamide: ¹H NMR (300 MHz, CDCl₃): 1.61-2.16 (m, 6H), 2.21-2.40 (m, 5H), 2.78-3.16 (m, 5H), 3.24-3.40 (m, 1H), 3.56-3.70 (m, 2H), 4.22-4.40 (br s, 1H), 5.42-5.56 (m, 1H), 6.80 (s, 1H), 7.13-7.22 (m, 5H), 7.60 (s, 1H), 7.75-7.82 (m, 2H).

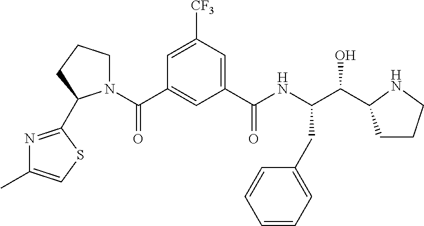 [SMILES: Cc1csc([C@H]2CCCN2C(=O)c2cc(C(=O)N[C@@H](Cc3ccccc3)[C@H](O)[C@H]3CCCN3)cc(C(F)(F)F)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-(trifluoromethyl)benzamide: ¹H NMR (300 MHz, CDCl₃): 1.61-2.18 (m, 6H), 2.21-2.42 (m, 5H), 2.81-3.21 (m, 5H), 3.32-3.42 (m, 1H), 3.62-3.76 (m, 2H), 4.24-4.40 (br s, 1H), 5.56-5.61 (m, 1H), 6.81 (s, 1H), 7.12-7.38 (m, 5H), 7.80 (br s, 2H), 7.88 (s, 1H).

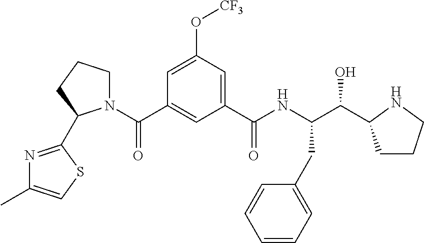 [SMILES: Cc1csc([C@H]2CCCN2C(=O)c2cc(OC(F)(F)F)cc(C(=O)N[C@@H](Cc3ccccc3)[C@H](O)[C@H]3CCCN3)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-(trifluoromethoxy)benzamide: ¹H NMR (300 MHz, CDCl₃) δ 7.91 (s, 1H), 7.68-7.60 (m, 3H), 7.46-7.32 (m, 7H), 7.11-6.88 (m, 2H), 5.81-5.77 (m, 0.7H), 5.20-5.18 (m, 0.3H), 4.58 (m, 1H), 3.88-3.85 (m, 3H), 3.64-3.60 (m, 1H), 3.45-3.03 (m, 8H), 2.63-2.46 (m, 6H), 2.35-2.24 (m, 2H), 2.17-1.89 (m, 7H).

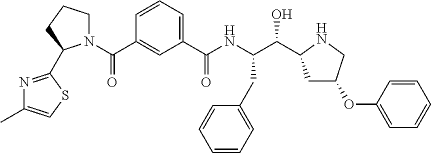 [SMILES: Cc1csc([C@H]2CCCN2C(=O)c2cccc(C(=O)N[C@@H](Cc3ccccc3)[C@H](O)[C@H]3C[C@@H](Oc4ccccc4)CN3)c2)n1]

N-((1R,2S)-1-hydroxy-1-((2R,4R)-4-phenoxypyrrolidin-2-yl)-3-phenylpropan-2-yl)-3-((R)-2-(4-methylthiazol-2-yl)pyrrolidine-1-carbonyl)benzamide: ¹H NMR (300 MHz, CDCl₃) δ 7.78 (s, 1H), 7.69-7.62 (m, 2H), 7.44-7.38 (m, 1H), 7.38-7.18 (m, 7H), 6.98-6.80 (m, 3H), 6.80 (s, 1H), 6.56-6.40 (m, 1H), 5.68-5.60 (m, 1H), 4.84-4.78 (m, 1H), 4.42-4.24 (m, 1H), 3.96-3.60 (m, 2H), 3.51-3.20 (m, 3H), 3.20-2.92 (m, 3H), 2.44 (s, 3H), 2.44-2.24 (m, 3H), 2.16-1.80 (m, 5H).

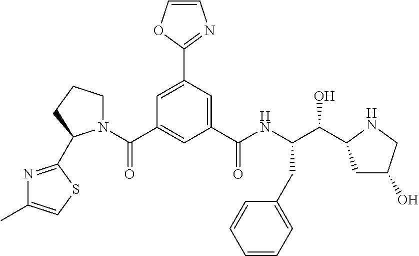 [SMILES: Cc1csc([C@H]2CCCN2C(=O)c2cc(C(=O)N[C@@H](Cc3ccccc3)[C@H](O)[C@H]3C[C@@H](O)CN3)cc(-c3ncco3)c2)n1]

N-((1R,2S)-1-hydroxy-1-((2R,4R)-4-hydroxypyrrolidin-2-yl)-3-phenylpropan-2-yl)-3-((R)-2-(4-methylthiazol-2-yl)pyrrolidine-1-carbonyl)-5-(oxazol-2-yl)benzamide.

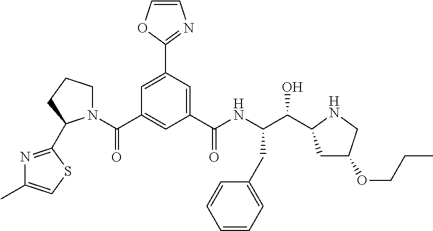 [SMILES: CCCO[C@H]1CN[C@@H]([C@@H](O)[C@H](Cc2ccccc2)NC(=O)c2cc(C(=O)N3CCC[C@@H]3c3nc(C)cs3)cc(-c3ncco3)c2)C1]

N-((1R,2S)-1-hydroxy-3-phenyl-1-((2R,4R)-4-propoxypyrrolidin-2-yl)propan-2-yl)-3-((R)-2-(4-methylthiazol-2-yl)pyrrolidine-1-carbonyl)-5-(oxazol-2-yl)benzamide: ¹H NMR (300 MHz, CDCl3+CD3OD), d: 8.309 (m, 2H), 7.724 (m, 1H), 7.208 (m, 6H), 6.786 (s, 0.9H), 6.652 (s, 0.1H), 5.581 (m, 0.8H), 5.084 (m, 0.2H), 4.326 (m, 1H), 3.942 (m, 1H), 3.749-3.608 (m, 2H), 3.496-3.194 (m, 5H), 3.091 (m, 1H), 2.934 (m, 1H), 2.676-2.259 (m, 3H), 2.415 (s, 3H), 2.099-1.864 (m, 4H), 1.575 (m, 2H), 0.898 (t, 3H).

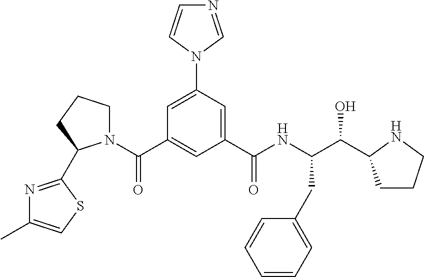 [SMILES: Cc1csc([C@H]2CCCN2C(=O)c2cc(C(=O)N[C@@H](Cc3ccccc3)[C@H](O)[C@H]3CCCN3)cc(-n3ccnc3)c2)n1]

N-((1R,2S)-1-hydroxy-3-phenyl-1-((R)-pyrrolidin-2-yl)propan-2-yl)-3-(1H-imidazol-1-yl)-5-((R)-2-(4-methylthiazol-2-yl)pyrrolidine-1-carbonyl)benzamide: ¹H NMR (300 MHz, CDCl₃): 1.98-2.45 (m, 11H), 3.02-3.98 (m, 8H), 4.43-4.58 (m, 1H), 5.56-5.60 (m, 1H), 6.81 (s, 1H), 7.12-7.42 (m, 8H), 7.62-7.80 (m, 3H).

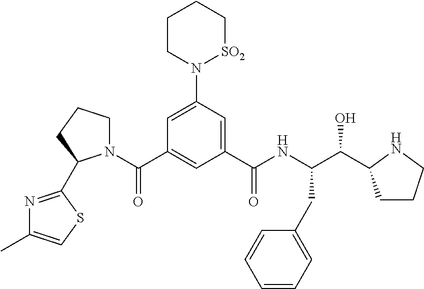 [SMILES: Cc1csc([C@H]2CCCN2C(=O)c2cc(C(=O)N[C@@H](Cc3ccccc3)[C@H](O)[C@H]3CCCN3)cc(N3CCCCS3(=O)=O)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-(thiazinanyl-S,S-dioxide)benzamide: ¹H NMR (300 MHz, CDCl₃): 1.62-2.40 (m, 15H), 2.76-3.79 (m, 12H), 4.24-4.38 (m, 1H), 5.56-5.60 (m, 1H), 6.81 (s, 1H), 7.13-7.42 (m, 5H), 7.60-7.78 (m, 3H).

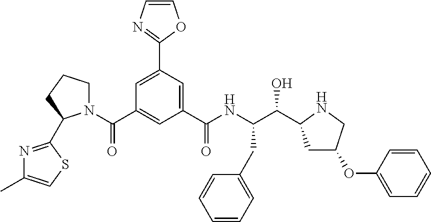 [SMILES: Cc1csc([C@H]2CCCN2C(=O)c2cc(C(=O)N[C@@H](Cc3ccccc3)[C@H](O)[C@H]3C[C@@H](Oc4ccccc4)CN3)cc(-c3ncco3)c2)n1]

N-((1R,2S)-1-hydroxy-1-((2R,4R)-4-phenoxypyrrolidin-2-yl)-3-phenylpropan-2-yl)-3-((R)-2-(4-methylthiazol-2-yl)pyrrolidine-1-carbonyl)-5-(oxazol-2-yl)benzamide: ¹H NMR (300 MHz, CDCl₃) δ 8.20-8.28 (m, 2H), 7.8 (s, 1H), 7.72 (s, 1H), 7.38-7.12 (m, 8H), 6.98-6.78 (m, 3H), 6.80 (s, 1H), 5.68-5.60 (m, 1H), 4.84-4.78 (m, 1H), 4.44-4.32 (m, 1H), 3.96-3.60 (m, 2H), 3.48-2.96 (m, 5H), 2.46 (s, 3H), 2.46-2.28 (m, 2H), 2.20-1.80 (m, 5H).

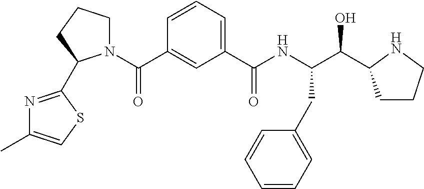 [SMILES: Cc1csc([C@H]2CCCN2C(=O)c2cccc(C(=O)N[C@@H](Cc3ccccc3)[C@@H](O)[C@H]3CCCN3)c2)n1]

N-((1S,2S)-1-hydroxy-3-phenyl-1-((R)-pyrrolidin-2-yl)propan-2-yl)-3-((R)-2-(4-methylthiazol-2-yl)pyrrolidine-1-carbonyl)benzamide: ¹H NMR (CDCl₃): d 7.99 (s, 1H), 7.48-7.82 (m, 1H), 7.45 (m, 1H), 7.16-7.28 (m, 6H), 6.94 (d, 1H), 6.75 (m, 1H), 5.63 (m, 1H), 4.37 (m, 1H), 2.80-3.94 (m, 9H), 2.71 (m, 1H), 2.21-2.50 (m, 5H), 1.48-2.19 (m, 4H).

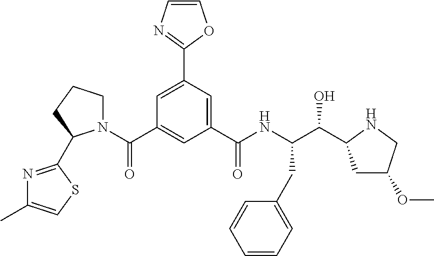

N-((1R,2S)-1-hydroxy-1-((2R,4R)-4-methoxypyrrolidin-2-yl)-3-phenylpropan-2-yl)-3-((R)-2-(4-methylthiazol-2-yl)pyrrolidine-1-carbonyl)-5-(oxazol-2-yl)benzamide: ¹H NMR (300 MHz, CDCl₃) δ 8.30-8.16 (m, 2H), 7.92-7.83 (m, 1H), 7.71-7.54 (m, 1H), 7.32-7.18 (m, 7H), 6.91-6.61 (m, 2H), 5.65-5.61 (m, 0.7H), 5.06-5.03 (m, 0.2H), 4.36-4.35 (m, 1H), 3.93-3.84 (m, 2H), 3.69-3.55 (m, 2H), 3.44-3.38 (m, 2H), 3.31 (s, 3H), 3.25-3.04 (m, 3H), 2.71-2.67 (m, 1H), 2.44-2.32 (m, 5H), 2.22 (s, 1H), 2.16-1.87 (m, 5H).

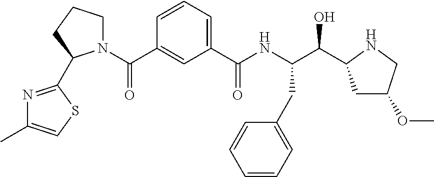

N-((1S,2S)-1-hydroxy-1-((2R,4R)-4-methoxypyrrolidin-2-yl)-3-phenylpropan-2-yl)-3-((R)-2-(4-methylthiazol-2-yl)pyrrolidine-1-carbonyl)benzamide: ¹H NMR (300 MHz, CDCl₃) δ 7.79 (s, 1H), 7.66-7.51 (m, 2H), 7.44-7.39 (m, 1H), 7.34-7.18 (m, 7H), 6.78-6.66 (m, 1H), 6.37-6.12 (m, 1H), 5.65-5.61 (m, 0.7H), 5.07-5.05 (m, 0.2H), 4.39-4.31 (m, 1H), 3.89 (m, 1H), 3.70-3.55 (m, 3H), 3.48-3.32 (m, 5H), 3.18-3.04 (m, 3H), 2.79-2.74 (m, 1H), 2.44-2.29 (m, 6H), 2.14-1.83 (m, 5H).

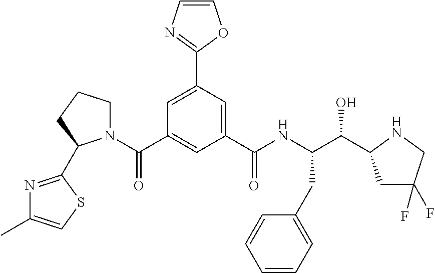 [SMILES: Cc1csc([C@H]2CCCN2C(=O)c2cc(C(=O)N[C@@H](Cc3ccccc3)[C@H](O)[C@H]3CC(F)(F)CN3)cc(-c3ncco3)c2)n1]

N-((1R,2S)-1-((R)-4,4-difluoropyrrolidin-2-yl)-1-hydroxy-3-phenylpropan-2-yl)-3-((R)-2-(4-methylthiazol-2-yl)pyrrolidine-1-carbonyl)-5-(oxazol-2-yl)benzamide: ¹H NMR (300 MHz, CDCl₃+CD₃OD), d: 8.193 (m, 2H), 7.720 (m, 1H), 7.221 (m, 6H), 6.799 (s, 0.9H), 6.634 (s, 0.1H), 5.577 (m, 0.8H), 5.037 (m, 0.2H), 4.303 (m, 1H), 3.829-3.588 (m, 2H), 3.521-3.359 (m, 2H), 3.308-2.873 (m, 4H), 2.505-2.192 (m, 4H), 2.419 (s, 3H), 2.056 (m, 2H), 1.912 (m, 2H).

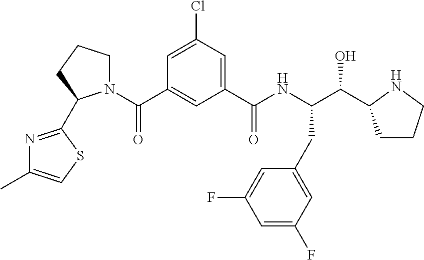 [SMILES: Cc1csc([C@H]2CCCN2C(=O)c2cc(Cl)cc(C(=O)N[C@@H](Cc3cc(F)cc(F)c3)[C@H](O)[C@H]3CCCN3)c2)n1]

3-chloro-N-((1R,2S)-3-(3,5-difluorophenyl)-1-hydroxy-1-((R)-pyrrolidin-2-yl)propan-2-yl)-5-((R)-2-(4-methylthiazol-2-yl)pyrrolidine-1-carbonyl)benzamide: ¹H NMR (300 MHz, CDCl₃): 1.72-2.36 (m, 6H), 2.27-2.41 (m, 5H), 2.46-2.97 (m, 5H), 3.38-3.45 (m, 1H), 3.56-3.72 (m, 2H), 4.28-4.44 (m, 1H), 5.52-5.58 (m, 1H), 7.40-7.60 (m, 7H).

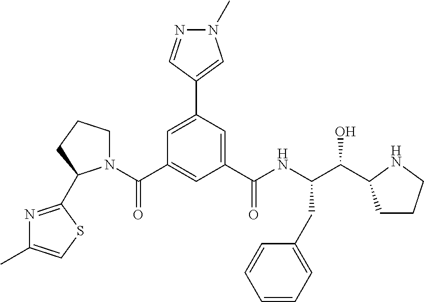 [SMILES: Cc1csc([C@H]2CCCN2C(=O)c2cc(C(=O)N[C@@H](Cc3ccccc3)[C@H](O)[C@H]3CCCN3)cc(-c3cnn(C)c3)c2)n1]

N-((1R,2S)-1-hydroxy-3-phenyl-1-((R)-pyrrolidin-2-yl)propan-2-yl)-3-(1-methyl-1H-pyrazol-4-yl)-5-((R)-2-(4-methylthiazol-2-yl)pyrrolidine-1-carbonyl)benzamide: ¹H NMR (300 MHz, CDCl₃): 1.64-2.08 (m, 6H), 2.08-2.39 (m, 4H), 2.42-3.21 (m, 5H), 3.40-3.46 (m, 1H), 3.44-3.66 (m, 2H), 3.64-3.90 (m, 4H), 4.34-4.44 (m, 1H), 5.57-5.62 (m, 1H), 6.78 (s, 1H), 7.22-7.33 (m, 7H), 7.38-7.68 (m, 3H).

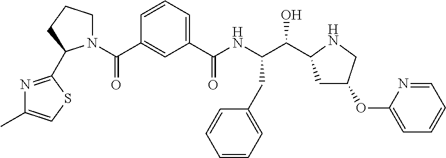 [SMILES: Cc1csc([C@H]2CCCN2C(=O)c2cccc(C(=O)N[C@@H](Cc3ccccc3)[C@H](O)[C@H]3C[C@@H](Oc4ccccn4)CN3)c2)n1]

N-((1R,2S)-1-hydroxy-3-phenyl-1-((2R,4R)-4-(pyridin-2-yloxy)pyrrolidin-2-yl)propan-2-yl)-3-((R)-2-(4-methylthiazol-2-yl)pyrrolidine-1-carbonyl)benzamide: ¹H NMR (300 MHz, CDCl₃) δ 8.16 (m, 1H), 7.78 (s, 1H), 7.69-7.48 (m, 2H), 7.44-7.38 (m, 1H), 7.38-7.16 (m, 6H), 6.88-6.60 (m, 3H), 6.40 (m, 1H), 5.66-5.60 (m, 1H), 5.46-5.40 (m, 1H), 4.44-4.24 (m, 1H), 3.94-3.60 (m, 2H), 3.51-3.20 (m, 2H), 3.20-3.01 (m, 4H), 2.44 (s, 3H), 2.44-2.22 (m, 3H), 2.16-1.80 (m, 4H).

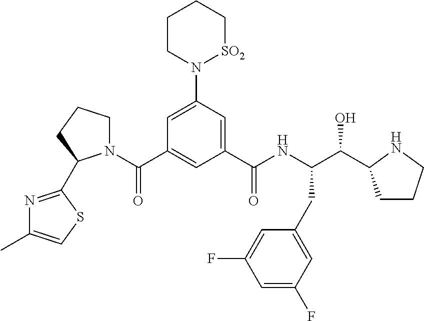 [SMILES: Cc1csc([C@H]2CCCN2C(=O)c2cc(C(=O)N[C@@H](Cc3cc(F)cc(F)c3)[C@H](O)[C@H]3CCCN3)cc(N3CCCCS3(=O)=O)c2)n1]

N-((1R,2S)-1-hydroxy-3-(3,5-difluorophenyl)-1-((R)-pyrrolidin-2-yl)propan-2-yl)-3-((R)-2-(4-methylthiazol-2-yl)pyrrolidine-1-carbonyl)-5-(thiazinanyl-S,S-dioxide)benzamide. ¹H NMR (300 MHz, CDCl₃) d 1.67-2.40 (m, 15H), 2.82-3.74 (m, 12H), 4.23-4.36 (m, 1H), 5.54-5.58 (m, 1H), 6.54-6.81 (m, 4H), 7.58 (s, 2H), 7.72 (s, 1H).

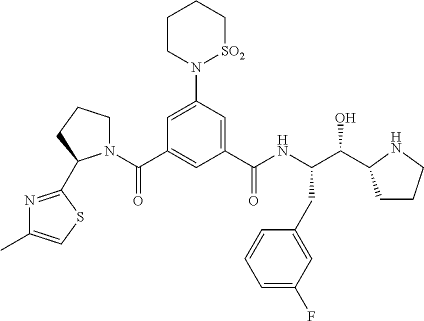 [SMILES: Cc1csc([C@H]2CCCN2C(=O)c2cc(C(=O)N[C@@H](Cc3cccc(F)c3)[C@H](O)[C@H]3CCCN3)cc(N3CCCCS3(=O)=O)c2)n1]

N-((1R,2S)-1-hydroxy-3-(5-fluorophenyl)-1-((R)-pyrrolidin-2-yl)propan-2-yl)-3-((R)-2-(4-methylthiazol-2-yl)pyrrolidine-1-carbonyl)-5-(thiazinanyl-S,S-dioxide)benzamide.

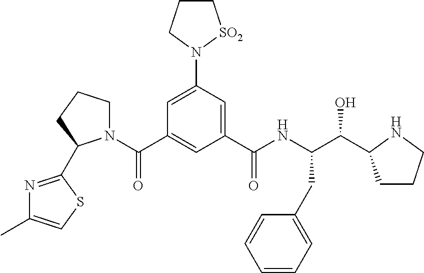 [SMILES: Cc1csc([C@H]2CCCN2C(=O)c2cc(C(=O)N[C@@H](Cc3ccccc3)[C@H](O)[C@H]3CCCN3)cc(N3CCCS3(=O)=O)c2)n1]

N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-([1,2]thiazolidyl-S,S-dioxide)benzamide. ¹H NMR (300 MHz, CDCl₃) d 1.63-2.45 (m, 13H), 2.79-3.64 (m, 12H), 4.32 (br s, 1H), 5.53-5.57 (m, 1H), 6.75 (s, 1H), 7.08-7.27 (m, 5H), 7.40 (s, 1H), 7.46 (s, 1H), 7.52 (s, 1H).

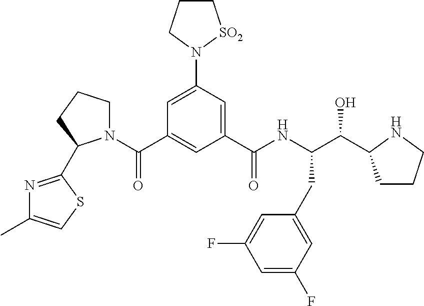 [SMILES: Cc1csc([C@H]2CCCN2C(=O)c2cc(C(=O)N[C@@H](Cc3cc(F)cc(F)c3)[C@H](O)[C@H]3CCCN3)cc(N3CCCS3(=O)=O)c2)n1]

N-((1R,2S)-1-hydroxy-3-(3,5-difluorophenyl)-1-((R)-pyrrolidin-2-yl)propan-2-yl)-3-((R)-2-(4-methylthiazol-2-yl)pyrrolidine-1-carbonyl)-5-([1,2]thiazolidyl-S,S-dioxide)benzamide. ¹H NMR (300 MHz, CDCl₃) d 1.72-2.52 (m, 13H), 2.81-3.70 (m, 12H), 4.29 (br s, 1H), 5.51-5.58 (m, 1H), 6.53-6.81 (m, 3H), 7.21-7.26 (m, 2H), 7.47-7.58 (m, 2H).

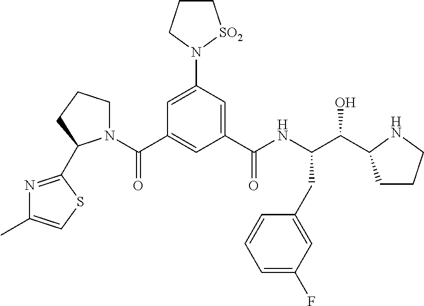 [SMILES: Cc1csc([C@H]2CCCN2C(=O)c2cc(C(=O)N[C@@H](Cc3cccc(F)c3)[C@H](O)[C@H]3CCCN3)cc(N3CCCS3(=O)=O)c2)n1]

N-((1R,2S)-1-hydroxy-3-(3-fluorophenyl)-1-((R)-pyrrolidin-2-yl)propan-2-yl)-3-((R)-2-(4-methylthiazol-2-yl)pyrrolidine-1-carbonyl)-5-([1,2]thiazolidyl-S,S-dioxide)benzamide. ¹H NMR (300 MHz, CDCl₃) d 7.68-7.60 (m, 1H), 7.48 (s, 1H), 7.38 (s, 1H), 7.28-7.12 (m, 3H), 7.03-6.64 (m, 5H), 5.61-5.57 (m, 0.8H), 5.11-5.06 (m, 0.2H), 4.33 (m, 1H), 3.83-3.67 (m, 3H), 3.57-3.14 (m, 8H), 3.03-2.86 (m, 4H), 2.54-2.21 (m, 8H), 2.13-2.03 (m, 2H), 1.98-1.69 (m, 6H).

### Example 3

### Inhibition of Memapsin 2 Beta-Secretase Activity

Potency of compounds were determined by measurement of their inhibition of memapsin 2 activity toward a fluorescent substrate. Kinetic inhibition experiments were performed using the procedure as described in Ermolieff, et al. (Biochemistry 39:12450-12456 (2000), the teachings of which are incorporated hereby in their entirety). Briefly, assays were performed at pH 4, 37° C., by pre-incubation of memapsin 2 enzyme with compound for 20 minutes. Activity measurements were initiated by addition of a fluorogenic substrate FS-2 (Bachem Americas, Torrance, Calif.) MCA-SEVNLDAEFR-DNP (SEQ ID NO.: 2). The substrate was derived from 10 amino acids of the human amyloid precursor protein (APP), with the Swedish variant amino acids at the beta-secretase cleavage site. The terminal amino acid was modified from arginine to lysine to facilitate derivatization with a functional group for detection by autofluorescence. The amino acid sequence of the “core” peptide of the substrate is SEVNLDAEFK (SEQ ID NO.: 3). The amino terminus was derivatized with (7-methoxycoumarin-4-yl)acetyl (MCA), and the epsilon amine of the lysine side chain of the terminal residue (K in sequence SEVNLDAEFK (SEQ ID NO.: 3)) was derivatized with 2,4-dinitrophenyl (DNP). Results are shown in Table 1 (“M2 Ki”).

TABLE 1
Compound Assay data.

| | | M2 | M2 | CD | M1 | | HLM |
|---|---|---|---|---|---|---|---|
| Ref # | Structure | Ki | IC50 | Ki | Ki | CYP3A4 Ki | CL |
| 1-1 | (R)-tert-butyl 2-((1S,2S)-1- | + | + | | | | |
| | hydroxy-2-(3-(methyl((4- | | | | | | |
| | methylthiazol-2- | | | | | | |
| | yl)methyl)carbamoyl)benzamido)- | | | | | | |
| | 3-phenylpropyl)pyrrolidine-1- | | | | | | |
| | carboxylate | | | | | | |
| 1-2 | N1-((1R,2S)-1-hydroxy-3-phenyl- | ++ | + | | | | |
| | 1-((R)-pyrrolidin-2-yl)propan-2- | | | | | | |
| | yl)-N3-methyl-N3-((4- | | | | | | |
| | methylthiazol-2- | | | | | | |
| | yl)methyl)isophthalamide | | | | | | |
| 1-3 | (R)-tert-butyl 2-((1S,2S)-1- | + | | | | | |
| | hydroxy-2-(3-((R)-2-(4- | | | | | | |
| | methyloxazol-2-yl)pyrrolidine-1- | | | | | | |
| | carbonyl)benzamido)-3- | | | | | | |
| | phenylpropyl)pyrrolidine-1- | | | | | | |
| | carboxylate | | | | | | |
| 1-4 | N-((1R,2S)-1-hydroxy-3-phenyl-1- | ++ | + | | | | |
| | ((R)-pyrrolidin-2-yl)propan-2-yl)- | | | | | | |
| | 3-((R)-2-(4-methyloxazol-2- | | | | | | |
| | yl)pyrrolidine-1- | | | | | | |
| | carbonyl)benzamide | | | | | | |
| 1-5 | (R)-tert-butyl 2-((1S,2S)-1- | + | + | | | | |
| | hydroxy-2-(3-(N- | | | | | | |
| | methylmethylsulfonamido)-5-((R)- | | | | | | |
| | 1- | | | | | | |
| | phenylethylcarbamoyl)benzamido)- | | | | | | |
| | 3-phenylpropyl)pyrrolidine-1- | | | | | | |
| | carboxylate | | | | | | |
| 1-6 | (R)-tert-butyl 2-((1S,2S)-1- | + | + | | | | |
| | hydroxy-2-(3-methyl-5-((R)-2-(4- | | | | | | |
| | methyloxazol-2-yl)pyrrolidine-1- | | | | | | |
| | carbonyl)benzamido)-3- | | | | | | |
| | phenylpropyl)pyrrolidine-1- | | | | | | |
| | carboxylate | | | | | | |
| 1-7 | (R)-tert-butyl 2-((1S,2S)-1- | + | + | | | | |
| | hydroxy-2-(3-(methyl((4- | | | | | | |
| | methylthiazol-2- | | | | | | |
| | yl)methyl)carbamoyl)-5-(oxazol-2- | | | | | | |
| | yl)benzamido)-3- | | | | | | |
| | phenylpropyl)pyrrolidine-1- | | | | | | |
| | carboxylate | | | | | | |
| 1-8 | N-((1R,2S)-1-hydroxy-3-phenyl-1- | ++ | + | | | +++ | +++ |
| | ((R)-pyrrolidin-2-yl)propan-2-yl)- | | | | | | |
| | 3-methyl-5-((R)-2-(4- | | | | | | |
| | methyloxazol-2-yl)pyrrolidine-1- | | | | | | |
| | carbonyl)benzamide | | | | | | |
| 1-9 | N1-((1R,2S)-1-((2R,4R)-4- | +++ | +++ | | | + | +++ |
| | (benzyloxy)pyrrolidin-2-yl)-1- | | | | | | |
| | hydroxy-3-phenylpropan-2-yl)-N3- | | | | | | |
| | methyl-N3-((4-methylthiazol-2- | | | | | | |
| | yl)methyl)isophthalamide | | | | | | |
| 1-10 | N1-((1R,2S)-1-hydroxy-1- | ++ | + | | | | |
| | ((2R,4R)-4-hydroxypyrrolidin-2- | | | | | | |
| | yl)-3-phenylpropan-2-yl)-N3- | | | | | | |
| | methyl-N3-((4-methylthiazol-2- | | | | | | |
| | yl)methyl)isophthalamide | | | | | | |
| 1-11 | N1-((1R,2S)-1-hydroxy-3-phenyl- | +++ | +++ | | | +++ | +++ |
| | 1-((R)-pyrrolidin-2-yl)propan-2- | | | | | | |
| | yl)-5-(N- | | | | | | |
| | methylmethylsulfonamido)-N3- | | | | | | |
| | ((R)-1-phenylethyl)isophthalamide | | | | | | |
| 1-12 | N1-((1R,2S)-1-hydroxy-3-phenyl- | +++ | + | | | | +++ |
| | 1-((R)-pyrrolidin-2-yl)propan-2- | | | | | | |
| | yl)-N3-methyl-N3-((4- | | | | | | |
| | methylthiazol-2-yl)methyl)-5- | | | | | | |
| | (oxazol-5-yl)isophthalamide | | | | | | |
| 1-13 | N1-((1R,2S)-1-hydroxy-3-phenyl- | +++ | ++ | | | ++ | +++ |
| | 1-((R)-pyrrolidin-2-yl)propan-2- | | | | | | |
| | yl)-N3-methyl-N3-((4- | | | | | | |
| | methylthiazol-2-yl)methyl)-5-(1H- | | | | | | |
| | pyrrol-1-yl)isophthalamide | | | | | | |
| 1-14 | 3-(difluoromethyl)-N-((1R,2S)-1- | +++ | +++ | | | + | +++ |
| | hydroxy-3-phenyl-1-((R)- | | | | | | |
| | pyrrolidin-2-yl)propan-2-yl)-5- | | | | | | |
| | ((R)-2-(4-methylthiazol-2- | | | | | | |
| | yl)pyrrolidine-1- | | | | | | |
| | carbonyl)benzamide | | | | | | |
| 1-15 | N1-((1R,2S)-1-hydroxy-3-phenyl- | +++ | +++ | | | ++ | +++ |
| | 1-((R)-pyrrolidin-2-yl)propan-2- | | | | | | |
| | yl)-N3-methyl-N3-((4- | | | | | | |
| | methylthiazol-2-yl)methyl)-5- | | | | | | |
| | (oxazol-2-yl)isophthalamide | | | | | | |
| 1-16 | N-((1R,2S)-1-hydroxy-3-phenyl-1- | +++ | ++ | | | + | +++ |
| | ((R)-pyrrolidin-2-yl)propan-2-yl)- | | | | | | |
| | 3-((R)-2-(4-methylthiazol-2- | | | | | | |
| | yl)pyrrolidine-1-carbonyl)-5-(1H- | | | | | | |
| | pyrrol-1-yl)benzamide | | | | | | |
| 1-17 | N-((1R,2S)-1-hydroxy-3-phenyl-1- | +++ | +++ | | | +++ | +++ |
| | ((R)-pyrrolidin-2-yl)propan-2-yl)- | | | | | | |
| | 3-((R)-2-(4-methyloxazol-2- | | | | | | |
| | yl)pyrrolidine-1-carbonyl)-5- | | | | | | |
| | (oxazol-2-yl)benzamide | | | | | | |
| 1-18 | N-((1R,2S)-1-((2R,4R)-4- | +++ | +++ | | | +++ | +++ |
| | (benzyloxy)pyrrolidin-2-yl)-1- | | | | | | |
| | hydroxy-3-phenylpropan-2-yl)-3- | | | | | | |
| | ((R)-2-(4-methyloxazol-2- | | | | | | |
| | yl)pyrrolidine-1- | | | | | | |
| | carbonyl)benzamide | | | | | | |
| 1-19 | N-((1R,2S)-1-hydroxy-3-phenyl-1- | +++ | +++ | + | | +++ | |
| | ((R)-pyrrolidin-2-yl)propan-2-yl)- | | | | | | |
| | 3-((R)-2-(4-methylthiazol-2- | | | | | | |
| | yl)pyrrolidine-1-carbonyl)-5- | | | | | | |
| | (oxazol-2-yl)benzamide | | | | | | |
| 1-20 | N-((1R,2S)-1-hydroxy-3-phenyl-1- | +++ | +++ | | | +++ | |
| | ((R)-pyrrolidin-2-yl)propan-2-yl)- | | | | | | |
| | 3-((R)-2-(4-methylthiazol-2- | | | | | | |
| | yl)pyrrolidine-1-carbonyl)-5- | | | | | | |
| | (pyrazin-2-yl)benzamide | | | | | | |
| 1-21 | N-((1R,2S)-1-hydroxy-3-phenyl-1- | +++ | +++ | | | | |
| | ((R)-pyrrolidin-2-yl)propan-2-yl)- | | | | | | |
| | 3-((R)-2-(4-methyloxazol-2- | | | | | | |
| | yl)pyrrolidine-1-carbonyl)-5- | | | | | | |
| | (pyrazin-2-yl)benzamide | | | | | | |
| 1-22 | N-((1R,2S)-1-((2R,4R)-4- | +++ | +++ | ++ | | + | |
| | (benzyloxy)pyrrolidin-2-yl)-1- | | | | | | |
| | hydroxy-3-phenylpropan-2-yl)-3- | | | | | | |
| | ((R)-2-(4-methylthiazol-2- | | | | | | |
| | yl)pyrrolidine-1- | | | | | | |
| | carbonyl)benzamide | | | | | | |
| 1-23 | N-((1R,2S)-1-hydroxy-3-phenyl-1- | +++ | +++ | + | | ++ | |
| | ((R)-pyrrolidin-2-yl)propan-2-yl)- | | | | | | |
| | 3-(N-methylmethylsulfonamido)-5- | | | | | | |
| | ((R)-2-(4-methylthiazol-2- | | | | | | |
| | yl)pyrrolidine-1- | | | | | | |
| | carbonyl)benzamide | | | | | | |
| 1-24 | N-((1R,2S)-1-hydroxy-3-phenyl-1- | +++ | +++ | | | | |
| | ((R)-pyrrolidin-2-yl)propan-2-yl)- | | | | | | |
| | 3-((R)-2-(4-methyloxazol-2- | | | | | | |
| | yl)pyrrolidine-1-carbonyl)-5- | | | | | | |
| | (pyridin-2-yl)benzamide | | | | | | |
| 1-25 | N-((1R,2S)-1-((2R,4R)-4- | +++ | +++ | +++ | + | | |
| | (benzyloxy)pyrrolidin-2-yl)-1- | | | | | | |
| | hydroxy-3-phenylpropan-2-yl)-3- | | | | | | |
| | ((R)-2-(4-methylthiazol-2- | | | | | | |
| | yl)pyrrolidine-1-carbonyl)-5-(1H- | | | | | | |
| | pyrrol-1-yl)benzamide | | | | | | |
| 1-26 | N-((1R,2S)-1-hydroxy-3-phenyl-1- | +++ | | | | ++ | |
| | ((R)-pyrrolidin-2-yl)propan-2-yl)- | | | | | | |
| | 3-((R)-2-(4-methyloxazol-2- | | | | | | |
| | yl)pyrrolidine-1-carbonyl)-5-(1H- | | | | | | |
| | pyrrol-1-yl)benzamide | | | | | | |
| 1-27 | N1-((1R,2S)-1-hydroxy-3-phenyl- | ++ | +++ | | | | |
| | 1-((R)-pyrrolidin-2-yl)propan-2- | | | | | | |
| | yl)-N3-methyl-5-(N- | | | | | | |
| | methylmethylsulfonamido)-N3-((4- | | | | | | |
| | methylthiazol-2- | | | | | | |
| | yl)methyl)isophthalamide | | | | | | |
| 1-28 | N-((1R,2S)-1-hydroxy-3-phenyl-1- | +++ | | | | +++ | |
| | ((R)-pyrrolidin-2-yl)propan-2-yl)- | | | | | | |
| | 3-(N-methylmethylsulfonamido)-5- | | | | | | |
| | ((R)-2-(4-methyloxazol-2- | | | | | | |
| | yl)pyrrolidine-1- | | | | | | |
| | carbonyl)benzamide | | | | | | |
| 1-29 | N-((1R,2S)-1-hydroxy-3-phenyl-1- | + | | | | | |
| | ((2R,5S)-5-phenylpyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)benzamide | | | | | | |
| 1-30 | 2′,4′-difluoro-N3-((1R,2S)-1- | +++ | | | | | |
| | hydroxy-3-phenyl-1-((R)- | | | | | | |
| | pyrrolidin-2-yl)propan-2-yl)-N5- | | | | | | |
| | methyl-N5-((4-methylthiazol-2- | | | | | | |
| | yl)methyl)biphenyl-3,5- | | | | | | |
| | dicarboxamide | | | | | | |
| 1-31 | N-((1R,2S)-1-hydroxy-3-phenyl-1- | + | | | | | |
| | ((2S,5R)-5-phenylpyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)benzamide | | | | | | |
| 1-32 | 3-fluoro-N-((1R,2S)-1-hydroxy-3- | +++ | + | | | ++ | +++ |
| | phenyl-1-((R)-pyrrolidin-2- | | | | | | |
| | yl)propan-2-yl)-5-((R)-2-(4- | | | | | | |
| | methylthiazol-2-yl)pyrrolidine-1- | | | | | | |
| | carbonyl)benzamide | | | | | | |
| 1-33 | N-((1R,2S)-1-hydroxy-3-phenyl-1- | + | + | | | ++ | |
| | ((R)-pyrrolidin-2-yl)propan-2-yl)- | | | | | | |
| | 3-((R)-2-(4-methylthiazol-2- | | | | | | |
| | yl)pyrrolidine-1-carbonyl)-5- | | | | | | |
| | morpholinobenzamide | | | | | | |
| 1-34 | N-((1R,2S)-1-hydroxy-3-phenyl-1- | +++ | + | | | ++ | |
| | ((R)-pyrrolidin-2-yl)propan-2-yl)- | | | | | | |
| | 3-methoxy-5-((R)-2-(4- | | | | | | |
| | methylthiazol-2-yl)pyrrolidine-1- | | | | | | |
| | carbonyl)benzamide | | | | | | |
| 1-35 | (2R,4R)-tert-butyl 4-(benzyloxy)- | | | | | | |
| | 2-((1S,2S)-2-(3-(fluoromethyl)-5- | | | | | | |
| | ((R)-2-(4-methylthiazol-2- | | | | | | |
| | yl)pyrrolidine-1- | | | | | | |
| | carbonyl)benzamido)-1-hydroxy-3- | | | | | | |
| | phenylpropyl)pyrrolidine-1- | | | | | | |
| | carboxylate | | | | | | |
| 1-36 | N-((1R,2S)-1-((2R,4R)-4- | +++ | +++ | | | +++ | |
| | (benzyloxy)pyrrolidin-2-yl)-1- | | | | | | |
| | hydroxy-3-phenylpropan-2-yl)-3- | | | | | | |
| | (fluoromethyl)-5-((R)-2-(4- | | | | | | |
| | methylthiazol-2-yl)pyrrolidine-1- | | | | | | |
| | carbonyl)benzamide | | | | | | |
| 1-37 | (2R,4S)-tert-butyl 4-fluoro-2- | | | | | | |
| | ((1S,2S)-1-hydroxy-2-(3-((R)-2-(4- | | | | | | |
| | methylthiazol-2-yl)pyrrolidine-1- | | | | | | |
| | carbonyl)-5-(oxazol-2- | | | | | | |
| | yl)benzamido)-3- | | | | | | |
| | phenylpropyl)pyrrolidine-1- | | | | | | |
| | carboxylate | | | | | | |
| 1-38 | N-((1R,2S)-1-((2R,4S)-4- | +++ | | | | | |
| | fluoropyrrolidin-2-yl)-1-hydroxy- | | | | | | |
| | 3-phenylpropan-2-yl)-3-((R)-2-(4- | | | | | | |
| | methylthiazol-2-yl)pyrrolidine-1- | | | | | | |
| | carbonyl)-5-(oxazol-2- | | | | | | |
| | yl)benzamide | | | | | | |
| 1-39 | N-((1R,2S)-1-hydroxy-3-phenyl-1- | +++ | +++ | | | | +++ |
| | ((R)-pyrrolidin-2-yl)propan-2-yl)- | | | | | | |
| | 3-((R)-2-(4-methylthiazol-2- | | | | | | |
| | yl)pyrrolidine-1-carbonyl)-5-(2- | | | | | | |
| | oxopyrrolidin-1-yl)benzamide | | | | | | |
| 1-40 | N-((1R,2S)-1-hydroxy-3-phenyl-1- | +++ | ++ | | | ++ | +++ |
| | ((R)-pyrrolidin-2-yl)propan-2-yl)- | | | | | | |
| | 3-((R)-2-(4-methylthiazol-2- | | | | | | |
| | yl)pyrrolidine-1- | | | | | | |
| | carbonyl)benzamide | | | | | | |
| 1-41 | N1-((1R,2S)-3-(3,5- | +++ | + | | | +++ | +++ |
| | difluorophenyl)-1-hydroxy-1-((R)- | | | | | | |
| | pyrrolidin-2-yl)propan-2-yl)-N3- | | | | | | |
| | methyl-N3-((4-methylthiazol-2- | | | | | | |
| | yl)methyl)isophthalamide | | | | | | |
| 1-42 | 3-(dimethylamino)-N-((1R,2S)-1- | ++ | + | | | | |
| | hydroxy-3-phenyl-1-((R)- | | | | | | |
| | pyrrolidin-2-yl)propan-2-yl)-5- | | | | | | |
| | ((R)-2-(4-methylthiazol-2- | | | | | | |
| | yl)pyrrolidine-1- | | | | | | |
| | carbonyl)benzamide | | | | | | |
| 1-43 | N-((1R,2S)-1-hydroxy-3-phenyl-1- | +++ | + | + | | | +++ |
| | ((R)-pyrrolidin-2-yl)propan-2-yl)- | | | | | | |
| | 5-((R)-2-(4-methylthiazol-2- | | | | | | |
| | yl)pyrrolidine-1-carbonyl)-3′- | | | | | | |
| | (trifluoromethyl)biphenyl-3- | | | | | | |
| | carboxamide | | | | | | |
| 1-44 | N-((1R,2S)-3-(3,5-difluorophenyl)- | +++ | ++ | + | | + | +++ |
| | 1-hydroxy-1-((R)-pyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)benzamide | | | | | | |
| 1-45 | N-((1R,2R)-1-((2R,4R)-4- | + | + | | | | |
| | (benzyloxy)pyrrolidin-2-yl)-1- | | | | | | |
| | hydroxy-3-phenylpropan-2-yl)-3- | | | | | | |
| | (fluoromethyl)-5-((R)-2-(4- | | | | | | |
| | methylthiazol-2-yl)pyrrolidine-1- | | | | | | |
| | carbonyl)benzamide | | | | | | |
| 1-46 | 3-(4,4-difluoropiperidin-1-yl)-N- | ++ | ++ | | | | |
| | ((1R,2S)-1-hydroxy-3-phenyl-1- | | | | | | |
| | ((R)-pyrrolidin-2-yl)propan-2-yl)- | | | | | | |
| | 5-((R)-2-(4-methylthiazol-2- | | | | | | |
| | yl)pyrrolidine-1- | | | | | | |
| | carbonyl)benzamide | | | | | | |
| 1-47 | N-((1R,2S)-1-((2R,4S)-4- | ++ | + | | | | |
| | fluoropyrrolidin-2-yl)-1-hydroxy- | | | | | | |
| | 3-phenylpropan-2-yl)-3-((R)-2-(4- | | | | | | |
| | methylthiazol-2-yl)pyrrolidine-1- | | | | | | |
| | carbonyl)benzamide | | | | | | |
| 1-48 | N-((1R,2S)-1-hydroxy-3-phenyl-1- | + | + | | | | |
| | ((S)-pyrrolidin-2-yl)propan-2-yl)- | | | | | | |
| | 3-((R)-2-(4-methylthiazol-2- | | | | | | |
| | yl)pyrrolidine-1- | | | | | | |
| | carbonyl)benzamide | | | | | | |
| 1-49 | N-((1R,2S)-3-(3,5-difluorophenyl)- | +++ | +++ | +++ | | | |
| | 1-hydroxy-1-((R)-pyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)-5-(oxazol-2- | | | | | | |
| | yl)benzamide | | | | | | |
| 1-50 | 3-cyclopropyl-N-((1R,2S)-1- | +++ | + | | | | |
| | hydroxy-3-phenyl-1-((R)- | | | | | | |
| | pyrrolidin-2-yl)propan-2-yl)-5- | | | | | | |
| | ((R)-2-(4-methylthiazol-2- | | | | | | |
| | yl)pyrrolidine-1- | | | | | | |
| | carbonyl)benzamide | | | | | | |
| 1-51 | N-((1R,2S)-1-hydroxy-1-((2R,5R)- | + | + | | | | |
| | 5-methylpyrrolidin-2-yl)-3- | | | | | | |
| | phenylpropan-2-yl)-3-((R)-2-(4- | | | | | | |
| | methylthiazol-2-yl)pyrrolidine-1- | | | | | | |
| | carbonyl)benzamide | | | | | | |
| 1-52 | N-((1R,2S)-1-((2R,4R)-4- | +++ | +++ | | | + | |
| | (benzyloxy)pyrrolidin-2-yl)-1- | | | | | | |
| | hydroxy-3-phenylpropan-2-yl)-3- | | | | | | |
| | ((R)-2-(4-methylthiazol-2- | | | | | | |
| | yl)pyrrolidine-1-carbonyl)-5- | | | | | | |
| | (oxazol-2-yl)benzamide | | | | | | |
| 1-53 | N-((1R,2S)-3-(3-fluorophenyl)-1- | +++ | ++ | | | | |
| | hydroxy-1-((R)-pyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)benzamide | | | | | | |
| 1-54 | N1-((1R,2S)-3-(3-fluorophenyl)-1- | ++ | | | | | |
| | hydroxy-1-((R)-pyrrolidin-2- | | | | | | |
| | yl)propan-2-yl)-N3-methyl-N3-((4- | | | | | | |
| | methylthiazol-2- | | | | | | |
| | yl)methyl)isophthalamide | | | | | | |
| 1-55 | N-((1R,2S)-1-hydroxy-3-phenyl-1- | +++ | | | | | |
| | ((R)-pyrrolidin-2-yl)propan-2-yl)- | | | | | | |
| | 3-((R)-2-(4-methylthiazol-2- | | | | | | |
| | yl)pyrrolidine-1-carbonyl)-5- | | | | | | |
| | nitrobenzamide | | | | | | |
| 1-56 | 3-chloro-N-((1R,2S)-1-hydroxy-3- | +++ | | | | | |
| | phenyl-1-((R)-pyrrolidin-2- | | | | | | |
| | yl)propan-2-yl)-5-((R)-2-(4- | | | | | | |
| | methylthiazol-2-yl)pyrrolidine-1- | | | | | | |
| | carbonyl)benzamide | | | | | | |
| 1-57 | N-((1S,2S)-1-((S)-3,3- | +++ | | | | | |
| | difluoropyrrolidin-2-yl)-1- | | | | | | |
| | hydroxy-3-phenylpropan-2-yl)-3- | | | | | | |
| | ((R)-2-(4-methylthiazol-2- | | | | | | |
| | yl)pyrrolidine-1- | | | | | | |
| | carbonyl)benzamide | | | | | | |
| 1-58 | N-((1R,2S)-1-hydroxy-1-((2R,5R)- | ++ | | | | | |
| | 5-methylpyrrolidin-2-yl)-3- | | | | | | |
| | phenylpropan-2-yl)-3-((R)-2-(4- | | | | | | |
| | methylthiazol-2-yl)pyrrolidine-1- | | | | | | |
| | carbonyl)-5-(1H-pyrrol-1- | | | | | | |
| | yl)benzamide | | | | | | |
| 1-59 | N-((1R,2S)-1-((2R,4S)-4- | +++ | | | | | |
| | fluoropyrrolidin-2-yl)-1-hydroxy- | | | | | | |
| | 3-phenylpropan-2-yl)-3-((R)-2-(4- | | | | | | |
| | methylthiazol-2-yl)pyrrolidine-1- | | | | | | |
| | carbonyl)-5-(1H-pyrrol-1- | | | | | | |
| | yl)benzamide | | | | | | |
| 1-60 | N1-((1R,2S)-1-((2R,4S)-4- | + | | | | | |
| | fluoropyrrolidin-2-yl)-1-hydroxy- | | | | | | |
| | 3-phenylpropan-2-yl)-N3-methyl- | | | | | | |
| | N3-((4-methylthiazol-2- | | | | | | |
| | yl)methyl)isophthalamide | | | | | | |
| 1-61 | N-((1R,2S)-3-(3,5-difluorophenyl)- | ++ | | | | | |
| | 1-hydroxy-1-((R)-pyrrolidin-2- | | | | | | |
| | yl)propan-2-yl)-2-((R)-2-(4- | | | | | | |
| | methylthiazol-2-yl)pyrrolidine-1- | | | | | | |
| | carbonyl)isonicotinamide | | | | | | |
| 1-62 | 3-cyclopropyl-N-((1R,2S)-3-(3,5- | +++ | | | | | |
| | difluorophenyl)-1-hydroxy-1-((R)- | | | | | | |
| | pyrrolidin-2-yl)propan-2-yl)-5- | | | | | | |
| | ((R)-2-(4-methylthiazol-2- | | | | | | |
| | yl)pyrrolidine-1- | | | | | | |
| | carbonyl)benzamide | | | | | | |
| 1-63 | N-((1R,2S)-3-(3,5-difluorophenyl)- | +++ | | | | | |
| | 1-hydroxy-1-((R)-pyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)-5-(pyrazin-2- | | | | | | |
| | yl)benzamide | | | | | | |
| 1-64 | N-((1R,2S)-3-(3,5-difluorophenyl)- | +++ | | + | | | |
| | 1-hydroxy-1-((R)-pyrrolidin-2- | | | | | | |
| | yl)propan-2-yl)-3-methyl-5-((R)-2- | | | | | | |
| | (4-methylthiazol-2-yl)pyrrolidine- | | | | | | |
| | 1-carbonyl)benzamide | | | | | | |
| 1-65 | N-((1R,2S)-3-(4-fluorophenyl)-1- | ++ | | | | | |
| | hydroxy-1-((R)-pyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)benzamide | | | | | | |
| 1-66 | N1-((1R,2S)-3-(4-fluorophenyl)-1- | + | | | | | |
| | hydroxy-1-((R)-pyrrolidin-2- | | | | | | |
| | yl)propan-2-yl)-N3-methyl-N3-((4- | | | | | | |
| | methylthiazol-2- | | | | | | |
| | yl)methyl)isophthalamide | | | | | | |
| 1-67 | N-((1R,2S)-1-((R)-5,5- | + | | | | | |
| | dimethylpyrrolidin-2-yl)-1- | | | | | | |
| | hydroxy-3-phenylpropan-2-yl)-3- | | | | | | |
| | ((R)-2-(4-methylthiazol-2- | | | | | | |
| | yl)pyrrolidine-1- | | | | | | |
| | carbonyl)benzamide | | | | | | |
| 1-68 | N-((1R,2S)-1-hydroxy-3-phenyl-1- | +++ | | | | | |
| | ((R)-pyrrolidin-2-yl)propan-2-yl)- | | | | | | |
| | 3-(methylsulfonyl)-5-((R)-2-(4- | | | | | | |
| | methylthiazol-2-yl)pyrrolidine-1- | | | | | | |
| | carbonyl)benzamide | | | | | | |
| 1-69 | N-((1R,2S)-3-(3,5-difluorophenyl)- | +++ | | ++ | | | |
| | 1-hydroxy-1-((R)-pyrrolidin-2- | | | | | | |
| | yl)propan-2-yl)-3-((R)-2-(4- | | | | | | |
| | methyloxazol-2-yl)pyrrolidine-1- | | | | | | |
| | carbonyl)-5-(oxazol-2- | | | | | | |
| | yl)benzamide | | | | | | |
| 1-70 | N-((1R,2S)-1-((2R,4R)-4- | +++ | | +++ | | | |
| | (allyloxy)pyrrolidin-2-yl)-1- | | | | | | |
| | hydroxy-3-phenylpropan-2-yl)-3- | | | | | | |
| | ((R)-2-(4-methylthiazol-2- | | | | | | |
| | yl)pyrrolidine-1-carbonyl)-5- | | | | | | |
| | (oxazol-2-yl)benzamide | | | | | | |
| 1-71 | N-((1R,2S)-1-hydroxy-3-phenyl- | +++ | | + | | | |
| | 1-((R)-pyrrolidin-2-yl)propan-2- | | | | | | |
| | yl)-3-iodo-5-((R)-2-(4- | | | | | | |
| | methylthiazol-2-yl)pyrrolidine-1- | | | | | | |
| | carbonyl)benzamide | | | | | | |
| 1-72 | N-((1R,2S)-1-hydroxy-3-phenyl- | +++ | | | | | |
| | 1-((R)-pyrrolidin-2-yl)propan-2- | | | | | | |
| | yl)-3-((R)-2-(4-methylthiazol-2- | | | | | | |
| | yl)pyrrolidine-1-carbonyl)-5- | | | | | | |
| | (trifluoromethyl)benzamide | | | | | | |
| 1-73 | N-((1R,2S)-1-hydroxy-3-phenyl- | | | | | | |
| | 1-((R)-pyrrolidin-2-yl)propan-2- | | | | | | |
| | yl)-3-((R)-2-(4-methylthiazol-2- | | | | | | |
| | yl)pyrrolidine-1-carbonyl)-5- | | | | | | |
| | (trifluoromethoxy)benzamide | | | | | | |
| 1-74 | N-((1R,2S)-1-hydroxy-1- | +++ | | + | | | |
| | ((2R,4R)-4-phenoxypyrrolidin-2- | | | | | | |
| | yl)-3-phenylpropan-2-yl)-3-((R)-2- | | | | | | |
| | (4-methylthiazol-2-yl)pyrrolidine- | | | | | | |
| | 1-carbonyl)benzamide | | | | | | |
| 1-75 | N-((1R,2S)-1-hydroxy-1-((2R,4R)- | | | | | | |
| | 4-hydroxypyrrolidin-2-yl)-3- | | | | | | |
| | phenylpropan-2-yl)-3-((R)-2-(4- | | | | | | |
| | methylthiazol-2-yl)pyrrolidine-1- | | | | | | |
| | carbonyl)-5-(oxazol-2- | | | | | | |
| | yl)benzamide | | | | | | |
| 1-76 | N-((1R,2S)-1-hydroxy-3-phenyl-1- | | | | | | |
| | ((2R,4R)-4-propoxypyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)-5-(oxazol-2- | | | | | | |
| | yl)benzamide | | | | | | |
| 1-77 | N-((1R,2S)-1-hydroxy-3-phenyl-1- | ++ | | | | | |
| | ((R)-pyrrolidin-2-yl)propan-2-yl)- | | | | | | |
| | 3-(1H-imidazol-1-yl)-5-((R)-2-(4- | | | | | | |
| | methylthiazol-2-yl)pyrrolidine-1- | | | | | | |
| | carbonyl)benzamide | | | | | | |
| 1-78 | N-((1R,2S)-1-hydroxy-3-phenyl-1- | +++ | +++ | + | | +++ | +++ |
| | ((R)-pyrrolidin-2-yl)propan-2-yl)- | | | | | | |
| | 3-((R)-2-(4-methylthiazol-2- | | | | | | |
| | yl)pyrrolidine-1-carbonyl)-5- | | | | | | |
| | (thiazinanyl-S,S- | | | | | | |
| | dioxide)benzamide | | | | | | |
| 1-79 | N-((1R,2S)-1-hydroxy-1-((2R,4R)- | +++ | | ++ | | | |
| | 4-phenoxypyrrolidin-2-yl)-3- | | | | | | |
| | phenylpropan-2-yl)-3-((R)-2-(4- | | | | | | |
| | methylthiazol-2-yl)pyrrolidine-1- | | | | | | |
| | carbonyl)-5-(oxazol-2- | | | | | | |
| | yl)benzamide | | | | | | |
| 1-80 | N-((1S,2S)-1-hydroxy-3-phenyl-1- | + | | | | | |
| | ((R)-pyrrolidin-2-yl)propan-2-yl)- | | | | | | |
| | 3-((R)-2-(4-methylthiazol-2- | | | | | | |
| | yl)pyrrolidine-1- | | | | | | |
| | carbonyl)benzamide | | | | | | |
| 1-81 | N-((1R,2S)-1-hydroxy-1-((2R,4R)- | +++ | | | | | |
| | 4-methoxypyrrolidin-2-yl)-3- | | | | | | |
| | phenylpropan-2-yl)-3-((R)-2-(4- | | | | | | |
| | methylthiazol-2-yl)pyrrolidine-1- | | | | | | |
| | carbonyl)-5-(oxazol-2- | | | | | | |
| | yl)benzamide | | | | | | |
| 1-82 | N-((1S,2S)-1-hydroxy-1-((2R,4R)- | +++ | | | | | |
| | 4-methoxypyrrolidin-2-yl)-3- | | | | | | |
| | phenylpropan-2-yl)-3-((R)-2-(4- | | | | | | |
| | methylthiazol-2-yl)pyrrolidine-1- | | | | | | |
| | carbonyl)benzamide | | | | | | |
| 1-83 | N-((1R,2S)-1-((R)-4,4- | | | | | | |
| | difluoropyrrolidin-2-yl)-1- | | | | | | |
| | hydroxy-3-phenylpropan-2-yl)-3- | | | | | | |
| | ((R)-2-(4-methylthiazol-2- | | | | | | |
| | yl)pyrrolidine-1-carbonyl)-5- | | | | | | |
| | (oxazol-2-yl)benzamide | | | | | | |
| 1-84 | 3-chloro-N-((1R,2S)-3-(3,5- | | | | | | |
| | difluorophenyl)-1-hydroxy-1-((R)- | | | | | | |
| | pyrrolidin-2-yl)propan-2-yl)-5- | | | | | | |
| | ((R)-2-(4-methylthiazol-2- | | | | | | |
| | yl)pyrrolidine-1- | | | | | | |
| | carbonyl)benzamide | | | | | | |
| 1-85 | N-((1R,2S)-1-hydroxy-3-phenyl-1- | | | | | | |
| | ((R)-pyrrolidin-2-yl)propan-2-yl)- | | | | | | |
| | 3-(1-methyl-1H-pyrazol-4-yl)-5- | | | | | | |
| | ((R)-2-(4-methylthiazol-2- | | | | | | |
| | yl)pyrrolidine-1- | | | | | | |
| | carbonyl)benzamide | | | | | | |
| 1-86 | N-((1R,2S)-1-hydroxy-3-phenyl-1- | | | | | | |
| | ((2R,4R)-4-(pyridin-2- | | | | | | |
| | yloxy)pyrrolidin-2-yl)propan-2-yl)- | | | | | | |
| | 3-((R)-2-(4-methylthiazol-2- | | | | | | |
| | yl)pyrrolidine-1- | | | | | | |
| | carbonyl)benzamide | | | | | | |
| 1-87 | N-((1R,2S)-1-hydroxy-3-(3,5- | +++ | +++ | + | | +++ | +++ |
| | difluorophenyl)-1-((R)-pyrrolidin- | | | | | | |
| | 2-yl)propan-2-yl)-3-((R)-2-(4- | | | | | | |
| | methylthiazol-2-yl)pyrrolidine-1- | | | | | | |
| | carbonyl)-5-(thiazinanyl-S,S- | | | | | | |
| | dioxide)benzamide. | | | | | | |
| 1-88 | N-((1R,2S)-1-hydroxy-3-(5- | | | | | | |
| | fluorophenyl)-1-((R)-pyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)-5-(thiazinanyl-S,S- | | | | | | |
| | dioxide)benzamide. | | | | | | |
| 1-89 | N-((1R,2S)-1-hydroxy-3-phenyl-1- | +++ | +++ | + | | | +++ |
| | ((R)-pyrrolidin-2-yl)propan-2-yl)- | | | | | | |
| | 3-((R)-2-(4-methylthiazol-2- | | | | | | |
| | yl)pyrrolidine-1-carbonyl)-5- | | | | | | |
| | ([1,2]thiazolidyl-S,S- | | | | | | |
| | dioxide)benzamide. | | | | | | |
| 1-90 | N-((1R,2S)-1-hydroxy-3-(3,5- | +++ | +++ | + | | | +++ |
| | difluorophenyl)-1-((R)-pyrrolidin- | | | | | | |
| | 2-yl)propan-2-yl)-3-((R)-2-(4- | | | | | | |
| | methylthiazol-2-yl)pyrrolidine-1- | | | | | | |
| | carbonyl)-5-([1,2]thiazolidyl-S,S- | | | | | | |
| | dioxide)benzamide. | | | | | | |
| 1-91 | N-((1R,2S)-1-hydroxy-3-(5- | +++ | ++ | + | | | +++ |
| | fluorophenyl)-1-((R)-pyrrolidin-2- | | | | | | |
| | yl)propan-2-yl)-3-((R)-2-(4- | | | | | | |
| | methylthiazol-2-yl)pyrrolidine-1- | | | | | | |
| | carbonyl)-5-([1,2]thiazolidyl-S,S- | | | | | | |
| | dioxide)benzamide. | | | | | | |

TABLE 2
Compound Assay data-Supplemental Compounds.

| | | M2 | M2 | CD | M1 | | HLM |
|---|---|---|---|---|---|---|---|
| Ref # | Structure | Ki | IC50 | Ki | Ki | CYP3A4 Ki | CL |
| 2-1 | N-((2S,3R)-4-((2-chloro-6- | +++ | +++ | | | | + |
| | (dimethylamino)pyridin-4- | | | | | | |
| | yl)methylamino)-3-hydroxy-1- | | | | | | |
| | phenylbutan-2-yl)-5-((R)-2-(4- | | | | | | |
| | methylthiazol-2-yl)pyrrolidine-1- | | | | | | |
| | carbonyl)nicotinamide | | | | | | |
| 2-2 | 3-(furan-2-yl)-N-((2S,3R)-3- | +++ | +++ | | | − | + |
| | hydroxy-4-((5-isopropylpyridin-3- | | | | | | |
| | yl)methylamino)-1-phenylbutan-2- | | | | | | |
| | yl)-5-((R)-2-(4-methylthiazol-2- | | | | | | |
| | yl)pyrrolidine-1- | | | | | | |
| | carbonyl)benzamide | | | | | | |
| 2-3 | N-((2S,3R)-4-((5-tert-butylpyridin- | +++ | +++ | | | − | + |
| | 3-yl)methylamino)-3-hydroxy-1- | | | | | | |
| | phenylbutan-2-yl)-3-methyl-5- | | | | | | |
| | ((R)-2-(4-methyloxazol-2- | | | | | | |
| | yl)pyrrolidine-1- | | | | | | |
| | carbonyl)benzamide | | | | | | |
| 2-4 | N-((2S,3R)-4-((5-tert-butylpyridin- | +++ | +++ | | | − | + |
| | 3-yl)methylamino)-3-hydroxy-1- | | | | | | |
| | phenylbutan-2-yl)-3-((R)-2-(4- | | | | | | |
| | methyloxazol-2-yl)pyrrolidine-1- | | | | | | |
| | carbonyl)benzamide | | | | | | |
| 2-5 | N-((2S,3R)-4-((1-ethyl-1H- | +++ | +++ | | | + | + |
| | pyrazol-4-yl)methylamino)-3- | | | | | | |
| | hydroxy-1-phenylbutan-2-yl)-3- | | | | | | |
| | methyl-5-((R)-2-(4-methyloxazol- | | | | | | |
| | 2-yl)pyrrolidine-1- | | | | | | |
| | carbonyl)benzamide | | | | | | |
| 2-6 | N-((2S,3R)-3-hydroxy-1-phenyl-4- | +++ | +++ | | | | + |
| | (3-(prop-1-en-2- | | | | | | |
| | yl)benzylamino)butan-2-yl)-3-((R)- | | | | | | |
| | 2-(4-methyloxazol-2- | | | | | | |
| | yl)pyrrolidine-1- | | | | | | |
| | carbonyl)benzamide | | | | | | |
| 2-7 | N-((2S,3R)-4-(3-tert- | +++ | +++ | | | + | + |
| | butylbenzylamino)-3-hydroxy-1- | | | | | | |
| | phenylbutan-2-yl)-3- | | | | | | |
| | (fluoromethyl)-5-((R)-2-(4- | | | | | | |
| | methyloxazol-2-yl)pyrrolidine-1- | | | | | | |
| | carbonyl)benzamide | | | | | | |
| 2-8 | N-((2S,3R)-3-hydroxy-4-((5- | +++ | +++ | | | − | |
| | isopropylpyridin-3- | | | | | | |
| | yl)methylamino)-1-phenylbutan-2- | | | | | | |
| | yl)-3-methyl-5-((R)-2-(4- | | | | | | |
| | methylthiazol-2-yl)pyrrolidine-1- | | | | | | |
| | carbonyl)benzamide | | | | | | |
| 2-9 | N1-cyclopropyl-N3-((2S,3R)-3- | +++ | +++ | | | − | |
| | hydroxy-1-phenyl-4-(3- | | | | | | |
| | (trifluoromethyl)benzylamino)butan- | | | | | | |
| | 2-yl)-N1-((4-methylthiazol-2- | | | | | | |
| | yl)methyl)isophthalamide | | | | | | |
| 2-10 | N-((2S,3R)-4-(3-tert- | +++ | +++ | | | − | |
| | butylbenzylamino)-3-hydroxy-1- | | | | | | |
| | phenylbutan-2-yl)-3-((R)-2-(4- | | | | | | |
| | methylthiazol-2-yl)pyrrolidine-1- | | | | | | |
| | carbonyl)benzamide | | | | | | |
| 2-11 | N-((2S,3R)-4-((5-tert-butylpyridin- | +++ | +++ | | | − | |
| | 3-yl)methylamino)-3-hydroxy-1- | | | | | | |
| | phenylbutan-2-yl)-3-methyl-5- | | | | | | |
| | ((R)-2-(4-methyloxazol-2- | | | | | | |
| | yl)pyrrolidine-1- | | | | | | |
| | carbonyl)benzamide | | | | | | |
| 2-12 | N1-((1R,2S)-1-hydroxy-3-phenyl- | + | | | | | |
| | 1-((R)-pyrrolidin-2-yl)propan-2- | | | | | | |
| | yl)-N3,N3-dipropylisophthalamide | | | | | | |
| 2-13 | N-((1R,2S)-1-hydroxy-3-phenyl-1- | + | | | | | |
| | ((R)-pyrrolidin-2-yl)propan-2-yl)- | | | | | | |
| | 3-((2-oxopyrrolidin-1- | | | | | | |
| | yl)methyl)benzamide | | | | | | |

In Tables 1 and 2, for the M2 Ki data, a “+” represents a Ki of greater than 750 nM, a “++” represents a Ki from 750 nM to 250 nM, and a “+++” represents a Ki of less than 250 nM. For the M2 IC50 data, a “+” represents an IC50 of greater than 1000 nM, a “++” represents an IC50 from 1000 nM to 500 nM, and a “+++” represents an IC50 of less than 500 nM. For the Cathepsin D (CD) Ki and M1 Ki data, a “+” represents a Ki of greater than >500 nM, a “++” represents a Ki from 500 nM to 300 nM, and a “+++” represents a Ki of less than 300 nM. For the CYP3A4 Ki data, a “−” represents a Ki of less than 1 μM, a “+” represents a Ki greater than 1 μM and less than 5 μM, a “++” represents a Ki from 5 μM to 10 μM and a “+++” represents Ki of greater than 10 μM. For the in vitro clearance data (HLM CL), a “+” represents a clearance value greater than 700 mL/min/kg, a “++” represents a clearance value from 700 to 400 mL/min/kg, and a “+++” represents clearance value less than 400 mL/min/kg. For example, N-((1R,2S)-1-((2R,4R)-4-(benzyloxy)pyrrolidin-2-yl)-1-hydroxy-3-phenylpropan-2-yl)-3-((R)-2-(4-methyloxazol-2-yl)pyrrolidine-1-carbonyl)benzamide has values for M2 Ki=50.38 nM, M2 IC50=290.7 nM, CYP3A4 Ki=11.5 μM, and HLM CL=337, represented in Table 1 as “+++”, “+++”, “+++”, and “+++”, respectively. In an additional example, N-((1R,2S)-1-((2R,4R)-4-(benzyloxy)pyrrolidin-2-yl)-1-hydroxy-3-phenylpropan-2-yl)-3-((R)-2-(4-methylthiazol-2-yl)pyrrolidine-1-carbonyl)-5-(1H-pyrrol-1-yl)benzamide has values for M2 Ki=9.21 nM, M2 IC50=55.6 nM, Cathepsin D Ki=48.85 nM, M1 Ki=623.91 nM, represented in Table 1 as “+++”, “+++”, “+++”, and “+”, respectively.

### Example 4

### Inhibition of Memapsin 1 Beta-Secretase Activity and Cathepsin D Activity

A substrate peptide NH₃-ELDLAVEFWHDR-CO₂ (SEQ ID NO.: 1) was dissolved at 2 mg/mL in 10% glacial acetic acid and diluted into 0.009M NaOH to obtain 1 μM concentration at pH 4.1. After equilibration at 37 degrees C., the reactions were initiated by the addition of an aliquot of memapsin 2. Aliquots were removed at time intervals, and combined with an equal volume of MALDI-TOF matrix (α-hydroxycinnamic acid in acetone, 20 mg/mL) and immediately spotted in duplicate onto a stainless-steel MALDI sample plate. MALDI-TOF mass spectrometry was performed on a PE Biosystems Voyager DE. The instrument was operated at 25,000 accelerating volts in positive mode with a 150 ns delay. Ions with a mass-to-charge ratio (m/z) were detected in the range of 650-2000 atomic mass units. Data were analyzed by the Voyager Data Explorer module to obtain ion intensity data for mass species of substrates and corresponding products in a given mixture. Relative product formation was calculated as the ratio of signal intensity of the product to the sum of signal intensities of both product and the corresponding substrate. Relative product formed per unit time was obtained from non-linear regression analysis of the data representing the initial 15% formation of product using the model:

1−e^{-kT},

where k was the relative hydrolytic rate constant and T was time in seconds. Initial rates were expressed relative to uninhibited controls and fit to a tight-binding model of competitive inhibition as above.

### Example 5

### Cellular AβIC50 Determinations

The potency of compounds against memapsin 2 activity was determined in a cellular assay of Aβ production. Compounds that successfully penetrate the cell membrane demonstrated their ability to inhibit memapsin 2 activity in endosomal compartments, thus blocking the production of Aβ. Chinese hamster ovary cells that over-express human APP695 with the London and Swedish mutations were seeded in multi-well plates at 10% confluency. Compounds are dissolved in DMSO to concentrations near 1 mM, and diluted into culture media to a final concentration near 4 μM (final 0.4% DMSO). Compounds were diluted serially and applied to cells in multi-well plates 48 h after seeding. Incubation was continued in 5% CO₂ at 37° C. for 24 h. Aliquots were removed and assayed for Aβ₄₀ content using a sandwich ELISA (BioSource International). Amount of Aβ₄₀ over the range of concentration of compounds, relative to control incubations, were fit to a 4-parameter IC₅₀ model. Results are provided in Table 1 above (“M2 IC50”).

### Example 6

### Determination of CYP3A4 Inhibition

To evaluate the drug-drug interaction potential for compounds, the potency to inhibit the major metabolic cytochrome CYP450 isoform 3A4 was assessed. The inhibition constant Ki was determined for inhibition of the metabolism of midazolam, a CYP3A4 substrate.

Assay Procedure

CYP3A4 Ki assays were performed following a recently published protocol with slight modifications (Di, L., Kerns, E. H., Li, S. Q., and Carter, G. T. (2007) Comparison of cytochrome P450 inhibition assays for drug discovery using human liver microsomes with LC-MS, rhCYP450 isozymes with fluorescence, and double cocktail with LC-MS. International Journal of Pharmaceutics 335: 1-11). The P450 inhibition assay was performed in 96-well plates at 37.2° C. in a shaking incubator. The compounds were diluted from 5 mM stocks in 100% DMSO and incubated at seven final concentrations from 0.078 to 10 μM (0.1% DMSO in each final incubation), with human liver microsomes (HLM) at a final protein concentration of 0.1 mg/mL protein and a substrate concentration ranging from 1.25 to 10 μM.

The assay was standardized for both phosphate buffer (100 mM, pH 7.4) and the NADPH regenerating system (MgCl₂, 3.3 mM; G6P, 3.3 mM; G6PD, 1 U/ml; NADP+, 1.3 mM). Eight replicate control samples (0.1% DMSO, no compound) were prepared. Assays (200 μL) were set up by mixing HLM+substrate stock, 10 μL of test article in 2% DMSO, and the substrate before initiating the reaction with the addition of the regenerating system mixture. Reactions were quenched following incubation for 20, 30 and 40 minutes as described following.

Reaction Quench and MS-Prep

After incubation for the specified time in a humidified shaking incubator, 20 μL of the reaction mixture was removed and the reactions were terminated by adding 200 pt of cold acetonitrile. Samples were centrifuged at 1000×g for 15 minutes in Solvinert filter plate. The receptor plate was dried via speed vacuum at 40° C. The sample was reconstituted with a reconstitution buffer composed of 10% acetonitrile, 10% DMSO, 80% H₂O with an internal standard added at a concentration of 100 ng/ml. MS-Analysis was completed using LC-MS/MS. Formation of 1′-hydroxymidazolam was measured by monitoring a specific SRM (342>203) transition for the CYP3A4 metabolite.

Determination of Ki

Data were expressed as the relative quantity of midazolam metabolite relative to control incubations. Initial velocities were obtained by multiplying the relative quantity by the initial substrate concentration and dividing by the incubation duration. Data were transformed to inverse of inivital velocity and expressed vs. inhibitor concentration [I] for determination of Ki by the Dixon method (Dixon, M. (1953) Biochemical Journal 55: 170-171) using where intercept [I]=−Ki was determined at multiple substrate concentrations. Results are provided in Table 1 above (“CYP 3A4 Ki”).

### Example 7

### Determination of Hepatic Intrinsic Clearance in Liver Microsomes

To 500 μl of 200 mM Na⁺ K⁺ phosphate buffer (pH 7.4), 100 μl of 1 mM EDTA solution was added followed by 100 μl of 2 mg protein/ml human liver microsomes. A 10 μl aliquot of test compound (20 mM stock in 50% acetonitrile) was diluted further with 40 μl of H₂O (total assay volume 750 μl. Assay mixture is incubated for 5 minutes at 37° C. The assay was initiated by addition of 250 μl of 4 mM NADPH solution. Incubation was continued at 37° C. Separate reaction mixtures were prepared for 0, 5, 10, 20, and 30 min durations. Individual reactions were halted by addition of 150 μl of 100% acetonitrile. Internal standard (10 μl of 100 ng/ml diazepam solution) was added. Concentration of the test compound in each separate reaction was measured using LC/MS/MS and a standard curve for the test compound according to standard procedures.

The hepatic intrinsic clearance in liver microsome was determined by the following procedure described by Davies and Morris (Davies, B. and Morris, T. (19930) Physiological parameters in laboratory animals and humans. Pharm Res. 10:1093-1095) detailing the relationship of in vitro clearance to physiological parameters in animals and human subjects.

The amount of compound remaining at each time point was expressed relative to the amount present in the initial (0 min) incubation. The relationship of relative amount vs. time was used to determine the half life of the compound in the microsome assay. The concentration independent rate constant, k₁, was determined from rectangular hyperbolic fit:

% remaining=100*(e^{-k}^{₁}^{T})

where T is time (in minutes), from the relationship of relative amount vs. time. Alternatively, k₁ was determined from a transform of the equation, where

k₁=0.693/t_{1/2}

Conversion to in vivo clearance was obtained from the relation (Davies and Morris, 1993):

$$\begin{matrix}CL = v / [ S ] \\ = ( {k}_{1} \times incubation volume ) / protein amount \\ = \frac{0.693}{t 1 / 2} \times \frac{incubation volume}{mg microsome} \times \frac{mg microsome}{g liver} \times \frac{liver weight ( g )}{Body weight ( kg )}\end{matrix}$$

where v=velocity of metabolism, [S] is the concentration of the compound, and volumes are expressed in mL. A lower intrinsic clearance rate demonstrates reduced propensity for metabolism and clearance in vivo. Results are provided in Table 1 (“HLM CL”).

1. A compound having the formula (I): 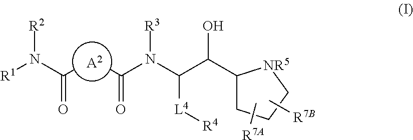
wherein
R¹ is A¹-L¹-; and
R² is hydrogen, —N(R⁸)R⁹, —S(O)₂R¹¹, —C(O)R¹², or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
or wherein R¹ and R² together with the nitrogen to which they are bonded form a 5-membered heterocycloalkyl ring substituted with A¹-L¹- and R⁶;
A¹ is an optionally substituted heteroaryl;
A² is a moiety selected from cycloalkylene, heterocycloalkylene, arylene, and heteroarylene, wherein the moiety is substituted with a cyclic sulfonamido;
R³ and R⁵ are each independently hydrogen, —N(R⁸)R⁹, —S(O)₂R¹¹, —C(O)R¹², or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
L¹ and L⁴ are each independently a bond, —N(R¹⁷)—, —S(O)_{q}—, or an optionally substituted alkylene;
R⁴, R⁶, R^{7A} and R^{7B} are each independently hydrogen, halogen, —OH, —NO₂, —N(R⁸)R⁹, —OR¹⁰, —S(O)ₙR¹¹, —C(O)R¹², or an optionally substituted moiety selected from alkyl, cycloalkyl, cycloalkyl-alkyl, -alkyl-OR¹⁰, -alkyl-N(R⁸)R⁹, heterocycloalkyl, heterocycloalkyl-alkyl, aryl, aralkyl, heteroaryl, and heteroaralkyl;
or wherein R^{7A} and R^{7B} together form an optionally substituted cycloalkyl ring;
R⁸ is independently hydrogen, —C(O)R¹³, —S(O)₂R¹⁴, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R⁹ is independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R¹⁰ is independently —C(O)R¹³, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R¹¹ is independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl, wherein if n is 2, then R¹¹ can also be —NR¹⁵R¹⁶, and wherein if n is 1 or 2, then R¹¹ is not hydrogen;
R¹² and R¹³ are each independently hydrogen, —N(R¹⁸)R¹⁹, —OR¹⁹, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl;
R¹⁴ is independently hydrogen, —N(R¹⁸)R¹⁹, or an optionally substituted moiety selected from alkyl, cycloalkyl, cycloalkyl-alkyl, heterocycloalkyl, heterocycloalkyl-alkyl, aryl, aralkyl, heteroaryl, or heteroaralkyl;
R¹⁵, R¹⁶, R¹⁷, R¹⁸, and R¹⁹ are each independently hydrogen, or an optionally substituted moiety selected from alkyl, cycloalkyl, cycloalkyl-alkyl, heterocycloalkyl, heterocycloalkyl-alkyl, aryl, aralkyl, heteroaryl, and heteroaralkyl; and
n and q are each independently 0, 1, or 2;
or a pharmaceutically acceptable salt or solvate thereof.

2-153. (canceled)

154. A compound according to claim 1, wherein A¹ is an optionally substituted 5 to 7 membered heteroaryl; or a pharmaceutically acceptable salt or solvate thereof.

155. A compound according to claim 1, wherein A¹ is an optionally substituted moiety selected form the group consisting of pyridyl, thiazolyl, oxazolyl, imidazolyl, pyrazolyl, isoxazolyl, pyrimidyl, oxadiazolyl, pyranyl, and furanyl; or a pharmaceutically acceptable salt or solvate thereof.

156. A compound according to claim 1, wherein L¹ is a bond, or an optionally substituted alkylene; or a pharmaceutically acceptable salt or solvate thereof.

157. A compound according to claim 1, having the formula (Ia): 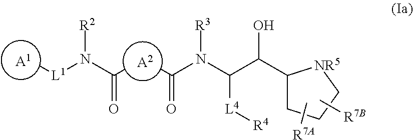
or a pharmaceutically acceptable salt or solvate thereof.

158. A compound according to claim 1, having the formula (Ib): 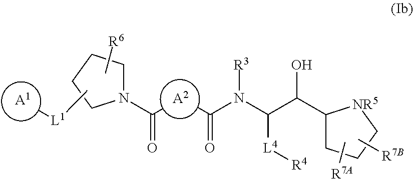
or a pharmaceutically acceptable salt or solvate thereof.

159. A compound according to claim 1, wherein A² is a moiety substituted with a cyclic sulfonamido, wherein the moiety is selected from the group consisting of phenylene, pyridinylene, oxazolylene, thioazolylene, pyrazolylene, pyranylene, and furanylene; or a pharmaceutically acceptable salt or solvate thereof.

160. A compound according to claim 159, wherein A² is phenylene substituted with a cyclic sulfonamido.

161. A compound according to claim 160, wherein A² has the formula: 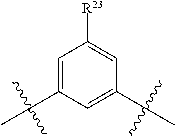
wherein R²³ is a cyclic sulfonamido.

162. A compound according to claim 1, wherein the cyclic sulfonamido is an 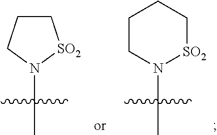 [SMILES: *N1CCCS1(=O)=O ; *N1CCCCS1(=O)=O]
or a pharmaceutically acceptable salt or solvate thereof.

163. A compound according to claim 1, wherein the compound is selected from the group consisting of:
N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-(thiazinanyl-S,S-dioxide)benzamide;
N-((1R,2S)-1-hydroxy-3-(3,5-difluorophenyl)-1-((R)-pyrrolidin-2-yl)propan-2-yl)-3-((R)-2-(4-methylthiazol-2-yl)pyrrolidine-1-carbonyl)-5-(thiazinanyl-S,S-dioxide)benzamide;
N-((1R,2S)-1-hydroxy-3-(5-fluorophenyl)-1-((R)-pyrrolidin-2-yl)propan-2-yl)-3-((R)-2-(4-methylthiazol-2-yl)pyrrolidine-1-carbonyl)-5-(thiazinanyl-S,S-dioxide)benzamide;
N-((1R,2S)-1-hydroxy-3-phenyl-1-((R)-pyrrolidin-2-yl)propan-2-yl)-3-((R)-2-(4-methylthiazol-2-yl)pyrrolidine-1-carbonyl)-5-([1,2]thiazolidyl-S,S-dioxide)benzamide;
N-((1R,2S)-1-hydroxy-3-(3,5-difluorophenyl)-1-((R)-pyrrolidin-2-yl)propan-2-yl)-3-((R)-2-(4-methylthiazol-2-yl)pyrrolidine-1-carbonyl)-5-([1,2]thiazolidyl-S,S-dioxide)benzamide; and
N-((1R,2S)-1-hydroxy-3-(5-fluorophenyl)-1-((R)-pyrrolidin-2-yl)propan-2-yl)-3-((R)-2-(4-methylthiazol-2-yl)pyrrolidine-1-carbonyl)-5-([1,2]thiazolidyl-S,S-dioxide)benzamide;
or a pharmaceutically acceptable salt or solvate thereof.

164. A formulation comprising a compound of claim 1 or a pharmaceutically acceptable salt or solvate thereof, and a pharmaceutically acceptable carrier.

165. A method of treating a condition mediated by memapsin 2 catalytic activity in an individual in need thereof by administering to the individual an effective amount of a compound of claim 1 or a pharmaceutically acceptable salt or solvate thereof.

166. A method of reducing memapsin 2 catalytic activity, the method comprising contacting memapsin 2 with an effective amount of a compound of claim 1 or a pharmaceutically acceptable salt or solvate thereof.

167. A method of treating Alzheimer's disease in an individual in need thereof, the method comprising administering to the individual an effective amount of a compound of claim 1 or a pharmaceutically acceptable salt or solvate thereof.


SEQUENCE LISTING (3 sequences)

SEQ ID NO: 1 - protein, 12 aa, Artificial Sequence
  Synthetic construct
ELDLAVEFWHDR

SEQ ID NO: 2 - protein, 10 aa, Artificial Sequence
  Synthetic Construct
XEVNLDAEFX

SEQ ID NO: 3 - protein, 10 aa, Artificial Sequence
  Synthetic construct
SEVNLDAEFK